(19) United States
(12) Patent Application Publication
(10) Pub. No.: US 2007/0117804 A1
(43) Pub. Date: May 24, 2007

(54) Imidazopyrazines as protein kinase inhibitors

(75) Inventors: Lianyun Zhao, Burlington, MA (US); Patrick J. Curran, Winthrop, MA (US); David B. Belanger, Cambridge, MA (US); Blake Hamann, Watertown, MA (US); Panduranga Adulla P. Reddy, Walpole, MA (US); Kamil Paruch, Garwood, NJ (US); Timothy J. Guzi, Chatham, NJ (US); Michael P. Dwyer, Scotch Plains, NJ (US); M. Arshad Siddiqui, Newton, MA (US); Praveen K. Tadikonda, Norwood, MA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/598,186

(22) Filed: Nov. 8, 2006

Related U.S. Application Data
(60) Provisional application No. 60/735,982, filed on Nov. 10, 2005.

(51) Int. Cl. A61K 31/498; C07D 487/04

(52) U.S. Cl. 514/249; 544/350

(57) ABSTRACT

In its many embodiments, the present invention provides a novel class of imidazopyrazine compounds as inhibitors of protein and/or checkpoint kinases, methods of preparing such compounds, pharmaceutical compositions including one or more such compounds, methods of preparing pharmaceutical formulations including one or more such compounds, and methods of treatment, prevention, inhibition, or amelioration of one or more diseases associated with the protein or checkpoint kinases using such compounds or pharmaceutical compositions.

### REFERENCE TO PRIORITY APPLICATION

This application claims the benefit of priority from U.S. Provisional Patent Application Ser. No. 60/735,982, filed on Nov. 10, 2005.

### FIELD OF THE INVENTION

The present invention relates to imidazo[1,2-a]pyrazine compounds useful as protein kinase inhibitors, regulators or modulators, pharmaceutical compositions containing the compounds, and methods of treatment using the compounds and compositions to treat diseases such as, for example, cancer, inflammation, arthritis, viral diseases, neurodegenerative diseases such as Alzheimer's disease, cardiovascular diseases, and fungal diseases.

### BACKGROUND OF THE INVENTION

Protein kinases are a family of enzymes that catalyze phosphorylation of proteins, in particular the hydroxyl group of specific tyrosine, serine, or threonine residues in proteins. Protein kinases are pivotal in the regulation of a wide variety of cellular processes, including metabolism, cell proliferation, cell differentiation, and cell survival. Uncontrolled proliferation is a hallmark of cancer cells, and can be manifested by a deregulation of the cell division cycle in one of two ways—making stimulatory genes hyperactive or inhibitory genes inactive. Protein kinase inhibitors, regulators or modulators, alter the function of kinases such as cyclin-dependent kinases (CDKs), mitogen activated protein kinase (MAPK/ERK), glycogen synthase kinase 3 (GSK3beta), Checkpoint (Chk) (e.g., CHK-1, CHK-2 etc.) kinases, AKT kinases, JNK, Aurora kinases (Aurora A, Aurora B, Aurora C etc), and the like. Examples of protein kinase inhibitors are described in WO02/22610 A1 and by Y. Mettey et al in J. Med. Chem., (2003) 46 222-236.

The cyclin-dependent kinases are serine/threonine protein kinases, which are the driving force behind the cell cycle and cell proliferation. Misregulation of CDK function occurs with high frequency in many important solid tumors. Individual CDK's, such as, CDK1, CDK2, CDK3, CDK4, CDK5, CDK6 and CDK7, CDK8 and the like, perform distinct roles in cell cycle progression and can be classified as either G1, S, or G2M phase enzymes. CDK2 and CDK4 are of particular interest because their activities are frequently misregulated in a wide variety of human cancers. CDK2 activity is required for progression through G1 to the S phase of the cell cycle, and CDK2 is one of the key components of the G1 checkpoint. Checkpoints serve to maintain the proper sequence of cell cycle events and allow the cell to respond to insults or to proliferative signals, while the loss of proper checkpoint control in cancer cells contributes to tumorgenesis. The CDK2 pathway influences tumorgenesis at the level of tumor suppressor function (e.g. p52, RB, and p27) and oncogene activation (cyclin E). Many reports have demonstrated that both the coactivator, cyclin E, and the inhibitor, p27, of CDK2 are either over- or underexpressed, respectively, in breast, colon, nonsmall cell lung, gastric, prostate, bladder, non-Hodgkin's lymphoma, ovarian, and other cancers. Their altered expression has been shown to correlate with increased CDK2 activity levels and poor overall survival. This observation makes CDK2 and its regulatory pathways compelling targets for the development of cancer treatments.

A number of adenosine 5′-triphosphate (ATP) competitive small organic molecules as well as peptides have been reported in the literature as CDK inhibitors for the potential treatment of cancers. U.S. Pat. No. 6,413,974, col. 1, line 23—col. 15, line 10 offers a good description of the various CDKs and their relationship to various types of cancer. Flavopiridol (shown below) is a nonselective CDK inhibitor that is currently undergoing human clinical trials, A. M. Sanderowicz et al, J. Clin. Oncol. (1998) 16, 2986-2999.

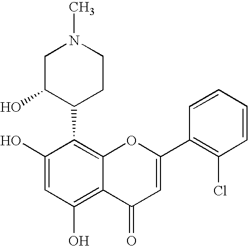

Other known inhibitors of CDKs include, for example, olomoucine (J. Vesely et al, Eur. J. Biochem., (1994) 224, 771-786) and roscovitine (I. Meijer et al, Eur. J. Biochem., (1997) 243, 527-536). U.S. Pat. No. 6,107,305 describes certain pyrazolo[3,4-b]pyridine compounds as CDK inhibitors. An illustrative compound from the '305 patent is:

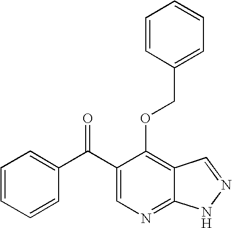

K. S. Kim et al., J. Med. Chem. 45 (2002) 3905-3927 and WO 02/10162 disclose certain aminothiazole compounds as CDK inhibitors. Imidazopyrazines are known. For example, U.S. Pat. No. 6,919,341 (the disclosure of which is incorporated herein by reference) and US2005/0009832 disclose various imidazopyrazines. Also being mentioned are the following: WO2005/047290; US2005/095616; WO2005/039393; WO2005/019220; WO2004/072081; WO2005/014599; WO2005/009354; WO2005/005429; WO2005/085252; US2005/009832; US2004/220189; WO2004/074289; WO2004/026877; WO2004/026310; WO2004/022562; WO2003/089434; WO2003/084959; WO2003/051346; US2003/022898; WO2002/060492; WO2002/060386; WO2002/028860; JP (1986) 61-057587; J. Burke et al., J. Biological Chem., Vol. 278(3), 1450-1456 (2003); and F. Bondavalli et al., J. Med. Chem., Vol. 45 (22), 4875-4887 (2002).

Another series of protein kinases are those that play an important role as a checkpoint in cell cycle progression. Checkpoints prevent cell cycle progression at inappropriate times, such as in response to DNA damage, and maintain the metabolic balance of cells while the cell is arrested, and in some instances can induce apoptosis (programmed cell death) when the requirements of the checkpoint have not been met. Checkpoint control can occur in the G1 phase (prior to DNA synthesis) and in G2, prior to entry into mitosis.

One series of checkpoints monitors the integrity of the genome and, upon sensing DNA damage, these “DNA damage checkpoints” block cell cycle progression in G₁ & G₂ phases, and slow progression through S phase. This action enables DNA repair processes to complete their tasks before replication of the genome and subsequent separation of this genetic material into new daughter cells takes place. Inactivation of CHK1 has been shown to transduce signals from the DNA-damage sensory complex to inhibit activation of the cyclin B/Cdc2 kinase, which promotes mitotic entry, and abrogate G.sub.2 arrest induced by DNA damage inflicted by either anticancer agents or endogenous DNA damage, as well as result in preferential killing of the resulting checkpoint defective cells. See, e.g., Peng et al., Science, 277, 1501-1505 (1997); Sanchez et al., Science, 277, 1497-1501 (1997), Nurse, Cell, 91, 865-867 (1997); Weinert, Science, 277, 1450-1451 (1997); Walworth et al., Nature, 363, 368-371 (1993); and Al-Khodairy et al., Molec. Biol. Cell., 5, 147-160 (1994).

Selective manipulation of checkpoint control in cancer cells could afford broad utilization in cancer chemotherapeutic and radiotherapy regimens and may, in addition, offer a common hallmark of human cancer “genomic instability” to be exploited as the selective basis for the destruction of cancer cells. A number of factors place CHK1 as a pivotal target in DNA-damage checkpoint control. The elucidation of inhibitors of this and functionally related kinases such as CDS1/CHK2, a kinase recently discovered to cooperate with CHK1 in regulating S phase progression (see Zeng et al., Nature, 395, 507-510 (1998); Matsuoka, Science, 282, 1893-1897 (1998)), could provide valuable new therapeutic entities for the treatment of cancer.

Another group of kinases are the tyrosine kinases. Tyrosine kinases can be of the receptor type (having extracellular, transmembrane and intracellular domains) or the non-receptor type (being wholly intracellular). Receptor-type tyrosine kinases are comprised of a large number of transmembrane receptors with diverse biological activity. In fact, about 20 different subfamilies of receptor-type tyrosine kinases have been identified. One tyrosine kinase subfamily, designated the HER subfamily, is comprised of EGFR (HER1), HER2, HER3 and HER4. Ligands of this subfamily of receptors identified so far include epithelial growth factor, TGF-alpha, amphiregulin, HB-EGF, betacellulin and heregulin. Another subfamily of these receptor-type tyrosine kinases is the insulin subfamily, which includes INS-R, IGF-IR, IR, and IR-R. The PDGF subfamily includes the PDGF-alpha and beta receptors, CSFIR, c-kit and FLK-II. The FLK family is comprised of the kinase insert domain receptor (KDR), fetal liver kinase-1 (FLK-1), fetal liver kinase-4 (FLK-4) and the fms-like tyrosine kinase-1 (flt-1). For detailed discussion of the receptor-type tyrosine kinases, see Plowman et al., DN&P 7(6): 334-339, 1994.

At least one of the non-receptor protein tyrosine kinases, namely, LCK, is believed to mediate the transduction in T-cells of a signal from the interaction of a cell-surface protein (Cd4) with a cross-linked anti-Cd4 antibody. A more detailed discussion of non-receptor tyrosine kinases is provided in Bolen, Oncogene, 8, 2025-2031 (1993). The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen, Oncogene, 8:2025-2031 (1993).

In addition to its role in cell-cycle control, protein kinases also play a crucial role in angiogenesis, which is the mechanism by which new capillaries are formed from existing vessels. When required, the vascular system has the potential to generate new capillary networks in order to maintain the proper functioning of tissues and organs. In the adult, however, angiogenesis is fairly limited, occurring only in the process of wound healing and neovascularization of the endometrium during menstruation. On the other hand, unwanted angiogenesis is a hallmark of several diseases, such as retinopathies, psoriasis, rheumatoid arthritis, age-related macular degeneration, and cancer (solid tumors). Protein kinases which have been shown to be involved in the angiogenic process include three members of the growth factor receptor tyrosine kinase family; VEGF-R2 (vascular endothelial growth factor receptor 2, also known as KDR (kinase insert domain receptor) and as FLK 1); FGF-R (fibroblast growth factor receptor); and TEK (also known as Tie-2).

VEGF-R2, which is expressed only on endothelial cells, binds the potent angiogenic growth factor VEGF and mediates the subsequent signal transduction through activation of its intracellular kinase activity. Thus, it is expected that direct inhibition of the kinase activity of VEGF-R2 will result in the reduction of angiogenesis even in the presence of exogenous VEGF (see Strawn et al, Cancer Research, 56, 3540-3545 (1996)), as has been shown with mutants of VEGF-R2 which fail to mediate signal transduction. Millauer et al, Cancer Research, 56, 1615-1620 (1996). Furthermore, VEGF-R2 appears to have no function in the adult beyond that of mediating the angiogenic activity of VEGF. Therefore, a selective inhibitor of the kinase activity of VEGF-R2 would be expected to exhibit little toxicity.

Similarly, FGFR binds the angiogenic growth factors aFGF and bFGF and mediates subsequent intracellular signal transduction. Recently, it has been suggested that growth factors such as bFGF may play a critical role in inducing angiogenesis in solid tumors that have reached a certain size. Yoshiji et al., Cancer Research, 57, 3924-3928 (1997). Unlike VEGF-R2, however, FGF-R is expressed in a number of different cell types throughout the body and may or may not play important roles in other normal physiological processes in the adult. Nonetheless, systemic administration of a small molecule inhibitor of the kinase activity of FGF-R has been reported to block bFGF-induced angiogenesis in mice without apparent toxicity. Mohammad et al., EMBO Journal, 17, 5996-5904 (1998).

TEK (also known as Tie-2) is another receptor tyrosine kinase expressed only on endothelial cells which has been shown to play a role in angiogenesis. The binding of the factor angiopoietin-1 results in autophosphorylation of the kinase domain of TEK and results in a signal transduction process which appears to mediate the interaction of endothelial cells with peri-endothelial support cells, thereby facilitating the maturation of newly formed blood vessels. The factor angiopoietin-2, on the other hand, appears to antagonize the action of angiopoietin-1 on TEK and disrupts angiogenesis. Maisonpierre et al., Science, 277, 55-60 (1997). The kinase, JNK, belongs to the mitogen-activated protein kinase (MAPK) superfamily. JNK plays a crucial role in inflammatory responses, stress responses, cell proliferation, apoptosis, and tumorigenesis. JNK kinase activity can be activated by various stimuli, including the proinflammatory cytokines (TNF-alpha and interleukin-1), lymphocyte costimulatory receptors (CD28 and CD40), DNA-damaging chemicals, radiation, and Fas signaling. Results from the JNK knockout mice indicate that JNK is involved in apoptosis induction and T helper cell differentiation.

Pim-1 is a small serine/threonine kinase. Elevated expression levels of Pim-1 have been detected in lymphoid and myeloid malignancies, and recently Pim-1 was identified as a prognostic marker in prostate cancer. K. Peltola, “Signaling in Cancer: Pim-1 Kinase and its Partners”, Annales Universitatis Turkuensis, Sarja—Ser. D Osa—Tom. 616, (Aug. 30, 2005), http://kiriasto.utu.fi/iulkaisupalvelut/annaalitV2004/D616.html. Pim-1 acts as a cell survival factor and may prevent apoptosis in malignant cells. K. Petersen Shay et al., Molecular Cancer Research 3:170-181 (2005).

There is a need for effective inhibitors of protein kinases in order to treat or prevent disease states associated with abnormal cell proliferation. Moreover, it is desirable for kinase inhibitors to possess both high affinity for the target kinase as well as high selectivity versus other protein kinases. Small-molecule compounds that may be readily synthesized and are potent inhibitors of cell proliferation are those, for example, that are inhibitors of one or more protein kinases, such as CHK1, CHK2, VEGF (VEGF-R2), Pim-1, CDKs or CDK/cyclin complexes and both receptor and non-receptor tyrosine kinases.

### SUMMARY OF THE INVENTION

In its many embodiments, the present invention provides a novel class of imidazo[1,2-a]pyrazine compounds, methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with protein kinases using such compounds or pharmaceutical compositions.

In one aspect, the present invention provides compounds represented by Formula I:

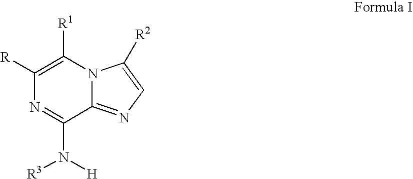

or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃ NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁵ (wherein the R⁵ and R⁶, together with the N of said —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
   said alkyl shown above for R³ can be unsubstituted or substituted with one or more moieties which can be the same or different each moiety being independently selected from the group consisting of —OR⁵, alkoxy, heteroaryl, and —NR⁵R⁶;
   said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and —S(O₂)R⁵; and
   said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
   further wherein in any —NR⁵R⁶ in Formula I, said R⁵ and R⁶ can optionally be joined together with the N of said —NR⁵R⁶ to form a heterocyclyl ring.

The compounds of Formula I can be useful as protein kinase inhibitors and can be useful in the treatment and prevention of proliferative diseases, for example, cancer, inflammation and arthritis, neurodegenerative diseases such Alzheimer's disease, cardiovascular diseases, viral diseases and fungal diseases.

### DETAILED DESCRIPTION

In an embodiment, the present invention provides imidazopyrazine compounds, especially imidazo[1,2-a]pyrazine compounds which are represented by structural Formula I, or pharmaceutically acceptable salts, solvates, esters or prodrug thereof, wherein the various moieties are as described above.

In another embodiment, the present invention provides compounds represented by Formula I: 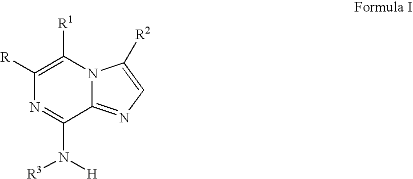
or a pharmaceutically acceptable salt, solvate, ester or prodrug thereof, wherein:
R is H, CN, —NR⁵R⁶, cycloalkenyl, heterocyclenyl, —C(O)NR⁵R⁶, —N(R⁵)C(O)R⁶, or alkyl substituted with one or more moieties which can be the same or different each moiety being independently selected from the group consisting of —OR⁵ and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is H, halo, or heteroaryl, wherein said heteroaryl can be unsubstituted or substituted with one or more moieties which can be the same or different each moiety being independently selected from the group consisting of halo, alkyl, alkenyl, alkynyl, cycloalkyl, aryl, heteroaryl and heterocyclyl;
R³ is H, alkyl, aryl or heteroaryl, wherein:
   said alkyl can be unsubstituted or substituted with one or more moieties which can be the same or different each moiety being independently selected from the group consisting of —OR⁵, alkoxy and —NR⁵R⁶;
   said aryl is substituted with heteroaryl which heteroaryl can be unsubstituted or substituted with alkyl; and
   said heteroaryl shown above for R³ can be unsubstituted or substituted with one or more moieties which can be the same or different with each moiety being independently selected from the group consisting of halo, —OR⁵, alkyl, alkenyl, alkynyl, cycloalkyl, aryl and heterocyclyl;
R⁵ is H, alkyl, aryl, heteroaryl, heterocyclyl or cycloalkyl; and
R⁶ is H, alkyl, aryl, heteroaryl, heterocyclyl or cycloalkyl.

In an embodiment, R, R¹, R² and R³ are not all H simultaneously.

In another embodiment, in Formula I, R² is unsubstituted heteroaryl or heteroaryl substituted with alkyl.

In another embodiment, in Formula I, R² is heteroaryl substituted with alkyl.

In another embodiment, in Formula I, R² is pyrazolyl.

In another embodiment, in Formula I, R² is pyrazolyl substituted with alkyl.

In another embodiment, in Formula I, R² is 1-methyl-pyrazol-4-yl.

In another embodiment, in Formula I, R is H.

In another embodiment, in Formula I, R is CN.

In another embodiment, in Formula I, R is —C(O)NR⁵R⁶.

In another embodiment, in Formula I, R is —C(O)NH₂.

In another embodiment, in Formula I, R is heterocyclenyl.

In another embodiment, in Formula I, R is tetrahydropyridinyl.

In another embodiment, in Formula I, R is 1,2,3,6-tetrahydropyridinyl.

In another embodiment, in Formula I, R is alkyl substituted with one or more moieties which can be the same or different each moiety being independently selected from the group consisting of —OR¹ and —NR⁵R⁶.

In another embodiment, in Formula I, R is alkyl substituted with one or more —NR⁵R⁶.

In another embodiment, in Formula I, R is alkyl substituted with —NH₂.

In another embodiment, in Formula I, R is alkyl substituted with —NH(methyl).

In another embodiment, R is unsubstituted alkyl.

In some embodiments, both R and R¹ are not H simultaneously.

In another embodiment, in Formula I, R³ is H.

In another embodiment, in Formula I, R³ is unsubstituted alkyl.

In another embodiment, in Formula I, R³ is alkyl substituted with one or more moieties which can be the same or different, each moiety being independently selected from the group consisting of halo, —OR¹, alkoxy and —NR⁵R⁶.

In another embodiment, in Formula I, R³ is unsubstituted heteroaryl.

In another embodiment, in Formula I, R³ is heteroaryl substituted with alkyl.

In another embodiment, in Formula I, R³ is heteroaryl substituted with methyl.

In another embodiment, in Formula I, R³ is unsubstituted isothiazolyl.

In another embodiment, in Formula I, R³ is isothiazolyl substituted with alkyl.

In another embodiment, in Formula I, R³ is isothiazolyl substituted with methyl.

In another embodiment, in Formula I, R³ is 5-methyl-isothiazol-3-yl.

In another embodiment, R³ is aryl substituted with heteroaryl.

In another embodiment, R³ is aryl substituted with imidazolyl.

In another embodiment, R³ is phenyl substituted with imidazolyl.

In another embodiment, this invention discloses a compound of the formula: 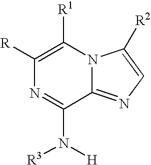
or a pharmaceutically acceptable salt, solvate or ester thereof, wherein R² is heteroaryl, R═R¹═H and R³ is unsubstituted alkyl, wherein said heteroaryl can be unsubstituted or substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (where R⁵ and R⁶ form a cyclic amine together with the N of said —NR⁵R⁶), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl, wherein R⁵ and R⁶ are as defined above.

In another embodiment, this invention discloses a compound of the formula: 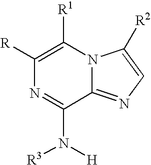
or a pharmaceutically acceptable salt, solvate or ester thereof, wherein R² is heteroaryl, wherein said heteroaryl can be unsubstituted or substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (where R⁵ and R⁶ form a cyclic amine together with the N of said —NR⁵R⁶), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₃—N(R⁵R⁶) and —NR⁵R⁶; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 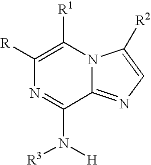
or a pharmaceutically acceptable salt, solvate or ester thereof, wherein R² is heteroaryl, wherein said heteroaryl can be unsubstituted or substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (where R⁵ and R⁶ form a cyclic amine together with the N of said —NR⁵R⁶), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 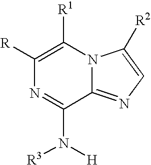
or a pharmaceutically acceptable salt, solvate or ester thereof, wherein R² is pyrazolyl, R═R¹═H and R³ is unsubstituted alkyl, wherein said pyrazolyl can be unsubstituted or substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (where R⁵ and R⁶ form a cyclic amine together with the N of said —NR⁵R⁶), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl, wherein R⁵ and R⁶ are as defined above.

In another embodiment, this invention discloses a compound of the formula: 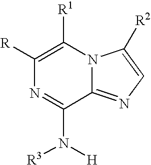
or a pharmaceutically acceptable salt, solvate or ester thereof, wherein R² is 1-methyl-pyrazol-4-yl, R═R¹═H and R³ is unsubstituted alkyl.

In another embodiment, this invention discloses a compound of the formula: 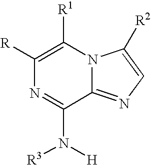
or a pharmaceutically acceptable salt, solvate or ester thereof, wherein R² is pyrazolyl, wherein said pyrazolyl can be unsubstituted or substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (where R⁵ and R⁶ form a cyclic amine together with the N of said —NR⁵R⁶), —CN, arylalkyl, —CH₂OR⁵, —S(O)R⁵, —S(O₂)R⁵, —CN, —CHO, —SR⁵, —C(O)R⁵, —C(O)R⁵, heteroaryl and heterocyclyl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₃—N(R⁵R⁶) and —NR⁵R⁶; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 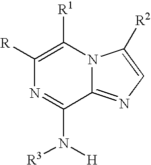
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 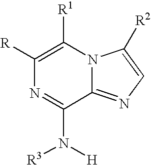
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 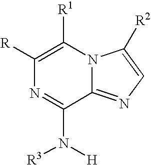
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is isothiazolyl wherein said isothiaozlyl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 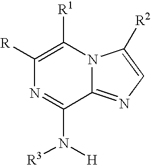
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is isothiazolyl wherein said isothiazolyl is substituted with one or more alkyl, wherein R⁵ and R⁶ are as defined above.

In another embodiment, this invention discloses a compound of the formula: 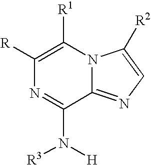
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is 5-methyl-isothiazol-3-yl, wherein R⁵ and R⁶ are as defined above.

In another embodiment, this invention discloses a compound of the formula: 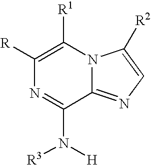
or a pharmaceutically acceptable salt, solvate or ester thereof, wherein R² is pyrazolyl, wherein said pyrazolyl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵; R is heterocyclenyl; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 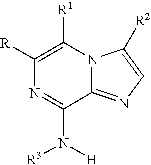
or a pharmaceutically acceptable salt, solvate or ester thereof, wherein R² is 1-methyl-pyrazol-4-yl; R is heterocyclenyl; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl.

In another embodiment, this invention discloses a compound of the formula: 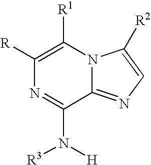
or a pharmaceutically acceptable salt, solvate or ester thereof, wherein R² is 1-methyl-pyrazol-4-yl; R is tetrahydropyridinyl; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl.

In another embodiment, this invention discloses a compound of the formula: 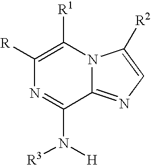
or a pharmaceutically acceptable salt, solvate or ester thereof, wherein R² is 1-methyl-pyrazol-4-yl; R is 1,2,3,6-tetrahydropyridinyl; R¹ is H and R³ is heteroaryl wherein said heteroaryl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl.

In another embodiment, this invention discloses a compound of the formula: 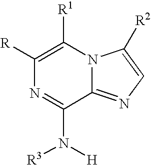
or a pharmaceutically acceptable salt, solvate or ester thereof, wherein R² is 1-methyl-pyrazol-4-yl; R is 1,2,3,6-tetrahydropyridinyl; R¹ is H and R³ is isothiaozlyl wherein said isothiazolyl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl.

In another embodiment, this invention discloses a compound of the formula: 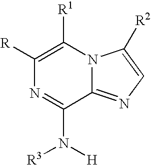
or a pharmaceutically acceptable salt, solvate or ester thereof, wherein R² is 1-methyl-pyrazol-4-yl; R is 1,2,3,6-tetrahydropyridinyl; R¹ is H and R³ is 5-methyl-isothiazol-3-yl.

In another embodiment, this invention discloses a compound of the formula: 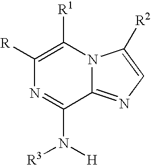
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is isothiazolyl wherein said isothiaozlyl can be unsubstituted or substituted with one or more moieties which can be the same or different each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl, wherein R⁵ and R⁶ are as defined above.

In another embodiment, this invention discloses a compound of the formula: 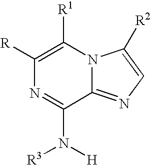
or a pharmaceutically acceptable salt, solvate or ester thereof, wherein R² is unsubstituted heteroaryl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 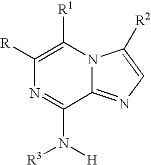
or a pharmaceutically acceptable salt, solvate or ester thereof, wherein R² is heteroaryl substituted with alkyl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 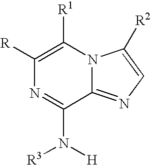
or a pharmaceutically acceptable salt, solvate or ester thereof, wherein R² is heteroaryl substituted with alkyl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 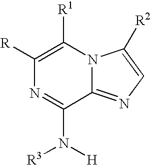
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₃—N(R⁵R⁶) and —NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 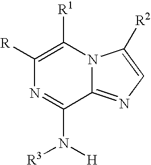
or a pharmaceutically acceptable salt, solvate or ester thereof, wherein R² is 1-methyl-pyrazol-4-yl; R is unsubstituted alkyl or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶; R¹ is H and R³ is aryl wherein said aryl is substituted with imidazolyl, wherein said imidazolyl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 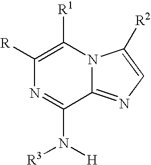
or a pharmaceutically acceptable salt, solvate or ester thereof, wherein R² is unsubstituted heteroaryl; R is —C(O)NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 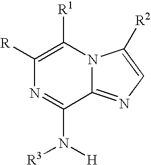
or a pharmaceutically acceptable salt, solvate or ester thereof, wherein R² is heteroaryl substituted with alkyl; R is —C(O)NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 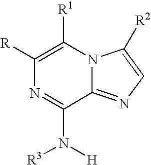
or a pharmaceutically acceptable salt, solvate or ester thereof, wherein R² is heteroaryl substituted with alkyl; R is —C(O)NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 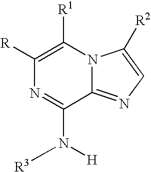
or a pharmaceutically acceptable salt, solvate or ester thereof, wherein R² is 1-methyl-pyrazol-4-yl; R is —C(O)NR⁵R⁶; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵ and wherein R⁵ and R⁶ are as defined above.

In another embodiment, this invention discloses a compound of the formula: 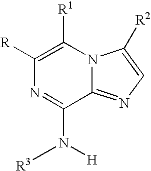
or a pharmaceutically acceptable salt, solvate or ester thereof, wherein R² is 1-methyl-pyrazol-4-yl; R is —C(O)NR⁵R⁶; R¹ is H and R³ is aryl wherein said aryl is substituted with imidazolyl, wherein said imidazolyl can be unsubstituted or optionally independently substituted with one or more moieties which can be the same or different each moiety being independently selected from alkyl, —OR⁵, —N(R⁵R⁶) and —S(O₂)R⁵, and wherein R⁵ and R⁶ are as defined above.

In another embodiment, this invention discloses a compound of the formula: 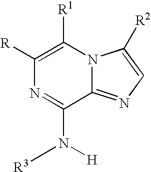
or a pharmaceutically acceptable salt, solvate or ester thereof, wherein R² is unsubstituted heteroaryl; R is heterocyclenyl; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁵) and —S(O₂)R⁵.

In another embodiment, this invention discloses a compound of the formula: 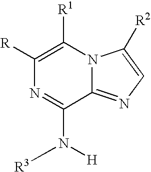
or a pharmaceutically acceptable salt, solvate or ester thereof, wherein R² is heteroaryl substituted with alkyl; R is heterocyclenyl; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵.

In another embodiment, this invention discloses a compound of the formula: 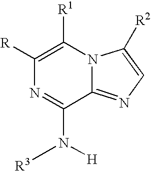
or a pharmaceutically acceptable salt, solvate or ester thereof, wherein R² is 1-methyl-pyrazol-4-yl; R is heterocyclenyl; R¹ is H and R³ is aryl wherein said aryl is substituted with a heteroaryl, wherein said heteroaryl can be unsubstituted or optionally independently substituted with one or more moieties which can be the same or different each moiety being independently selected from alkyl, —OR⁵, —N(R⁵R⁶) and —S(O₂)R⁵.

In another embodiment, this invention discloses a compound of the formula: 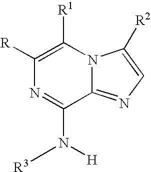
or a pharmaceutically acceptable salt, solvate or ester thereof, wherein R² is 1-methyl-pyrazol-4-yl; R is heterocyclenyl; R¹ is H and R³ is aryl wherein said aryl is substituted with imidazolyl, wherein said imidazolyl can be can be unsubstituted or optionally independently substituted with one or more moieties which can be the same or different each moiety being independently selected from alkyl, —OR⁵, —N(R⁵R⁵) and —S(O₂)R⁵.

In another embodiment, this invention discloses a compound of the formula: 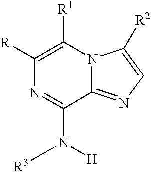
or a pharmaceutically acceptable salt, solvate or ester thereof, wherein R² is 1-methyl-pyrazol-4-yl; R is 1,2,3,6-tetrahydropyridinyl; R¹ is H and R³ is aryl wherein said aryl is substituted with imidazolyl, wherein said imidazolyl can be can be unsubstituted or optionally independently substituted with one or more moieties which can be the same or different each moiety being independently selected from alkyl, —OR⁵, —N(R⁵R⁶) and —S(O₂)R⁵.

Non-limiting examples of compounds of Formula I include:

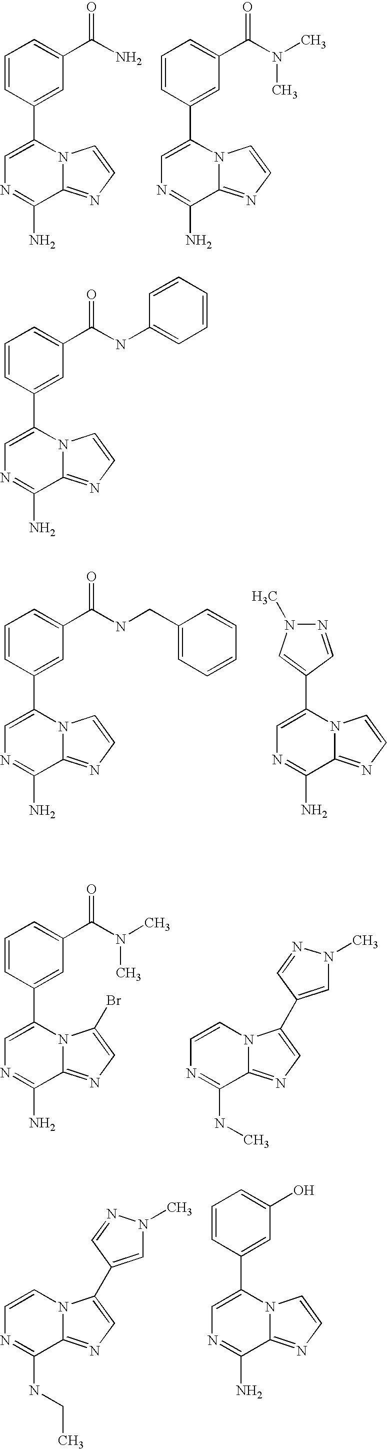

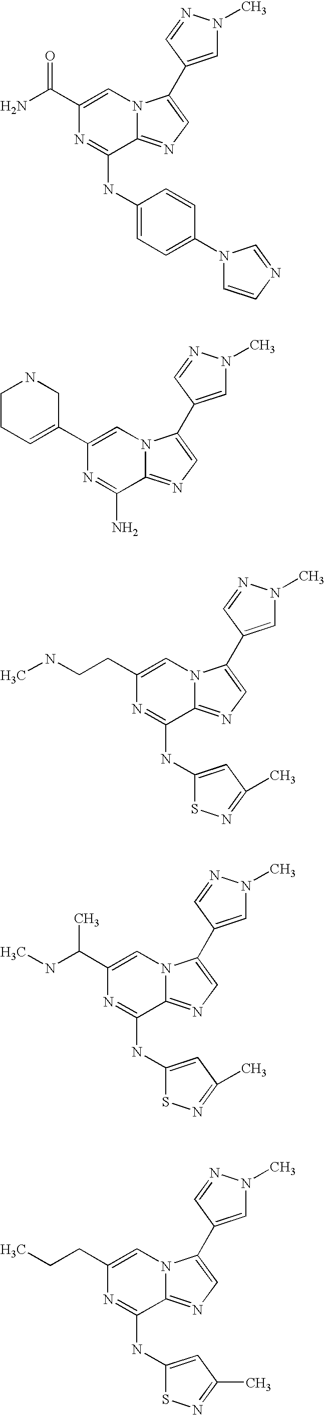

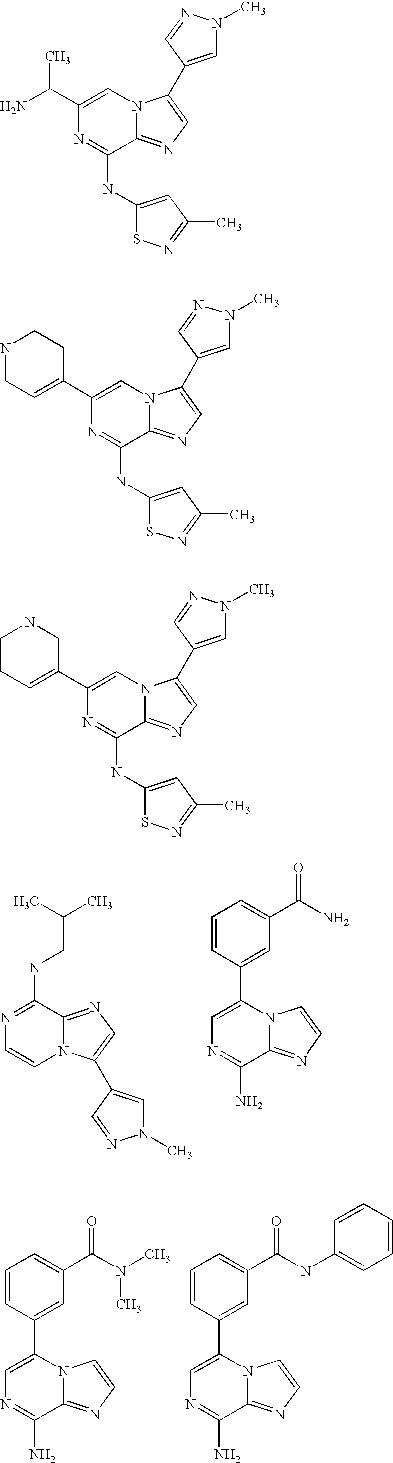

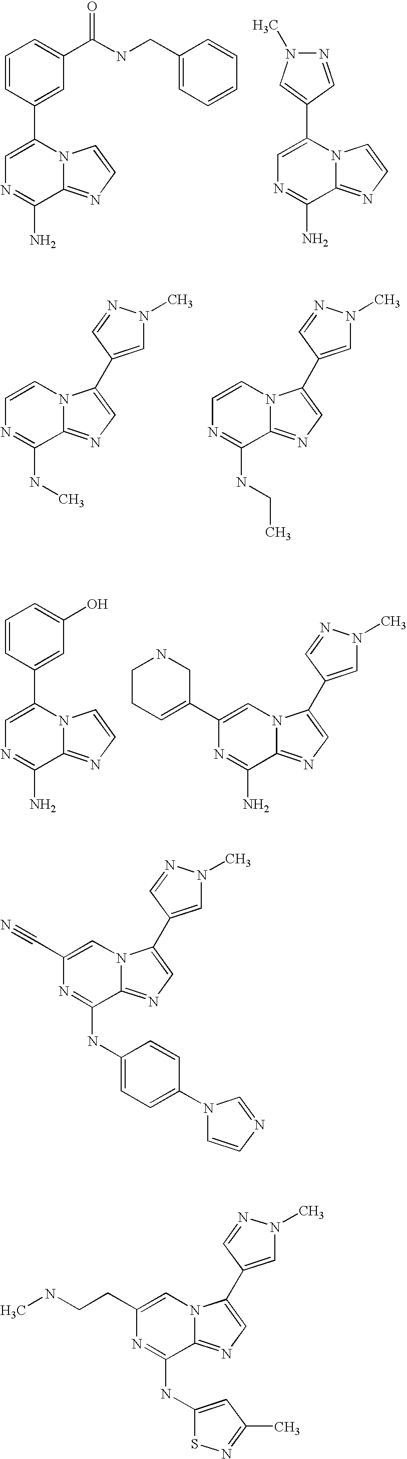

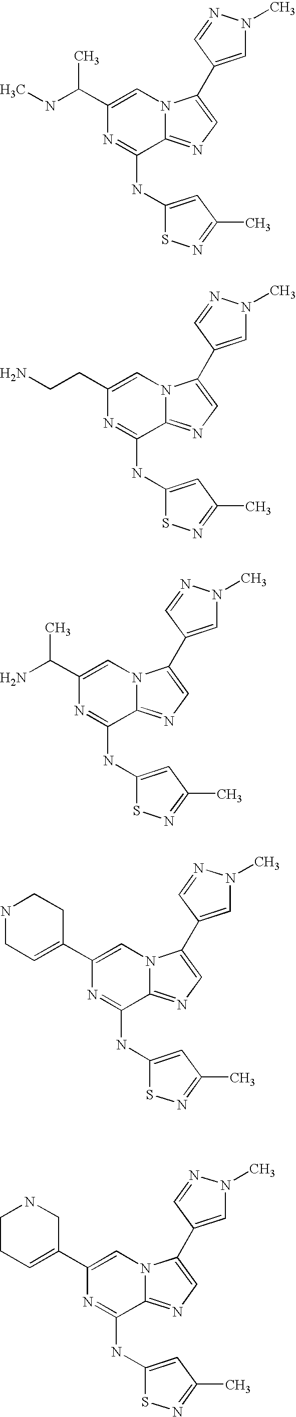

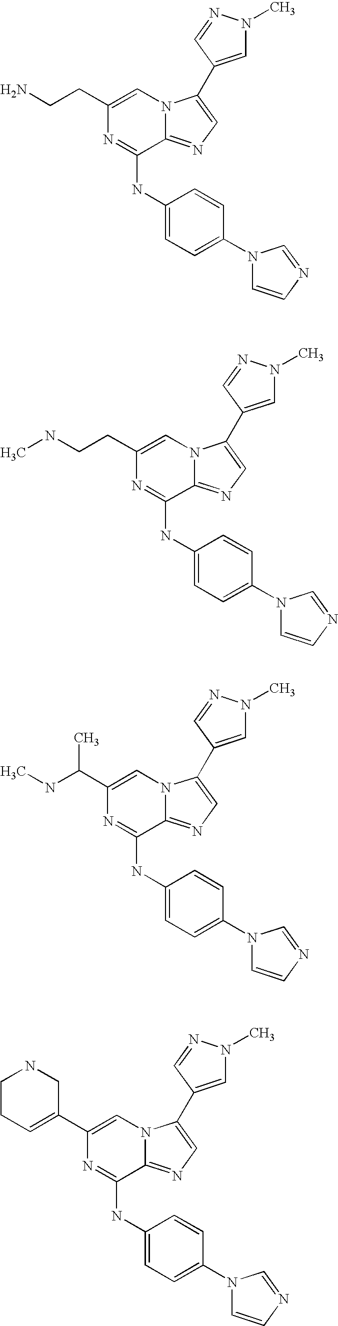

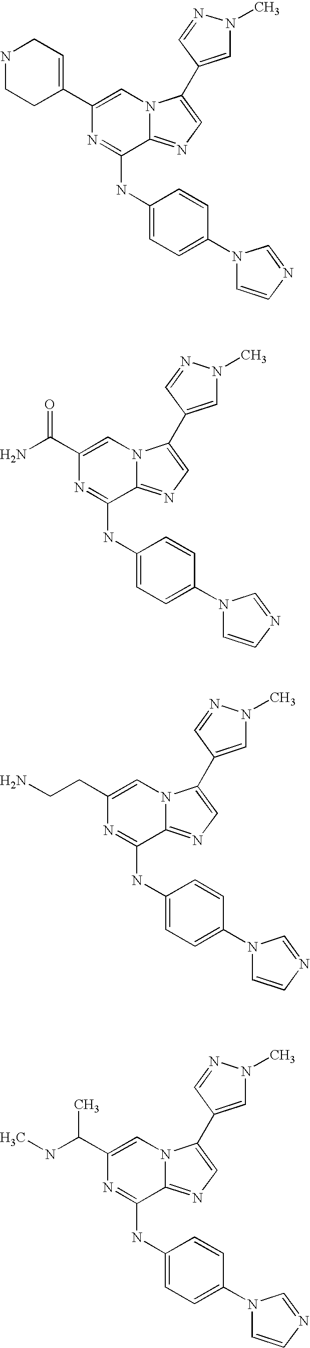

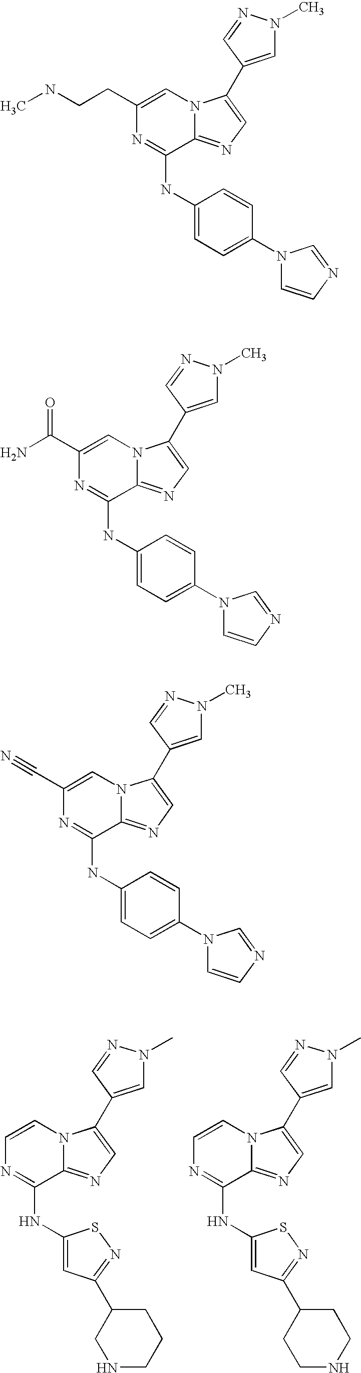

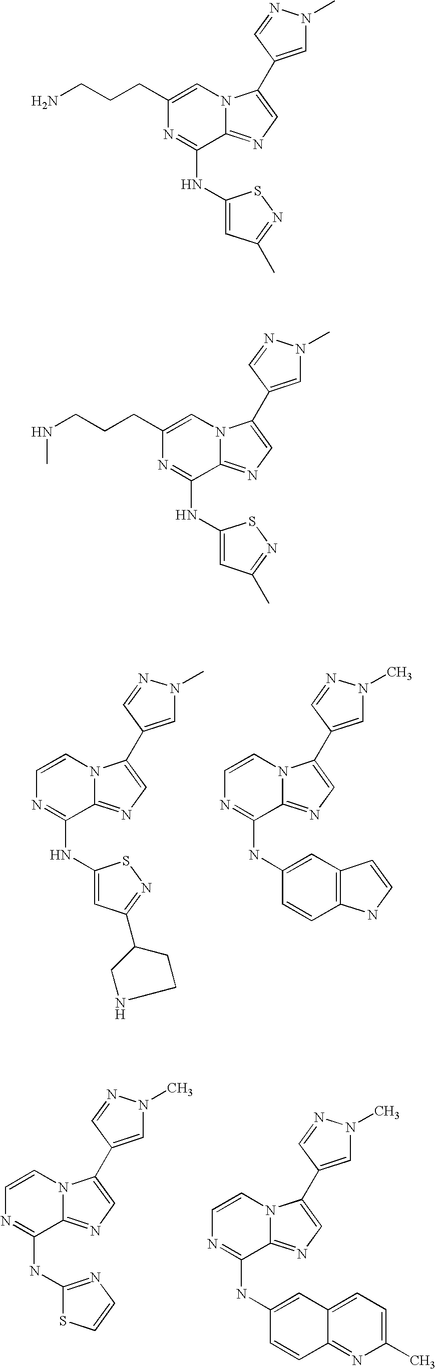

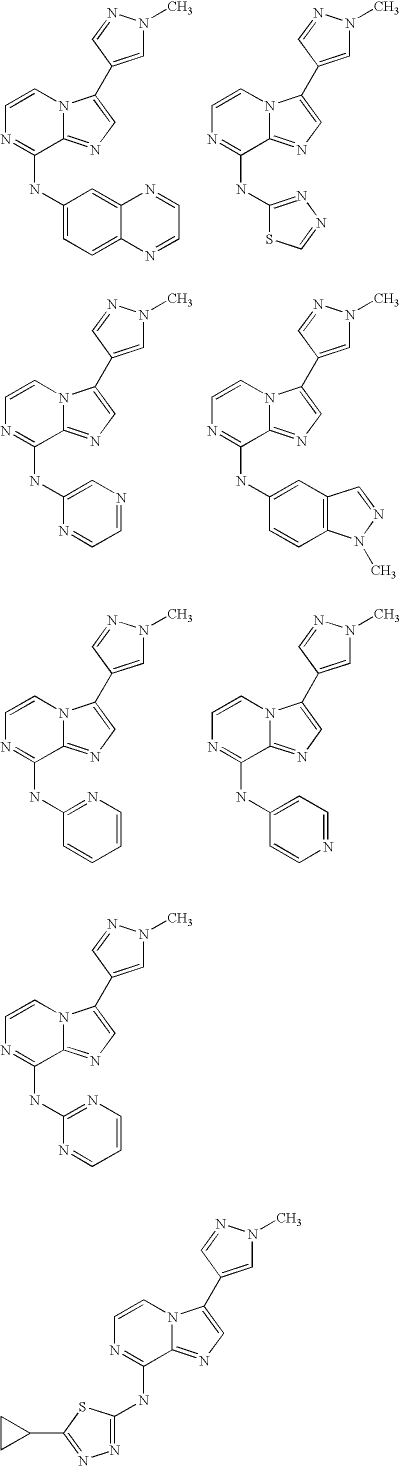

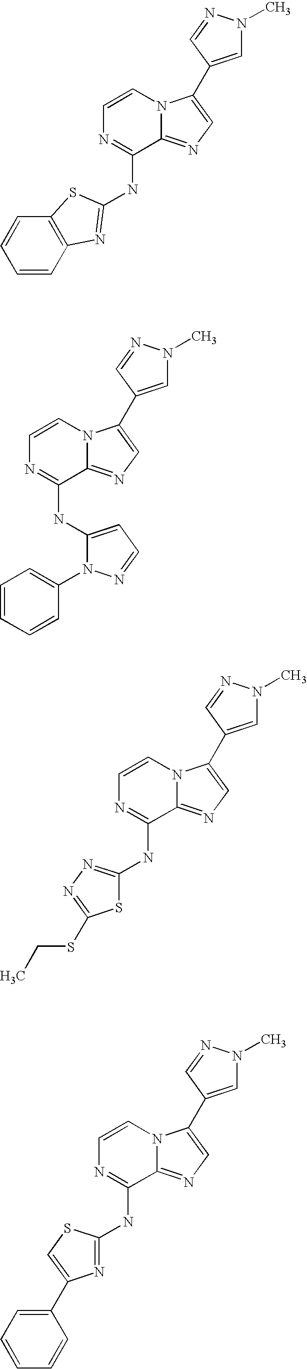

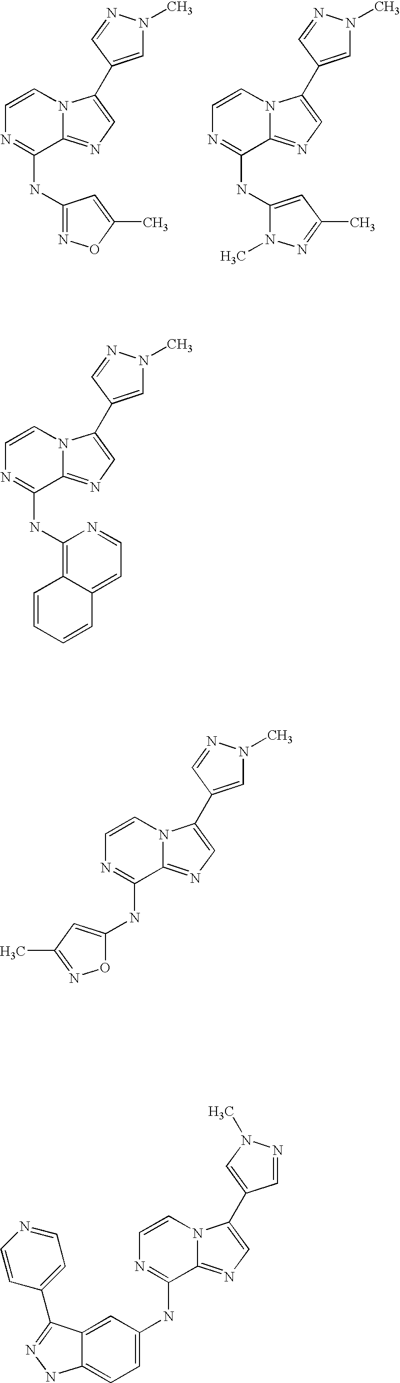

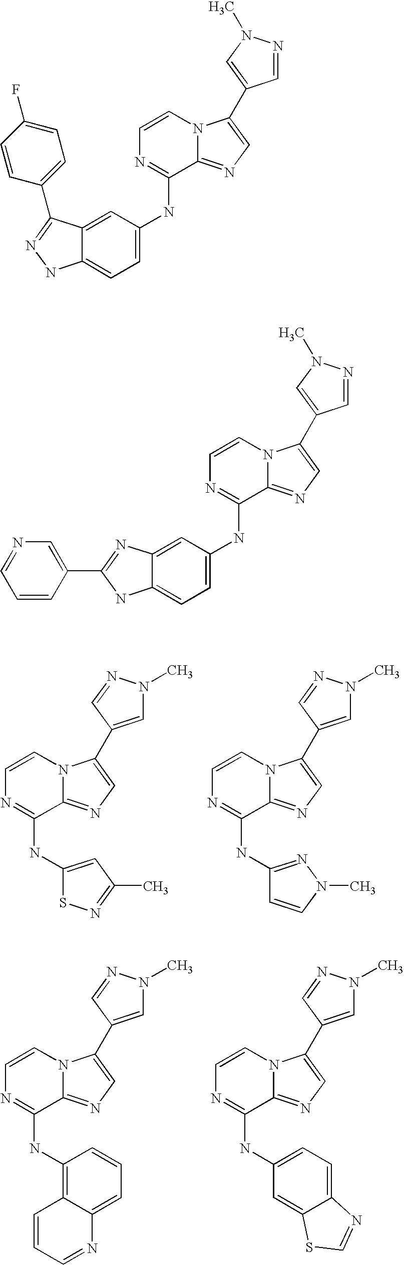

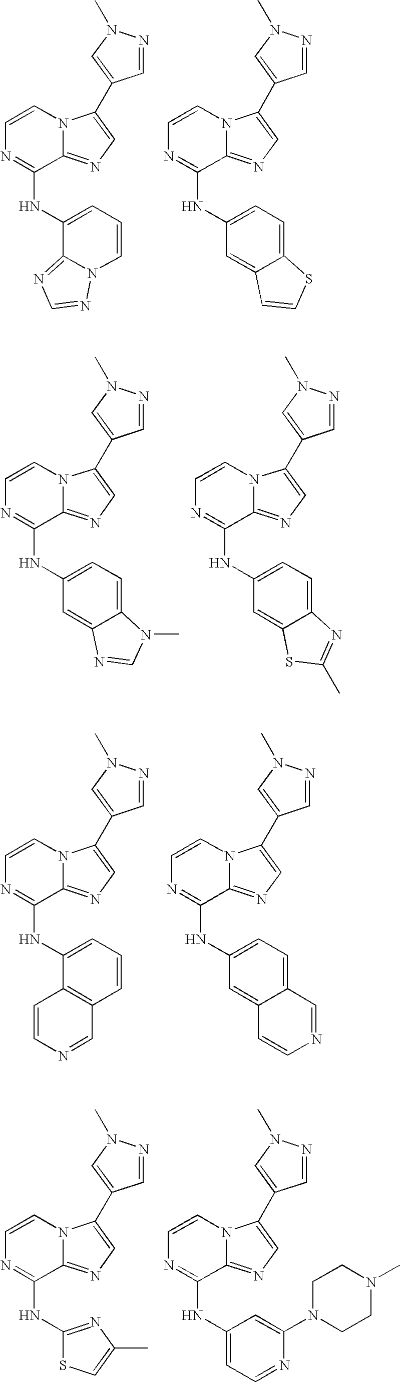

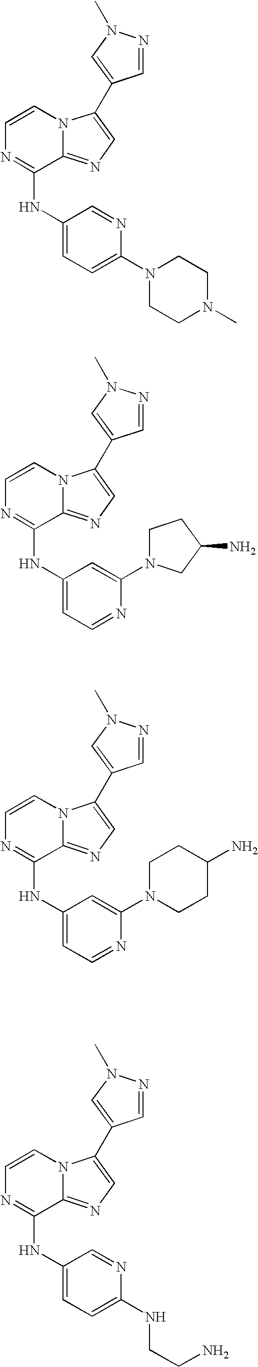

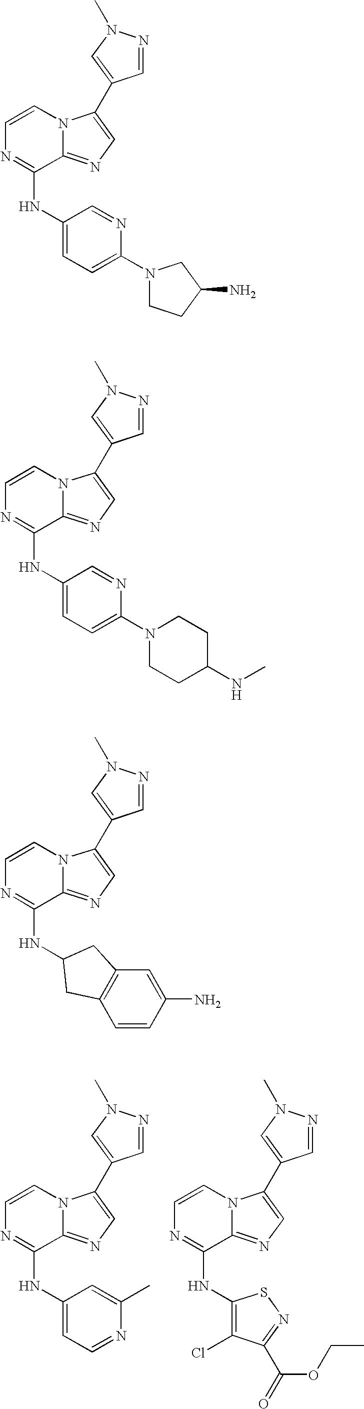

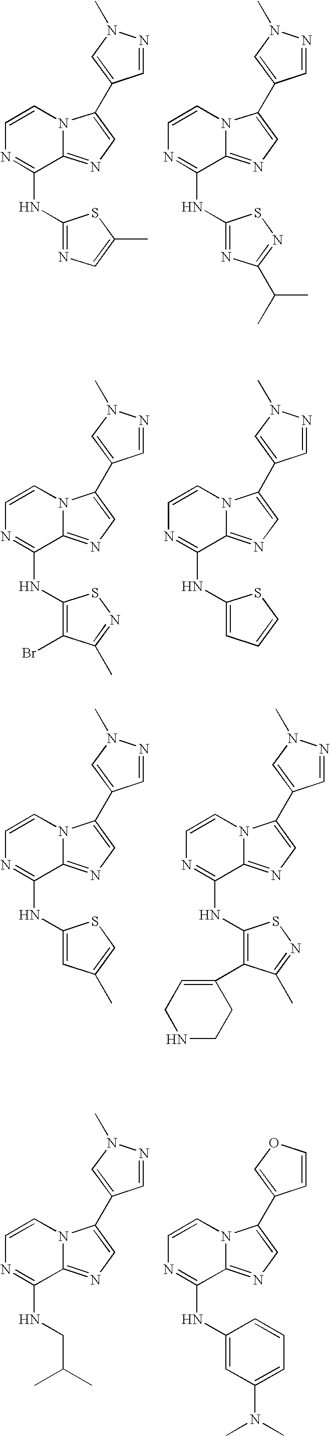

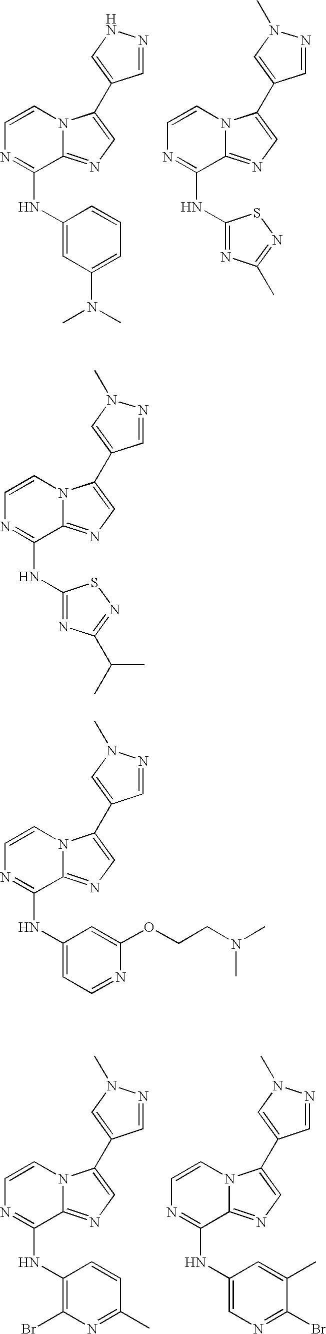

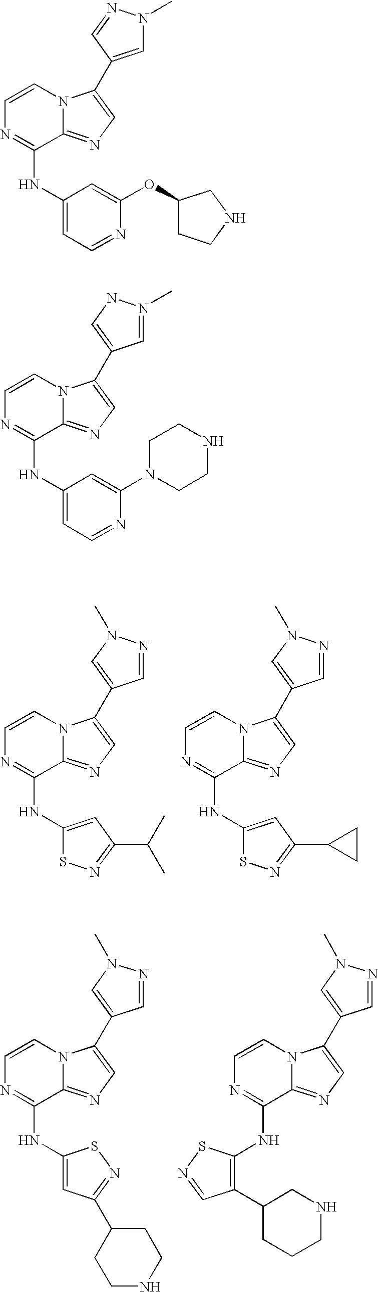

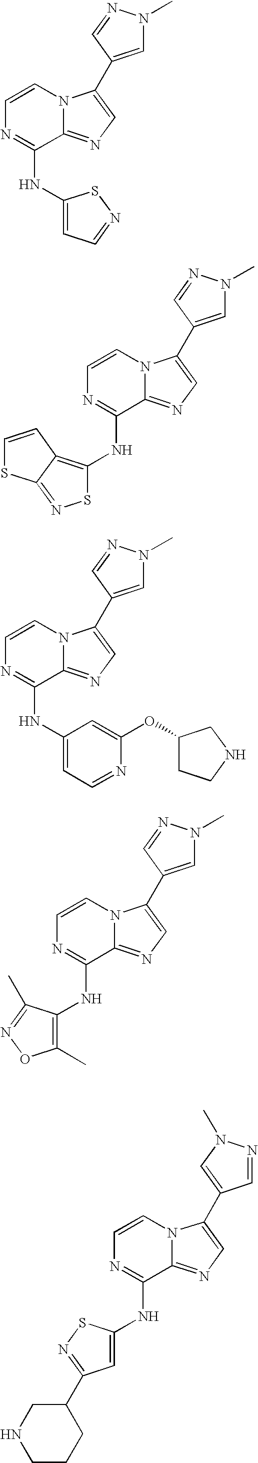

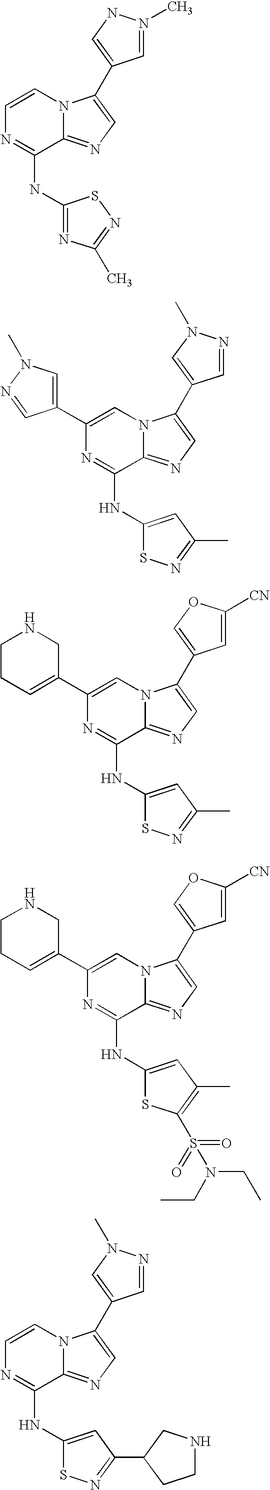

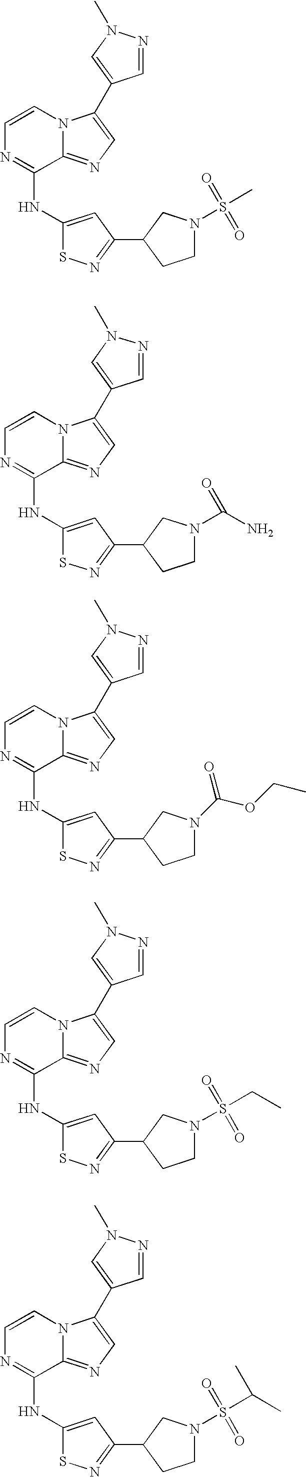

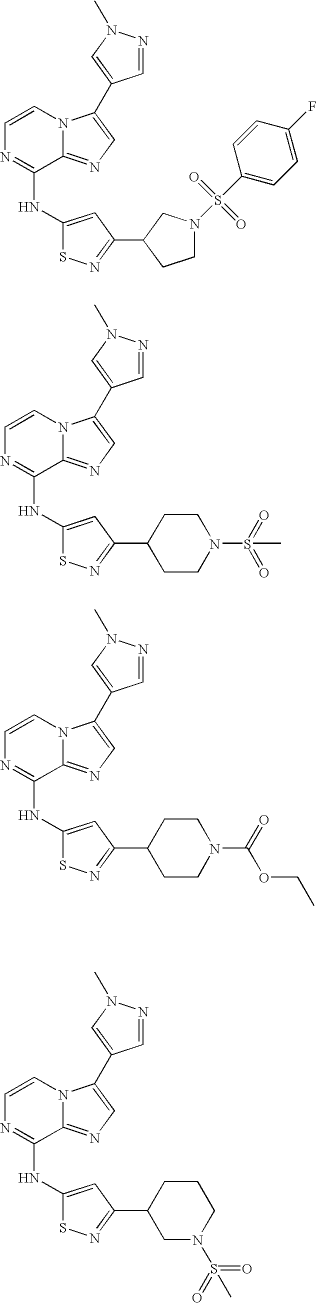

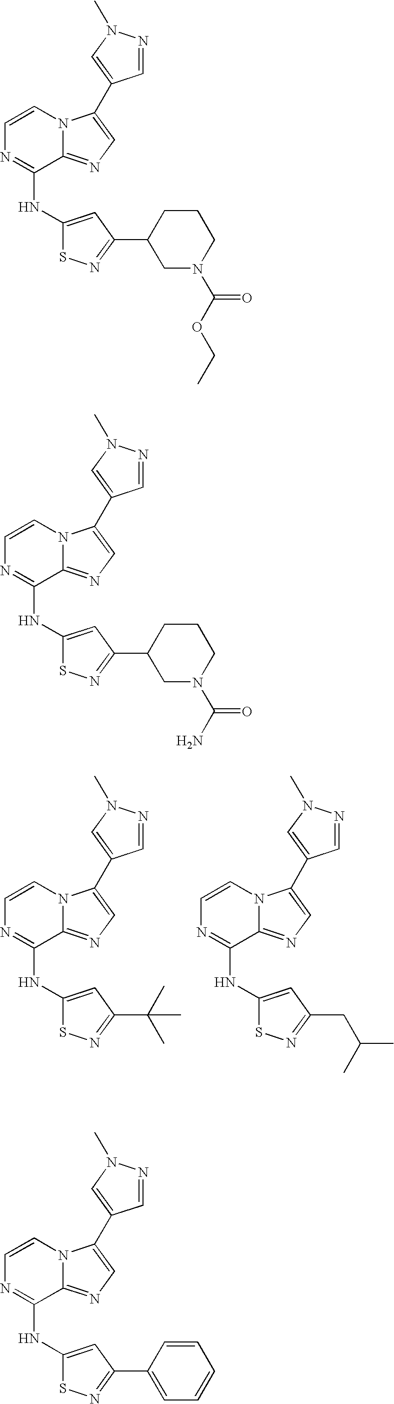

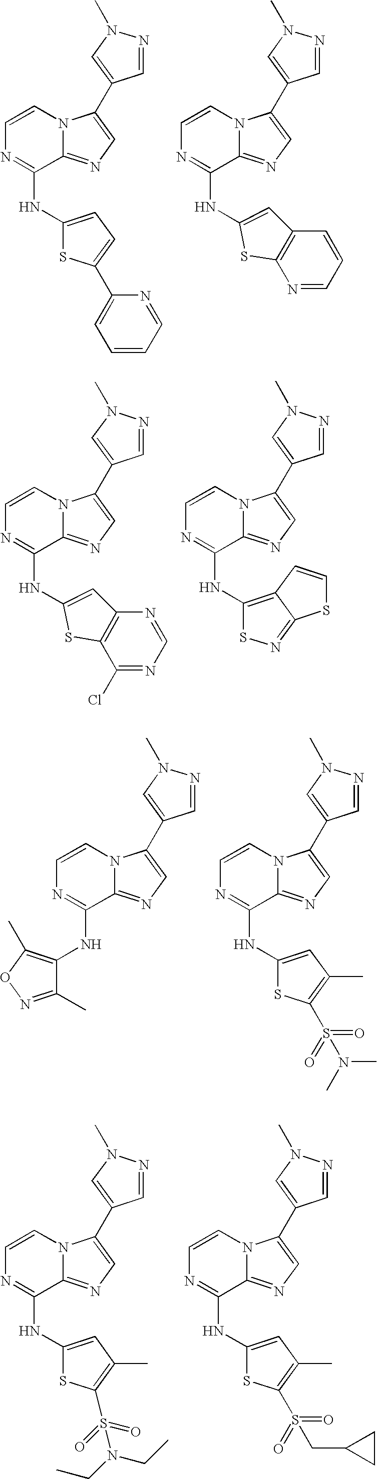

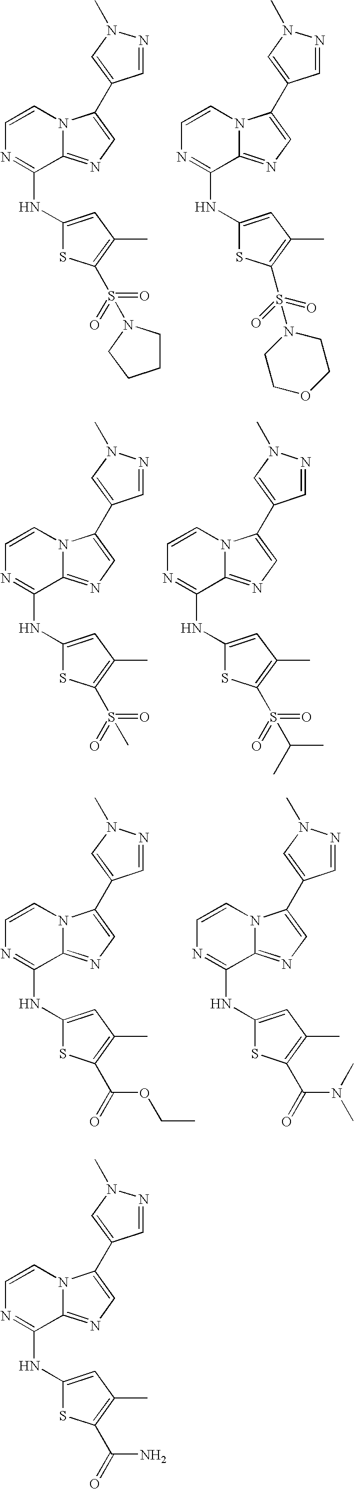

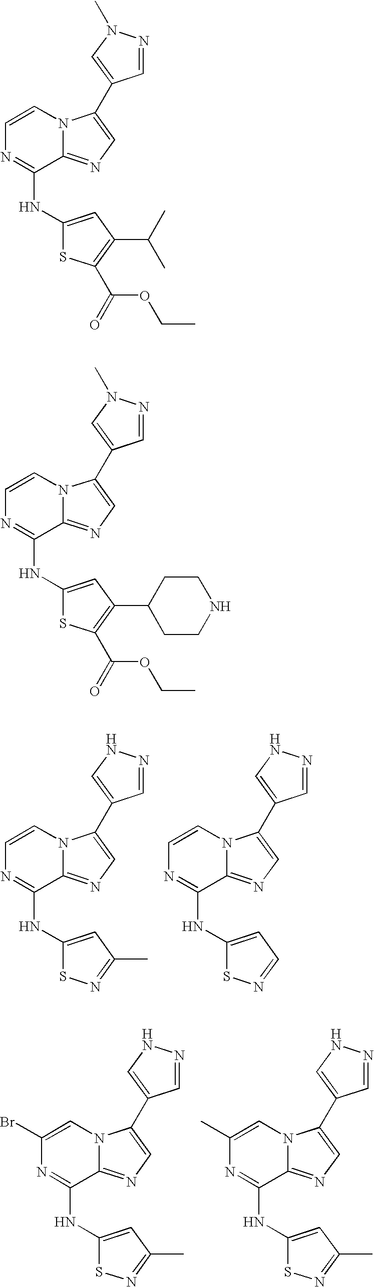

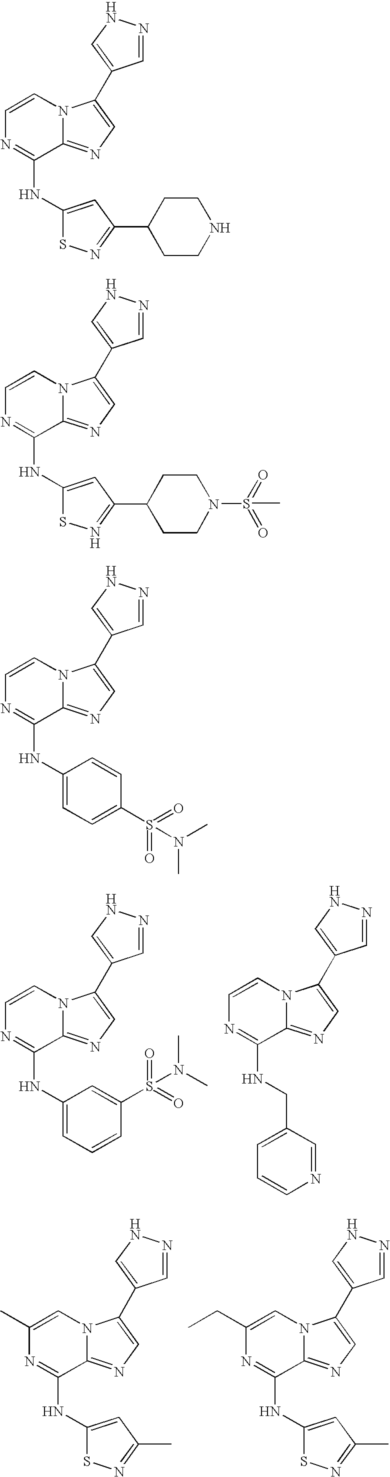

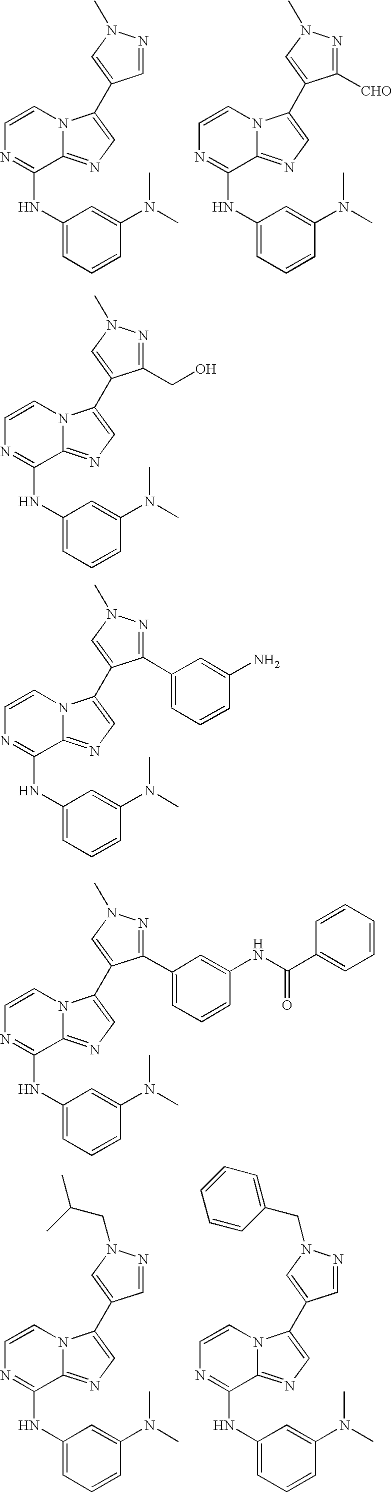

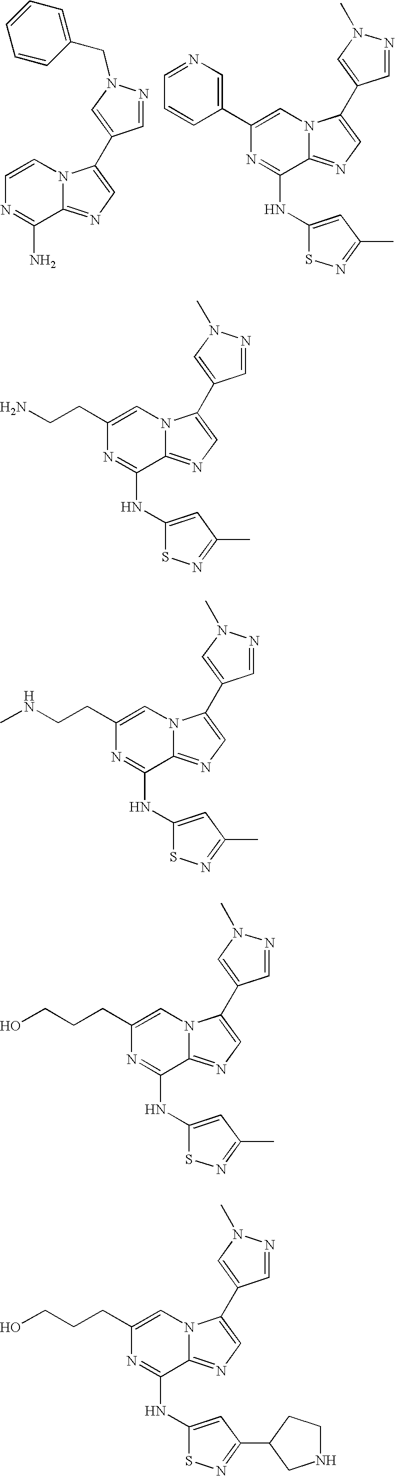

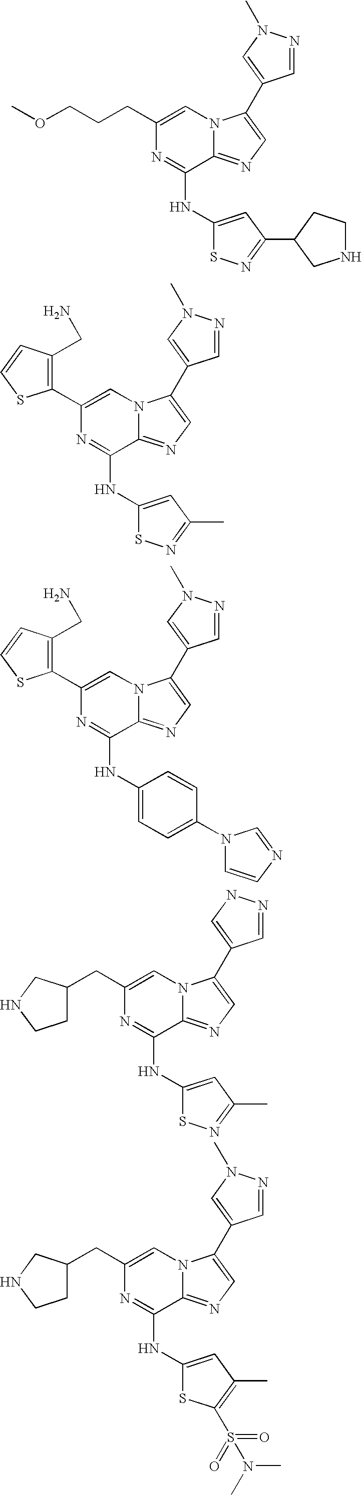

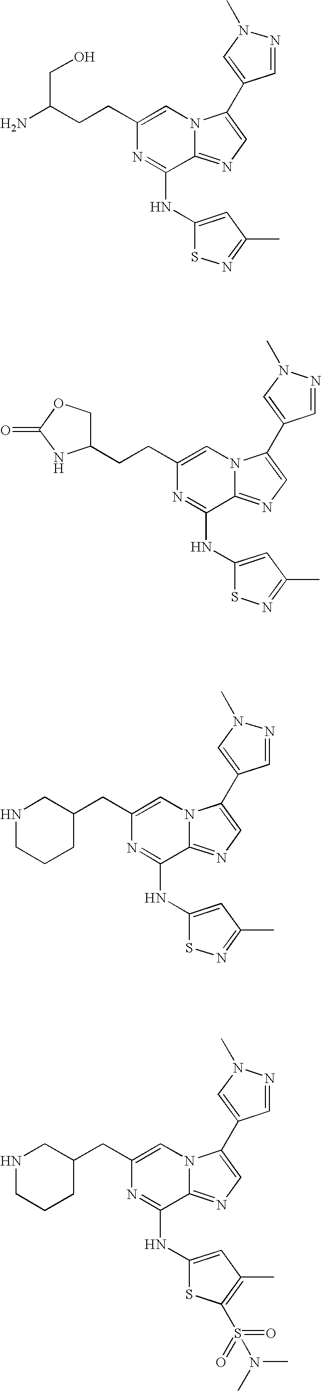

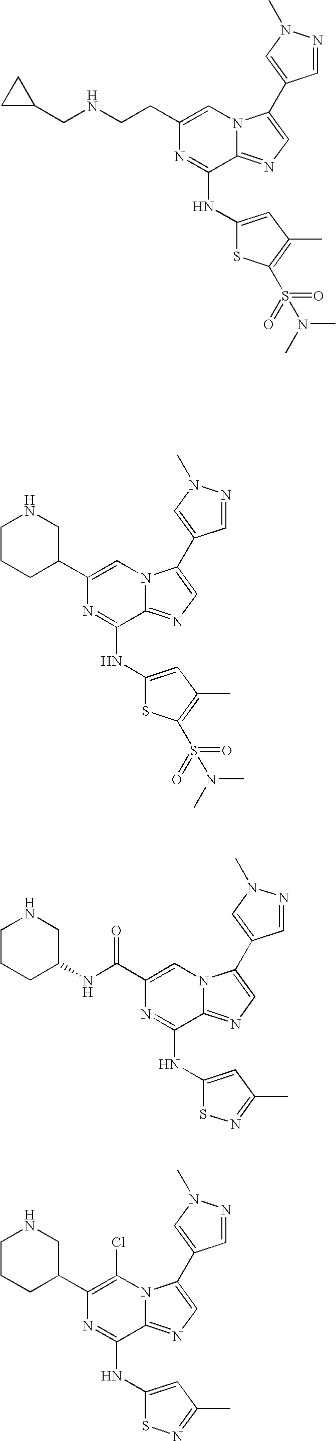

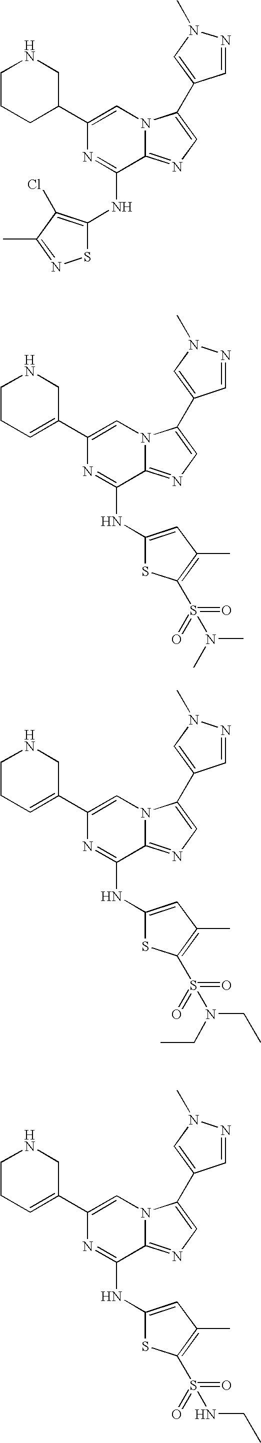

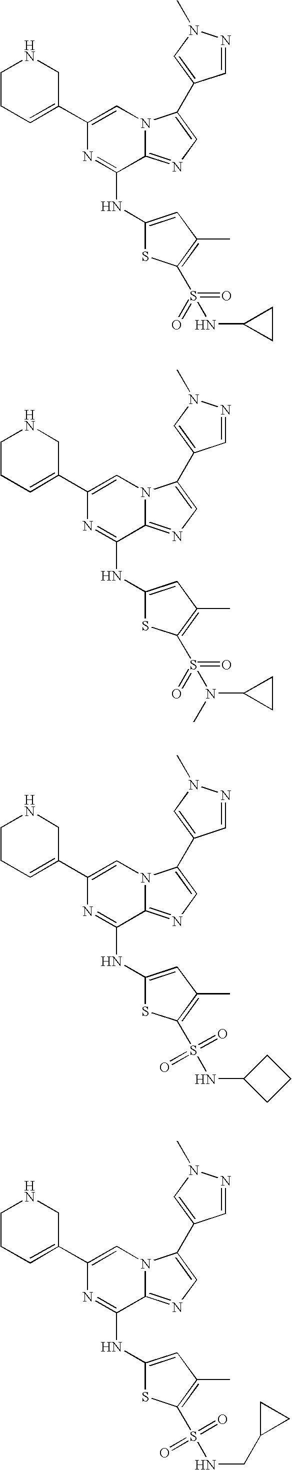

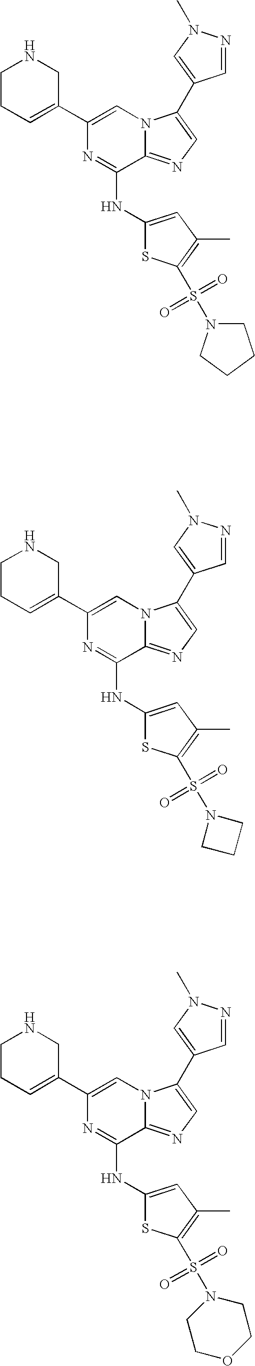

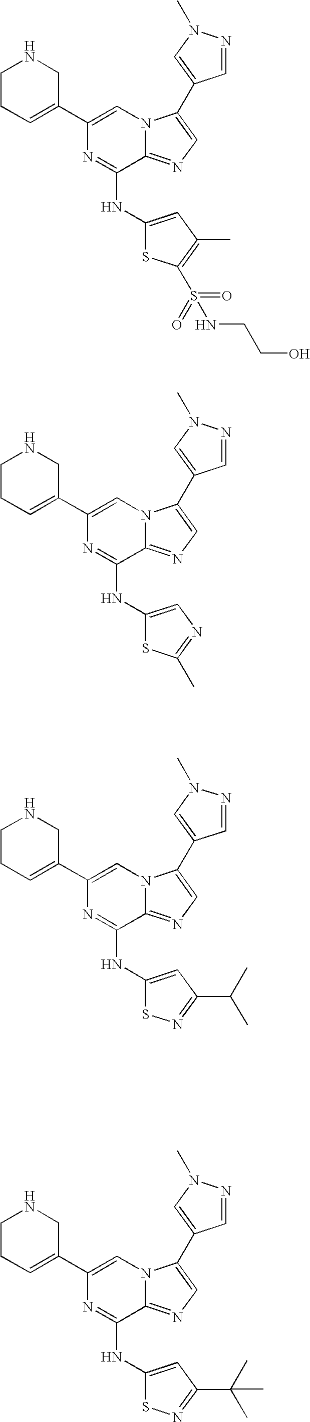

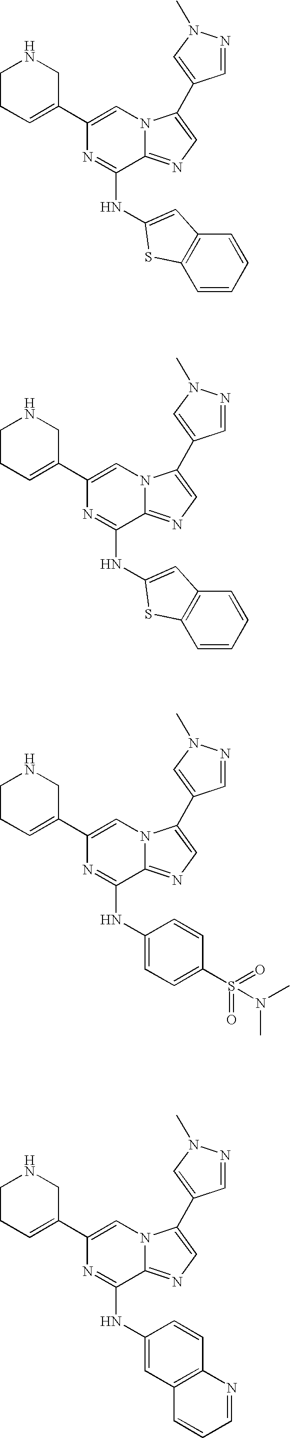

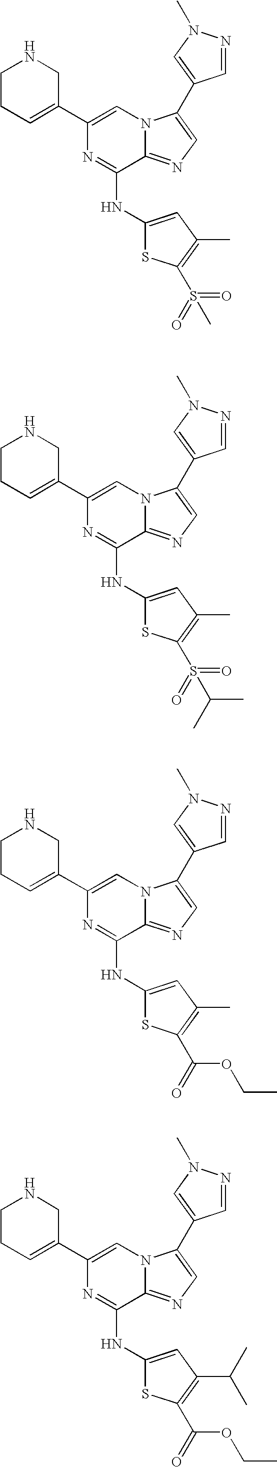

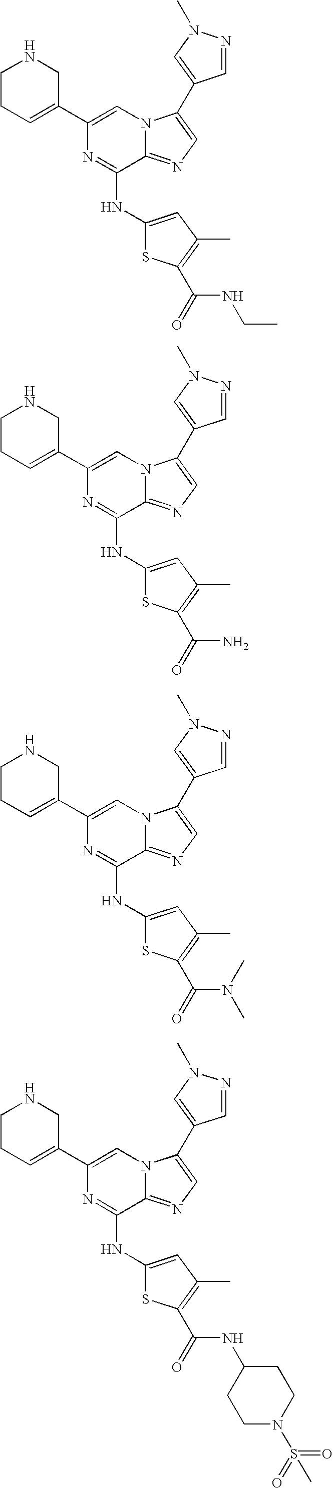

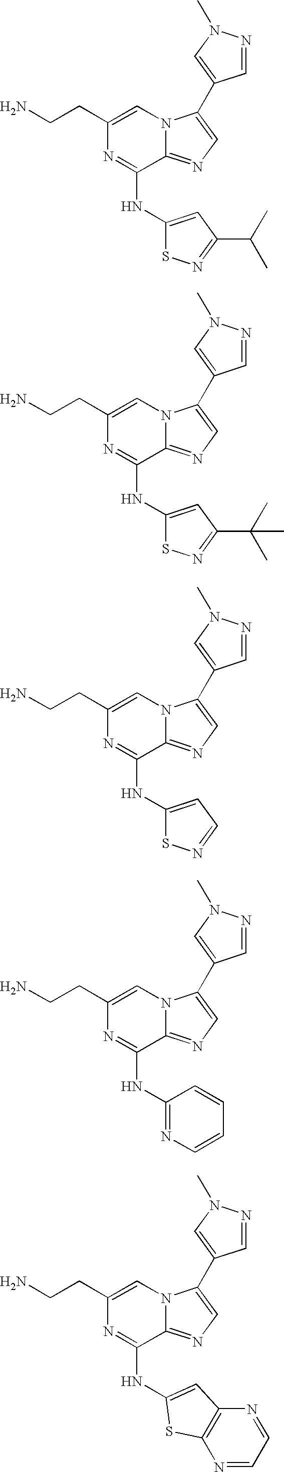

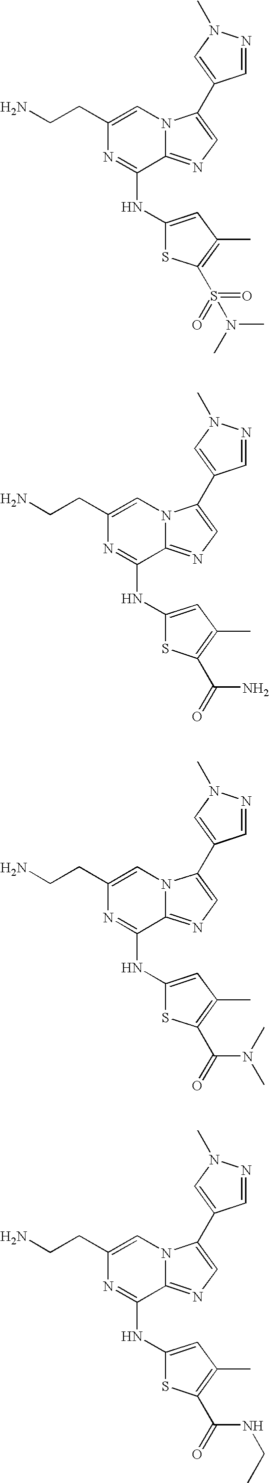

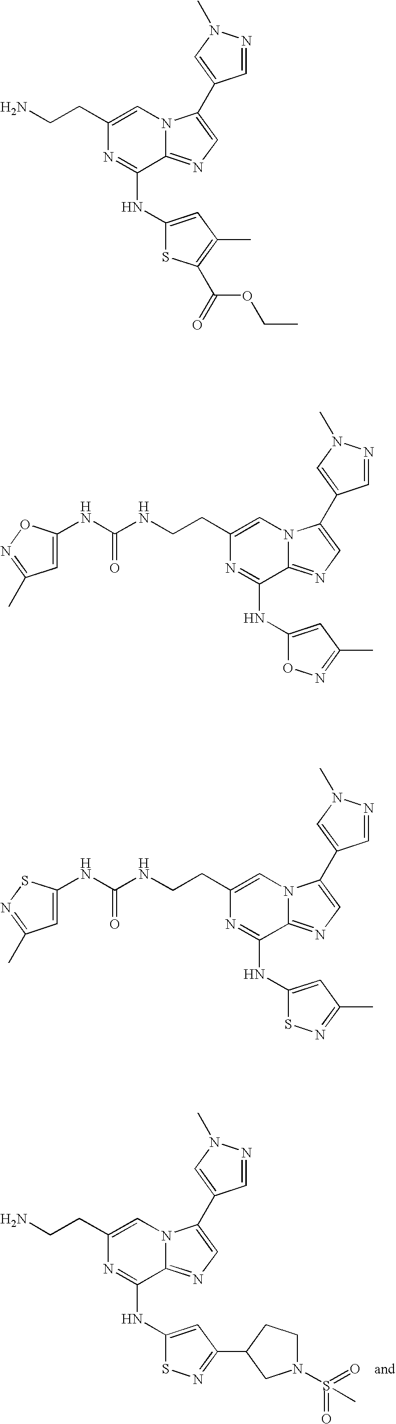

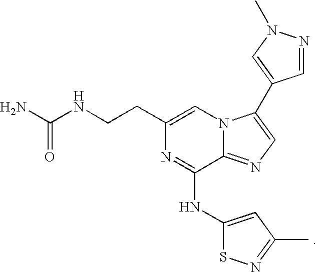

As used above, and throughout this disclosure, the following terms, unless otherwise indicated, shall be understood to have the following meanings, including any possible substitutions of the stated groups or moieties:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, amide, —CHO, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

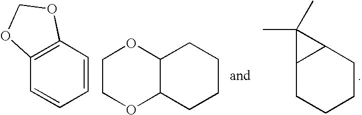

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

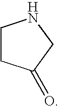

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

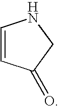

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring: 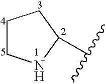
there is no —OH attached directly to carbons marked 2 and 5.

It should also be noted that tautomeric forms such as, for example, the moieties: 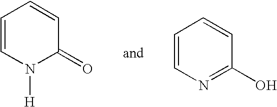
are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention have pharmacological properties; in particular, the compounds of Formula I can be inhibitors, regulators or modulators of protein kinases. Non-limiting examples of protein kinases that can be inhibited, regulated or modulated include cyclin-dependent kinases (CDKs), such as, CDK1, CDK2, CDK3, CDK4, CDK5, CDK6 and CDK7, CDK8, mitogen activated protein kinase (MAPK/ERK), glycogen synthase kinase 3 (GSK3beta), Pim-1 kinases, Chk kinases, such as Chk1 and Chk2, tyrosine kinases, such as the HER subfamily (including, for example, EGFR (HER1), HER2, HER3 and HER4), the insulin subfamily (including, for example, INS-R, IGF-IR, IR, and IR-R), the PDGF subfamily (including, for example, PDGF-alpha and beta receptors, CSFIR, c-kit and FLK-II), the FLK family (including, for example, kinase insert domain receptor (KDR), fetal liver kinase-1 (FLK-1), fetal liver kinase-4 (FLK-4) and the fms-like tyrosine kinase-1 (fit-1)), non-receptor protein tyrosine kinases, for example LCK, Src, Frk, Btk, Csk, Abl, Zap70, Fes/Fps, Fak, Jak, Ack, and LIMK, growth factor receptor tyrosine kinases such as VEGF-R2, FGF-R, TEK, Akt kinases and the like.

The compounds of Formula (I) can be inhibitors of protein kinases such as, for example, the inhibitors of the checkpoint kinases such as Chk1, Chk2 and the like. Preferred compounds can exhibit IC₅₀ values of less than about 5 μm, preferably about 0.001 to about 1.0 μm, and more preferably about 0.001 to about 0.1 μm. The assay methods are described in the Examples set forth below.

The compounds of Formula I can be useful in the therapy of proliferative diseases such as cancer, autoimmune diseases, viral diseases, fungal diseases, neurological/neurodegenerative disorders, arthritis, inflammation, anti-proliferative (e.g., ocular retinopathy), neuronal, alopecia and cardiovascular disease. Many of these diseases and disorders are listed in U.S. Pat. No. 6,413,974 cited earlier, incorporated by reference herein.

More specifically, the compounds of Formula I can be useful in the treatment of a variety of cancers, including (but not limited to) the following:

carcinoma, including that of the bladder, breast, colon, kidney, liver, lung, including small cell lung cancer, non-small cell lung cancer, head and neck, esophagus, gall bladder, ovary, pancreas, stomach, cervix, thyroid, prostate, and skin, including squamous cell carcinoma;

hematopoietic tumors of lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;

hematopoietic tumors of myeloid lineage, including acute and chronic myelogenous leukemias, myelodysplastic syndrome and promyelocytic leukemia;

tumors of mesenchymal origin, including fibrosarcoma and rhabdomyosarcoma;

tumors of the central and peripheral nervous system, including astrocytoma, neuroblastoma, glioma and schwannomas; and

other tumors, including melanoma, seminoma, teratocarcinoma, osteosarcoma, xenoderoma pigmentosum, keratoctanthoma, thyroid follicular cancer and Kaposi's sarcoma.

Due to the key role of CDKs in the regulation of cellular proliferation in general, inhibitors could act as reversible cytostatic agents which may be useful in the treatment of any disease process which features abnormal cellular proliferation, e.g., benign prostate hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections. Compounds of Formula I may also be useful in the treatment of Alzheimer's disease, as suggested by the recent finding that CDK5 is involved in the phosphorylation of tau protein (J. Biochem, (1995) 117, 741-749). Compounds of Formula I may induce or inhibit apoptosis. The apoptotic response is aberrant in a variety of human diseases. Compounds of Formula I, as modulators of apoptosis, will be useful in the treatment of cancer (including but not limited to those types mentioned hereinabove), viral infections (including but not limited to herpevirus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer's disease, AIDS-related dementia, Parkinson's disease, amyotrophic lateral sclerosis, retinitis pigmentosa, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis) aspirin-sensitive rhinosinusitis, cystic fibrosis, multiple sclerosis, kidney diseases and cancer pain.

Compounds of Formula I, as inhibitors of the CDKs, can modulate the level of cellular RNA and DNA synthesis. These agents would therefore be useful in the treatment of viral infections (including but not limited to HIV, human papilloma virus, herpesvirus, poxvirus, Epstein-Barr virus, Sindbis virus and adenovirus).

Compounds of Formula I may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

Compounds of Formula I may also be useful in inhibiting tumor angiogenesis and metastasis.

Compounds of Formula I may also act as inhibitors of other protein kinases, e.g., protein kinase C, her2, raf 1, MEK1, MAP kinase, EGF receptor, PDGF receptor, IGF receptor, PI3 kinase, wee1 kinase, Src, Abl and thus be effective in the treatment of diseases associated with other protein kinases. Another aspect of this invention is a method of treating a mammal (e.g., human) having a disease or condition associated with the CDKs by administering a therapeutically effective amount of at least one compound of Formula I, or a pharmaceutically acceptable salt, solvate, ester or prodrug of said compound to the mammal.

A preferred dosage is about 0.001 to 1000 mg/kg of body weight/day of the compound of Formula I. An especially preferred dosage is about 0.01 to 25 mg/kg of body weight/day of a compound of Formula I, or a pharmaceutically acceptable salt, solvate, ester or prodrug of said compound. The compounds of this invention may also be useful in combination (administered together or sequentially) with one or more of anti-cancer treatments such as radiation therapy, and/or one or more anti-cancer agents different from the compound of Formula I. The compounds of the present invention can be present in the same dosage unit as the anti-cancer agent or in separate dosage units.

Another aspect of the present invention is a method of treating one or more diseases associated with cyclin dependent kinase, comprising administering to a mammal in need of such treatment an amount of a first compound, which is a compound of claim 1, or a pharmaceutically acceptable salt, solvate, ester or prodrug thereof; and an amount of at least one second compound, the second compound being an anti-cancer agent different from the compound of claim 1, wherein the amounts of the first compound and the second compound result in a therapeutic effect.

Non-limiting examples of suitable anti-cancer agents include cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methoxtrexate); alkylating agents (such as temozolomide (TEMODAR™ from Schering-Plough Corporation, Kenilworth, N.J.), cyclophosphamide); Farnesyl protein transferase inhibitors (such as, SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide, or SCH 66336 from Schering-Plough Corporation, Kenilworth, New Jersey), tipifarnib (Zarnestra® or R115777 from Janssen Pharmaceuticals), L778,123 (a farnesyl protein transferase inhibitor from Merck & Company, Whitehouse Station, New Jersey), BMS 214662 (a farnesyl protein transferase inhibitor from Bristol-Myers Squibb Pharmaceuticals, Princeton, N.J.); signal transduction inhibitors (such as, Iressa (from Astra Zeneca Pharmaceuticals, England), Tarceva (EGFR kinase inhibitors), antibodies to EGFR (e.g., C225), GLEEVEC™ (C-abl kinase inhibitor from Novartis Pharmaceuticals, East Hanover, N.J.); interferons such as, for example, intron (from Schering-Plough Corporation), Peg-Intron (from Schering-Plough Corporation); hormonal therapy combinations; aromatase combinations; ara-C, adriamycin, cytoxan, and gemcitabine.

Other anti-cancer (also known as anti-neoplastic) agents include but are not limited to Uracil mustard, Chlormethine, Ifosfamide, Melphalan, Chlorambucil, Pipobroman, Triethylenemelamine, cytostatic agents, cytotoxic agents (such as for example, but not limited to, DNA interactive agents (such as cisplatin or doxorubicin)); taxanes (e.g. taxotere, taxol); topoisomerase II inhibitors (such as etoposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methoxtrexate); alkylating agents (such as temozolomide (TEMODAR™ from Schering-Plough Corporation, Kenilworth, New Jersey), cyclophosphamide); Farnesyl protein transferase inhibitors (such as, SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide, or SCH 66336 from Schering-Plough Corporation, Kenilworth, New Jersey), tipifarnib (Zarnestra® or R115777 from Janssen Pharmaceuticals), L778,123 (a farnesyl protein transferase inhibitor from Merck & Company, Whitehouse Station, New Jersey), BMS 214662 (a farnesyl protein transferase inhibitor from Bristol-Myers Squibb Pharmaceuticals, Princeton, N.J.); signal transduction inhibitors (such as, Iressa (from Astra Zeneca Pharmaceuticals, England), Tarceva (EGFR kinase inhibitors), antibodies to EGFR (e.g., C225), GLEEVEC™ (C-abl kinase inhibitor from Novartis Pharmaceuticals, East Hanover, N.J.); interferons such as, for example, intron (from Schering-Plough Corporation), Peg-Intron (from Schering-Plough Corporation); hormonal therapy combinations; aromatase combinations; ara-C, adriamycin, cytoxan, Clofarabine (Clolar® from Genzyme Oncology, Cambridge, Mass.), cladribine (Leustat® from Janssen-Cilag Ltd.), aphidicolon, rituxan (from Genentech/Biogen Idec), sunitinib (Sutent® from Pfizer), dasatinib (or BMS-354825 from Bristol-Myers Squibb), tezacitabine (from Aventis Pharma), Sml1, fludarabine (from Trigan Oncology Associates), pentostatin (from BC Cancer Agency), triapine (from Vion Pharmaceuticals), didox (from Bioseeker Group), trimidox (from ALS Therapy Development Foundation), amidox, 3-AP (3-aminopyridine-2-carboxaldehyde thiosemicarbazone), MDL-101,731 ((E)-2′-deoxy-2′-(fluoromethylene)cytidine) and gemcitabine.

Other anti-cancer (also known as anti-neoplastic) agents include but are not limited to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaceuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, Herceptin, Bexxar, Velcade, Zevalin, Trisenox, Xeloda, Vinorelbine, Profimer, Erbitux, Liposomal, Thiotepa, Altretamine, Melphalan, Trastuzumab, Lerozole, Fulvestrant, Exemestane, Fulvestrant, Ifosfomide, Rituximab, C225 and Campath.

If formulated as a fixed dose, such combination products employ the compounds of this invention within the dosage range described herein and the other pharmaceutically active agent or treatment within its dosage range. For example, the CDC2 inhibitor olomucine has been found to act synergistically with known cytotoxic agents in inducing apoptosis (J. Cell Sci., (1995) 108, 2897. Compounds of Formula I may also be administered sequentially with known anticancer or cytotoxic agents when a combination formulation is inappropriate. The invention is not limited in the sequence of administration; compounds of Formula I may be administered either prior to or after administration of the known anticancer or cytotoxic agent. For example, the cytotoxic activity of the cyclin-dependent kinase inhibitor flavopiridol is affected by the sequence of administration with anticancer agents. Cancer Research, (1997) 57, 3375. Such techniques are within the skills of persons skilled in the art as well as attending physicians.

Accordingly, in an aspect, this invention includes combinations comprising an amount of at least one compound of Formula I, or a pharmaceutically acceptable salt, solvate, ester or prodrug thereof, and an amount of one or more anti-cancer treatments and anti-cancer agents listed above wherein the amounts of the compounds/treatments result in desired therapeutic effect.

Another aspect of the present invention is a method of inhibiting one or more Checkpoint kinases in a patient in need thereof, comprising administering to the patient a therapeutically effective amount of at least one compound of claim 1 or a pharmaceutically acceptable salt, solvate, ester or prodrug thereof.

Another aspect of the present invention is a method of treating, or slowing the progression of, a disease associated with one or more Checkpoint kinases in a patient in need thereof, comprising administering a therapeutically effective amount of at least one compound of claim 1 or a pharmaceutically acceptable salt, solvate, ester or prodrug thereof.

Yet another aspect of the present invention is a method of treating one or more diseases associated with Checkpoint kinase, comprising administering to a mammal in need of such treatment an amount of a first compound, which is a compound of claim 1, or a pharmaceutically acceptable salt, solvate, ester or prodrug thereof; and an amount of at least one second compound, the second compound being an anti-cancer agent, wherein the amounts of the first compound and the second compound result in a therapeutic effect.

Another aspect of the present invention is a method of treating, or slowing the progression of, a disease associated with one or more Checkpoint kinases in a patient in need thereof, comprising administering a therapeutically effective amount of a pharmaceutical composition comprising in combination at least one pharmaceutically acceptable carrier and at least one compound according to claim 1, or a pharmaceutically acceptable salt, solvate, ester or prodrug thereof.

In the above methods, the checkpoint kinase to be inhibited can be Chk1 and/or Chk2.

Another aspect of the present invention is a method of inhibiting one or more tyrosine kinases in a patient in need thereof, comprising administering to the patient a therapeutically effective amount of at least one compound of claim 1 or a pharmaceutically acceptable salt, solvate, ester or prodrug thereof.

Yet another aspect of the present invention is a method of treating, or slowing the progression of, a disease associated with one or more tyrosine kinases in a patient in need thereof, comprising administering a therapeutically effective amount of at least one compound of claim 1 or a pharmaceutically acceptable salt, solvate, ester or prodrug thereof.

Another aspect of the present invention is a method of treating one or more diseases associated with tyrosine kinase, comprising administering to a mammal in need of such treatment an amount of a first compound, which is a compound of claim 1, or a pharmaceutically acceptable salt, solvate, ester or prodrug thereof; and an amount of at least one second compound, the second compound being an anti-cancer agent, wherein the amounts of the first compound and the second compound result in a therapeutic effect.

Another aspect of the present invention is a method of treating, or slowing the progression of, a disease associated with one or more tyrosine kinases in a patient in need thereof, comprising administering a therapeutically effective amount of a pharmaceutical composition comprising in combination at least one pharmaceutically acceptable carrier and at least one compound according to claim 1 or a pharmaceutically acceptable salt, solvate, ester or prodrug thereof.

In the above methods, the tyrosine kinase can be VEGFR (VEGF-R2), EGFR, HER2, SRC, JAK and/or TEK.

Another aspect of the present invention is a method of inhibiting one or more Pim-1 kinases in a patient in need thereof, comprising administering to the patient a therapeutically effective amount of at least one compound of claim 1 or a pharmaceutically acceptable salt, solvate, ester or prodrug thereof.

Yet another aspect of the present invention is a method of treating, or slowing the progression of, a disease associated with one or more Pim-1 kinases in a patient in need thereof, comprising administering a therapeutically effective amount of at least one compound of claim 1 or a pharmaceutically acceptable salt, solvate, ester or prodrug thereof.

Another aspect of the present invention is a method of treating one or more diseases associated with Pim-1 kinase, comprising administering to a mammal in need of such treatment an amount of a first compound, which is a compound of claim 1, or a pharmaceutically acceptable salt, solvate, ester or prodrug thereof; and an amount of at least one second compound, the second compound being an anti-cancer agent, wherein the amounts of the first compound and the second compound result in a therapeutic effect.

Another aspect of the present invention is a method of treating, or slowing the progression of, a disease associated with one or more Pim-1 kinases in a patient in need thereof, comprising administering a therapeutically effective amount of a pharmaceutical composition comprising in combination at least one pharmaceutically acceptable carrier and at least one compound according to claim 1 or a pharmaceutically acceptable salt, solvate, ester or prodrug thereof.

The pharmacological properties of the compounds of this invention may be confirmed by a number of pharmacological assays. The exemplified pharmacological assays which are described herein below have been carried out with compounds according to the invention and their salts, solvates, esters or prodrugs.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally or intravenously.

Also contemplated are delivery methods that are combinations of the above-noted delivery methods. Such methods are within the skill of, or typically decided, by, those skilled in the art.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Yet another aspect of this invention is a kit comprising an amount of at least one compound of Formula I, or a pharmaceutically acceptable salt, solvate, ester or prodrug of said compound and an amount of at least one anticancer therapy and/or anti-cancer agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art.

Where NMR data are presented, ¹H spectra were obtained on either a Varian VXR-200 (200 MHz, ¹H), Varian Gemini-300 (300 MHz) or XL-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A L column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The retention time and observed parent ion are given.

The following solvents and reagents may be referred to by their abbreviations in parenthesis:

Thin layer chromatography: TLC

dichloromethane: CH₂Cl₂

ethyl acetate: AcOEt or EtOAc

methanol: MeOH

trifluoroacetate: TFA

triethylamine: Et₃N or TEA

butoxycarbonyl: n-Boc or Boc

nuclear magnetic resonance spectroscopy: NMR

liquid chromatography mass spectrometry: LCMS

high resolution mass spectrometry: HRMS

milliliters: mL

millimoles: mmol

microliters: μl

grams: g

milligrams: mg

room temperature or rt (ambient): about 25° C.

dimethoxyethane: DME

The synthesis of the inventive compounds is illustrated below. Also, it should be noted that the disclosure of commonly-owned U.S. Pat. No. 6,919,341 is incorporated herein by reference.

### SYNTHESIS

### Example 100

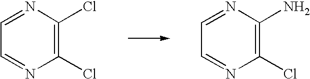

A mixture 2,3-dichloropyrazine (50 g, 0.34 mmol) and concentrated aqueous ammonium hydroxide (200 mL) was stirred at 85° C. in a closed pressure vessel for 4 days. The mixture was cooled to 25° C., water (200 mL) was added, and the mixture was filtered. The solid was washed with water (400 mL), then with dichloromethane (400 mL) and dried under vacuum. Compound 100 was isolated as a white solid 32.5 g (73%). ¹H NMR (400 MHz, DMSO-d₆ δ 7.93 (d, 1H), 7.55 (d, 1H), 6.79 (bs, 2H).

### Example 101

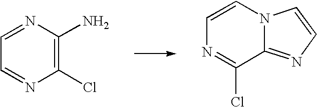

α-Bromo diethyl acetal (51.6 mL, 332.7 mmol, 2.5 eq) was added to a solution of 7.7 mL HBr (conc.) and 80 mL of H₂O. The reaction was heated at reflux for 1 h. The reaction was cooled and extracted 2× with Et₂O (200 mL). The Et₂O extracts were combined, washed with brine, and dried over Na₂SO₄ before being concentrated. The material was not left on the rotavap for an extended time or put under high vacuum. The oily residue was mixed with DME (200 mL) and the 2-amino-3-chloropyrazine (2, 17.240 g, 133.1 mmol) was added. HBr conc. (1-1.5 mL) was added and the reaction was heated at reflux. The reaction is heterogeneous reaction mixture, becomes homogenous after 10-15 minutes. After approximately 30 minutes a precipitate begins to form. After 1 hour at reflux the black reaction was cooled to room temperature, filtered, and washed with Et₂O (4×, 75 mL) to give compound 101 ¹H NMR (DMSO-d₆, 400 MHz) □ 8.70 (d, J=2.0 Hz, 1H), 8.32 (s, 1H), 7.93 (s, 1H), 7.79 (d, J=3.0 Hz, 1H). LC/MS shows a mixture of two products (one product by LC and two by MS). By MS there is a mass for X=Cl (major) MH⁺=154 (m/z) and one for X=Br (minor) MH⁺ 198 (m/z). This mixture gave the product in approximately 90% yield as the HBr salt.

### Example 102

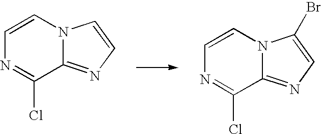

The 7-halo compound 101 (4.92 g, 20.2 mmol) was mixed with Br₂ (1.54 mL, 30.0 mmol) in AcOH (100 mL) at room temperature. After 5-10 minutes the reaction became homogeneous. After 1.5 hours a precipitate began to form. The reaction stirred at room temperature for 3 days. The reaction was concentrated in vacuo. The residue was taken up in 10% iso-PrOH in CH₂Cl₂ (300 mL) and washed with sat. NaHCO₃ (2×, 100 mL), 1M Na₂S₂O₃ (100 mL), and brine (100 mL). The organic layer was dried with Na₂SO₄ and concentrated in vacuo to give 4.460 g of the product, compound 102 (91% yield). ¹H NMR (DMSO-d₆, 400 MHz) □ 8.47 (d, J=4.8 Hz, 1H), 8.02 (s, 1H), 7.84 (d, J=4.4 Hz, 1H).

### Example 103

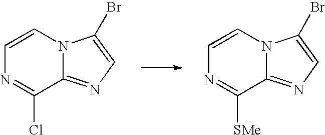

To a solution of compound 102 (13.0 g, 55.9 mmol) in DMSO (150 mL) was added sodium methanethiolate (4.70 g, 67.08 mmol) as a DMSO solution (100 mL) at room temperature. The reaction mixture was stirred at 100° C. for 16 hours. The mixture was cooled to 25° C. and added to a brine solution (300 mL), and extracted with 10% IPA/dichloromethane (300 mL, 3×). The combined organic layer was dried over anhydrous sodium sulfate and concentrated. Purification by column chromatography (SiO₂, ethyl acetate/hexanes (1:1)) afforded compound 103 as a yellow solid 10 g (70%). ¹H-NMR (400 MHz, DMSO-d₆ δ 8.15 (d, 1H), 7.88 (d, 1H), 7.83 (s, 1H), 2.6 (s, 3H).

### Example 104

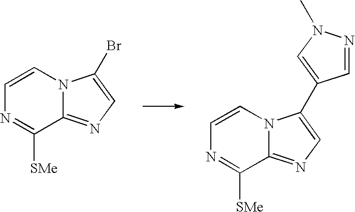

A mixture of compound 103 (5.0 g, 17.8 mmol), 1-methyl-4-(4,4,5,5-teramethyl-1,3,2-dioxaborolan-2-yl)-1H-pyrazole (7.44 g, 35.7 mmol), Pd(dppf)Cl₂ (1.46 g, 10 mol %), sodium carbonate (9.50 g, 89.5 mmol) in 1,2-dimethoxyethane (150 mL) and water (37 mL) was stirred at 70° C. under Argon for 16 hours. The solvents were evaporated and the residue was purified by column chromatography (SiO₂, ethyl acetate to 5% methanol/ethyl acetate) to afford compound 104 as a beige solid 3.80 g (86%). ¹H NMR (400 MHz, DMSO-d₆ δ 8.35 (s, 1H), 8.27 (d, 1H), 7.96 (d, 1H), 7.82 (s, 1H), 7.81 (d, 1H), 3.93 (s, 3H), 2.59 (s, 3H).

### Example 105

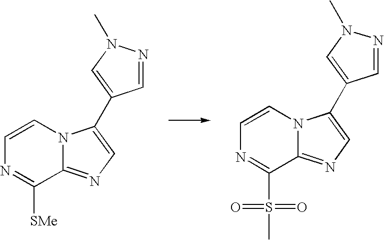

To a solution of compound 104 (3.0 g, 12.2 mmol) in dichloromethane (100 mL) at room temperature was added m-CPBA (5.75 g, 25.6 mmol) in one portion. The mixture was stirred at room temperature for 1 hour at which time thin layer chromatography (10% MeOH/ethyl acetate) indicated that the reaction was complete. The reaction mixture was poured into saturated aqueous sodium bicarbonate (100 mL). The layers were separated and the aqueous layer was extracted with dichloromethane (2×100 mL). The organic layers were combined and washed with brine (150 mL). The organic layer was dried over sodium sulfate, filtered, and concentrated under reduced pressure to yield a dark yellow oil. Purification by column chromatography (SiO₂, 10% methanol/ethyl acetate) afforded compound 105 as a yellow solid 2.10 g (62%). ¹H NMR (400 MHz, DMSO-d₆ δ 8.83 (d, 2H), 8.45 (s, 1H), 8.21 (s, 1H), 8.11 (d, 1H), 8.06 (d, 1H), 3.96 (s, 3H), 3.61 (s, 3H). HPLC-MS t_{R}=0.75 min (UV₂₅₄ₙₘ). Mass calculated for formula C₁₁H₁₁N₅O₂S 277.06; observed MH⁺ (LCMS) 278.1 (m/z).

### Example 106

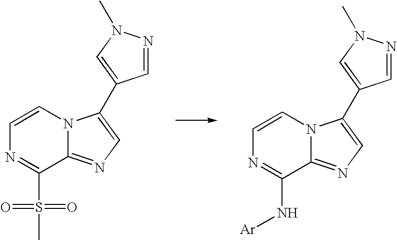

A solution of the respective aromatic amine (2 equivalents) in DMSO (1 mL) was treated with NaH (60% dispersion in oil, 2 equivalents) for 15 minutes at room temperature. Compound 105 (1 equivalent) was then added to this solution at room temperature and this solution was stirred at room temperature for 1 hour at which time thin layer chromatography (10% methanol/ethyl acetate) indicate the reaction was complete. The reaction mixture was diluted with sat. ammonium chloride (0.5 mL) and acetonitrile (0.5 mL). Purification by Prep-LC and conversion to a hydrochloric salt afforded compound 106.

### Examples 106-1-106-83

By essentially the same procedure given in Preparative Example 106, compounds given in Column 2 of Table 8 can be prepared from compound 105.

TABLE 8

| | | | LCMS | |
|---|---|---|---|---|
| | | | MH⁺ | HPLC |
| Example | Column 2 | MW | m/z | MS t_{R} |
| 106-1 | 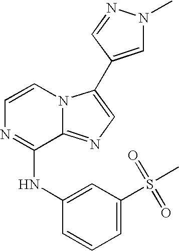 | 368.4 | 369.1 | 2.73 |
| 106-2 | 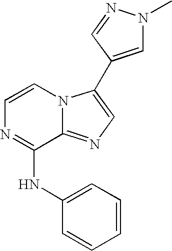 | 290.3 | 291.1 | 2.47 |
| 106-3 | 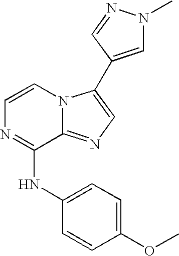 | 320.3 | 321.1 | 2.34 |
| 106-4 | 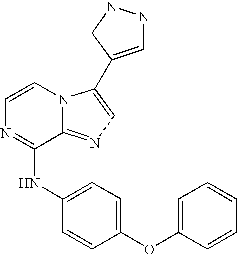 | 382.4 | 383.1 | 3.84 |
| 106-5 | 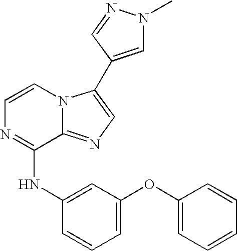 | 382.4 | 383.1 | 4.24 |
| 106-6 | 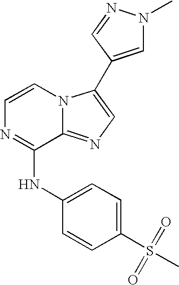 | 368.4 | 369.1 | 2.91 |
| 106-7 | 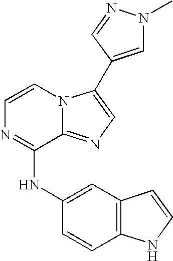 | 329.3 | 330.1 | 2.44 |
| 106-8 | 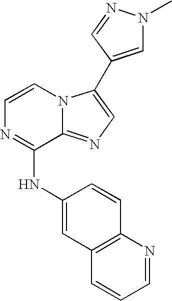 | 341.3 | 342.1 | 2.45 |
| 106-9 | 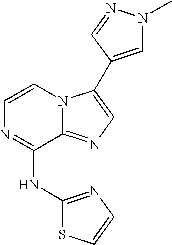 | 297.3 | 298.1 | 2.46 |
| 106-10 | 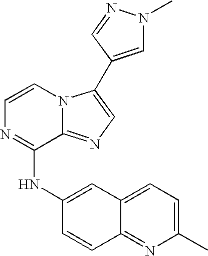 | 355.4 | 356.2 | 2.57 |
| 106-11 | 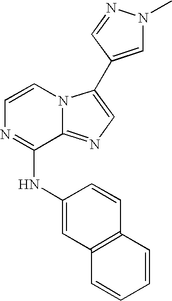 | 340.3 | 341.2 | 3.54 |
| 106-12 | 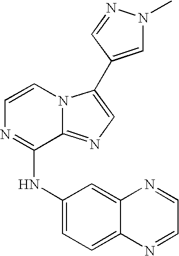 | 342.3 | 343.1 | 2.96 |
| 106-13 | 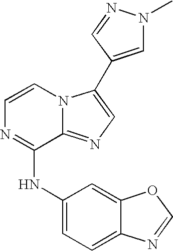 | 331.3 | 332.2 | 1.93 |
| 106-14 | 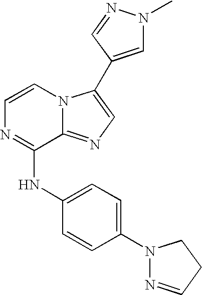 | 356.3 | 357.2 | 2.89 |
| 106-15 | 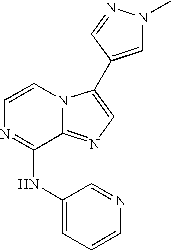 | 291.3 | 292.1 | 2.10 |
| 106-16 | 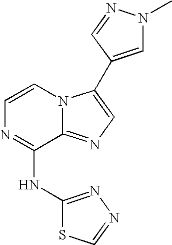 | 298.3 | 299.2 | 2.45 |
| 106-17 | 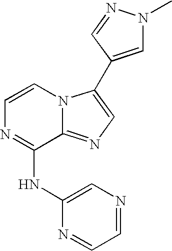 | 292.3 | 293.2 | 2.00 |
| 106-18 | 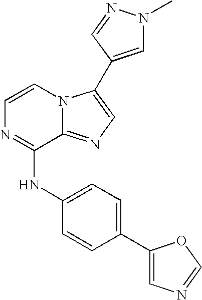 | 357.3 | 358.1 | 2.98 |
| 106-19 | 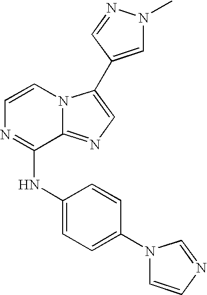 | 356.3 | 357.2 | 2.18 |
| 106-20 | 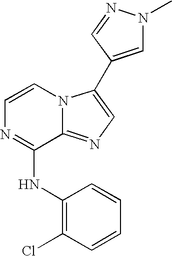 | 324.7 | 325.1 | 3.36 |
| 106-21 | 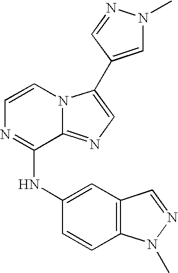 | 344.3 | 345.2 | 2.35 |
| 106-22 | 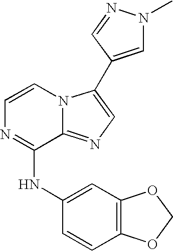 | 334.3 | 335.2 | 2.40 |
| 106-23 | 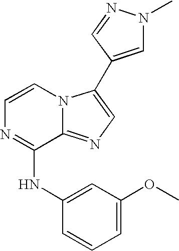 | 320.3 | 321.2 | 2.35 |
| 106-24 | 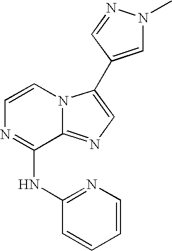 | 291.3 | 292.1 | 2.20 |
| 106-25 | 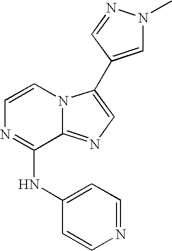 | 291.3 | 292.1 | 2.15 |
| 106-26 | 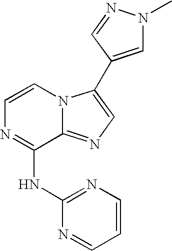 | 292.3 | 293.2 | 2.05 |
| 106-27 | 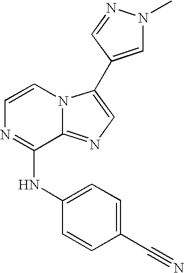 | 315.3 | 316.1 | 2.82 |
| 106-28 | 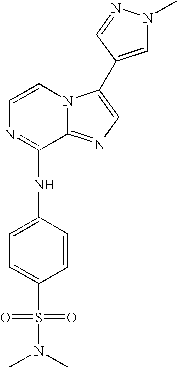 | 397.4 | 398.2 | 3.49 |
| 106-29 | 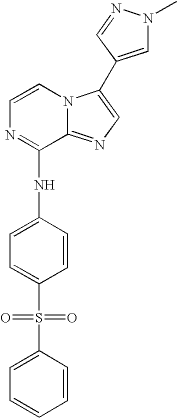 | 430.4 | 431.2 | 4.05 |
| 106-30 | 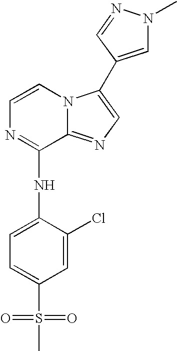 | 402.8 | 403.1 | 3.67 |
| 106-31 | 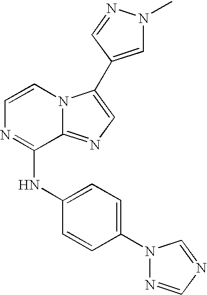 | 357.3 | 358.1 | 1.94 |
| 106-32 | 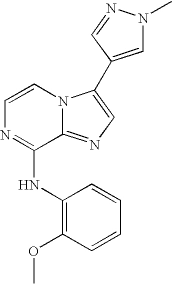 | 320.3 | 321.2 | 2.70 |
| 106-33 | 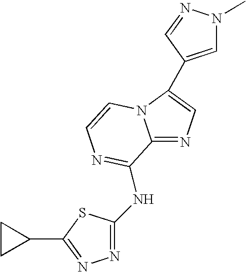 | 338.3 | 339.1 | 3.24 |
| 106-34 | 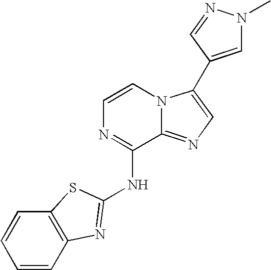 | 347.4 | 348.1 | 2.34 |
| 106-35 | 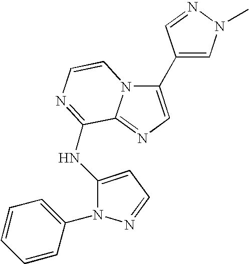 | 356.3 | 357.2 | 2.96 |
| 106-36 | 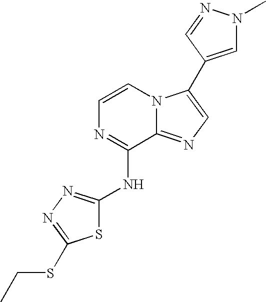 | 358.4 | 359.1 | 3.75 |
| 106-37 | 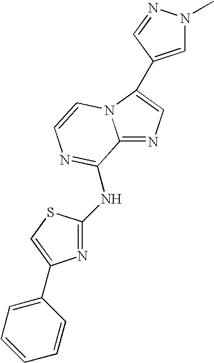 | 373.4 | 374.2 | 4.30 |
| 106-38 | 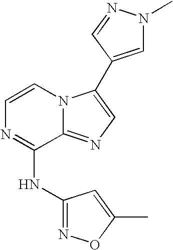 | 295.3 | 296.2 | 2.05 |
| 106-39 | 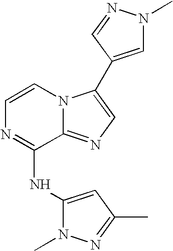 | 308.3 | 309.2 | 2.32 |
| 106-40 | 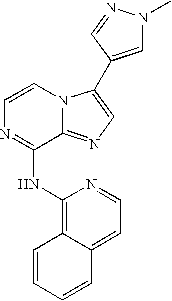 | 341.3 | 342.3 | 2.96 |
| 106-41 | 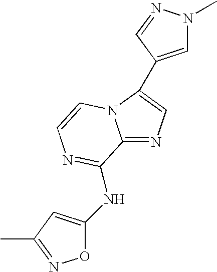 | 295.3 | 296.2 | 3.04 |
| 106-42 | 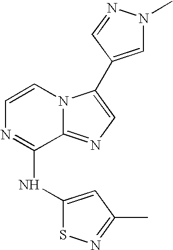 | 311.3 | 312.1 | 2.52 |
| 106-43 | 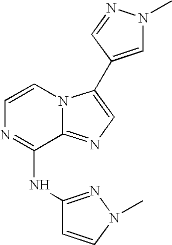 | 294.3 | 295.1 | 2.19 |
| 106-44 | 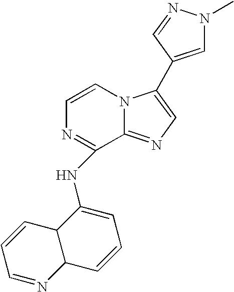 | 341.3 | 342.3 | 2.09 |
| 106-45 | 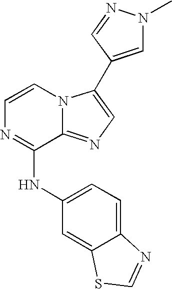 | 347.4 | 348.1 | 2.75 |
| 106-46 | 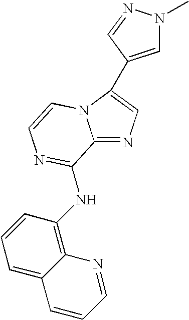 | 341.3 | 342.3 | 3.83 |
| 106-47 | 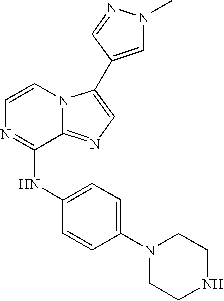 | 374.5 | 375.2 | 1.78 |
| 106-48 | 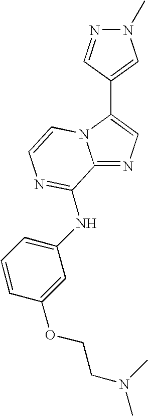 | 377.4 | 378.3 | 2.07 |
| 106-49 | 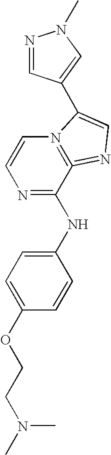 | 377.4 | 378.3 | 1.81 |
| 106-50 | 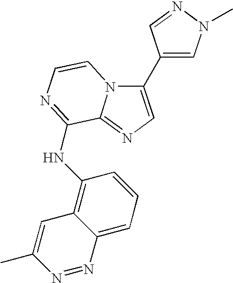 | 356.3 | 357.2 | 2.46 |
| 106-51 | 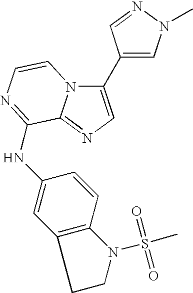 | 409.4 | 410.2 | 2.55 |
| 106-52 | 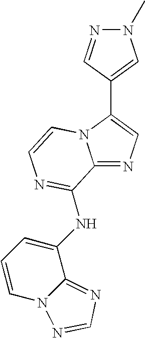 | 331.3 | 332.2 | 2.87 |
| 106-53 | 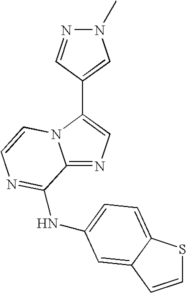 | 346.4 | 347.2 | 3.12 |
| 106-54 | 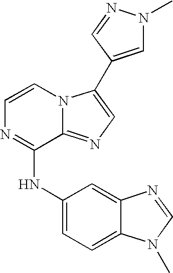 | 344.3 | 345.2 | 2.02 |
| 106-55 | 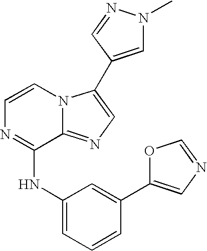 | 357.3 | 358.1 | 2.97 |
| 106-56 | 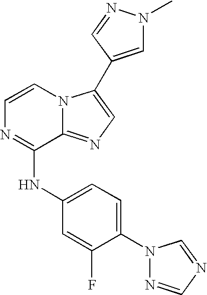 | 375.3 | 376.1 | 3.21 |
| 106-57 | 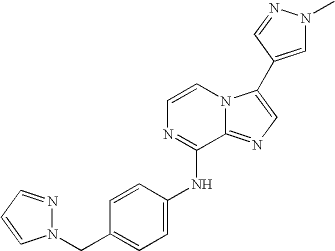 | 370.4 | 371.2 | 2.71 |
| 106-58 | 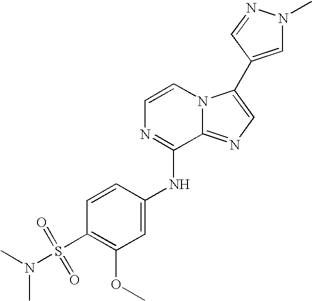 | 427.4 | 428.2 | 3.50 |
| 106-59 | 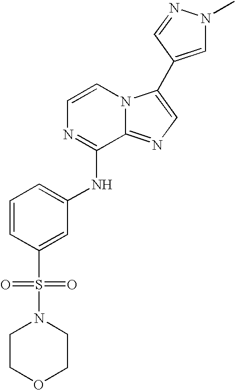 | 439.4 | 440.2 | 2.33 |
| 106-60 | 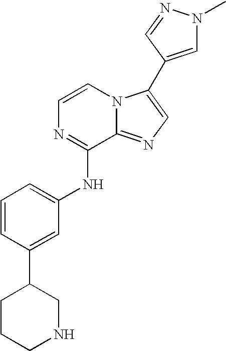 | 373.4 | 374.2 | 2.19 |
| 106-61 | 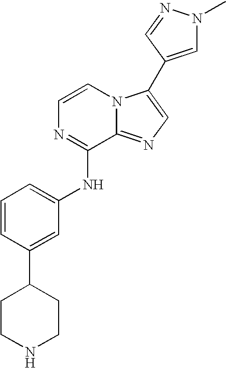 | 373.4 | 374.2 | 2.10 |
| 106-62 | 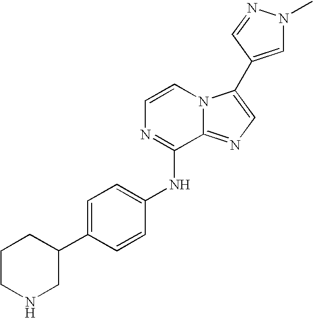 | 373.4 | 374.2 | 2.10 |
| 106-63 | 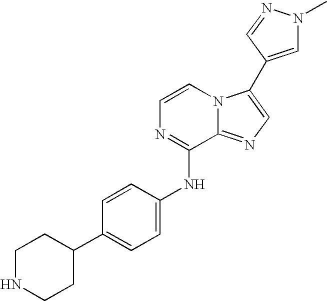 | 373.4 | 374.2 | 1.99 |
| 106-64 | 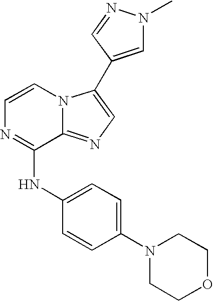 | 375.4 | 376.1 | 2.21 |
| 106-65 | 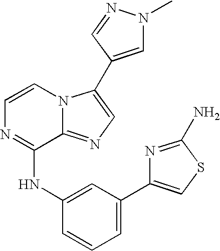 | 388.4 | 389.2 | 2.51 |
| 106-66 | 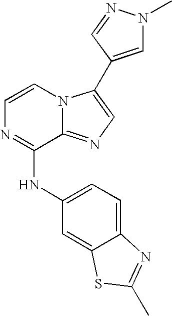 | 361.4 | 362.1 | 2.51 |
| 106-67 | 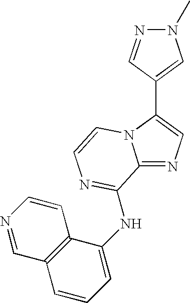 | 341.3 | 342.1 | 2.10 |
| 106-68 | 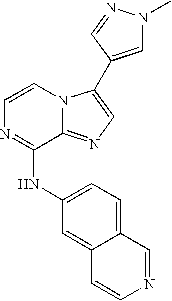 | 341.3 | 342.2 | 2.35 |
| 106-69 | 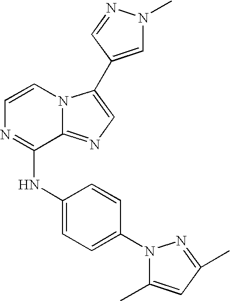 | 384.4 | 385.1 | 3.49 |
| 106-69 | 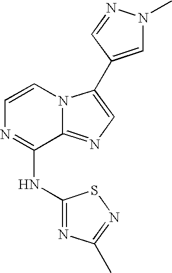 | 312.3 | 313.1 | 2.97 |
| 106-70 | 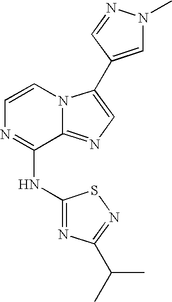 | 340.4 | 341.2 | 3.80 |
| 106-71 | 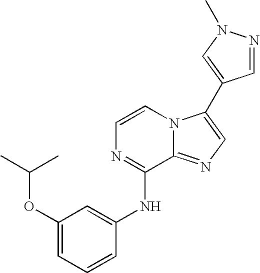 | 348.2 | 349.2 | 3.49 |
| 106-72 | 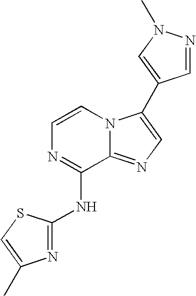 | 311.1 | 312.1 | 2.87 |
| 106-73 | 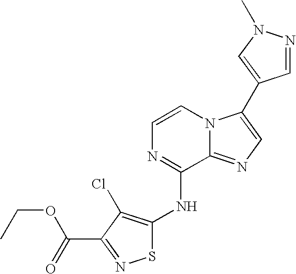 | 403.1 | 404.1 | 5.16 |
| 106-74 | 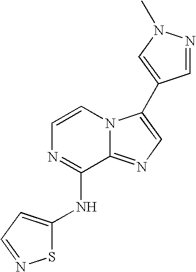 | 297.07 | 298.1 | 2.71 |
| 106-75 | 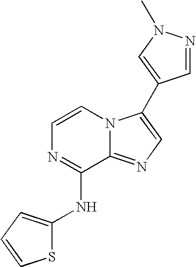 | 296.08 | 297.1 | 3.03 |
| 106-76 | 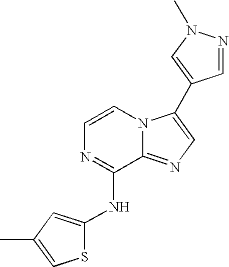 | 310.10 | 311.1 | 3.55 |
| 106-77 | 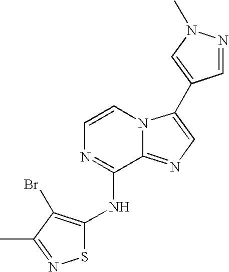 | 389.00 | 390.0 | 4.41 |
| 106-78 | 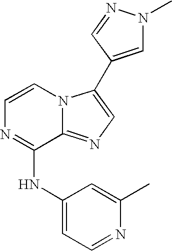 | 389.5 | 390.3 | 1.80 |
| 106-79 | 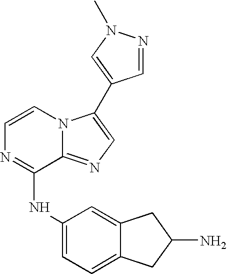 | 345.17 | 346.2 | 0.85 |
| 106-80 | 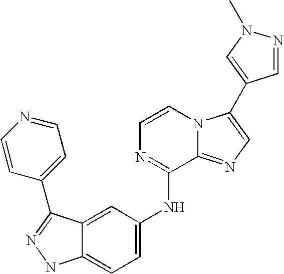 | 407.44 | 408.4 | 2.15 |
| 106-81 | 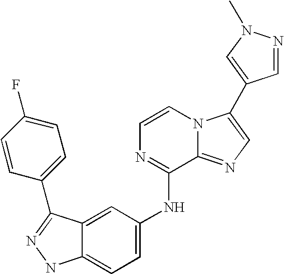 | 424.44 | 425.4 | 2.30 |
| 106-82 | 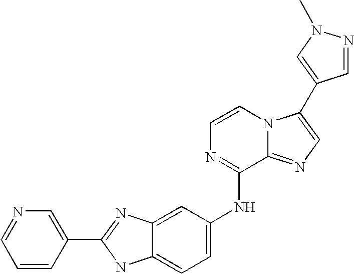 | 407.44 | 408.4 | 1.85 |
| 106-83 | 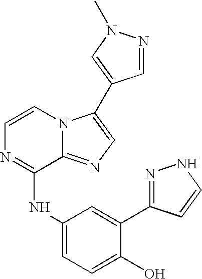 | 372.29 | 373.1 | 1.05 |

### Example 107

The compounds shown in column 2 of Table 9 were prepared as follows.

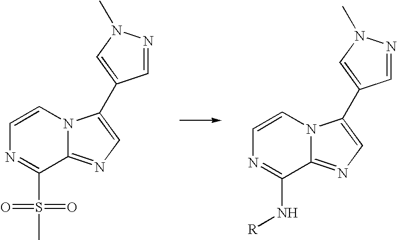

To a solution of compound 105 (1 equivalent) in NMP (0.5 mL) was added DIEA (10 equivalents), and the respective aliphatic amine (2 equivalents) at room temperature. The reaction was heated to 50° C. overnight. LC-MS analysis of the reaction indicates the reaction is complete. The crude reaction mixture was concentrated. Purification by Prep-LC and conversion to a hydrochloric salt afforded compound 107-1 to 107-13 as a white solid.

TABLE 9

| | | | LCMS | |
|---|---|---|---|---|
| Exam- | | | MH⁺ | HPLC |
| ple | Column 2 | MW | m/z | MS t_{R} |
| 107-1 | 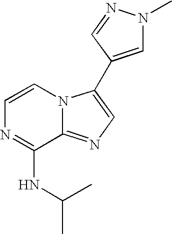 | 256.3 | 257.3 | 1.60 |
| 107-2 | 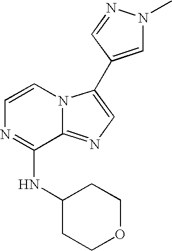 | 298.3 | 299.3 | 1.90 |
| 107-3 | 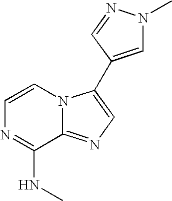 | 228.2 | 229.2 | 1.49 |
| 107-4 | 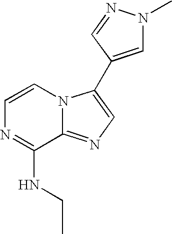 | 242.3 | 243.2 | 1.81 |
| 107-5 | 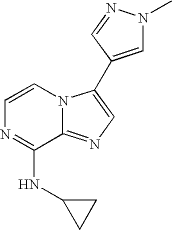 | 254.3 | 255.1 | 1.82 |
| 107-6 | 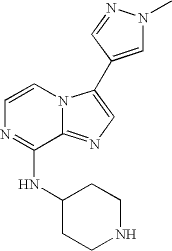 | 297.4 | 298.2 | 1.41 |
| 107-7 | 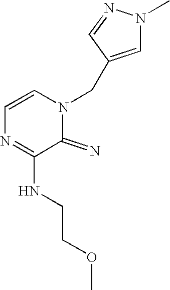 | 272.3 | 273.2 | 1.85 |
| 107-8 | 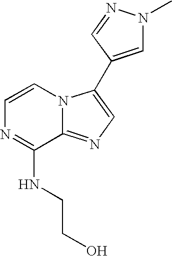 | 258.3 | 259.2 | 1.47 |
| 107-9 | 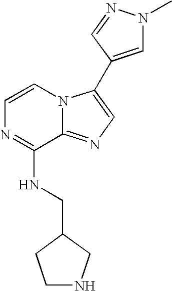 | 297.4 | 298.2 | 1.39 |
| 107-10 | 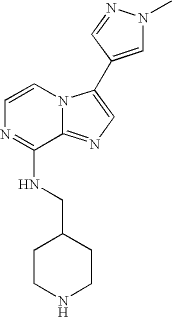 | 311.4 | 312.3 | 1.42 |
| 107-11 | 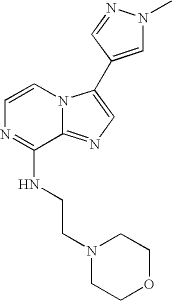 | 327.4 | 328.2 | 1.55 |
| 107-12 | 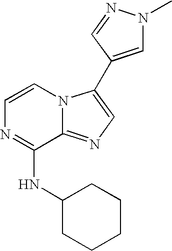 | 296.4 | 297.3 | 2.70 |
| 107-13 | 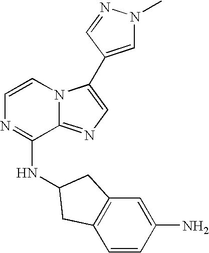 | 345.17 | 346.2 | 0.85 |

### Example 108

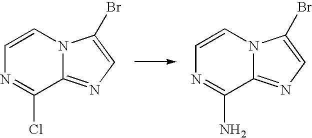

A mixture of compound 102 (2.00 g, 8.6 mmol), conc. aqueous NH₄OH (60 mL) and 2-propanol (6 mL) was stirred in a closed pressure vessel at 85° C. for 3 days. The reaction mixture was cooled to 25° C., diluted with water (120 mL) and stirred at 25° C. for 10 minutes. The resulting heterogeneous solution was filtered, the solid was washed with water (3×) and air dried overnight. This gave compound 108 as a beige solid 1.50 g (82%). ¹H-NMR (400 MHz, DMSO-d₆) δ 7.66 (s, 1H), 7.56 (d, 1H), 7.35 (d, 1H), 7.1 (bs, 2H).

### Example 109

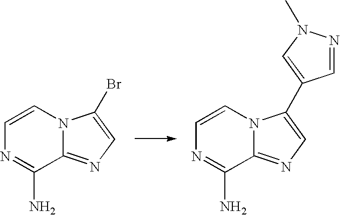

A mixture of compound 108 (1.50 g, 7.10 mmol), 1-methyl-4-(4,4,5,5-teramethyl-1,3,2-dioxaborolan-2-yl)-1H-pyrazole (2.94 g, 14.2 mmol), Pd (dppf)Cl₂ (0.58 g, 10 mol %), sodium carbonate (3.75 g, 35.4 mmol) in 1,2-dimethoxyethane (60 mL) and water (15 mL) was stirred at 80° C. under Argon for 16 hours. The solvents were evaporated and the residue purified by column chromatography (SiO₂ 5% methanol/ethyl acetate→15% methanol/ethyl acetate) to afford compound 109 as a grey solid 1.50 g (99%). ¹H NMR (400 MHz, DMSO-d₆ δ 8.27 (s, 1H), 7.88 (s, 1H), 7.72 (d, 1H), 7.64 (s, 1H), 7.26 (d, 1H), 6.91 (bs, 2H), 3.92(s, 1H)HPLC-MS t_{R}=0.3 nm (UV₂₅₄ₙₘ). Mass calculated for formula C₁₀H₁₀N₆, 214.1; observed MH⁺ (LC/MS) 215.2 (m/z).

### Example 110

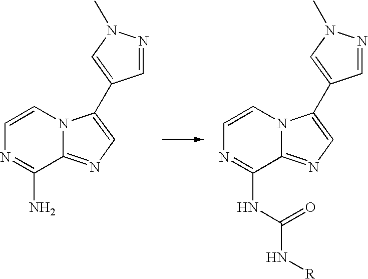

A solution of compound 109 (1 equivalent) in DMF (1 mL) was treated with NaH (60% dispersion in oil, 1.2 equivalents) for 15 minutes at room temperature. The respective isocyanate (1 equivalent) was then added to this solution at room temperature and the resultant solution was stirred overnight. When LC-MS analysis indicated the reaction was complete, the reaction mixture was concentrated. Purification by Prep-LC and conversion to a hydrochloric salt afforded compounds 110-1 to 110-4.

TABLE 10

| | | | LCMS | |
|---|---|---|---|---|
| | | | MH⁺ | HPLC |
| Example | Column 2 | MW | m/z | MS t_{R} |
| 110-1 | 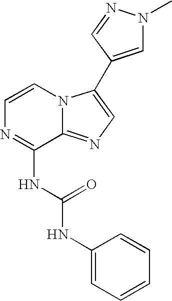 | 333.4 | 334.1 | 4.10 |
| 110-2 | 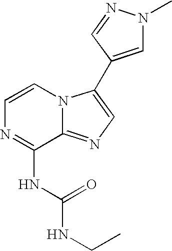 | 285.3 | 286.2 | 2.30 |
| 110-3 | 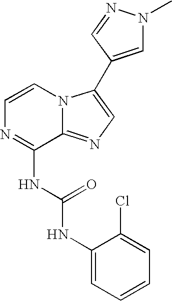 | 367.8 | 368.2 | 3.60 |
| 110-4 | 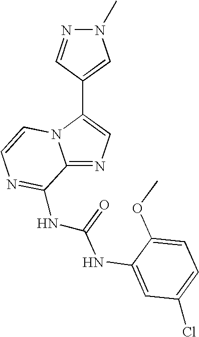 | 397.8 | 398.2 | 3.60 |

### Example 111

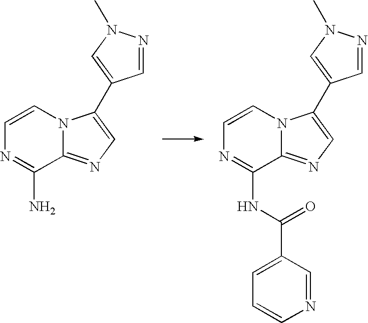

To a solution of nicotinic acid (25.0 mg, 0.203 mmol) in DMF (1.5 mL) was added compound 109 (65.2 mg, 0.304 mmol) and diisopropylethylamine (0.159 mL, 0.91 mmol). The reaction mixture was stirred at room temperature for 10 minutes, cooled to 0° C. (ice-bath) and then added HATU (115.6 mg, 0.304 mmol) and catalytic DMAP. The reaction mixture was allowed to warm to room temperature and then heated to 70° C., stirred overnight. LC-MS analysis indicated the reaction was complete. The reaction mixture was concentrated. Purification by Prep-LC and conversion to a hydrochloric salt afforded compound 111. HPLC-MS t_{R}=1.78 min (UV₂₅₄ₙₘ). Mass calculated for formula C₁₆H₁₃N₇O, 319.12; observed MH⁺ (LC/MS) 320.2 (m/z).

### Example 112

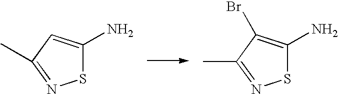

5-Amino-3-methyl isothiazole hydrochloride (5.00 g, 33.2 mmol) was added to water (35 mL). The insolubles were filtered and the filtrate's pH was adjusted to 10 with the addition of 2N NaOH. The mixture was stirred for five minutes and extracted with ethyl ether. The organic layer was separated and the aqueous layer was saturated with NaCl, extracted with ethyl ether (10 mL, 2×). The combined ether extracts were washed with brine, dried over sodium sulfate and then concentrated to afford compound 112 as dark orange oil, 3.12 g (82%). ¹H-NMR (400 MHz, DMSO-d₆ δ 6.5 (bs, 2H), 5.9 (s, 1H), 2.1 (s, 3H).

5-amino-3-methyl isothiazole (1.00 g, 8.75 mmol) was slurried in CCl₄ (30 mL) under an atmosphere of argon. N-Bromosuccinimide (1.56 g, 8.75 mmol) was added portion-wise to the amine slurry over a 10 minute period at room temperature. The reaction stirred at 65° C. for 1.5 hours. Thin layer chromatography (DCM/Hexanes 1:1) indicates the reaction is complete. The reaction mixture was cooled to room temperature and diluted with ethyl ether (40 mL). The resulting mixture was cooled to 5° C. for 30 minutes and filtered to remove any solid material. The filtrate was concentrated to yield a dark red solid that was dissolved in ethyl acetate and washed with water (100 mL, 2×). The organic layer was separated, washed with brine, dried over anhydrous sodium sulfate, and concentrated under vacuum to afford compound 112 as a dark red solid (1.49 g, 88%). This was used without further purification. ¹H-NMR (400 MHz, DMSO-d₆) δ 6.7 (bs, 2H), 2.2 (s, 3H).

### Example 113

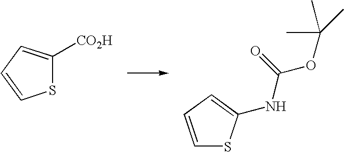

A solution of thiophene2-carboxylic acid (1.00 g, 7.8 mmol), diphenylphosphoryl azide (2.15 g, 7.80 mmol) and triethylamine (1.1 mL, 7.8 mmol) in tert-butanol (20 mL) was heated at reflux for 5 hours, at which time thin layer chromatography (DCM/Hexanes) indicates the reaction is complete. The reaction mixture was cooled to room temperature, poured into water and extracted with diethyl ether (3×). The combined ether extracts were washed with brine, dried over anhydrous sodium sulfate, and then concentrated to afford a beige solid. Purification by column chromatography (SiO₂, DCM/Hexanes) afforded compound 113 as a white solid 1.07 g (69%). ¹H-NMR (400 MHz, DMSO-d₆)δ 6.87 (dd, 1H), 6.77 (m, 1H), 6.5 (dd, 1H), 1.46 (s, 9H).

### Example 114

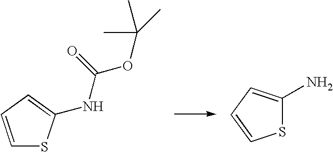

A solution of compound 113 (0.20 g, 1.00 mmol) was stirred in 4 M HCl solution in 1,4-dioxane (3 mL) at 50° C. for 2 hrs at which time thin layer chromatography (DCM/Hexanes) indicated the reaction was complete. The reaction mixture was cooled to room temperature and concentrated under vacuum. The residue was diluted with acetonitrile, sonicated, and concentrated to afford compound 114 as a grey solid 0.13 g (96%). ¹H-NMR (400 MHz, DMSO-d₆) δ 7.38 (m, 1H), 7.02 (m, 1H), 6.97 (m, 2H).

### Example 115

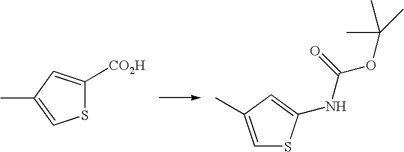

A solution of 4-methyl thiophene-2carboxylic acid (1.00 g, 7.03 mmol), diphenylphosphoryl azide (1.94 g, 7.03 mmol) and triethylamine (0.98 mL, 7.03 mmol) in tert-butanol (20 mL) was heated at reflux for 5 hours, at which time thin layer chromatography (DCM/Hexanes) indicates the reaction is complete. The reaction mixture was cooled to room temperature, poured into water and extracted with diethyl ether (3×). The combined ether extracts were washed with brine, dried over anhydrous sodium sulfate and then concentrated to afford a beige solid. Purification by column chromatography (SiO₂ DCM/Hexanes) afforded compound 115 as a white solid 0.96 g (64%). ¹H-NMR (400 MHz, DMSO-d₆) δ 6.42(s, 1H), 6.35 (d, 1H), 1.46 (s, 9H).

### Example 116

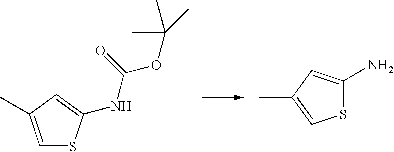

A solution of compound 115 (0.21 g, 1.00 mmol) was stirred in 4 M HCl solution in 1,4-dioxane (3 mL) at 50° C. for 2 hrs at which time thin layer chromatography (DCM/Hexanes) indicated the reaction was complete. The reaction mixture was cooled to room temperature and concentrated under vacuum. The residue was diluted with acetonitrile, sonicated, and concentrated to afford compound 116 as a grey solid 0.14 g (91%). ¹H-NMR (400 MHz, DMSO-d₆) δ 11.6 (bs, 2H) 6.83 (d, 1H), 6.7 (d, 1H), 4.55 (s, 3H).

### Example 117

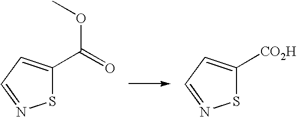

To a solution of isothiazole-5-carboxylic acid methyl ester (0.50 g, 3.49 mmol) in THF/MeOH (20 mL/5 mL) was added 1N NaOH (5.24 mL, 5.24 mmol) at room temperature. The reaction mixture was stirred at room temperature for 16 hours at which time thin layer chromatography indicated the reaction was complete. The reaction mixture was acidified to pH 2 with 1N HCl resulting in the formation of a precipitate, this was filtered and dried to afford compound 2 as a beige solid 0.35 g (76%). ¹H-NMR (400 MHz, DMSO-d₆) δ 8.69 (d, 1H), 7.85 (d, 1H).

### Example-118

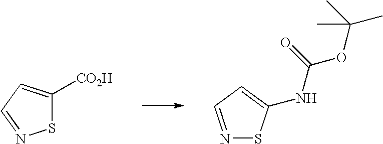

A solution of compound 117 (0.35 g, 2.67 mmol), diphenylphosphoryl azide (0.57 mL, 2.67 mmol) and triethylamine (0.37 mL, 2.67 mmol) in tert-butanol (10 mL) was heated at reflux for 5 hours, at which time thin layer chromatography (DCM/Hexanes) indicates the reaction is complete. The reaction mixture was cooled to room temperature, poured into water and extracted with diethyl ether (3×). The combined ether extracts were washed with brine, dried over sodium sulfate, and concentrated to afford a beige solid. Purification by column chromatography (SiO₂, 40% ethyl acetate/hexanes) afforded compound 121 as a white solid 0.245 g (46%). ¹H-NMR (400 MHz, DMSO-d₆) δ 8.15(d, 1H), 6.72 (d, 1H), 1.48 (s, 9H).

### Example 119

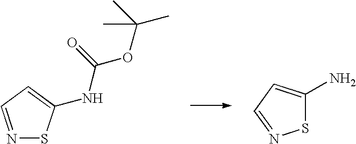

A solution of compound 118 (0.25 g, 1.22 mmol) was stirred in 4 M HCl solution in 1,4-dioxane (3 mL) at 50° C. for 2 hrs at which time thin layer chromatography (DCM/Hexanes) indicated the reaction was complete. The reaction mixture was cooled to room temperature and concentrated under vacuum. The residue was diluted with acetonitrile, sonicated, and concentrated to afford compound 119 as a grey solid 0.15 g (93%). ¹H-NMR (400 MHz, DMSO-d₆) δ 8.09 (d, 1H), 6.26 (d, 1H).

### Example 120

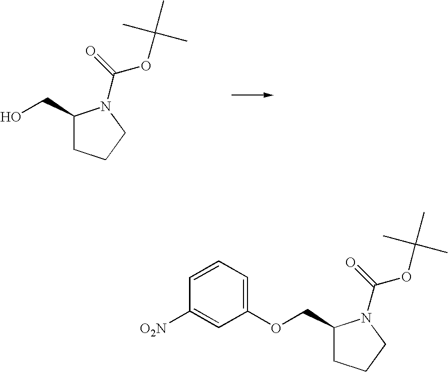

To a solution of 3-nitrophenol (0.35 g, 2.48 mmol, 1.00 equiv), triphenyl phosphine (0.68 g, 2.61 mmol, 1.05 equiv) and Boc-L-prolinol (0.53 g, 2.61 mmol, 1.05 equiv) in THF (10 mL) at rt was added drop wise diisopropyl azodicarboxylate (0.51 mL, 2.61 mmol, 1.05 equiv). The resulting solution was allowed to stir overnight at rt. Concentration and purification by chromatography (30% ethyl acetate in hexanes) afforded the title compound as a viscous oil (0.39 g, 48%).

### Example 121

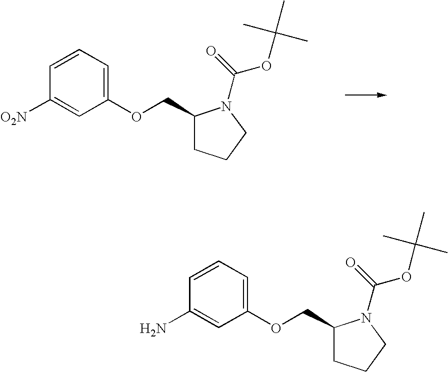

A suspension of (S)-2-(3-nitro-phenoxymethyl)-pyrrolidine-1-carboxylic acid tert-butyl ester (0.39 g) and 10% Pd/C (0.20 g) in ethanol was stirred under an hydrogen atmosphere (1 atm at balloon pressure) for 3.5 hr. The reaction mixture was filtered through a bed of Celite using ethyl acetate as solvent. Concentration afforded the title compound as a clear oil (0.316 g, 90%). ¹H NMR (400 MHz, DMSO-d₆) □ 6.85 (t, 1H), 6.10 (appt, 3H), 5.00 (br s, 2H), 3.91 (app t, 1H), 3.71 (app t, 1H), 3.28-3.19 (m, 2H), 1.95-1.75 (m, 4H), 1.38 (s, 9H). LCMS: (MH−C₄H₈)⁺=237.3.

### Example 122

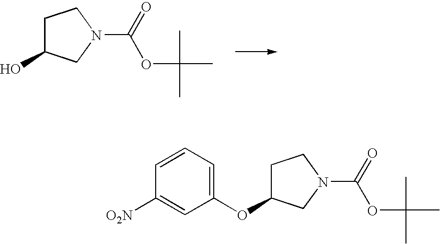

To a suspension of NaH (0.17 g, 4.4 mmol, 1.1 equiv) in DMSO (4 mL) at rt was added (3S)-1-Boc-3-pyrrolidinol (0.75 g, 4.0 mmol, 1.00 equiv) in one portion. After stirring for 20 min, 3-fluoronitrobenzene (0.51 g, 3.6 mmol, 0.90 equiv) was added drop wise and the resulting suspension was stirred an additional 1.5 hours at rt. The reaction mixture was quenched with the addition of saturated, aqueous NH₄Cl and extracted with ethyl acetate (3×). The combined organic layers were washed with brine, dried (Na₂SO₄), and concentrated. Purification of the crude residue by chromatography (30% ethyl acetate in hexanes) afforded 3-(3-nitro-phenoxy)-pyrrolidine-1-carboxylic acid tert-butyl ester as a bright yellow oil (0.676 g, 60%).

### Example 123

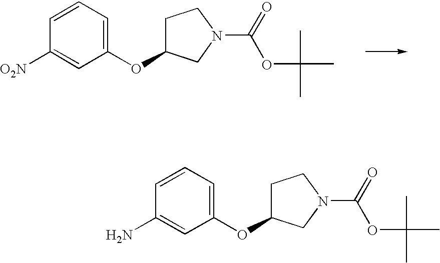

A suspension of 3-(3-nitro-phenoxy)-pyrrolidine-1-carboxylic acid tert-butyl ester (0.676 g) and 10% Pd/C (0.200 g) in ethanol was stirred under an hydrogen atmosphere (1 atm at balloon pressure) for 16 hr. The reaction mixture was filtered through a bed of Celite using ethyl acetate as solvent. Concentration afforded the title compound as a clear oil (0.529 g, 87%). ¹H NMR (400 MHz, DMSO-d₆) □ 6.87 (t, 1H), 6.14-6.03 (m, 3H), 5.04 (br s, 2H), 4.81 (br s, 1H), 3.52-3.23 (m, 4H), 2.10-1.95 (m, 2H), 1.38 (d, 9H). LCMS: (MH−C₄H₈)⁺=223.1.

### Example 124

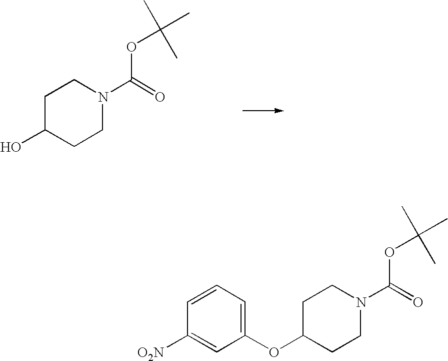

To a suspension of NaH (0.165 g, 4.14 mmol, 1.1 equiv) in DMSO (4 mL) at rt was added 1-BOC-4-hydroxypiperidine (0.794 g, 3.94 mmol, 1.00 equiv) in one portion. After stirring for 20 min, 3-fluoronitrobenzene (0.62 g, 4.34 mmol, 1.10 equiv) was added dropwise and the resulting suspension was stirred an additional 16 hours at rt. The reaction mixture was quenched with the addition of saturated, aqueous NH₄Cl and extracted with ethyl acetate (50 mL, 3×). The combined organic layers were washed with brine, dried with sodium sulfate and concentrated. Purification of the crude residue by chromatography (30% ethyl acetate in hexanes) afforded 4-(3-nitro-phenoxy)-piperidine-1-carboxylic acid tert-butyl ester as a dark orange oil (0.390 g, 31%).

### Example 125

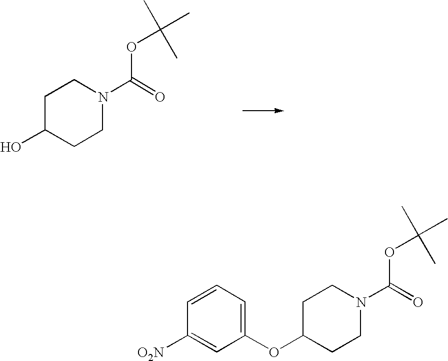

A suspension of 4-(3-nitro-phenoxy)-piperidine-1-carboxylic acid tert-butyl ester (0.390 g) and 10% Pd/C (0.100 g) in ethanol was stirred under an hydrogen atmosphere (1 atm at balloon pressure) for 16 hr. The reaction mixture was filtered through a bed of Celite using ethyl acetate as solvent. Concentration afforded 4-(3-amino-phenoxy)-piperidine-1-carboxylic acid tert-butyl ester as a clear oil (0.353 g, 90%). ¹H NMR (400 MHz, DMSO-d₆) E16.85 (t, 1H), 6.15-6.05 (m, 3H), 4.99 (br s, 2H), 4.43-4.30 (m, 1H), 3.67-3.53 (m, 2H), 3.20-3.06 (m, 2H), 1.89-1.80 (m, 2H), 1.53-1.4 (m, 2H), 1.38 (s, 9H).

### Example 126

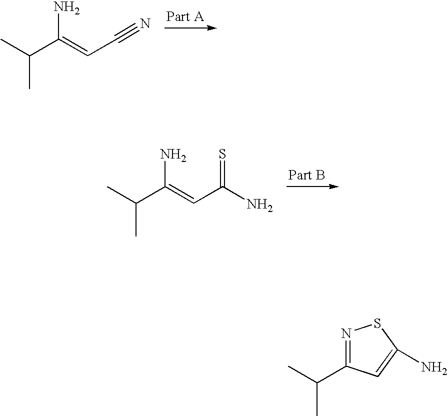 Part A:

A solution of 3-amino-4-methyl-pent-2-enenitrile (Hackler, R. E., et. al. J. Heterocyclic Chem. 1989, 1575-1578) (0.700 g, 6.35 mmol, 1.00 equiv) in 1/1 THF/ethanol (5 mL) was cooled to 0° C. and treated with hydrogen sulfide gas for ca. 5 min. The tube was sealed and heated at 90° C. (16 hr). The reaction vessel was cooled in an ice-bath, carefully vented and the reaction mixture was concentrated. The crude residue was used in Part B without further purification.

Part B:

A suspension of the crude residue from Part A and potassium carbonate (1.34 g, 9.71 mmol, 2.0 equiv) in diethyl ether (7 mL) was heated at reflux. To the reaction mixture was added drop wise a solution of iodine (1.2 g, 4.85 mmol, 1.00 equiv) in ether (7 mL). The mixture was heated at reflux for an additional 2 hr. Water and ethyl acetate were added. The aqueous phase was washed with ethyl acetate and the combined organic phases were washed with water, brine, and dried with sodium sulfate. Purification of the residue by chromatography (30% ethyl acetate in hexanes) afforded 449 mg (50% yield based on 3-amino-4-methyl-pent-2-enenitrile) of 3-isopropyl-isothiazol-5-ylamine as a waxy, orange solid. ¹H NMR (400 MHz, DMSO-d₆) □ 6.46 (br s, 2H), 5.97 (s, 1H), 3.31 (dq, 1H), 1.12 (d, 6H), (MH)⁺ (LCMS) 143.1 (m/z)

### Example 127

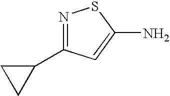

The compound of example 127 was prepared by the same procedure set forth in the above example 126, MH⁺ (LCMS) 141.1 (m/z).

### Example 128

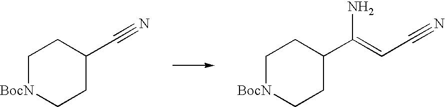

4-(1-Amino-2-cyano-vinyl)-piperidine-1-carboxylic acid tert-butyl ester was prepared from 4-cyano-piperidine-1-carboxylic acid tert-butyl ester (10.0 mmol) according to the procedure described in WO 2004/014910 A1 (p. 32). The crude residue was used in the next step without purification.

### Example 129

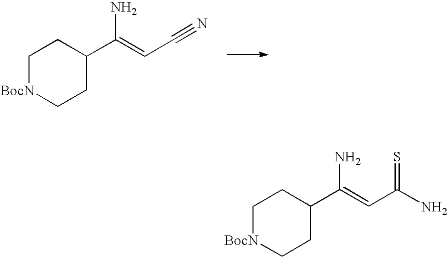

A solution of crude 4-(1-amino-2-cyano-vinyl)-piperidine-1-carboxylic acid tert-butyl ester (compound 128) in 1:1 THF/Ethanol (10 mL) was cooled to 0° C. and treated with hydrogen sulfide gas for ca. 5 min. The tube was sealed and heated at 85° C. for 4 hr. The reaction vessel was cooled in an ice-bath, carefully vented and the reaction mixture was concentrated. The crude residue was used in the next step without purification.

### Example 130

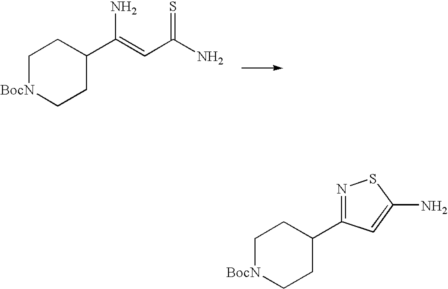

To the crude product from example 129 and potassium carbonate (2.1 g, 15.0 mmol) in diethyl ether (15 mL) at rt was added drop wise a solution of iodine (1.02 g, 4.0 mmol) in ether (6 mL). The mixture was stirred at rt for an additional 2 hr. Water and ethyl acetate were added. The aqueous phase was washed with ethyl acetate and the combined organic extracts were washed with water, brine and dried with sodium sulfate. Purification of the residue by chromatography (40% ethyl acetate in hexanes) afforded 250 mg of 4-(5-amino-isothiazol-3-yl)-piperidine-1-carboxylic acid tert-butyl ester (9% yield based on 4-cyano-piperidine-1-carboxylic acid tert-butyl ester). ¹H NMR (400 MHz, DMSO-d₆) □ 6.51 (br s, 2H), 5.98 (s, 1H), 4.02-3.88 (m, 2H), 2.82-2.68 (m, 2H), 2.68-2.58 (m, 2H), 2.82-2.75 (m, 2H), 2.60-2.51 (m, 1H), 1.38 (s, 9H). LCMS: (M−C₄H₈)⁺=228.1.

### Example 131

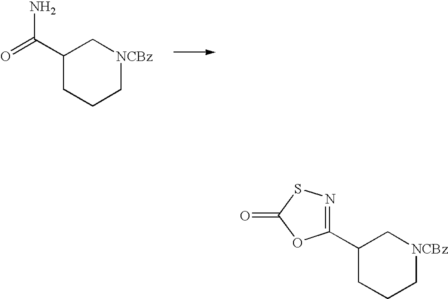

To a suspension of benzyl 4-(amino carbonyl)tetrahydro-1(2H)-pyridinecarboxylate (2.79 g, 10.6 mmol, 1.00 equiv) in toluene (50 mL) was added chlorocarbonylsulfonyl chloride (0.97 mL, 11.7 mmol, 1.1 equiv) drop wise. The resulting suspension was refluxed for one hour, allowed to cool and then concentrated. The residue was dissolved in ethyl acetate and washed with saturated sodium bicarbonate, water, brine and dried with sodium sulfate. Concentration afforded 3-(2-oxo-[1,3,4]oxathiazol-5-yl)-piperidine-1-carboxylic acid benzyl ester as a clear, pale yellow oil, MH⁺ (LCMS) 321.1 (m/z).

### Example 132

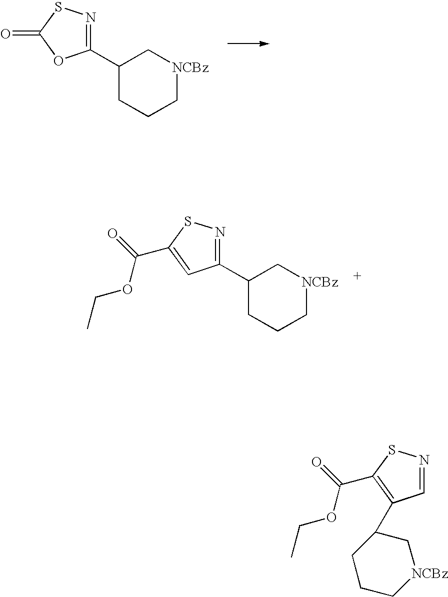

A solution of the crude residue from example 131 and ethyl propiolate (2 mL) in xylenes (15 mL) was heated in a sealed tube at 150° C. for 4 hr. Concentration and chromatographic purification (25% ethyl acetate and hexanes) afforded 3-(5-ethoxycarbonyl-isothiazol-3-yl)-piperidine-1-carboxylic acid benzyl ester and 3-(4-ethoxycarbonyl-isothiazol-3-yl)-piperidine-1-carboxylic acid benzyl ester as a 1:1 mixture (1.24 g), MH⁺ (LCMS) 375.1 (m/z).

### Example 133

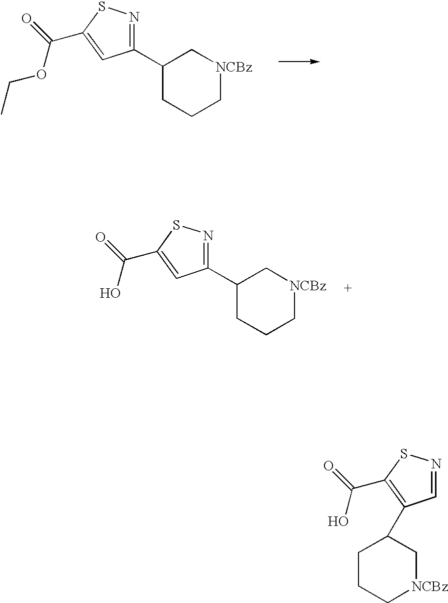

A solution of the residue from example 132 in THF (20 mL) and 1 N LiOH (6.7 mL) was heated at 50° C. for 4 hr. The reaction mixture was poured into ethyl acetate and acidified to pH 3 with 1 N HCl. The aqueous phase was extracted with ethyl acetate and the combined organic extracts were washed with water, brine, and dried with sodium sulfate. Concentration afforded 3-(5-carboxy-isothiazol-3-yl)-piperidine-1-carboxylic acid benzyl ester and 3-(4-carboxy-isothiazol-3-yl)-piperidine-1-carboxylic acid benzyl ester as a 1:1 mixture (1.02 g), MH⁺ (LCMS) 347.1 (m/z).

### Example 134 and 134-1

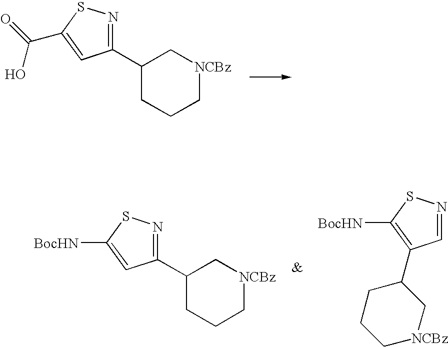

To a solution of crude residue from example 133 (1.02 g, 2.94 mmol, 1.00 equiv), N,N-diisopropylethylamine (0.56 mL, 3.23 mmol, 1.1 equiv) in tert-BuOH (25 mL) at rt was added diphenylphosphoryl azide (0.7 mL, 3.2 mmol, 1.1 equiv) drop wise. The resulting solution was refluxed for one hour and concentrated. The regioisomers were separated chromatographically (15% ethyl acetate in hexanes) affording 3-(5-tert-butoxycarbonylamino-isothiazol-3-yl)-piperidine-1-carboxylic acid benzyl ester (134; R_{f}=0.50 (15% ethyl acetate in hexanes), LCMS: (MH)⁺=418.1 m/z) and 3-(4-tert-butoxycarbonylamino-isothiazol-3-yl)-piperidine-1-carboxylic acid benzyl ester (134-1; R_{f}=0.31 (15% ethyl acetate in hexanes), MH⁺ (LCMS) 418.1 (m/z).

### Example 135

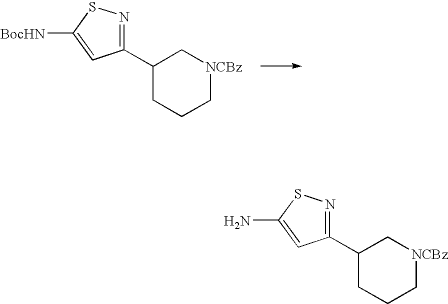

The crude residue from 134-1 was treated with 4 N HCl in dioxane at rt for 4 hours and then was concentrated. The residue was freeze-dried from a solution of acetonitrile and water. 3-(5-Amino-isothiazol-3-yl)-piperidine-1-carboxylic acid benzyl ester was used without further purification, MH⁺ (LCMS) 318.2 (m/z). 3-(4-Amino-isothiazol-3-yl)-piperidine-1-carboxylic acid benzyl ester was prepared using the same method, MH⁺ (LCMS) 318.2 (m/z).

### Example 135-1

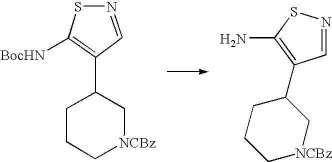

The crude residue from 134-1 was treated with 4 N HCl in dioxane at rt for 4 hours and then was concentrated. The residue was freeze-dried from a solution of acetonitrile and water. 3-(5-Amino-isothiazol-3-yl)-piperidine-1-carboxylic acid benzyl ester was used without further purification. MH⁺ (LCMS) 318.2 (m/z). 3-(4-amino-isothiazol-3-yl)-piperidine-1-carboxylic acid benzyl ester was prepared using the same method, MH⁺ (LCMS) 318.2 (m/z).

### Examples 136-141

By essentially the same procedure set forth in Example 106, the compounds shown in column 3 were prepared from compounds given in column 2.

TABLE 11

| Ex- | | | | LCMS | |
|---|---|---|---|---|---|
| am- | | | | MH⁺ | HPLC |
| ple | Column 2 | Column 3 | MW | m/z | MS t_{R} |
| 136 | 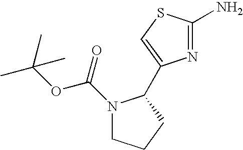 | 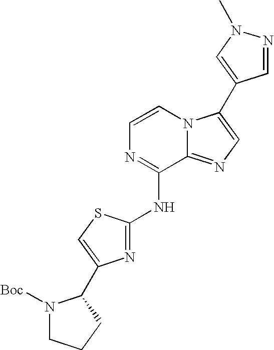 | 466.1 | 467.2 | 1.66 |
| 137 | 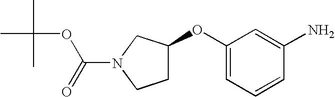 | 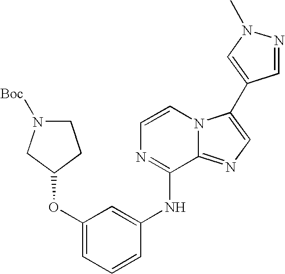 | 475.2 | 476.2 | 1.80 |
| 138 | 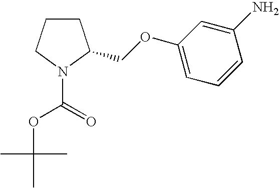 | 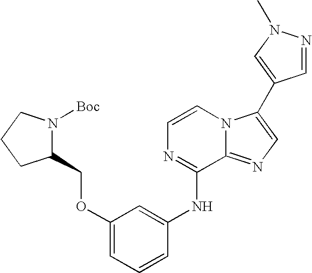 | 489.2 | 490.3 | 2.02 |
| 139 | 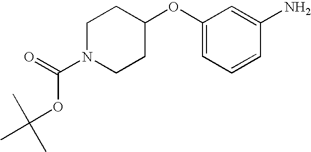 | 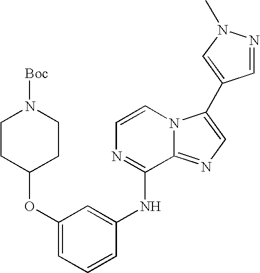 | 489.2 | 490.3 | 2.02 |
| 140 | 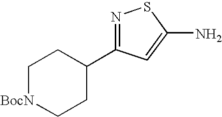 | 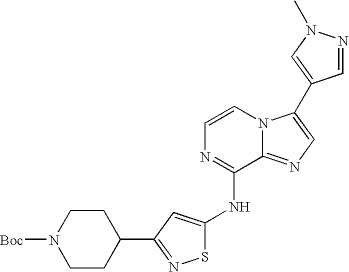 | 480.2 | 481.1 | 1.84 |
| 141 | 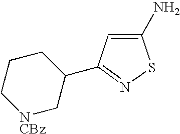 | 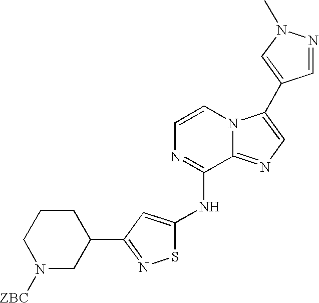 | 514.1 | 515.2 | 1.93 |
| 141-1 | 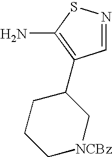 | 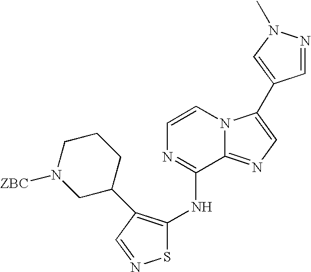 | 514.1 | 515.2 | 2.02 |

### Example 142

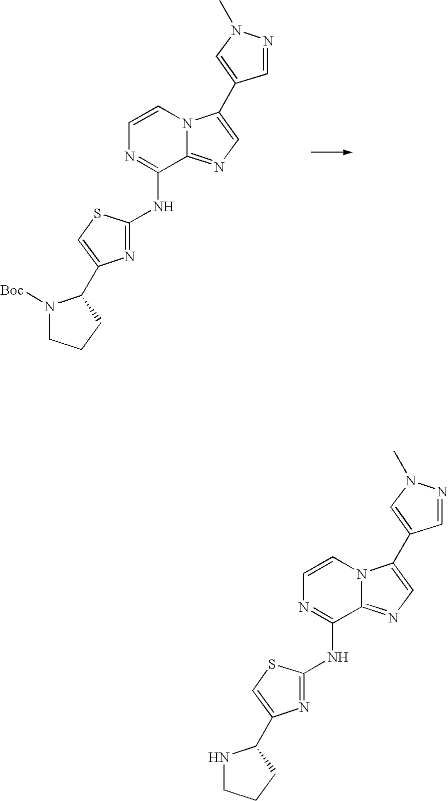

A solution of compound from example 121 (0.25 g,) was stirred in 4 N HCl solution in 1,4-dioxane (3 mL) at room temperature for 2 hrs at which time LC MS analysis indicated the reaction was complete. The reaction mixture concentrated under vacuum. The residue was diluted with acetonitrile, water, and lyophilized to afford compound 142; HPLC t_{R}=20.50 min, calculated molecular formula weight, 366.10; observed MH⁺ (LCMS) 367.2 (m/z).

By essentially the same procedure given in example 142, starting from compounds given in column 2, compounds given in column 3 in Table 12 can be prepared:

TABLE 12

| | | | | LCMS | |
|---|---|---|---|---|---|
| Exam- | | | | MH⁺ | HPLC |
| ple | Column 2 | Column 3 | MW | m/z | MS t_{R} |
| 143 | 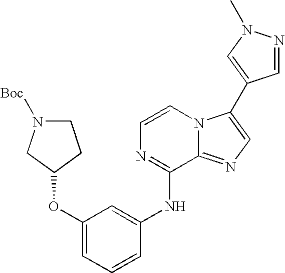 | 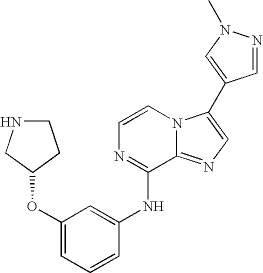 | 375.2 | 376.2 | 2.18 |
| 144 | 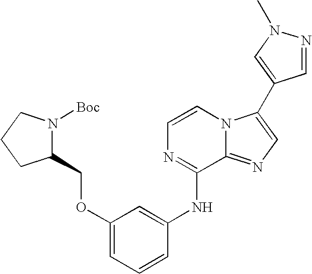 | 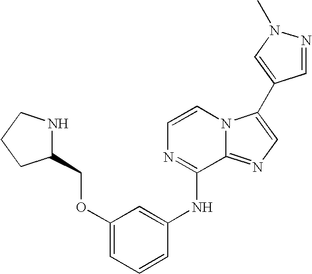 | 389.2 | 390.2 | 2.27 |
| 145 | 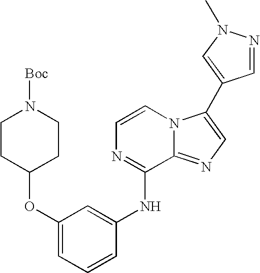 | 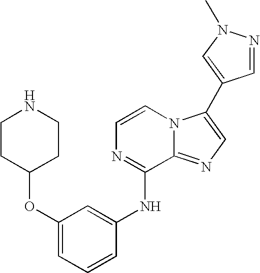 | 389.2 | 390.2 | 2.26 |
| 146 | 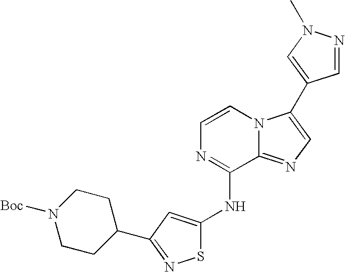 | 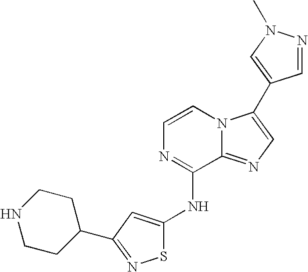 | 380.2 | 381.2 | 2.23 |
| 147 | 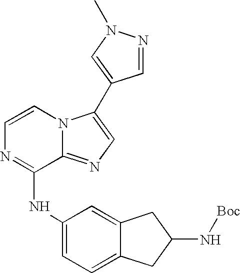 | 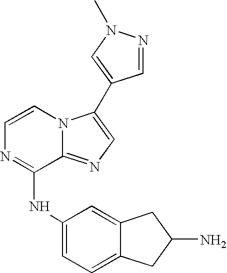 | 345.2 | 346.2 | 0.85 |

### Example 148

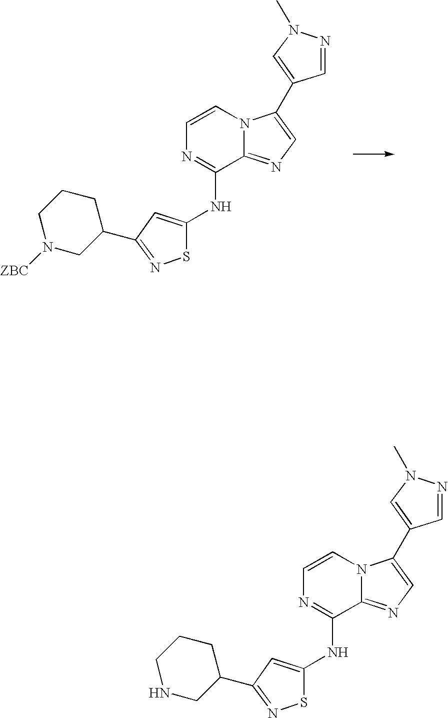

A suspension of compound from example 141 (0.05 g) and 4 N HCl in dioxane was stirred at 60° C. for 1 hr. The reaction mixture evaporated to dryness, dissolved in acetonitrile-water (1:1), and lyophilized to give the product 148. HPLC t_{R}=2.49 min, calculated molecular formula weight 380.2, observed MH⁺ (LCMS) 381.2 (m/z).

### Example 148-1

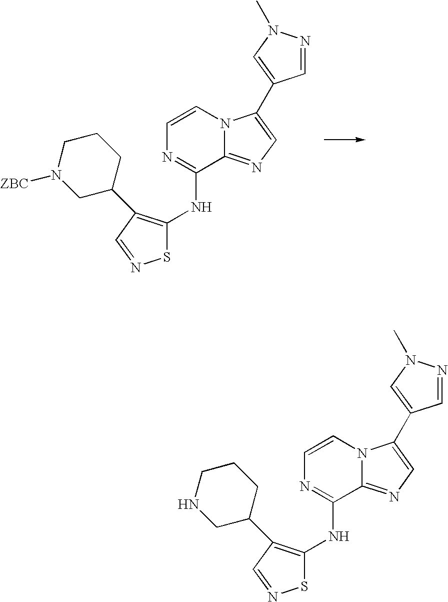

By essentially the process in example 148-1 can be prepared from the procedure described in example 148. HPLC t_{R}=2.66 min, calculated molecular weight, 380.2, observed MH⁺ (LCMS) 381.2 (m/z).

### Example 149

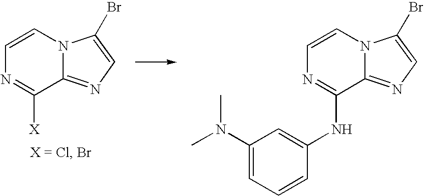

The mixed halo-products (3:1 Cl:Br) from Preparative Example 102 (3.67 g, 15.0 mmol), were combined with N,N-dimethyl-m-phenylenediamine.2HCl (4.71 g, 22.5 mmol), i-Pr₂NEt (15.7 mL, 90.2 mmol), and NMP solvent (75 mL). The reaction was heated in an oil bath at 160° C. for 18 hours. The reaction was cooled and concentrated under vacuum. The crude material was purified by column chromatography; 2 columns using a gradient of 20% EtOAc/Hexanes increasing to 50% EtOAc/Hexanes. The product 149 was isolated in 95% purity as determined by ¹H NMR (400 MHz DMSO-d₆,) □ 9.36 (s, 1H), 7.77 (s, 1H), 7.74 (d, J=4.4 Hz, 1H), 7.54 (d, J=4.8 Hz, 1H), 7.47 (m, 1H), 7.42 (t, J=2.0 Hz), 7.09 (t, J=8.0 Hz, 1H), 6.40 (dd, J=8.0 Hz, 2.0 Hz, 1H), 2.87 (s, 6H). Product was isolated in 77% yield, 3.83 g.

### Example 150-1 to 150-30

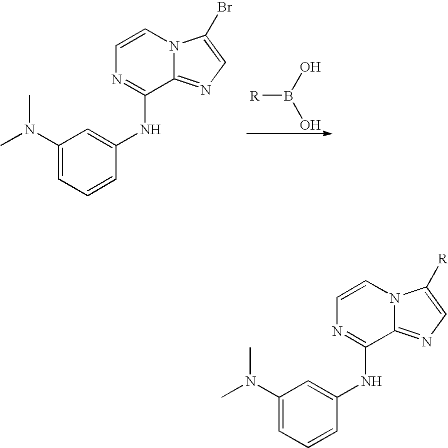

A 1.5 M solution of Na₂CO₃ in H₂O (0.5 mL) was added to 4 mL vials containing 10 mol % Pd(dppf)Cl₂ and 1.5 eq. of the appropriate boronic acid. The product from example 149 was added last as a 0.06 M solution in DME (2.0 mL). The reactions were flushed with Argon, capped, and placed in a sand bath at 80° C. overnight. The reactions were cooled, concentrated, and purified via preparative HPLC to give products 150.

TABLE 13

| | | | LCMS | HPLC |
|---|---|---|---|---|
| Exam- | | | MH⁺ | MS t_{R} |
| ple | Product | MW | m/z | (min) |
| 150-1 | 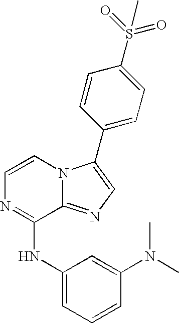 | 407.5 | 408.3 | 1.30 |
| 150-2 | 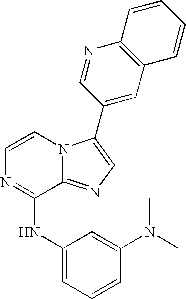 | 380.5 | 381.2 | 1.50 |
| 150-3 | 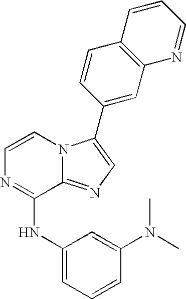 | 380.5 | 381.2 | 1.42 |
| 150-4 | 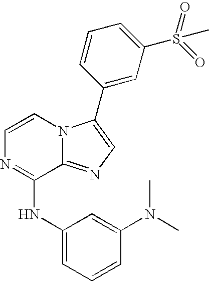 | 407.5 | 408.1 | 1.29 |
| 150-5 | 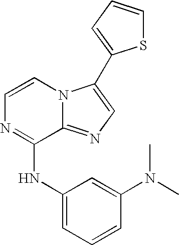 | 335.4 | 336.2 | 3.15 |
| 150-6 | 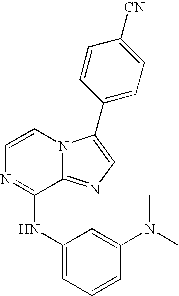 | 354.4 | 355.2 | 3.23 |
| 150-7 | 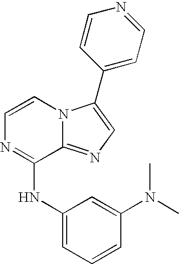 | 330.4 | 331.2 | 1.79 |
| 150-8 | 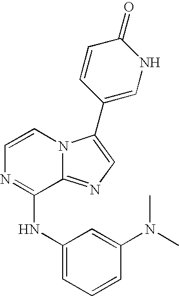 | 346.4 | 347.2 | 1.98 |
| 150-9 | 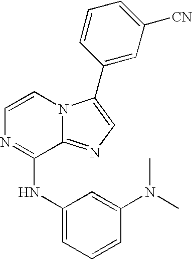 | 354.4 | 355.2 | 3.25 |
| 150-10 | 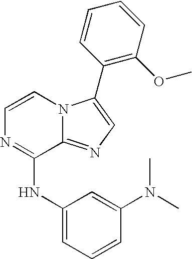 | 359.4 | 360.3 | 3.41 |
| 150-11 | 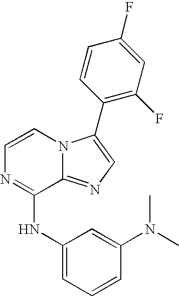 | 365.4 | 366.3 | 3.65 |
| 150-12 | 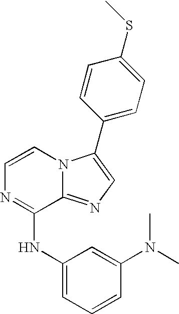 | 375.5 | 376.2 | 3.86 |
| 150-13 | 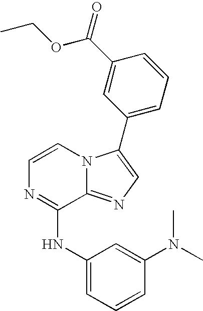 | 401.5 | 402.2 | 3.93 |
| 150-14 | 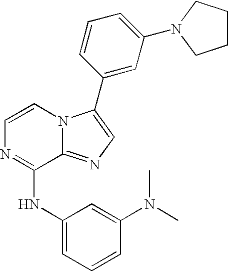 | 398.5 | 399.3 | 4.23 |
| 150-15 | 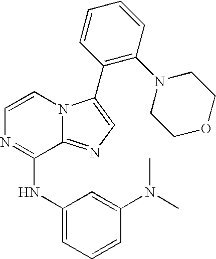 | 414.5 | 415.3 | 3.52 |
| 150-16 | 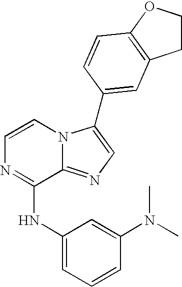 | 371.4 | 372.2 | 3.42 |
| 150-17 | 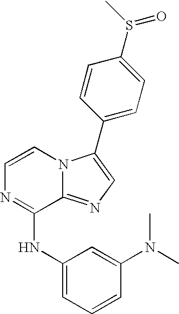 | 391.5 | 392.2 | 2.55 |
| 150-18 | 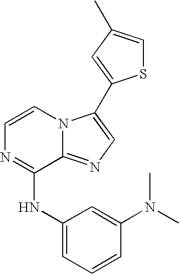 | 349.5 | 350.2 | 3.85 |
| 150-19 | 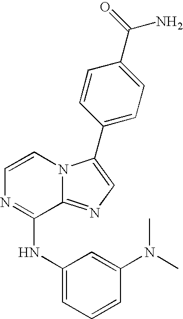 | 372.4 | 373.2 | 2.39 |
| 150-20 | 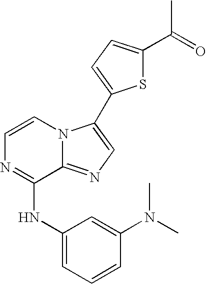 | 377.5 | 378.2 | 3.29 |
| 150-21 | 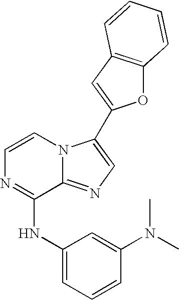 | 369.4 | 370.2 | 4.23 |
| 150-22 | 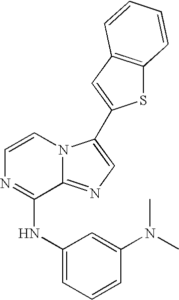 | 385.5 | 386.2 | 4.36 |
| 150-23 | 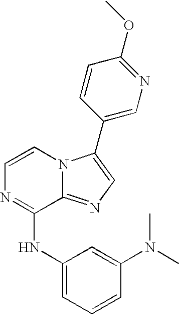 | 360.4 | 361.2 | 3.05 |
| 150-24 | 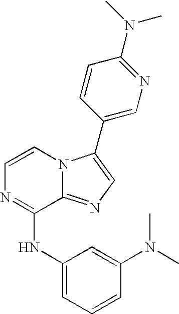 | 373.5 | 374.2 | 2.83 |
| 150-25 | 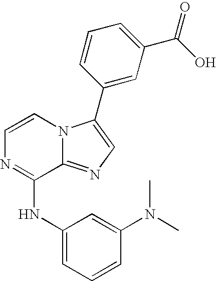 | 373.4 | 374.3 | 2.02 |
| 150-26 | 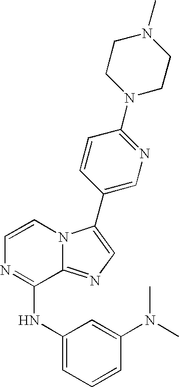 | 428.5 | 429.3 | 2.10 |
| 150-27 | 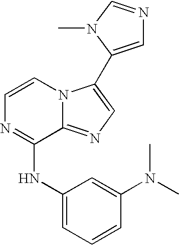 | 333.4 | 334.2 | 0.72 |
| 150-28 | 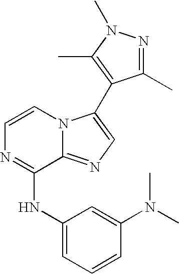 | 361.5 | 362.2 | 2.68 |
| 150-29 | 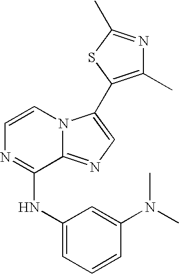 | 364.5 | 365.2 | 3.05 |
| 150-30 | 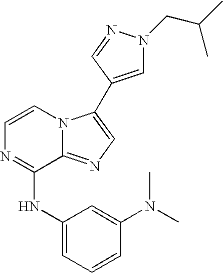 | 375.2 | 376.3 | 1.51 |
| 150-31 | 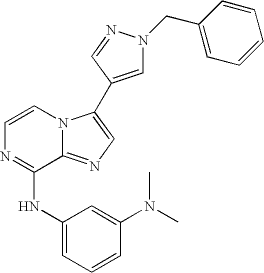 | 409.2 | 410.2 | 1.53 |

### Example 151

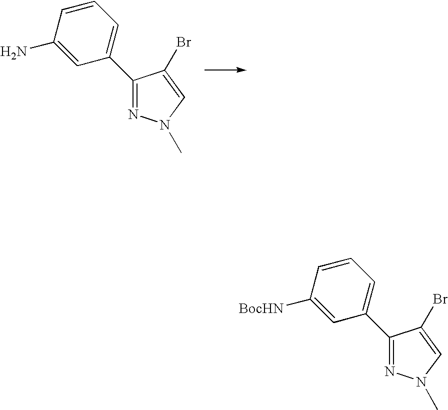

To the mixture of 3-(4-bromo-1-methyl-1H-pyrazol-3-yl-)phenyl amine (1.78 g, 7.1 mmol), imidazole (1.36 g, 20 mmol), and catalytic amount DMAP in DMF (12 mL), (BOC)₂O (1.7 g, 7.8 mmol) was added at room temperature. The mixture was stirred overnight and diluted with EtOAc (200 mL), the organics were washed with H₂O, brine and dried over Na₂SO₄. After concentration, the residue was purified with column chromatography (silica gel, hexane/EtOAc=70/30) to give the product 151 (2.52 g) as white solid. HPLC-MS t_{R}=2.00 min (UV₂₅₄ₙₘ). Mass calculated for formula C₁₅H₁₈BrN₃O₂, 351.1; observed MH⁺ LC/MS 352.1 (m/z).

### Example 152

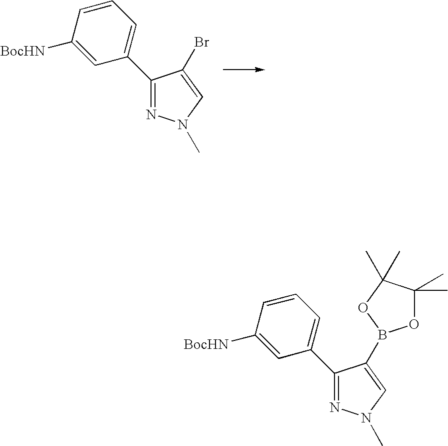

To a 25 mL round bottom flask charged with bis(pinacolato)diboron (1.0 g, 4.0 mmol), KOAC (960 mg, 10 mmol), Pd(dppf)Cl₂ (240 mg, 0.30 mmol) and product from example 151 (1.16 g, 3.30 mmol) was added DMSO (6 mL) under argon. The mixture was degassed thoroughly. This resulting mixture was then heated at 80° C. overnight, diluted by EtOAc (40 mL) and filtered through celite. After concentration, the residue was purified with column chromatography (silica gel, hexane/EtOAc=80/20) to give the product 152 (997 mg) as an oil. HPLC-MS t_{R}=2.11 min (UV_{254 nm}); mass calculated for formula C₂₁H₃₀BN₃O₄, 399.2; observed MH⁺ LCMS 400.3 (m/z).

### Example 153

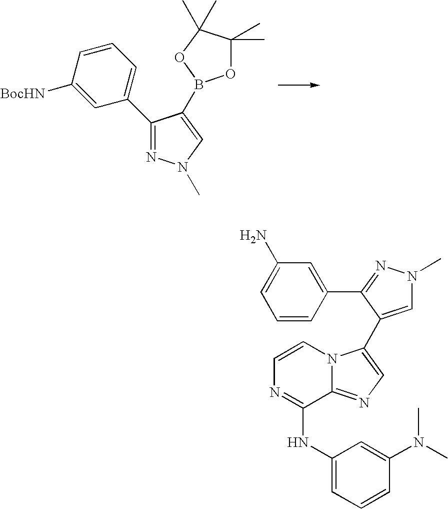

Under argon, the boronate compound 152 (120 mg, 0.3 mmol) in THF (3.0 mL, 5% H₂O) was added to the flask which was charged with Pd(dppf)Cl₂ (8.0 mg, 10 mol %), K₂CO₃ (138 mg, 1.0 mmol), and 3-bromoimidazopyrazine 149 (51 mg, 0.15 mmol). The mixture was degassed thoroughly with argon. The resulting solution was heated up to 80° C. and stirred overnight. After cooling to room temperature, the mixture was diluted with EtOAc (50 mL) and the solid was removed by filter through Celite and washed with some EtOAc. Concentration resulted in a residue 153 and was used in the next step directly without further purification. HPLC-MS t_{R}=2.05 min (UV_{254 nm}); mass calculated for formula C₂₉H₃₂N₈O₂; 524.3, observed MH⁺ (LCMS) 525.2.1 (m/z).

### Example 154

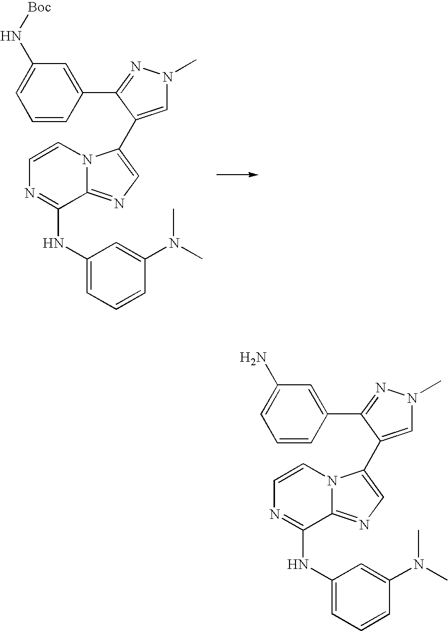

To the product from example 153 was added HCl (6 N, 3 mL), and the mixture was stirred at room temperature for 10 min. The reaction was concentrated, and the residue purified with HPLC to give the compound 154 (48 mg). HPLC-MS t_{R}=1.16 min (UV_{254 nm}); mass calculated for formula C₂₄H₂₄N₈, 424.2; Observed MH⁺ (LCMS) 425.2 (m/z).

### Example 155

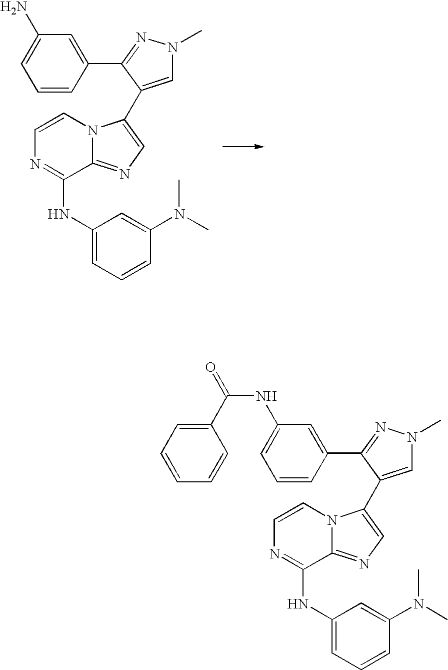

To a mixture of hydroxy benzotriazole (7 mg, 0.05 mmol and benzoic acid (6 mg, 0.05 mmol) in DMF (1 mL), EDC (10 mg, 0.05 mmol) was added and the mixture was stirred at room temperature for 10 min. Then product 154 (21 mg, 0.05 mmol) in DMF (1 mL) was added and the resulting mixture was heated up to 50° C. and stirred overnight. The mixture was diluted with EtOAc (50 mL), washed with H₂O, brine and dried over Na₂SO₄. After concentration the residue was purified by prep-LC to give the product 155. HPLC-MS t_{R}=1.54 min (UV₂₅₄ₙₘ); mass calculated for formula C₃₁H₂₈N₈O, 528.2; observed MH⁺ (LCMS) 529.3 (m/z).

### Example 156

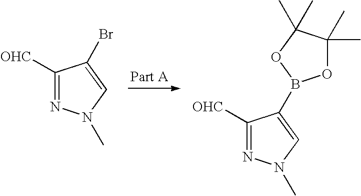

Compound 156 was prepared using the boronation conditions described in Example 152. HPLC-MS t_{R}=1.83 min (UV_{254 nm}); mass calculated for formula C₁₁H₁₇BN₂O₃, 236.1; observed MH⁺ (LCMS) 237.3 (m/z).

### Example 157

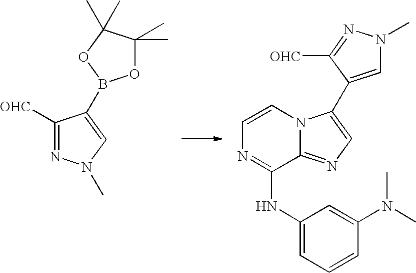

Compound 157 was prepared using the coupling conditions described in example 153.HPLC-MS t_{R}=1.18 min (UV_{254 nm}); mass calculated for formula C₁₉H₁₉N₇O, 361.2; observed MH⁺ (LCMS) 362.1 (m/z).

### Example 158

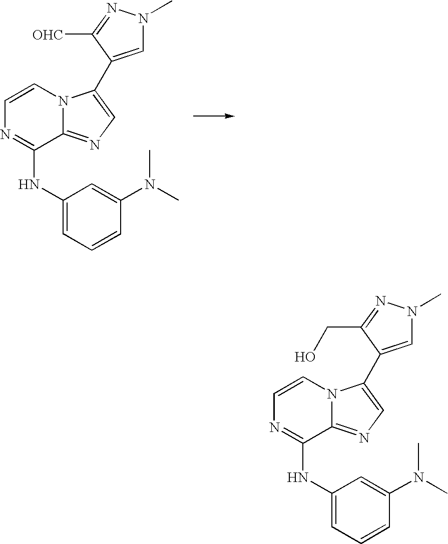

Product from example 157 (50 mg, 0.14 mmol) was dissolved in MeOH (5 mL) and the mixture cooled to 0° C. NaBH₄ (38 mg, 1.0 mmol) was added and the resulting mixture was stirred at 0° C. for 30 min. After concentration, the residue was purified with prep-LC gave the product 158. HPLC-MS t_{R}=0.92 min. (UV₂₅₄ₙₘ); mass calculated for formula C₁₉H₂₁N₇O, 363.2; observed MH⁺ (LCMS) 364.3 (m/z).

### Example 159

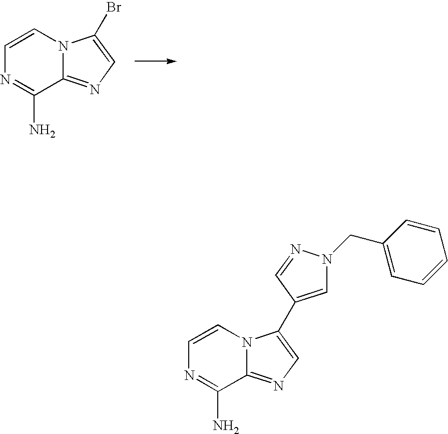

Product of example 159 was prepared using the coupling condition described in 153. HPLC-MS t_{R}=0.94 min (UV_{254 nm}); mass calculated for formula C₁₆H₁₄N₆ 290.1, observed MH⁺ (LCMS) 291.3 (m/z).

### Example 160

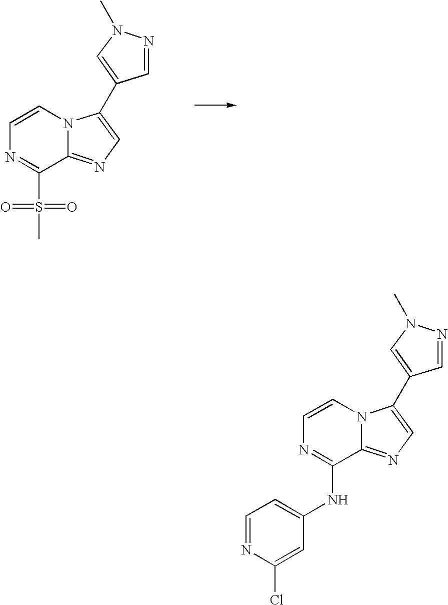

By essentially the same procedure given in example 106, combining the product from example 105 and 2-chloro-4-amino pyridine to give the product 160. HPLC t_{R}=1.45 min. Calculated molecular weight, 325.1, observed MH⁺ (LCMS) 326.0 (m/z).

### Example 161

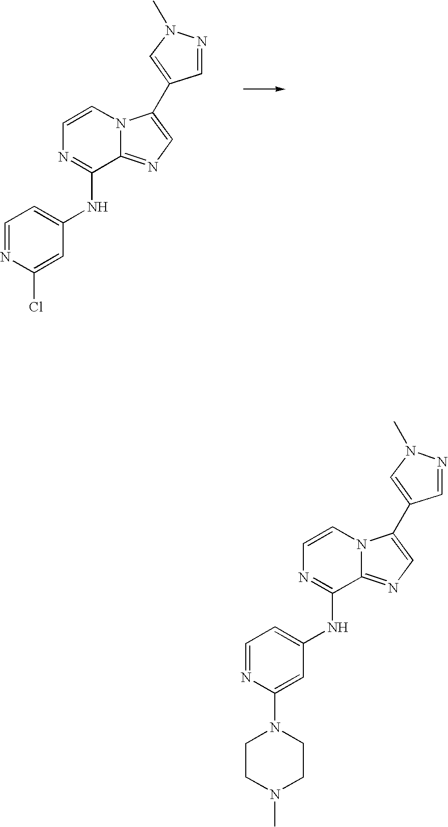

A mixture of the product from example 160, 1-methyl piperazine (excess) is stirred and heated at 100° C. for 72 hrs. The mixture poured in to 10% aqueous Na₂CO₃ and extracted with ethyl acetate. The extracts dried over sodium sulfate, filtered and evaporated. Preparative HPLC purification afford the product, HPLC t_{R}=1.92 min. Calculated molecular weight=389.5, observed MH⁺ (LCMS) 390.30 (m/z).

By essentially the same procedure given in example 161, combining intermediates from preparative example 160 with the amines given in column 1, compounds given in column 2 were prepared. The compounds obtained were purified by preparative HPLC. The purified products were treated with 4 N HCl in dioxane to remove the BOC protecting group. The volatiles were removed under vacuum. The product was dissolved in acetonitrile-water and lyophilized to give the product(s).

TABLE 14

| | | | | LCMS | |
|---|---|---|---|---|---|
| Exam- | | | | MH⁺ | HPLC |
| ple | Column 1 | Column 2 | MW | m/z | MS t_{R} |
| 163 | 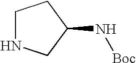 | 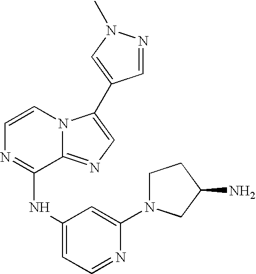 | 375.1 | 376.1 | 0.75 |
| 164 | 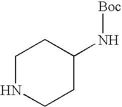 | 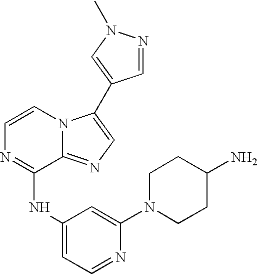 | 389.2 | 390.2 | 0.75 |
| 164-1 | 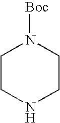 | 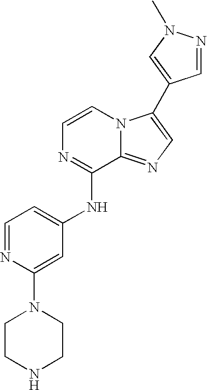 | 375.1 | 376.0 | 1.94 |

### Example 165

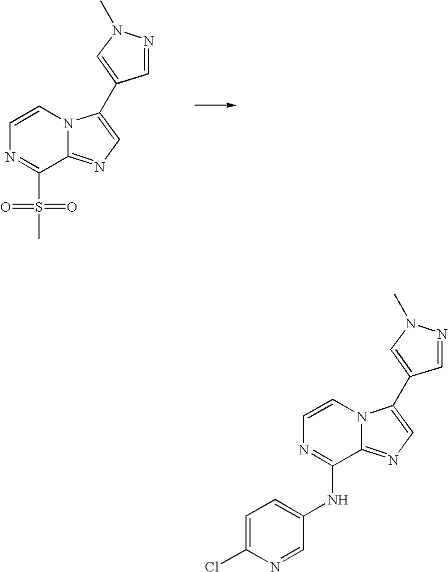

By essentially the same procedure given in example 106, combining the product from example 105 and 2-chloro-4-amino pyridine to give the product 165.

HPLC t_{R}=1.48 min. Calculated molecular weight, 325.1; observed MH⁺ (LCMS), 326.0 (m/z).

### Example 166

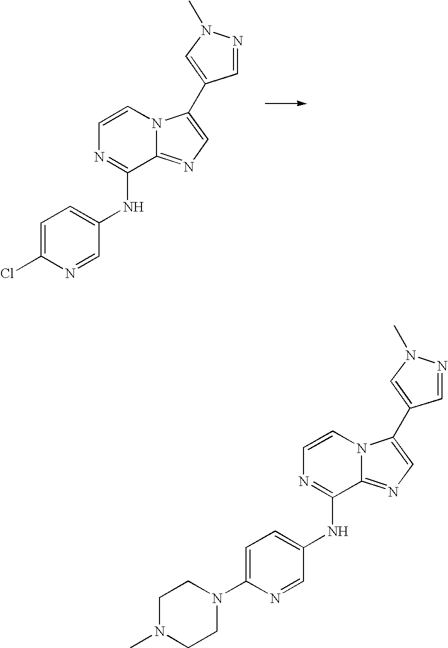

A mixture of the product from example 165, 1-methyl piperazine (excess) is stirred and heated at 100° C. for 72 hrs. The mixture poured into 10% aqueous Na₂CO₃ and extracted with ethyl acetate. The extracts were dried over sodium sulfate, filtered and evaporated. Preparative HPLC purification afforded the product. HPLC t_{R}=1.80 min. Calculated molecular weight, 389.5.1; observed MH⁺ (LCMS) 390.23 (m/z).

By essentially the same procedure given in example 161, combining intermediates from preparative example 160 with the amines given in column 1, the compounds given in column 2 were prepared. The compounds obtained were purified by preparative HPLC. The purified products obtained were treated with 4 N HCl dioxane to remove the BOC protecting group and volatiles were removed under vacuum. The product was dissolved in acetonitrile-water and lyophilized to give the product(s).

TABLE 15

| | | | | LCMS | |
|---|---|---|---|---|---|
| Exam- | | | | MH⁺ | HPLC |
| ple | Column 1 | Column 2 | MW | m/z | MS t_{R} |
| 167 | 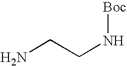 | 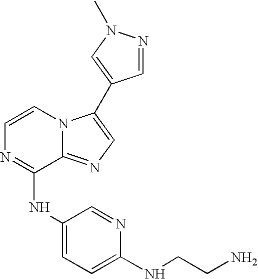 | 349.1 | 350.1 | 0.50 |
| 168 | 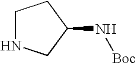 | 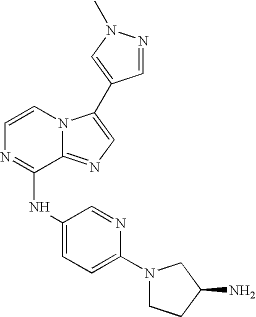 | 375.4 | 376.2 | 0.80 |
| 169 | 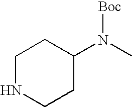 | 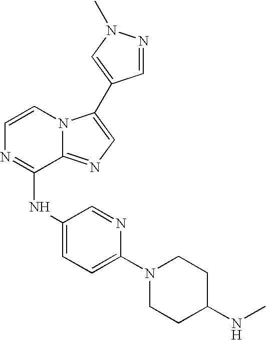 | 403.4 | 404.2 | 0.85 |

### Example 170

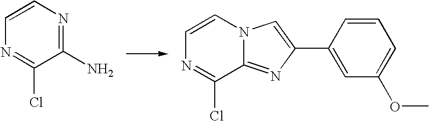

To a solution of 2-amino-3-chloropyrazine (0.20 g, 1.5 mmol, 1.00 equiv) and 3-methoxyphenacyl bromide (0.71 g, 3.1 mmol, 2.0 equiv) in dioxane (10 mL) was heated at 90° C. for 3 hr. The resulting mixture was cooled to rt and filtered. The filtrate was partitioned between 10% IPA/DCM and 1 N NaOH. The aqueous extract was washed with 10% IPA/DCM (2×) and the combined organic extracts were washed with brine and dried with sodium sulfate. Concentration afforded 8-chloro-2-(3-methoxy-phenyl)-imidazo[1,2-a]pyrazine (76 mg, 19%). MH⁺ (LCMS) 260.1 (m/z).

### Example 171

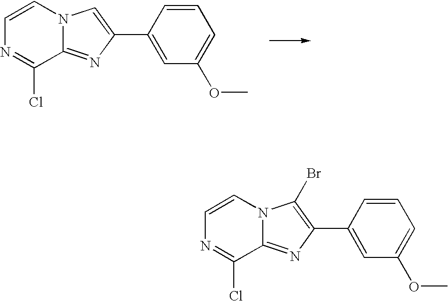

To the product from example 170 in acetic acid (10 mL) was added a solution of bromine in acetic acid (0.25 mmol, 1 mL). Concentration of the reaction mixture afforded crude 3-bromo-8-chloro-2-(3-methoxy-phenyl)-imidazo[1,2-a]pyrazine. MH⁺ (LCMS) 338.0 (m/z).

### Example 172

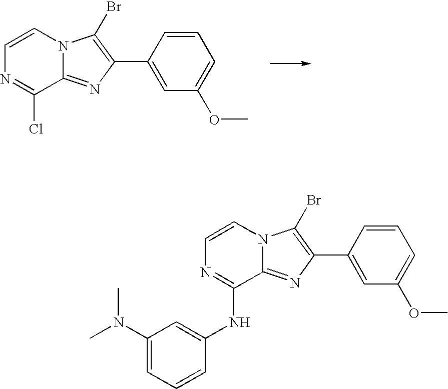

A solution of 3-bromo-8-chloro-2-(3-methoxy-phenyl)-imidazo[1,2-a]pyrazine (0.13 g, 0.38 mmol, 1.00 equiv) product from example 171, N,N-dimethyl-m-phenylenediamine hydrochloride (0.15 g, 0.71 mmol, 1.9 equiv) and N,N-diisopropylethylamine (0.33 mL, 1.9 mmol, 5.0 equiv) in NMP (2 mL) was heated at 140° C. for 20 h. Concentration and purification by chromatography (25% ethyl acetate in hexanes) afforded the title compound. MH⁺ (LCMS) 438.1 (m/z).

### Example 173

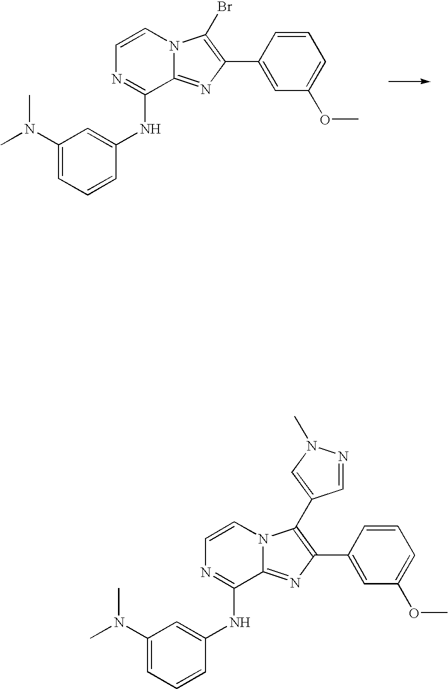

A suspension of 3-bromo-8-chloro-2-(3-methoxy-phenyl)-imidazo[1,2-a]pyrazine (38.2 mg, 0.0871 mmol, 1.00 equiv), [1,1′-bis(diphenylphosphino) ferrocene]dichloropalladium(II) (3 mg, 0.004 mmol, 5 mol %), 1-methyl-4-(4,4,5,5-tetramethyl-1,3,2-dioxaborolan-2-yl)-1H-pyrazole (0.036 g, 0.17 mmol, 2.0 equiv) and sodium carbonate (0.028 g, 0.26 mmol, 3.0 equiv) in 1,2-dimethoxy ethane/water (0.4 mL/0.1 mL) was heated at 90° C. for 2.5 hr. The mixture was allowed to cool, filtered, concentrated and purified using chromatography (25% ethyl acetate in hexanes). The title compound was obtained as a colorless solid. HPLC t_{R}=1.68 min), MH⁺ (LCMS) 440.2 (m/z).

### Example 174

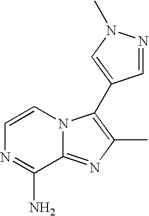

The title compound, example 174 was prepared by the same procedure set forth in the above example 173 HPLC (t_{R}=0.64 min). Calculated M.Wt. 228.1, observed MH⁺ (LCMS) 229.1 (m/z).

### Example 175

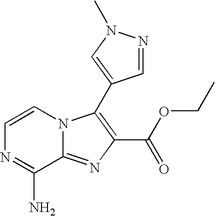

The title compound, example 175 was prepared by the same procedure set forth in the above example 173. HPLC (t_{R}=0.75 min). Calculated M.Wt. 286.2, observed MH⁺ (LCMS) 287.2 (m/z).

### Example 176

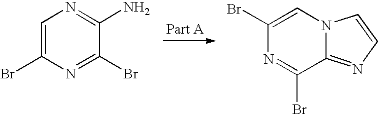

The mixture of bromoacetaldehyde diethyl acetal (5.2 mL, 33.3 mmol) and HBr (0.8 mL, 48% in H₂O) in H₂O (8 mL) was heated at reflux and stirred for 1 hour. After cooling to room temperature. The mixture was extracted with ethyl ether (100 mL, 5×). The ether was dried over Na₂SO₄ and concentrated to give the crude bromoacetaldehyde. To the crude acetaldehyde, 2-amino-3,5-dibromopyrazine (4.30 g, 17 mmol) and DME (120 mL) were added followed by the addition of HBr (1 mL, 48% in H₂O). The mixture was heated at reflux with stirring overnight. After cooling to room temperature the solid was collected with filtration and washed with DME. After drying under vacuum, the product 176 (4.50 g) obtained as HBr salt, a black solid. HPLC-MS t_{R}=1.13 min (UV_{254 nm}); mass calculated for formula C₆H₃Br₂N₃, 274.9; observed MH⁺ (LCMS) 276.0 (m/z).

### Example 177

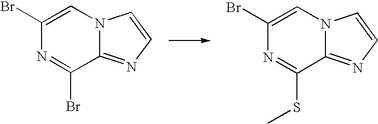

The dibromo compound 176 (2.16 g, 6.0 mmol) was dissolved in MeOH (20 mL). NaSMe (840 mg, 12 mmol) was added. The mixture was stirred for 2 hours at room temperature and concentrated. The residue was taken up in H₂O (20 mL) and extracted with DCM/iso-PrOH (9/1) (50 mL, 3×). The combined organic layers were dried over Na₂SO₄ and concentrated. The crude compound was purified with column chromatography (silica gel, EtOAc/hexane=40/60 to 100% EtOAc) to give the pure compound 177 (1.12 g) as yellowish solid. ¹H NMR (400 MHz, CDCl₃) δ7.97 (s, 1H), 7.68 (d, 1H), 7.57 (d, 1H), 2.66 (s, 3H). HPLC-MS t_{R}=1.40 min (UV_{254 nm}); mass calculated for formula C₇H₆BrN₃S, 242.9; observed MH⁺ (LCMS) 244.1 (m/z).

### Example 178

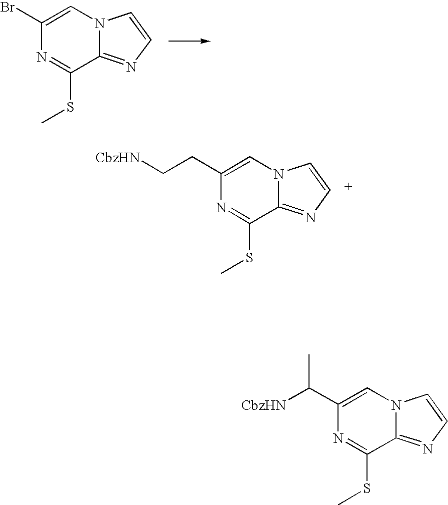

Under Ar, a solution of 9-BBN (10 mL, 0.5 M in THF) was added drop wise to the solution of benzyl N-vinylcarbamate (875 mg, 5.00 mmol) in THF (10 mL) at room temperature and stirred for 2 hours. The resulting mixture was transferred to another flask that was charged with product from example 177 (610 mg, 2.5 mmol), K₃PO₄ (850 mg, 4.0 mmol) and Pd(dppf)Cl₂ (160 mg, 0.2 mmol) in THF (20 mL, together with 1 mL of water) under Argon. The resulting mixture was heated to 60° C. and stirred overnight under Argon. The reaction was cooled to room temperature. EtOAc (200 mL) was added to the reaction mixture and filtered through celite. After concentration the residue was purified with column (silica gel, EtOAc/hexane=50/50) to give the product 178 (457 mg) and 178 A (150 mg) as oil.

178: ¹H NMR (400 MHz, CDCl₃) δ 7.65 (s, 1H), 7.63 (d, 1H), 7.51 (d, 1H), 7.34 (m, 5H), 5.43 (s, 1H), 5.10(s, 2H), 3.64 (m, 2H), 2.89 (t, 2H), 2.62 (s, 3H). HPLC-MS t_{R}=1.59 min (UV_{254 nm}); mass calculated for formula C₁₇H₁₈N₄O₂S 342.1; observed MH⁺ (LCMS) 343.1 (m/z).

178 A: HPLC-MS t_{R}=1.50 min (UV_{254 nm}); mass calculated for formula C₁₇H₁₈N₄O₂S, 342.1; observed MH⁺ (LCMS) 343.1 (m/z).

### Example 179

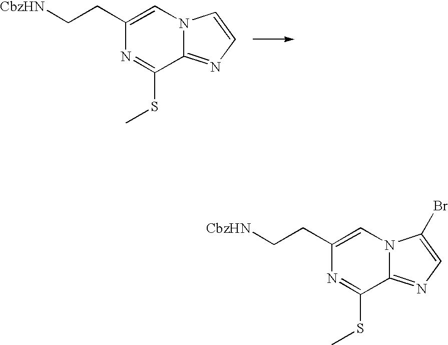

NBS (104 mg, 0.59 mmol) was added to a solution of compound 178 (200 mg, 0.59 mmol) in EtOH (10 mL), at room temperature. The mixture was stirred for 30 min and concentrated. The residue was diluted with EtOAc and washed with saturated aq.NaHCO₃ (30 mL, 2×), brine and dried over Na₂SO₄. After concentrating, the crude product 179 was used in the next step directly without further purification. HPLC-MS t_{R}=1.88 min (UV_{254 nm}); mass calculated for formula C₁₇H₁₇BrN₄O₂S, 420.0; observed MH⁺ (LCMS) 421.0 (m/z).

### Example 180

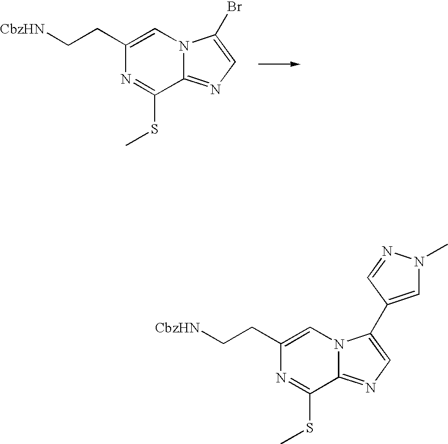

The boronate (122 mg, 0.585 mmol), was mixed with Pd(dppf)Cl₂ (50 mg, 0.06 mmol), K₃PO₄ (318 mg, 1.5 mmol), and the product from example 179 (246 mg, 0.585 mmol) in dioxane (5 mL) was added. The mixture was degassed thoroughly and kept under argon blanket. The resulting solution was heated at 80° C. and stirred overnight. After cooling to room temperature the mixture was diluted with EtOAc (50 mL). The solid was removed by filter through Celite and washed with EtOAc. The solvent was removed under reduced pressure and the resulting residue was purified with column chromatography (silica gel, EtOAc to MeOH/EtOAc=5/95) gave the product 180 (212 mg) as oil. HPLC-MS t_{R}=1.62 min (UV_{254 nm}); mass calculated for formula C₂₁H₂₂N₆O₂S, 422.2; observed MH⁺ (LCMS) 423.3 (m/z).

### Example 181

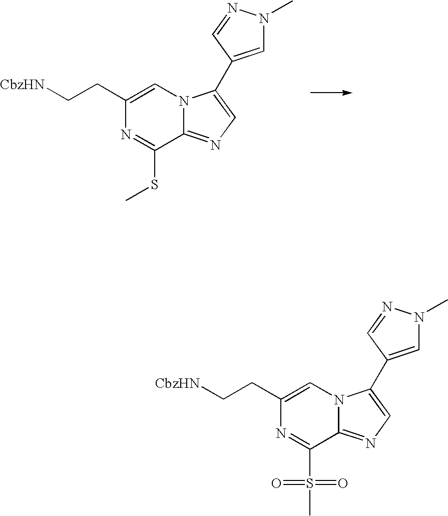

A mixture of compound 180 (212 mg, 0.5 mmol) and m-CPBA (224 mg, 77%, 1.0 mmol) in DCM (10 mL) was stirred at room temperature for 30 min then diluted with EtOAc (100 mL). The organics were washed with NaHCO₃ (sat. aq., 10 ml×2), brine and dried over Na₂SO₄. After concentration, the crude product 181 was used in the next step directly without further purification. HPLC-MS t_{R}=1.36 min (UV_{254 nm}); mass calculated for formula C₂₁H₂₂N₆O₄S, 454.1; observed MH⁺ (LCMS) 455.2 (m/z).

### Example 182

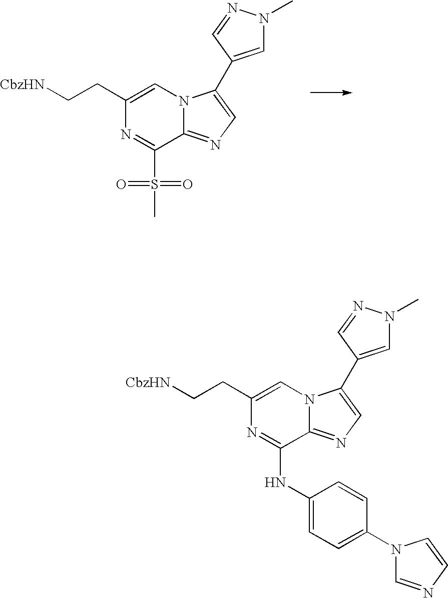

The aniline (16 mg, 0.21 mmol) was dissolved in dry DMSO (2 mL) with NaH (60% in oil, 4 mg, 0.1 mmol) under argon. The mixture was stirred for 10 min at room temperature and sulfone 181 (25 mg, 0.05 mmol) in dry DMSO (0.5 mL) was added. The reaction mixture was heated at 80° C. and stirred for 10 min. After cooling to room temperature, the mixture was purified by prep-LC to give the product 182 as a TFA salt. HPLC-MS t_{R}=1.15 min (UV_{254 nm}); mass calculated for formula C₂₉H₂₇N₉O₂, 533.2; observed MH⁺ (LCMS) 534.2 (m/z).

### Example 183

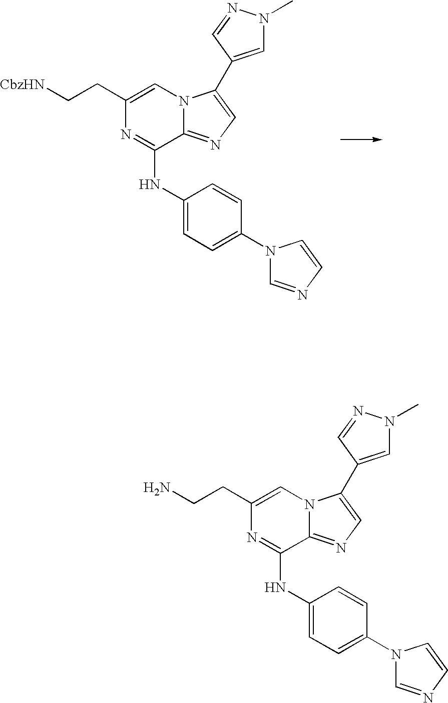

The TFA salt of compound 182 (20 mg, 0.038 mmol) was treated with 4 N HCl (2 mL) and the mixture was stirred at room temperature for 30 min. After concentration the residue was dried by lyophilization gave the final compound 183. HPLC-MS t_{R}=0.75 min (UV_{254 nm}); mass calculated for formula C₂₁H₂₁N₉, 399.2; observed MH⁺ (LCMS) 400.1 (m/z).

By essentially the same procedures given in examples 178-183 to give compound 184 and 185.

TABLE 16

| | | | LCMS | |
|---|---|---|---|---|
| Exam- | | | MH⁺ | HPLC |
| ple | Column 2 | MW | m/z | MS t_{R} |
| 184 | 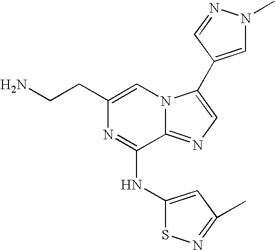 | 354.1 | 355.1 | 0.87 |
| 185 | 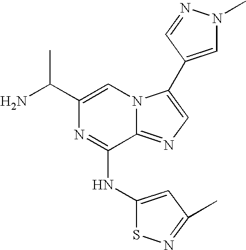 | 354.1 | 355.1 | 0.90 |

### Example 186

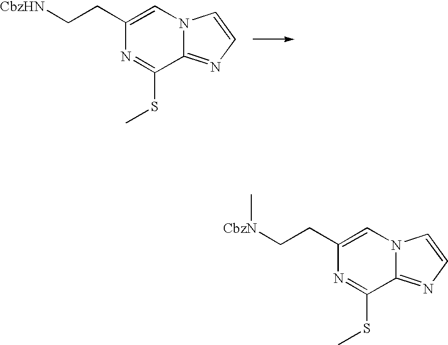

To a solution of NaH (24 mg, 60% in oil, 0.6 mmol), compound 178 (200 mg, 0.585 mmol) in dry DMF (5 mL) was added carefully. The mixture was stirred at room temperature for 10 min. Iodomethane (100 μL) was added to the above reaction mixture. The resulting mixture was stirred overnight, cooled to 0° C. and water was added carefully to quench the reaction. The aqueous was extracted with EtOAc and the organics was dried over Na₂SO₄. After concentration, the crude product was purified with column chromatography (silica gel, hexane/EtOAc=70/30) to give the product 186 (201 mg). HPLC-MS t_{R}=1.65 min (UV_{254 nm}), mass calculated for formula C₁₈H₂₀N₄O₂S, 356.1; observed MH⁺ (LCMS) 357.2 (m/z).

### Example 187

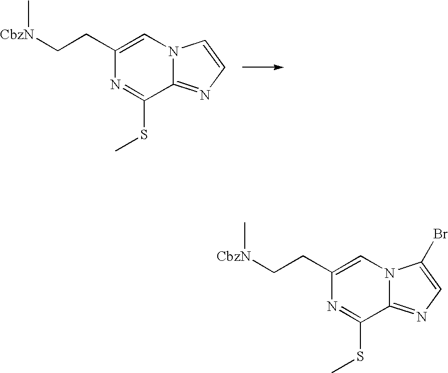

Compound 187 was prepared using the brominating conditions described in example 179. HPLC-MS t_{R}=2.01 min (UV_{254 nm}); mass calculated for formula C₁₈H₁₉BrN₄O₂S, 434.0; observed MH⁺ (LCMS) 435.1 (m/z).

### Example 188

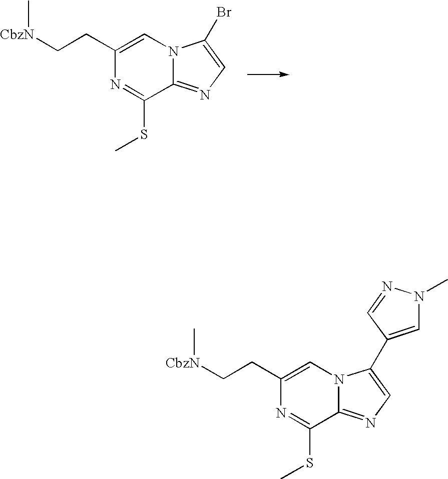

Compound 188 was synthesized using the same coupling condition described in example 180. HPLC-MS t_{R}=1.73 min (UV_{254 nm}); mass calculated for formula C₂₂H₂₄N₆O₂S, 436.2; observed MH⁺ (LCMS) 437.2 (m/z).

### Example 189

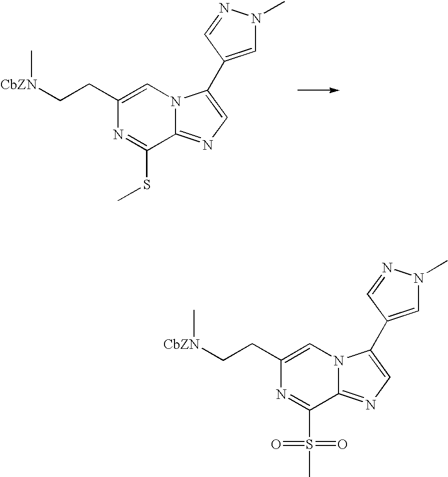

Compound 189 was prepared using the oxidation conditions described in example 181. HPLC-MS t_{R}=1.43 min (UV_{254 nm}); mass calculated for formula C₂₂H₂₄N₆O₄S, 468.2; observed MH⁺ (LCMS) 469.1 (m/z).

### Example 190

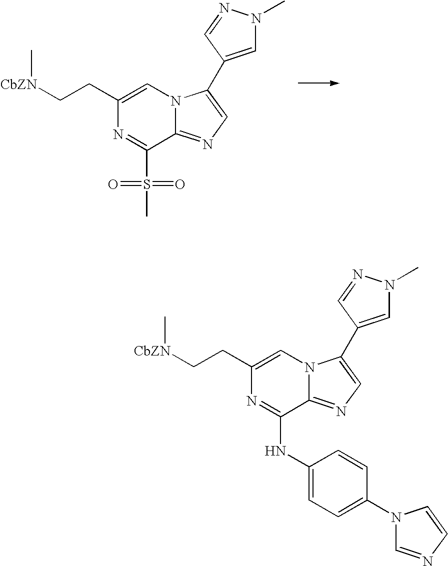

Compound 190 was prepared using the amination conditions described in example 182. HPLC-MS t_{R}=1.25 min (UV_{254 nm}); mass calculated for formula C₃₀H₂₉N₉O₂, 547.2; observed MH⁺ (LCMS) 548.2 (m/z).

### Example 191

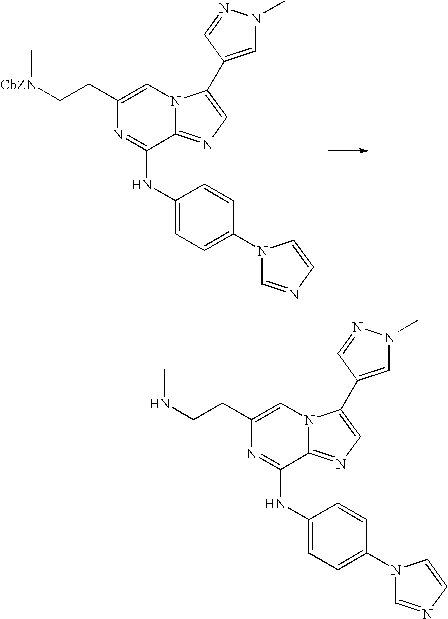

Compound 190 was synthesized using the deprotecting conditions described in example 183. HPLC-MS t_{R}=0.75 min (UV_{254 nm}); mass calculated for formula C₂₂H₂₃N₉, 413.2; observed MH⁺ (LCMS) 414.2 (m/z). By essentially the same procedure given in Preparative Example 186-191, compounds given in Column 2 can be prepared from 183 and 185.

TABLE 17

| | | | LCMS | |
|---|---|---|---|---|
| Exam- | | | MH⁺ | HPLC |
| ple | Column 2 | MW | m/z | MS t_{R} |
| 192 | 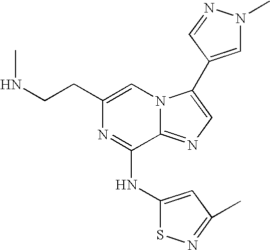 | 368.2 | 355.1 | 0.87 |
| 193 | 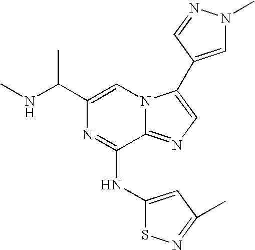 | 368.2 | 369.1 | 0.90 |
| 194 | 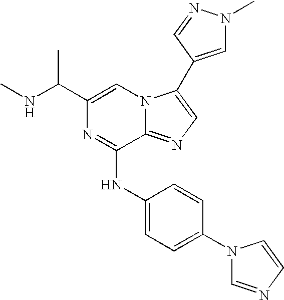 | 413.2 | 414.2 | 0.78 |

### Example 195

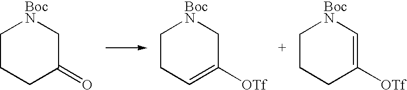

A solution of LDA (28.6 mmol) was prepared from iso-Pr₂NH (4.03 mL, 28.6 mmol) and n-BuLi (11.40 mL, 2.5 M in hexane, 28.6 mmol) in THF (50 mL). The solution was cooled at −78° C. and N-Boc-3-piperidone (4.0 g, 20 mmol) in THF (10 mL) was added with a syringe. After 15 min, N-phenyltriflimide (8.60 g, 24.0 mmol) in THF (20 mL) was added. The reaction mixture was then warmed up to room temperature slowly and stirred overnight. After evaporation, of the solvent under vacuum, the residue was dissolved in DCM (120 mL). The solution was then filtered on neutral alumina and evaporated. Flash chromatography (hexane/EtOAc 80/20) of the crude oil on silica gel gave products 195 and 196.

Product 195: HPLC-MS t_{R}=1.65 min (UV_{254 nm}); mass calculated for formula C₁₁H₁₆F₃NO₅S, 231.1; observed MH⁺ (LCMS) 232.1 (m/z).

Product 196: HPLC-MS t_{R}=1.68 min (UV_{254 nm}); mass calculated for formula C₁₁H₁₆F₃NO₅S, 231.1; observed MH⁺ (LCMS) 232.1 (m/z).

### Example 197

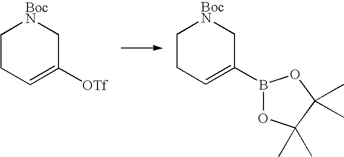

To a 25 mL round bottom flask charged with bis(pinacolato)diboron (1.50 g, 6 mmol), potassium acetate (1.5 g, 15 mmol), Pd(dppf)Cl₂ (408 mg, 0.5 mmol) and DPPF (277 mg, 0.5 mmol). Compound 195 (1.55 g, 5.0 mmol) in dioxane 20 mL) was added to the above mixture. The mixture was degassed thoroughly and placed under argon. This resulting mixture was then heated at 80° C. for overnight, diluted with EtOAc (40 mL) and filtered through celite. After concentration, the residue was purified with column chromatography (silica gel, Hexane/EtOAc=60/40) to give the product (832 mg) as an oil. HPLC-MS t_{R}=2.41 min (UV_{254 nm}), mass calculated for formula C₁₆H₂₈BNO₄, 309.2; observed MH⁺; -t-Bu (LCMS) 254.2 (m/z).

### Example 198

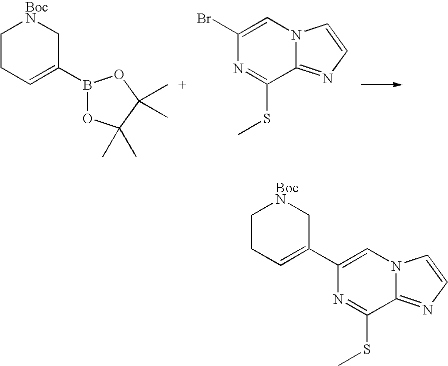

To a 25 mL round bottom flask charged with boronate 197 (456 mg, 1.5 mmol), K₂CO₃ (800 mg, 6 mmol), and Pd(dppf)Cl₂ (160 mg, 0.2 mmol) was added a solution of product from example 177 (360 mg, 1.5 mmol) in DMF (10 mL). The mixture was degassed thoroughly and placed under argon. This resulting mixture was then heated at 80° C. overnight. The reaction mixture was diluted with EtOAc (40 mL) and filtered through Celite. After concentration, the residue was purified by column chromatography (silica gel, Hexane/EtOAc=60/40) to give the product 198 (258 mg) as an oil. HPLC-MS t_{R}=1.91 min (UV_{254 nm}); mass calculated for formula C₁₇H₂₂N₄O₂S, 346.1; observed MH⁺ (LCMS) 347.2 (m/z).

### Example 199

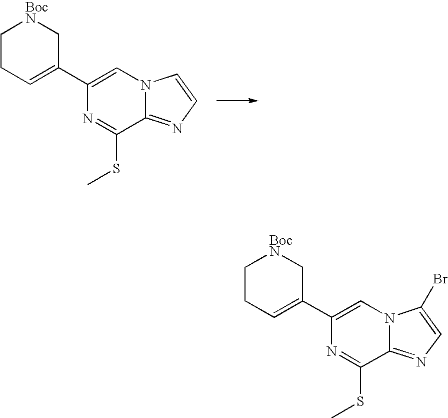

Compound 199 was prepared using brominating conditions described in example 179. HPLC-MS t_{R}=2.26 min (UV_{254 nm}); mass calculated for formula C₁₇H₂₁BrN₄O₂S, 424.1; observed MH⁺ (LCMS) 425.0 (m/z).

### Example 200

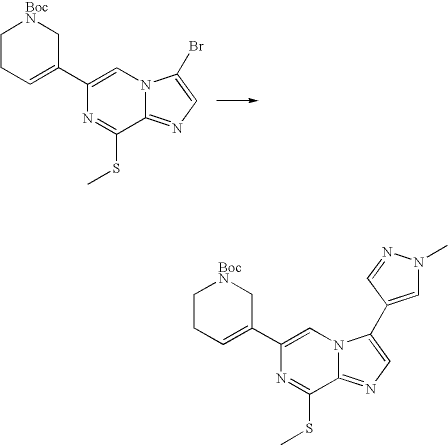

By essentially, example product 200 was synthesized using the same coupling conditions described in example 180. HPLC-MS t_{R}=1.96 min (UV_{254 nm}); mass calculated for formula C₂₁H₂₆N₆O₂S, 426.2; observed MH⁺ (LCMS) 427.1 (m/z).

### Example 201

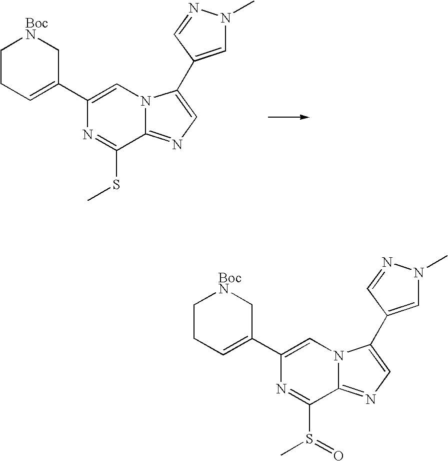

The mixture of compound 200 (130 mg, 0.305 mmol) and m-CPBA (68 mg, 77%, 0.305 mmol) in DCM (5 mL) was stirred at 0° C. for 30 min and then diluted with EtOAc (100 mL). The organics were washed with saturated aqueous NaHCO₃ (10 mL, 2×), brine, and dried over Na₂SO₄. After concentration the crude product 201 was used in the next step directly without further purification. HPLC-MS t_{R}=1.48 min (UV_{254 nm}); mass calculated for formula C₂₁H₂₆N₆O₃S, 442.2; observed MH⁺ (LCMS) 443.2 (m/z).

### Example 202

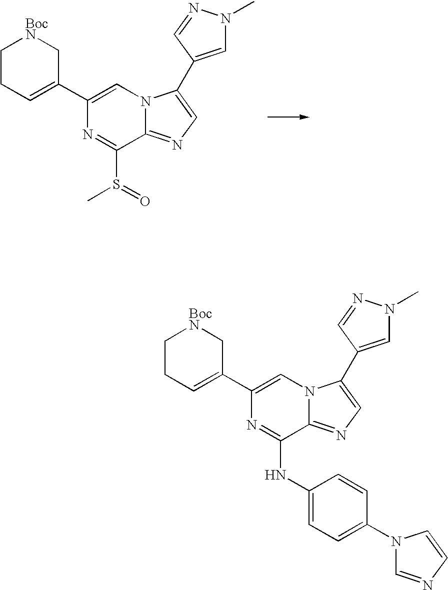 The product example 202 was prepared using the similar experimental conditions described in product example 182. HPLC-MS t_{R}=1.44 min (UV_{254 nm}); mass calculated for formula C₂₉H₃₁N₉O₂, 537.3; observed MH⁺ (LCMS) 538.3 m/z).

### Example 203

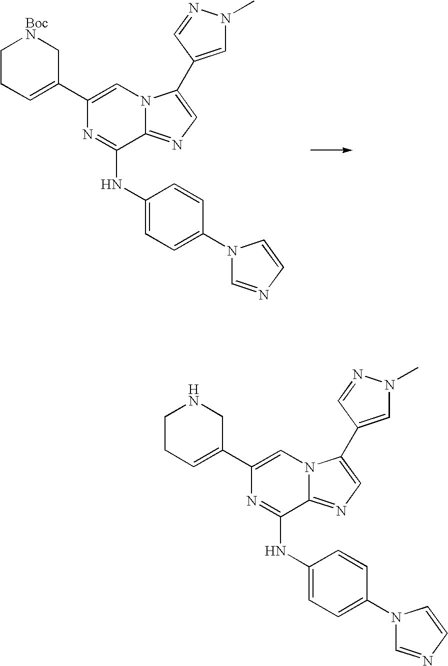

The product from example 202 (20 mg) was treated with 4 N HCl in dioxane (4 mL) and stirred at room temperature for 10 min. After concentration, the residue was dried by lyophilization gave compound 203. HPLC-MS t_{R}=0.75 min (UV_{254 nm}); mass calculated for formula C₂₄H₂₃N₉, 437.2; observed MH⁺ (LCMS) 438.3 (m/z).

By essentially the same procedures given in Preparative Example 203, compounds given in Column 2 of Table 18 can be prepared from example 195 through 203.

TABLE 18

| | | | LCMS | |
|---|---|---|---|---|
| Exam- | | | MH⁺ | HPLC |
| ple | Column 2 | MW | m/z | MS t_{R} |
| 204 | 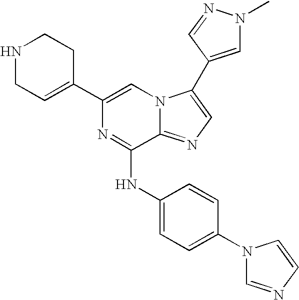 | 437.2 | 438.3 | 0.74 |
| 205 | 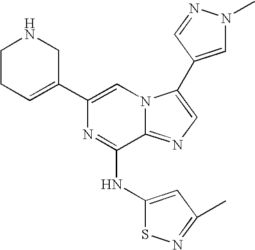 | 392.2 | 393.1 | 0.97 |
| 206 | 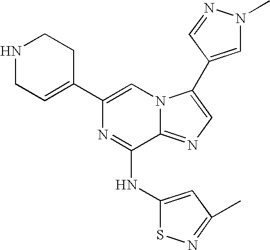 | 392.2 | 393.2 | 0.95 |

### Example 207

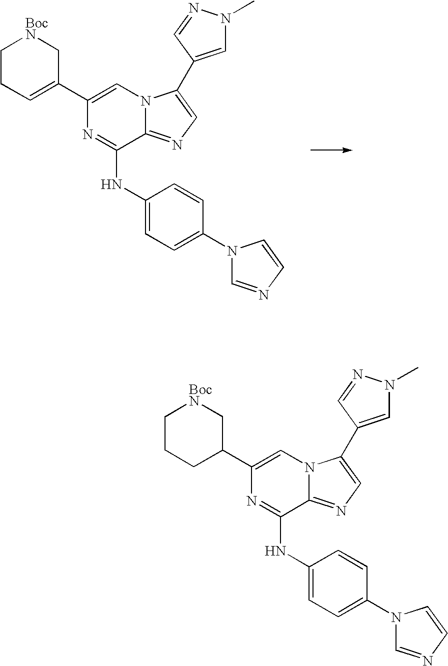

The product from example 202 (20 mg, TFA salt) was dissolved in THF (5 mL), and DIEA (500 μL) was added. To this mixture, 10% Pd/C (5 mg) was added and the resulting mixture was hydrogenated under H₂ atm. while stirring for overnight. After filtration and concentration the residue was purified by prep-LC to give the product 207. HPLC-MS t_{R}=1.45 min (UV_{254 nm}); mass calculated for formula C₂₉H₃₃N₉O₂, 539.3; observed MH⁺ (LCMS) m/z 540.3 (m/z).

### Example 208

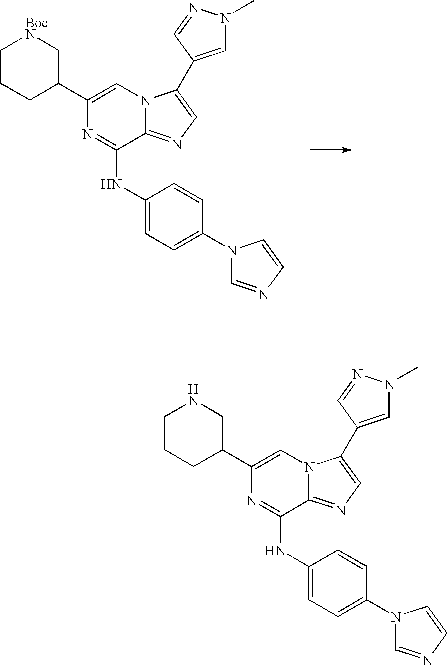

Product from example 207 was treated with was treated with 4 N HCl in dioxane (4 mL) and stirred at room temperature for 10 min. After concentration, the residue was dried with lyophilization to give 208. HPLC-MS t_{R}=0.80 min (UV_{254 nm}); mass calculated for formula C₂₄H₂₅N₉, 439.2; observed MH⁺ (LCMS) 440.2 (m/z).

By essentially the same procedure given in Preparative Example 208, compounds given in Column 2 of Table 19 can be prepared.

TABLE 19

| | | | LCMS | |
|---|---|---|---|---|
| Exam- | | | MH⁺ | HPLC |
| ple | Column 2 | MW | m/z | MS t_{R} |
| 209 | 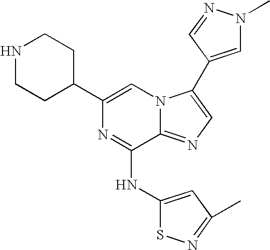 | 394.2 | 395.2 | 0.95 |

### Example 210

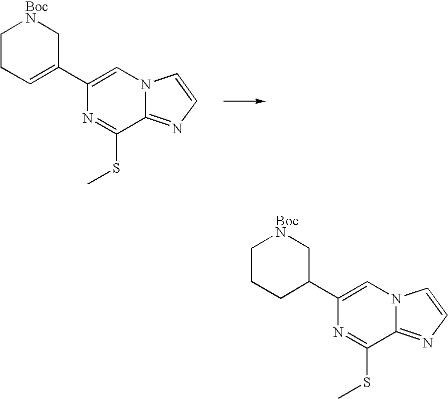

The product from example 198 (175 mg, 0.50 mmol) was dissolved in 20 mL of DME and 4 mL of water. To the mixture was added p-toluenesulfonyl hydrazide (1.86 g, 10 mmol). The mixture was heated up to 90° C. following the addition of NaOAc (1.64 g, 20.0 mmol) to the reaction. After stirring at reflux for 4 hours, additional p-toluenesulfonyl hydrazide (1.86 g, 10.0 mmol) and NaOAc (1.64 g, 20 mmol) were added. The mixture was at reflux overnight. After cooling to room temperature, the mixture was diluted with EtOAc (200 mL) and washed with H₂O, and brine. The organics were dried over Na₂SO₄ and concentrated. The resulting residue was purified by prep-LC to give the product 210. HPLC-MS t_{R}=1.92 min (UV_{254 nm}); mass calculated for formula C₁₇H₂₄N₄O₂S, 348.2; observed MH⁺ (LCMS) 349.2 (m/z).

### Example 211

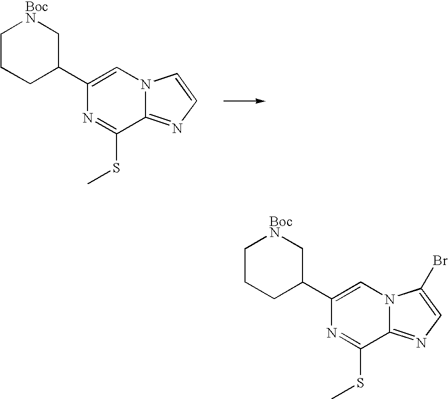

Product from example 211 was prepared using brominating conditions described in example 179. HPLC-MS t_{R}=5.89 min (UV_{254 nm}); mass calculated formula C₁₇H₂₃BrN₄O₂S, 426.1; observed MH⁺ (LCMS) 427.0 (m/z).

### Example 212

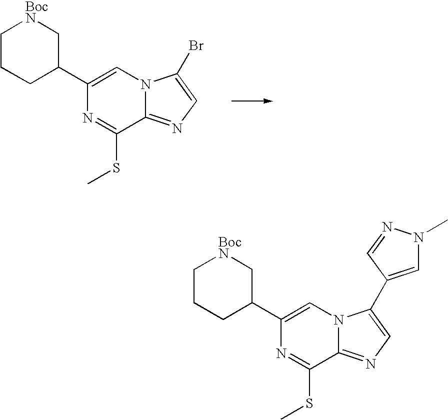

Compound 212 was synthesized using coupling conditions described in example 180. HPLC-MS t_{R}=1.99 min (UV_{254 nm}); mass calculated for formula C₂₁H₂₈N₆O₂S, 428.2; observed MH⁺ (LCMS) 429.2 (m/z).

### Example 213

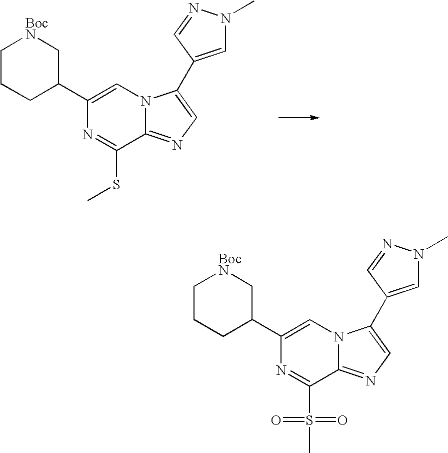

Compound 213 was synthesized using oxidation conditions described in example 181. HPLC-MS t_{R}=1.64 min (UV_{254 nm}); mass calculated for formula C₂₁H₂₈N₆O₄S; 460.2, observed MH⁺ (LCMS) 461.2 (m/z).

### Example 214

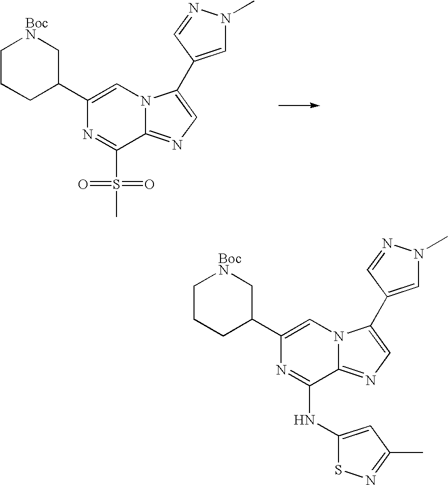

Compound 214 was prepared using the experimental condition described in example 182. HPLC-MS t_{R}=1.84 min (UV_{254 nm}); mass calculated for formula C₂₄H₃₀N₈O₂S; 494.2, observed MH⁺ (LCMS) 495.2 (m/z).

### Example 215

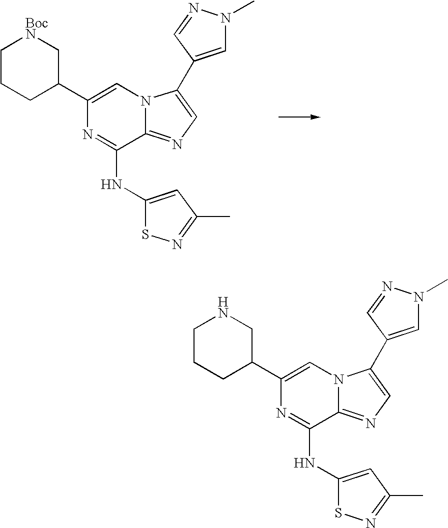

The compound 214 (20 mg) was treated with HCl (4 N in dioxane, 4 mL) and stirred at room temperature for 10 min. After concentrating, the residue was dried by lyophilization to give compound 215. HPLC-MS t_{R}=0.98 min (UV_{254 nm}); mass calculated for formula C₁₉H₂₂N₈S, 394.2; observed MH⁺ (LCMS) 395.2 (m/z).

### Example 216

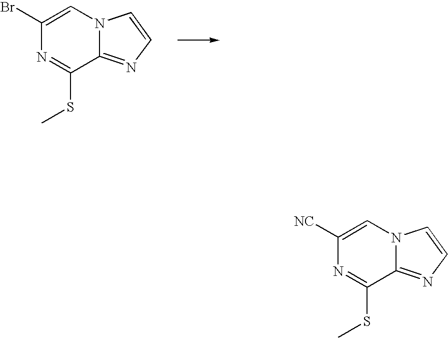

To a 25 mL round bottom flask charged with product from example 177 (486 mg, 2.0 mmol), Pd₂(dba)₃ (180 mg, 0.2 mmol), dppf (235 mg, 0.4 mmol), and Zn(CN)₂ (500 mg, 4.2 mmol) was added DME (10 ml) as solvent. The mixture was degassed thoroughly and placed under argon. This resulting mixture was then heated at 80° C. overnight. The reaction was diluted with EtOAc (100 mL) and filtered through Celite. After concentrating, the residue was purified with column chromatography (silica gel, Hexane/EtOAc=60/40) to give the product 216 (399 mg) as yellowish solid. ¹H NMR (400 MHz, CDCl₃) δ 8.31 (s, 1H), 7.80 (d, 1H), 7.69 (d, 1H), 2.66 (s, 3H). HPLC-MS t_{R}=1.15 min (UV_{254 nm}); mass calculated for formula C₈H₆N₄S; 190.0, observed MH⁺ (LCMS) 191.1 (m/z).

### Example 217

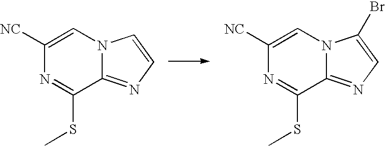

Product of the example 217 was prepared using brominating conditions described in example 179. HPLC-MS t_{R}=1.53 min (UV_{254 nm}); mass calculated for formula C₈H₅BrN₄S, 267.9; observed MH⁺ (LCMS) 269.0 (m/z).

### Example 218

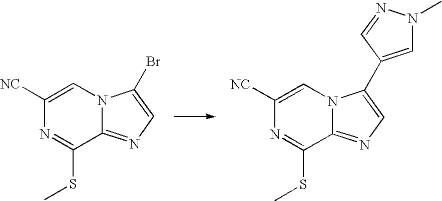

Compound 218 was synthesized using the coupling condition described in example 180. HPLC-MS t_{R}=1.36 min (UV_{254 nm}); mass calculated for formula C₁₂H₁₀N₆S, 270.1; observed MH⁺ (LCMS) 271.0 (m/z).

### Example 219, 220

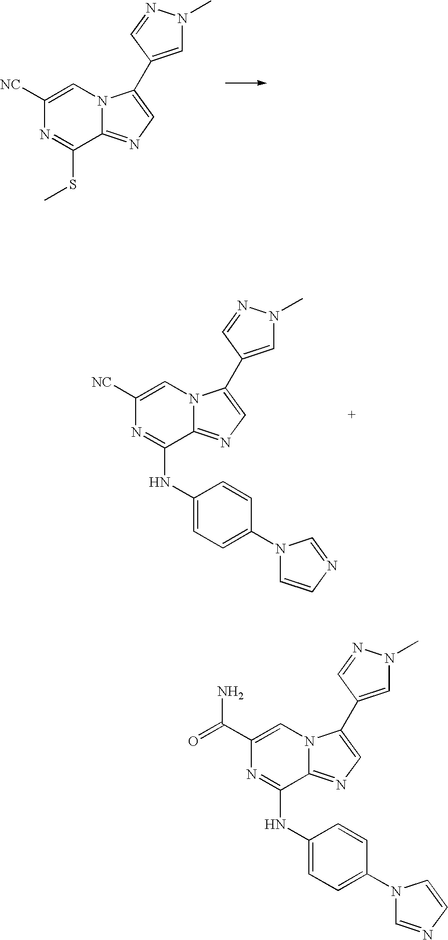

The aniline (32 mg, 0.42 mmol) was dissolved in dry DMSO (2 mL) and NaH (60% in oil, 8 mg, 0.2 mmol) was added under argon. The mixture was stirred for 10 min at room temperature then, sulfide 219 (27 mg, 0.1 mmol) in dry DMSO (0.5 mL) was added. The resulting mixture was heated up to 80° C. and stirred for 10 min. After cooling and LCMS analysis shown the formation of two products. The mixture was purified with Prep-LC to give the product 219 and 220 as TFA salt.

219: HPLC-MS t_{R}=0.77 min (UV_{254 nm}); mass calculated for formula C₂₀H₁₅N₉, 381.1; observed MH⁺ (LCMS) 382.1 (m/z).

220: HPLC-MS t_{R}=0.63 min (UV_{254 nm}); mass calculated for formula C₂₀H₁₇N₉O 399.2; observed MH⁺ (LCMS) 400.1 (m/z).

### Example 221

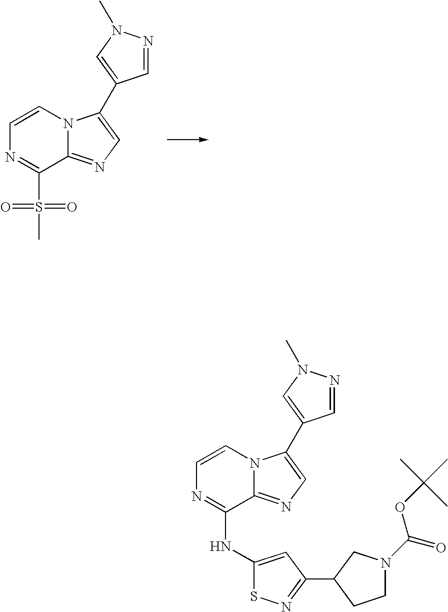

Compound 105 was synthesized via the synthetic method described in Preparative Example 105 described above. Also disclosed on page 71 in US20060 0106023 (A1).

3-(5-aminoisothiazol-3-yl) pyrrolidine-1-carboxylic-tert-butyl ester was prepared similar to the procedures described above for the synthesis in Examples 128-130.

A solution of the 3-(5-aminoisothiazol-3-yl)pyrrolidine-1-carboxylic-tert-butyl ester, (2 equivalents) in DMSO (10 mL) was treated with NaH (60% dispersion in oil, 2 equivalents) for 15 min at room temperature. Compound 105 (1 equivalent, 300 mg, 1.08 mmol) was then added to this solution at rt and the resultant solution was stirred at room temperature for 1 hr at which time LC-MS analysis indicated the reaction was complete. The reaction mixture was diluted with sat. ammonium chloride (10 mL) and extracted with 10% i-propylalcohol/dichloromethane (×3). The combined organic layers were washed with water, brine, dried over anhydrous sodium sulfate and concentrated. Purification by column chromatography ((SiO₂ 10% methanol/ethyl acetate) afforded compound 221 as a red solid 0.46 g (91%).

### Example 222

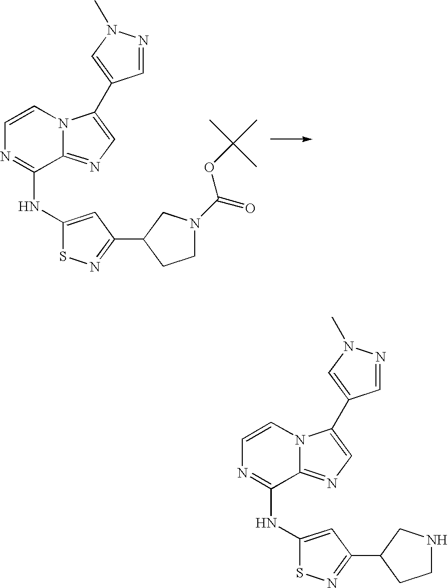

To compound 221 in THF (8 mL) was added 4N HCl in dioxane (2 mL). The resulting solution was stirred at room temperature for 16 hr at which time LC-MS analysis indicated that the reaction was complete. The solvent was evaporated. Purification by Prep-LC and conversion to a hydrochloric salt afforded compound 222. HPLC-MS t_{R}=2.55 Min (UV₂₅₄ₙₘ). Mass calculated for formula C₁₇H₁₈N₈S 366.1, observed LC/MS m/z 367.1 (M+H).

### Example 223

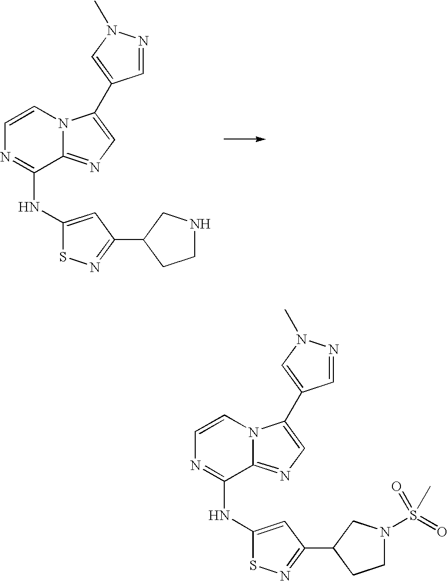

To compound 222 (50 mg, 0.14 mmol) in DCM (2 mL) was added DIEA (2.5 equivalents) at room temperature and the resulting heterogeneous solution was stirred at room temperature, then added methanesulfonyl chloride (1.5 equivalents). The resulting solution was stirred at room temperature for 15 min at which time LC-MS analysis indicated that the reaction was complete. After concentration the residue was purified by Prep-LC and conversion to a hydrochloric salt afforded compound 223. HPLC-MS t_{R}=3.34 Min (UV₂₅₄ₙₘ). Mass calculated for formula C₁₈H₂₀N₈O₂S₂ 444.12, observed LC/MS m/z 445.1 (M+H).

### Example 224

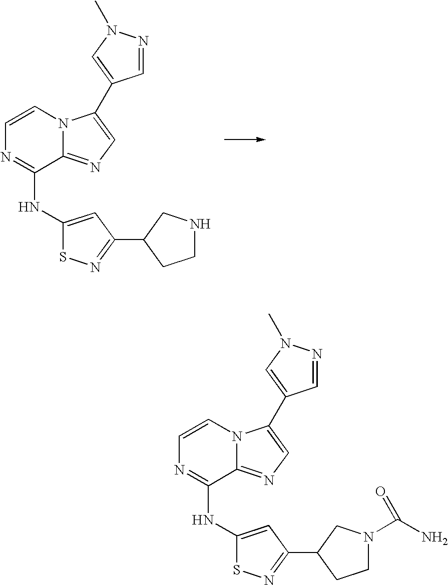

To compound 222 (50 mg, 0.14 mmol) in DCM (2 mL) was added trimethylsilyl isocyanate (2.1 equivalents) at room temperature. The resulting solution was stirred at room temperature for 15 min at which time LC-MS analysis indicated that the reaction was completed. After concentration the residue was purified by Prep-LC and conversion to a hydrochloric salt afforded compound 223. HPLC-MS t_{R} 2.72=Min (UV₂₅₄ₙₘ). Mass calculated for formula C₁₈H₁₉N₉OS 409.1, observed LC/MS m/z 410.1 (M+H).

### Example 225

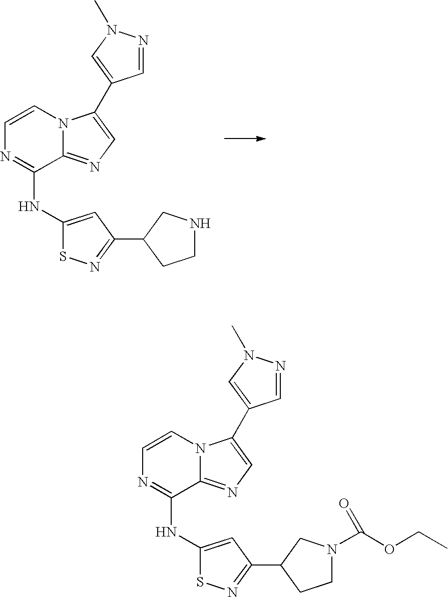

To compound 222 (50 mg, 0.14 mmol) in DCM (2 mL) was added DIEA (2.5 equivalents) at room temperature and the resulting heterogeneous solution was stirred at room temperature for 10 min. Then added ethyl chloroformate (1.5 equivalents) at room temperature. The resulting solution was stirred at room temperature for 15 min at which time LC-MS analysis indicated that the reaction was complete. After concentration the residue was purified by Prep-LC and conversion to a hydrochloric salt afforded compound 225. HPLC-MS t_{R}=3.88 Min (UV₂₅₄ₙₘ). Mass calculated for formula C₂₀H₂₂N₈O₂S 438.16, observed LC/MS m/z 439.1 (M+H).

The compounds 226-1 through 226-8 in Table 20 were prepared from the free amine and the appropriate reagents.

TABLE 20

| | | | MS | |
|---|---|---|---|---|
| Exam- | | Exact | m/z | HPLC |
| ple | Column 2 | mass | (MH)⁺ | MS t_{R} |
| 226-1 | 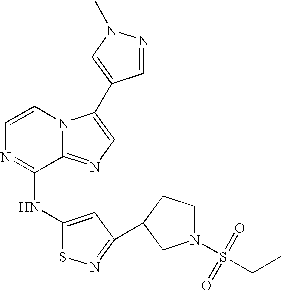 | 458 | 459 | 3.49 |
| 226-2 | 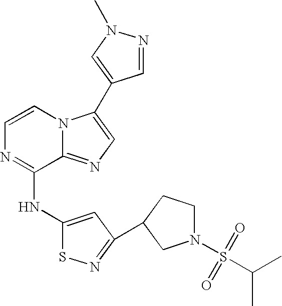 | 472 | 473 | 3.75 |
| 226-3 | 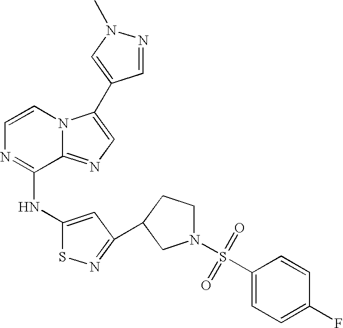 | 524 | 525 | 4.25 |
| 226-4 | 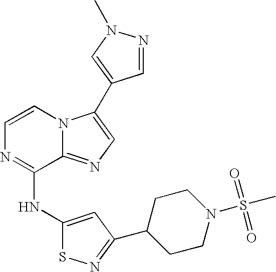 | 458 | 459 | 3.44 |
| 226-5 | 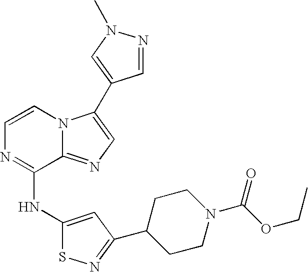 | 452 | 453 | 4.10 |
| 226-6 | 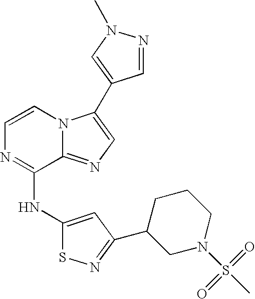 | 458 | 459 | 3.59 |
| 226-7 | 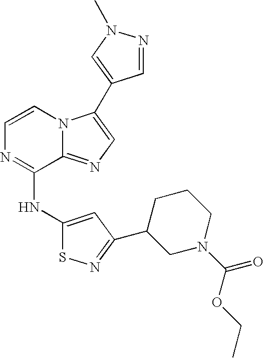 | 452 | 453 | 4.22 |
| 226-8 | 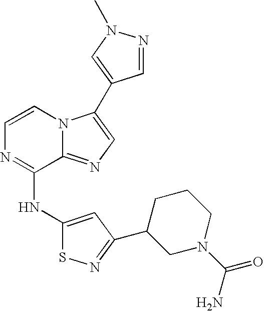 | 423 | 424 | 2.97 |

### Example 227

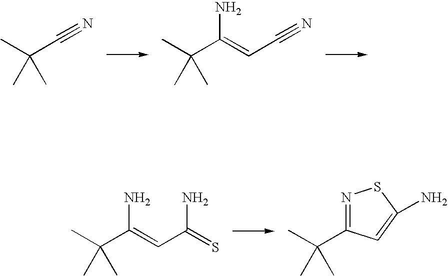

Compound 227 was synthesized from compound 1 via the synthetic method described by Hackler et al., Journal of Heterocyclic Chemistry (1989), 26 (6), 1575-8.

### Example 228

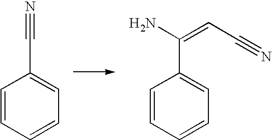

A 2.5M n-BuLi solution (20.4 mL, 50.9 mmol) was slowly added to a solution of diisopropylamine (7.2 mL, 50.9 mmol) in anhydrous THF (75 mL) under argon at −78° C. After stirring at −78° C., the solution was treated with acetonitrile (2.5 mL, 48.5 mmol) dissolved in anhydrous THF (10 mL). After 10 minutes, Benzonitrile was added dropwise to the above solution at −78° C. The resulting suspension was allowed to warm to room temperature. The reaction mixture was stirred at room temperature overnight at which time thin layer chromatography (40% ethyl acetate/hexanes) indicated that the reaction was complete. The reaction mixture was poured into ice water (200 mL), and then concentrated to remove the organic solvent. The resulting emulsion was extracted twice with diethyl ether. The combined organic layers were dried over anhydrous sodium sulfate and concentration afforded the title compound 228 that was used directly in the next step.

### Example 229

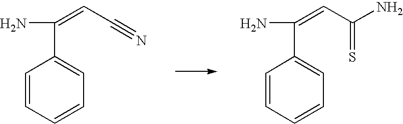

A solution of compound 228 (1 g, 6.9 mmol) in THF/ethanol (1:1, 10 mL) in a high pressure vessel was cooled to 0° C. (ice-bath) and treated with hydrogen sulfide gas for 5 minutes. The tube was sealed and heated to 90° C. for 2 hr. LC-MS analysis indicated the reaction was complete; concentration afforded the title compound 229 that was used directly in the next step.

### Example 230

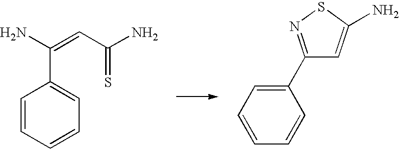

To compound 229 (1.15 g, 3.47 mmol) and potassium carbonate (2 equivalents) in diethyl ether (20 mL) was added an ethereal solution of iodine (1 equivalent) dropwise at reflux. The resulting solution was heated at reflux for 2 hr at which time LC-MS analysis indicated that the reaction was complete. The mixture was cooled to 25° C. and concentrated. Purification by column chromatography (SiO₂, 40% ethyl acetate/hexanes) afforded compound 230 as a red/orange solid 0.29 g (48%). HPLC-MS t_{R}=1.38 Min (UV₂₅₄ₙₘ). Mass calculated for formula C₉H₈N₂S 176.0, observed LC/MS m/z 177.1 (M+H).

### Example 231 & 232

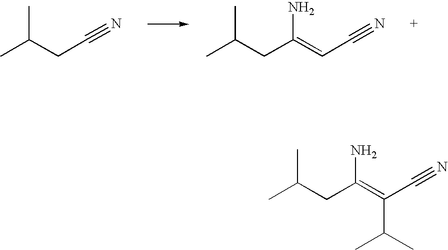

A 2.5M n-BuLi solution (20.4 mL, 50.9 mmol) was slowly added to a solution of diisopropylamine (7.2 mL, 50.9 mmol) in anhydrous THF (75 mL) under argon at −78° C. After stirring at −78° C., the solution was treated with acetonitrile (2.5 mL, 48.5 mmol) dissolved in anhydrous THF (10 mL). After 10 minutes, a solution of 3-methyl butyronitrile (5.1 mL, 40 mmol) in anhydrous THF (75 mL), under argon at −78 C, was added drop wise to the above solution. The resulting suspension was allowed to warm to room temperature. The reaction mixture was stirred at room temperature overnight at which time thin layer chromatography (40% ethyl acetate/hexanes) indicated that the reaction was complete. The reaction mixture was poured into ice water (200 mL), and then concentrated to remove the organic solvent. The resulting emulsion was extracted twice with diethyl ether. The combined organic layers were dried over anhydrous sodium sulfate and concentration afforded the a mixture of two compounds 231 and 232 in 1:3 ratio. These two compounds separated by column chromatography and the compound 231, HPLC-MS t_{R}=Min (UV₂₅₄ₙₘ). Mass calculated for formula C₇H₁₂N₂, M+124.18, observed LC/MS m/z 125.20.10 (M+H), is used in the next step. Undesired compound, 232 HPLC-MS t_{R}=Min (UV₂₅₄ₙₘ). Mass calculated for formula C₁₀H₁₈N₂, M+ 166.26, observed LC/MS m/z 167.40 (M+H). was discarded.

### Example 233

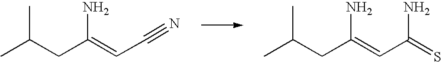

A solution of compound 231 (1 g, mmol) in THF/ethanol (1:1, 10 mL) in a high pressure vessel was cooled to 0° C. (ice-bath) and treated with hydrogen sulfide gas for 5 minutes. The tube was sealed and heated to 90° C. for 2 h. LC-MS analysis indicated the reaction was complete, concentration afforded the title compound 233 that was used directly in the next step. HPLC-MS t_{R}=Min (UV₂₅₄ₙₘ). Mass calculated for formula C₇H₁₄N₂S, M+ 158.26, observed LC/MS m/z 159.30 (M+H).

### Example 234

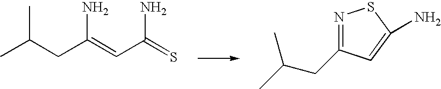

To compound 233 (1.15 g, mmol) and potassium carbonate (2 equivalents) in diethyl ether (20 mL) was added an ethereal solution of iodine (1 equivalent) dropwise at reflux. The resulting solution was heated at reflux for 2 hr. at which time LC-MS analysis indicated that the reaction was complete. The mixture was cooled to 25° C. and concentrated. Purification by column chromatography (SiO₂, 40% ethyl acetate/hexanes) afforded compound 234 as a viscous liquid 0.29 g (48%). HPLC-MS t_{R}=Min (UV₂₅₄ₙₘ). Mass calculated for formula C₇H₁₂N₂S, M+ 156.25, observed LC/MS m/z 157.40 (M+H).

### Example 235

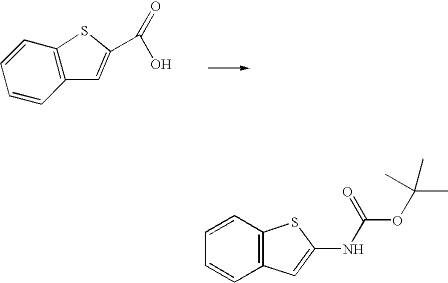

A solution of benzo[b]thiophene-2 carboxylic acid (1.25 g, 7.03 mmol), diphenylphosphoryl azide (1.94 g, 7.03 mmol) and triethylamine (0.98 mL, 7.03 mmol) in tert-butanol (20 mL) was heated at reflux for 5 hours, at which time thin layer chromatography (DCM/Hexanes) indicates the reaction is complete. The reaction mixture was cooled to room temperature, poured into water and extracted with diethyl ether (3×). The combined ether extracts were washed with brine, dried over anhydrous sodium sulfate and then concentrated to afford a beige solid. Purification by column chromatography (SiO₂ DCM/Hexanes) afforded compound 235 as a white solid 0.96 g (64%). HPLC-MS t_{R}=2.7 Min (UV₂₅₄ₙₘ). Mass calculated for formula C₁₃H₁₅NO₂S, M+ 249.33, observed LC/MS m/z 250.40 (M+H).

### Example 236

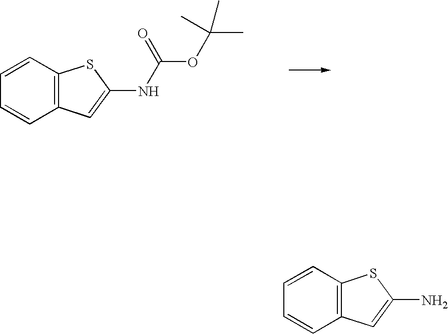

A solution of compound 235 (0.250 g, 1.00 mmol) was stirred in 4 M HCl solution in 1,4-dioxane (3 mL) at room temperature for 2 hrs at which time thin layer chromatography (DCM/Hexanes) indicated the reaction was complete. The reaction mixture was cooled to room temperature and concentrated under vacuum. The residue was diluted with acetonitrile, sonicated, and concentrated to afford compound 236 as a grey solid 0.24 g (91%). HPLC-MS t_{R}=1.5 Min (UV₂₅₄ₙₘ). Mass calculated for formula C₈H₇NS, M+ 149.21, observed LC/MS m/z 150.40 (M+H).

### Example 237

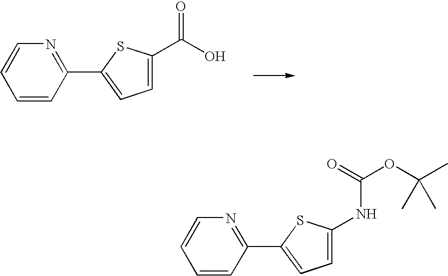

By essentially the same procedure given in Preparative Example 235, 237 can be prepared from compound, 5-pyridin-2yl-thiophene-2carboxylic acid.

### Example 238

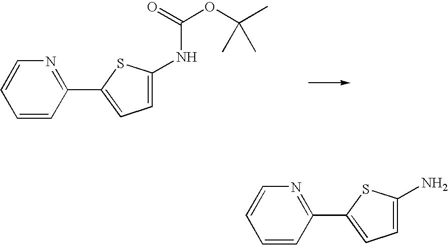

By essentially the same procedure given in Preparative Example 236, 238 can be prepared from compound 237.

### Example 239

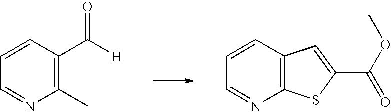

Compound 2-methylpyridine-3-carboxaldehyde (2.5 g, 17.7 mmol) was dissolved in DMF (25 mL) and water (2.5 mL). Potassium carbonate (1.1 equivalents) and methyl thioglycolate (1.1 equivalents) are added portion wise resulting in a bright orange solution which was heated at 40° C. for 16 hr. LC-MS analysis indicated that the reaction was complete. The reaction mixture was allowed to cool to room temperature and then quenched with ice-cold water (150 mL) and placed in an ice-bath to enhance precipitation. The precipitate was isolated by filtration, affording compound 242 as an off-white solid 1.87 g (55%).

### Example 240

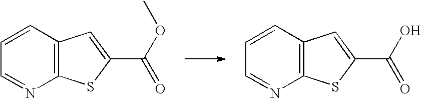

By essentially following the same procedure given in Preparative Example 133, compound 240 can be prepared from compound 239.

### Example 241

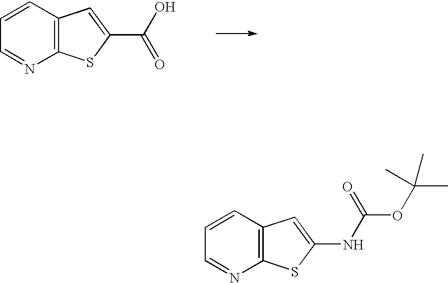

By essentially following the same procedure given in Preparative Example 237, compound 241 can be prepared from compound 240.

### Example 242

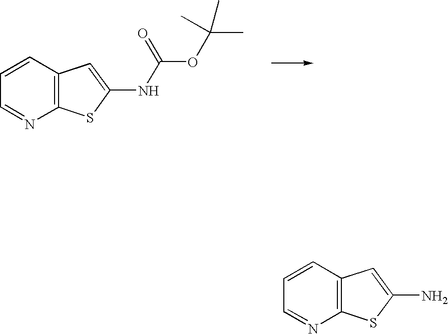

By essentially following the same procedure given in Preparative Example 238, compound 242 can be prepared from compound 241.

### Example 243

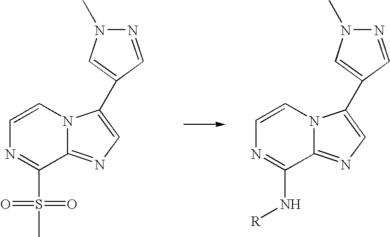

By essentially the same procedure given in Preparative Example 106, the compounds given in Column 2 of Table 21 can be prepared from compound 105.

TABLE 21

| | | | MS | |
|---|---|---|---|---|
| Exam- | | Exact | m/z | HPLC |
| ple | Column 2 | mass | (M + H) | MS t_{R} |
| 243-1 | 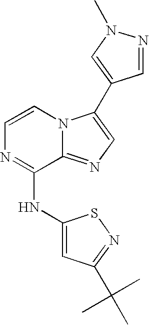 | 353.1 | 354.1 | 4.37 |
| 243-2 | 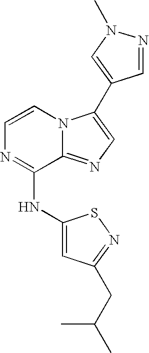 | 353.14 | 354.10 | 4.50 |
| 243-3 | 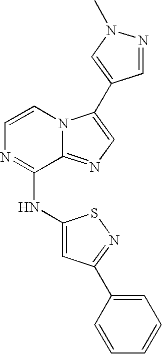 | 373.1 | 374.1 | 4.76 |
| 243-4 | 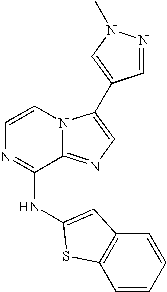 | 346.1 | 347.1 | 4.60 |
| 243-5 | 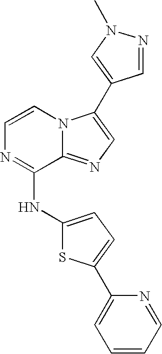 | 373.1 | 374.0 | 2.96 |
| 243-6 | 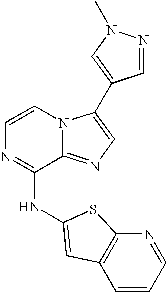 | 347.1 | 348.0 | 3.05 |
| 243-7 | 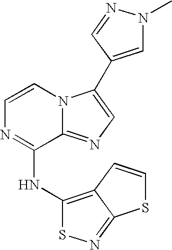 | 353.1 | 354.1 | 4.20 |
| 243-8 | 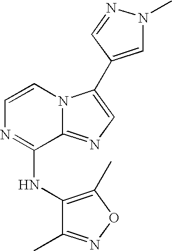 | 309.1 | 310.2 | 2.19 |
| 243-9 | 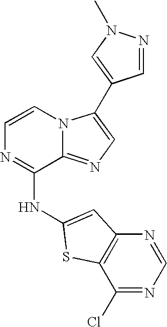 | 382 | 383 | 1.97 |

### Example 244

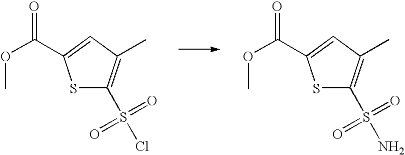

5-Chlorosulfonyl-4-methyl-thiophene-2-carboxylic acid methyl ester (1.76 g, 6.92 mmol) was dissolved in 1,4-dioxane (40 mL) and cooled in an ice-bath. Ammonia gas was bubbled into the reaction mixture until thin layer chromatography indicated the reaction was complete (ca ˜10 minutes). The reaction mixture was filtered, the solids were rinsed with dichloromethane and the filtrate was concentrated to afford the title compound 231 as a white solid 1.53 g (94%).

### Example 245

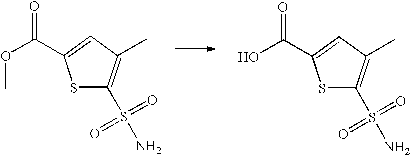

To a solution of compound 231 (1.50 g, 6.37 mmol) in THF/water (80 mL/20 mL) was added 1N LiOH (12.8 mL, 12.8 mmol) at room temperature. The reaction mixture was stirred at room temperature for 16 hr at which time thin layer chromatography indicated the reaction was complete. The reaction mixture was concentrated, the residue acidified to pH 4 with 1N HCl and extracted with ethyl acetate (×4). The combined organic layer was dried over anhydrous Na₂SO₄ and concentrated to afford compound 232 as a white solid 1.29 g (92%).

### Example 246

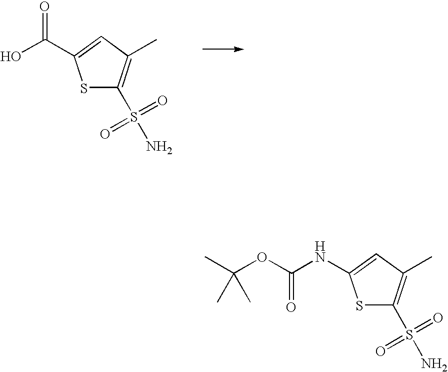

A solution of compound 232 (0.59 g, 2.69 mmol), diphenylphosphoryl azide (0.58 mL, 2.69 mmol) and triethylamine (0.37 mL, 2.69 mmol) in t-butanol (20 mL) was heated at reflux for 5 hr, at which time thin layer chromatography (DCM/Hexanes) indicated that the reaction is complete. The reaction mixture was cooled to room temperature, poured into water and extracted with diethyl ether (×3). The combined ether extracts were washed with brine, dried over anh. sodium sulfate and then concentrated to afford a beige solid. Purification by column chromatography (SiO₂ 40% ethyl acetate/hexanes) afforded compound 233 as a white solid 0.36 g (46%).

### Example 247

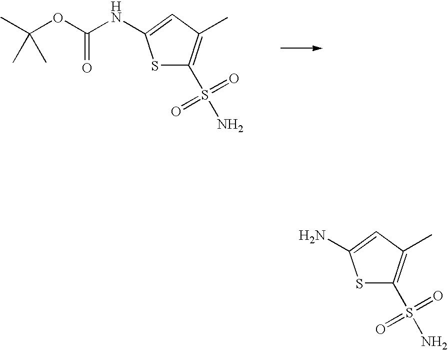

A solution of compound 233 (0.20 g, 0.68 mmol) was stirred in 4M HCl solution in 1,4-dioxane (3 mL) at room temperature for 2 h at which time thin layer chromatography (DCM/Hexanes) indicated that the reaction was complete. The reaction mixture was concentrated under vacuum. The residue was diluted with acetonitrile, sonicated and concentrated to afford compound 234 as a grey solid 0.15 g (96%).

### Preparative Examples 248-1-10

By essentially using the same procedures set forth in Preparative Example 244 through 247 by using amines listed in column 1 compounds in column 2 of the table 22, are prepared.

TABLE 22

| | | | | LCMS MH⁺ |
|---|---|---|---|---|
| Serial No. | Column 1 | Column 2 | MW | m/z |
| 248-1 | 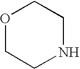 | 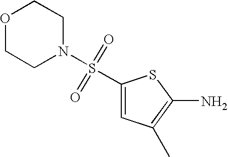 | 262.04 | 263.1 |
| 248-2 | 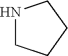 | 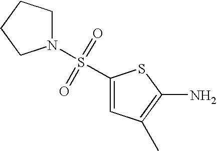 | 246.05 | 247.1 |
| 248-3 | 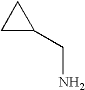 | 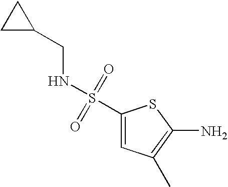 | 246.05 | 247.1 |
| 248-4 | 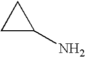 | 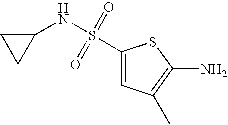 | 232.03 | 233.1 |
| 248-5 | 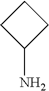 | 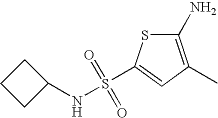 | 246.05 | 247.1 |
| 248-6 | 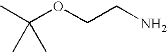 | 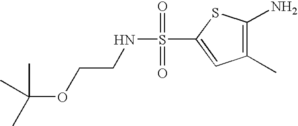 | 236.03 | 237.1 |
| 248-7 | 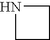 | 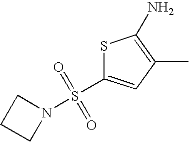 | 232.03 | 233.1 |
| 248-8 | 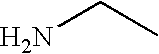 | 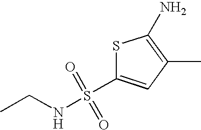 | 220.03 | 221.1 |
| 248-9 | 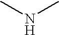 | 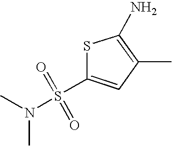 | 220.03 | 221.10 |
| 248-10 | 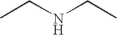 | 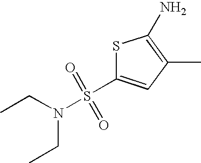 | 248.07 | 249.20 |

### Example 249

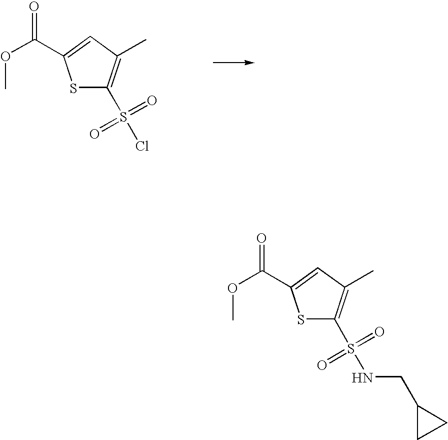

5-(cyclopropylmethyl-sulfamoyl)-4-methyl-thiophene-2-carboxylic acid methyl ester prepared as in example 244.

### Example 250

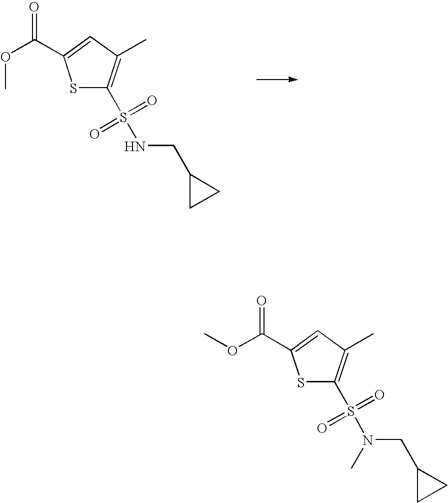

Compound of preparative 249 (0.275 g, 1.0 mmol) in THF (5 mL) was added to the suspension of NaH (60% dispersion in oil) (0.040 g, 1.5 mmol) in THF (5 mL) at 0° C. and stirred for 10 minutes. Then the Iodomethane 0.284 g, 2 mmol) in THF (1 mL) was added the reaction mixture. The reaction was stirred for 2 hours at room temperature. After the completion of the reaction (LCMS analysis), reaction is quenched with NH₄Cl soln. and extracted with ethyl acetate. The organic layer was washed with brine and dried over anhydrous Na₂SO₄. Filtered and concentrated to obtain crude product 250 (0.250 g. 86%). HPLC-MS t_{R}=1.826 min (UV_{254 nm}); mass calculated for formula C₁₁H₁₅NO₄S₂, 289.04; observed MH⁺ (LCMS) 290.0 (m/z).

### Example 251

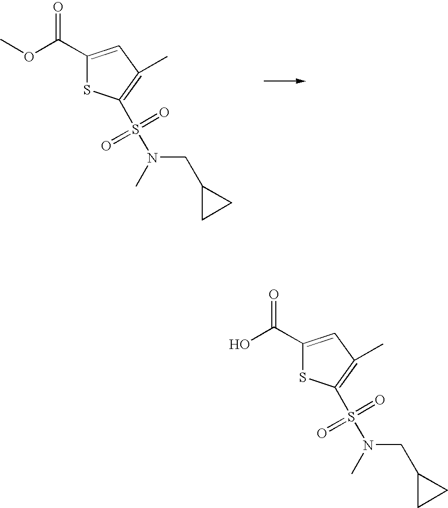

By essentially the same procedure given in Preparative Example 245, the compound 251 can be prepared from compound 250.

### Example 252

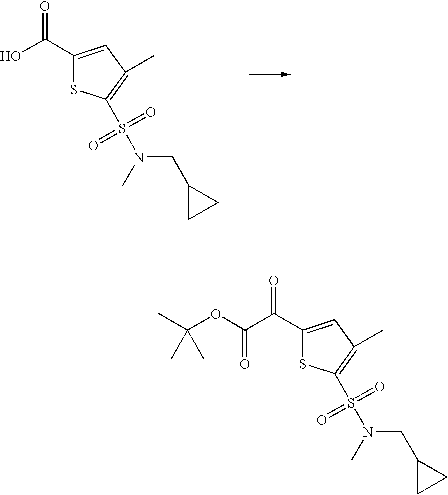

By essentially the same procedure given in Preparative Example 246, the compound 252 can be prepared from compound 251

### Example 253

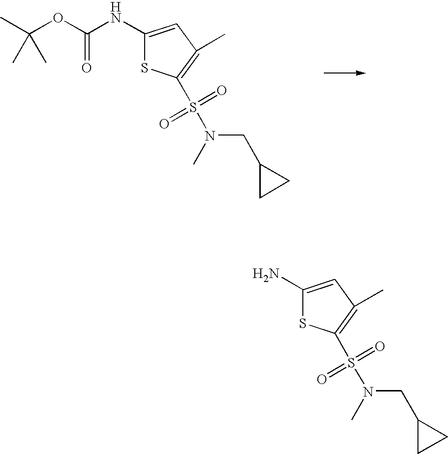

By essentially the same procedure given in Preparative Example 247, the compound 253 can be prepared from compound 252

Compounds listed in column 2 (254-1 through 254-7) of Table-23 were essentially prepared from the amines ranging from 247 and 248-1 through 10 following the procedure described in preparation of compound 106.

TABLE 23

| | | | MS | |
|---|---|---|---|---|
| Ex- | | Exact | m/z | HPLC |
| ample | Column 2 | Mass | (M + H) | MS t_{R} |
| 254-1 | 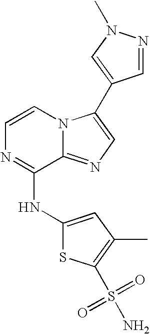 | 389.1 | 390.0 | 2.87 |
| 254-2 | 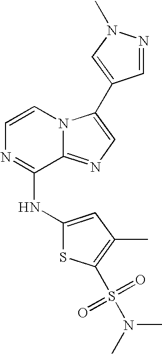 | 417.1 | 418.1 | 3.82 |
| 254-3 | 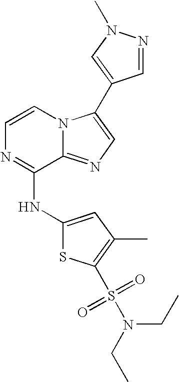 | 445.16 | 446.20 | 4.10 |
| 254-4 | 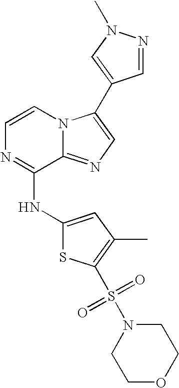 | 459.11 | 460.23 | 4.02 |
| 254-5 | 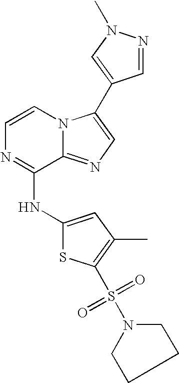 | 443.12 | 444.23 | 4.38 |
| 254-6 | 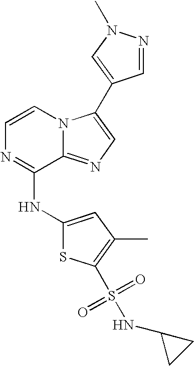 | 429.10 | 430.20 | 3.91 |
| 254-7 | 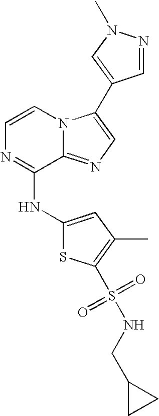 | 443.12 | 444.20 | 4.19 |
| 254-8 | 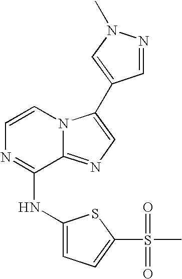 | 374.1 | 375.1 | 3.09 |

### Example 255

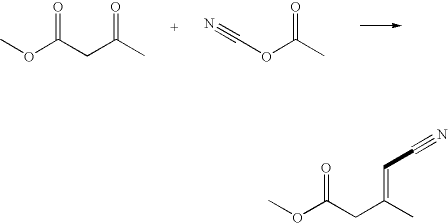

Acetoacetate (45.4 g, 458 mmol), cyanoacetic acid (39 g, 458 mmol), NH₄OAc (7.3 g, 94.7 mmol), AcOH (13.0 mL), and benzene (130 mL) was stirred for 24 hr at reflux with a Dean-Stark trap. The mixture was cooled to room temperature, washed with sat. NaHCO₃, brine, dried with Na₂SO₄, and conc. in vacuo. The crude product was distilled at 65° C. at 0.5 Torr: to give compound, methyl 4-Cyano-3-methylbut-3-enoate (44.27 g, 70%) as a mixture E/Z isomers. ¹H NMR DMSO_{d6}: 5.69 (q, J=0.6 Hz, 1H), 5.62 (q, J=0.6 Hz, 1H), 3.61 (s, 3H), 3.60 (s, 3H), 3.42 (s, 2H), 3.35 (d, J=1.2 Hz, 2H), 2.01 (d, J=1.2 Hz, 3H), 1.93 (d, J=1.2 Hz, 3H).

### Example 256

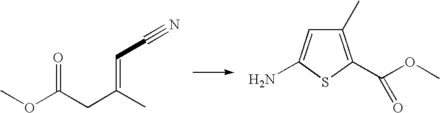

Et₂NH (36.2 mL, 350 mmol) was added dropwise to a mixture of compound methyl 4-Cyano-3-methylbut-3-enoate (44.27 g, 318 mmol) and S-flakes (10.20 g, 318 mmol) in EtOH (250 mL). The reaction stirred at room temperature for 3 hr. The mixture was concentrated to a minimal volume and placed in an ice bath. HCl (conc.) was slowly added to the mixture to give a yellow/orange solid. The precipitate was collected by vacuum filtration and washed with Et₂O to give compound (256) Methyl 5-Amino-3-methylthiophene-2-carboxylate Hydrochloride (41.22 g, 62%). ¹H NMR DMSO_{d6}: 6.91 (s, 2H), 5.76(s, 1H), 3.61 (s, 3H), 2.62 (s, 3H).

### Example 257

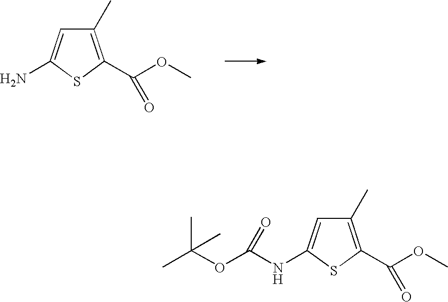

Compound (256) Methyl 5-Amino-3-methylthiophene-2-carboxylate Hydrochloride (1.25 g, 6.75) was mixed with tert-BOC anhydride (1.62 g, 7.42 mmol), diisopropyl ethyl amine (1.29 mL, 7.42 mmol), and a catalytic amount of dimethylaminopyridine (10 mg) in DMF (50 mL). The reaction was heated at 60° C. for 3 hr. The reaction was concentrated and the residue dissolved in EtOAc (100 mL). This solution was washed with water followed by brine. The organic layer was then dried over Na₂SO₄ and conc. in vacuo. The crude material was purified via column chromatography using a gradient of 5% EtOAc/Hexanes to 40% EtOAc/Hexanes. Compound, 5-tert-Butoxycarbonylamino-3-methyl-thiophene-2-carboxylic acid ethyl ester, was isolated in 32% yield (0.612 g). 0.304 g of the starting material was also recovered. ¹H NMR CDCl₃: 7.29, (bs, 1H), 6.30, (s, 1H), 4.26 (q, J=6.8 Hz, 2H) 2.46 (s, 3H), 1.52 (s, 9H), 1.32 (t, J=6.8 Hz, 3H).

### Example 258

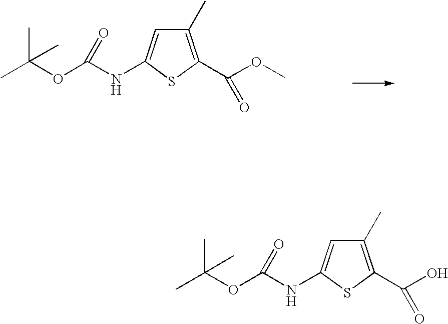

5-tert-Butoxycarbonylamino-3-methyl-thiophene-2-carboxylic acid ethyl ester (0.600 g, 2.10 mmol) was mixed with 1M NaOH (2.3 mL) in MeOH (15 mL) and H₂O (5 mL). The solution was heated to reflux for 48 h. The reaction was cooled to 0° C. and 1M HCl was added until the solution had a pH between 4 to 5. The reaction was washed with EtOAc (3×, 50 mL). The organic layer was dried with Na₂SO₄ and conc. in vacuo. This material was used without further purification.

### Example 259

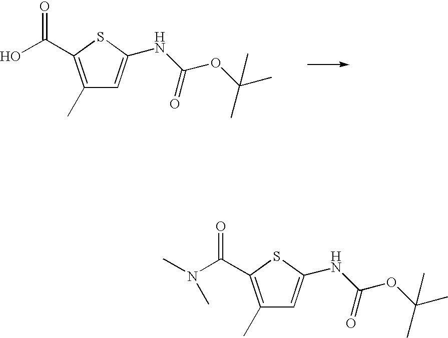

5-tert-Butoxycarbonylamino-3-methyl thiophene-2-carboxylic acid (258,1 mmol, 257 mg) was dissolved in dichloromethane and added with, 1.5 eq of EDCl, and 4.0 eq. of DIEA in CH₂Cl₂ at room temperature. After 10 minutes, the NN-dimethylamine.HCl salt (3 eq.) was added. The reaction stirred at room temperature for 3 hrs. Then the crude reaction material was concentrated, was dissolved in EtOAc (25 mL), and washed with H₂O (2×, 25 mL), followed by brine (25 mL). The organic layer was dried over Na₂SO₄, filtered, and concentrated to give the crude product which was chromatographed to give the product 259. HPLC-MS t_{R}=2.4 Min (UV₂₅₄ₙₘ). Mass calculated for formula C₁₃H₂₀N₂O₃S, M+ 284.37, observed LC/MS m/z 285.40 (M+H).

### Example 260

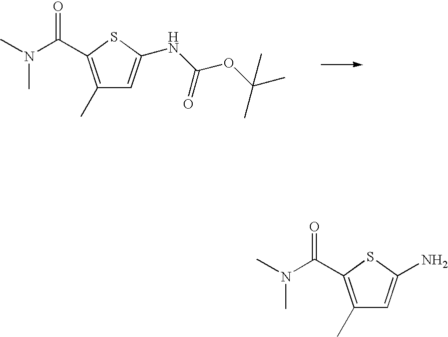

The compound 259 from the above step was dissolved in dichloromethane (2 mL) and cooled to 0° C. To this solution, a 50% TFA-DCM (2 mL) was added and the reaction mixture stirred for 30 minutes at room temperature. The reaction was concentrated and dried under vacuum to give the TFA salt of the 5-amino 3-methyl thiophene-2-carboxylic acid dimethyl amide, HPLC-MS t_{R}=0.6 Min (UV₂₅₄ₙₘ). Mass calculated for formula C₈H₁₂N₂OS, M+ 184.26, observed LC/MS m/z 185.40 (M+H).

### Example 261

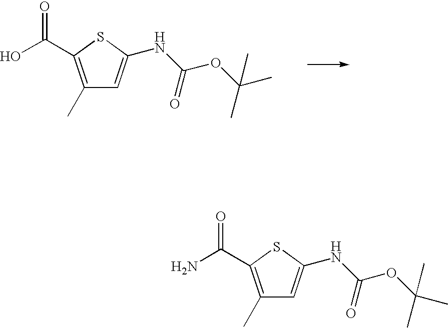

By essentially the same procedure given in Preparative Example, 259 compound 261 can be prepared from compound 258.

### Example 262

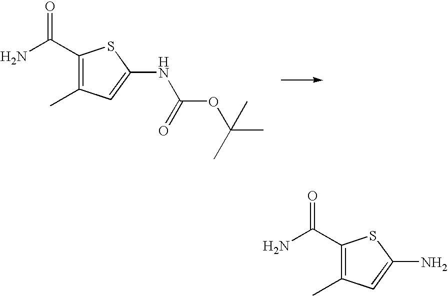

By essentially the same procedure given in Preparative Example, 260 compound 262 can be prepared from compound 261.

### Example 263

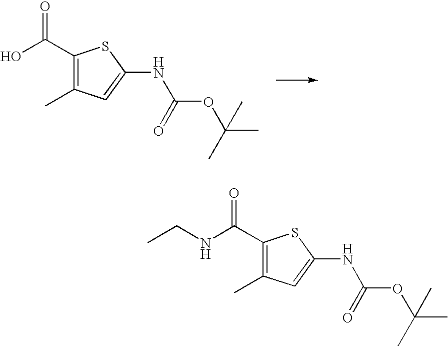

By essentially the same procedure given in Preparative Example, 259 compound 261 can be prepared from compound 258.

### Example 264

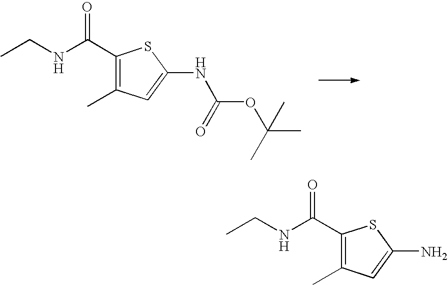

By essentially the same procedure given in Preparative Example, 260 compound 264 can be prepared from compound 263.

### Example 265

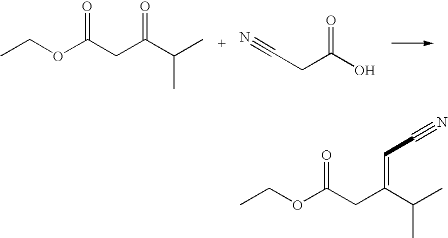

By essentially following the procedure in the example 255, the compound, 265 can be prepared.

### Example 266

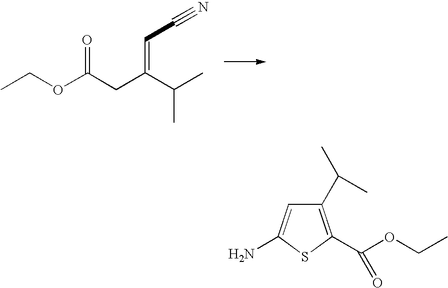

By essentially following the procedure in the example 256, the compound, 266 can be prepared.

### Example 267

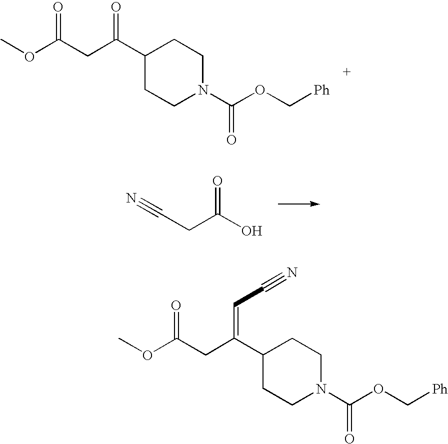

By essentially following the procedure in the example 255, the compound, 267 can be prepared.

### Example 268

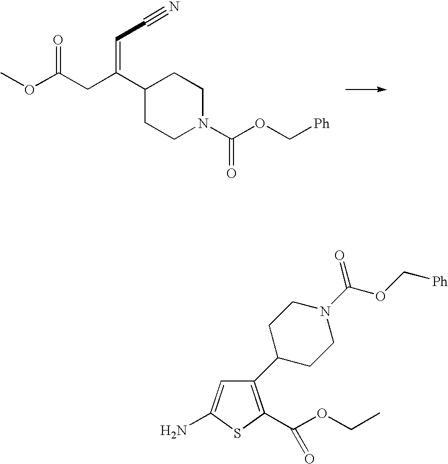

By essentially following the procedure in the example 256, the compound, 268 can be prepared.

Compounds (269-1 through 269-7) listed in column 2 of Table-24 were essentially prepared from the amines ranging from—following the procedure described in preparation of compound 106.

TABLE 24

| Ex- | | | MS | |
|---|---|---|---|---|
| am- | | Exact | m/z | HPLC |
| ple | Column 2 | Mass | (M + H) | MS t_{R} |
| 269-1 | 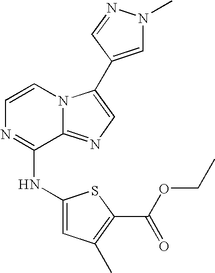 | 382.1 | 383.1 | 4.68 |
| 269-2 | 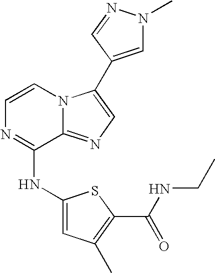 | 381.14 | 382.20 | 4.35 |
| 269-3 | 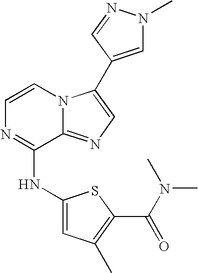 | 381.14 | 382.20 | 4.50 |
| 269-4 | 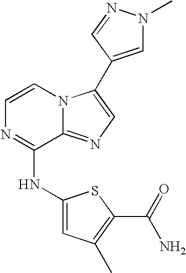 | 353.11 | 354.20 | 3.25 |
| 269-5 | 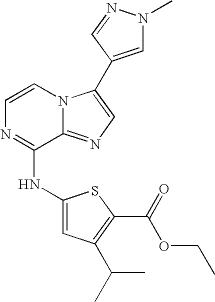 | 410.15 | 411.30 | 5.10 |
| 269-6 | 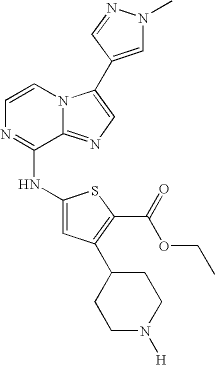 | 451.18 | 452.20 | 4.30 |
| 269-7 | 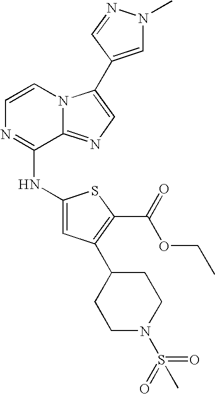 | 529.16 | 530.20 | 3.50 |

### Example 270

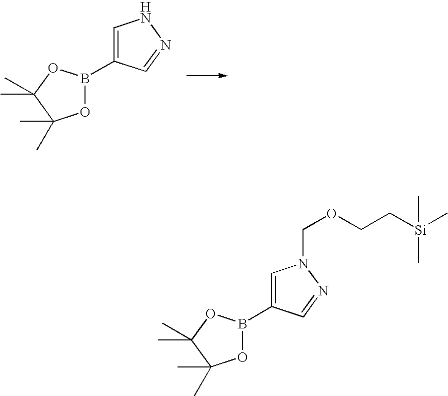

To a suspension of potassium carbonate (5.85 g, 1.5 equiv) and 1H-pyrazole-4-boronate (5.48 g, 1.0 equiv) in NMP (50 mL) at room temperature was added SEMCl (5.2 mL, 1.05 equiv) dropwise (mildly exothermic). The resulting mixture was allowed to stir for an additional 45 min at room temperature. The reaction was diluted with ethyl acetate, rinsed with water (×2), brine and dried (sodium sulfate). Filtration and concentration afforded the title compound (270) that used directly in the next step.

### Example 271

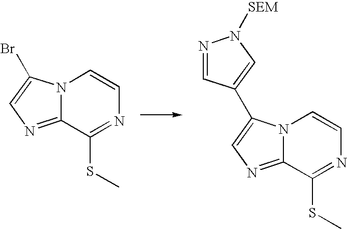

A flask was charged with compound 103 (1.83 g, 1.00 equiv), Bpin-compound 270 (2.08 g, 1.3 equiv), PdCl2(dppf) (0.4 g, 0.1 equiv) and potassium phosphate monohydrate (3.4 g, 3.0 equiv). After purging the flask with argon, 1,4-dioxane (50 mL) and water (5 mL) were added and the resulting mixture was heated at 40° C. overnight (23 hr). The reaction was cooled to room temperature. EtOAc was added to the reaction mixture and filtered through Celite. After concentration the residue was purified by column chromatography (silica gel, 25% EtOAc/hexane) to give the title compound 271 (46%).

### Example 272

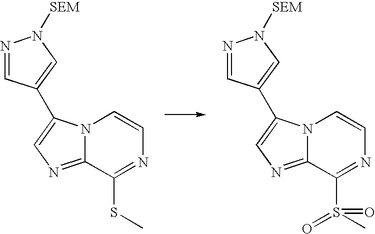

To a solution of compound 271 (1.02 g, 1.0 equiv) in DCM (10 mL) was added m-CPBA (1.1 g, 77%, 2.05 equiv) in one portion. The resulting mixture was stirred at room temperature for 30 min. The mixture was concentrated and then partitioned between EtOAc and water. The organic layer was washed with NaHCO₃ (sat. aq., ×2), brine and dried (Na₂SO₄). After concentration, the crude product compound 272 was used in the next step directly without further purification.

### Example 273

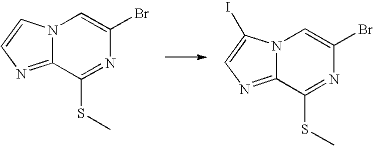

To a solution of compound 177 (2.00 g, 8.19 mmol) in DMF (50 mL) was added N-iodosuccinimide (1.84 g, 8.19 mmol). The reaction mixture was stirred at 60° C. for 16 hr. The mixture was cooled to 25° C. and concentrated. Purification by column chromatography (SiO₂, 40% ethyl acetate/hexanes) afforded compound 273 as a white solid 2.30 g (76%). ¹H-NMR (400 MHz, DMSO-d₆) δ 8.3 (s, 1H), 7.8 (s, 1H), 2.6 (s, 3H). HPLC-MS t_{R}=1.87 Min (UV_{254 nm}). Mass calculated for formula C₇H₅BrIN₃S, 370.01, observed LC/MS m/z 370.9 (M+H).

### Example 274

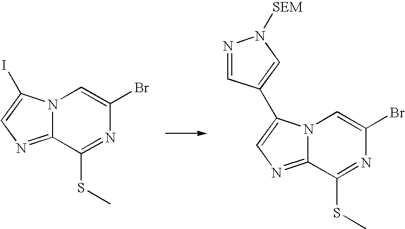

A flask was charged with iodo-compound 273 (1.83 g, 1.00 equiv), Bpin-compound 270 (2.08 g, 1.3 equiv), PdCl2(dppf) (0.4 g, 0.1 equiv) and potassium phosphate monohydrate (3.4 g, 3.0 equiv). After purging the flask with argon, 1,4-dioxane (50 mL) and water (5 mL) were added and the resulting mixture was heated at 40° C. overnight (23 hr). The reaction was cooled to rt. EtOAc was added to the reaction mixture and filtered through Celite. After concentration the residue was purified by column chromatography (silica gel, 25% EtOAc/hexane) to give the title compound 274 (46%).

### Example 275

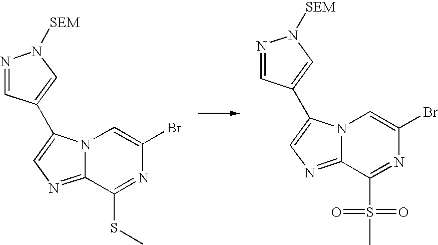

To a solution of compound 274 (1.02 g, 1.0 equiv) in DCM (10 mL) was added m-CPBA (1.1 g, 77%, 2.05 equiv) in one portion. The resulting mixture was stirred at room temperature for 30 min. The mixture was concentrated and then partitioned between EtOAc and water. The organic layer was washed with NaHCO₃ (sat. aq., ×2), brine and dried (Na₂SO₄). After concentration, the crude product compound 275 was used in the next step directly without further purification.

### Example 276

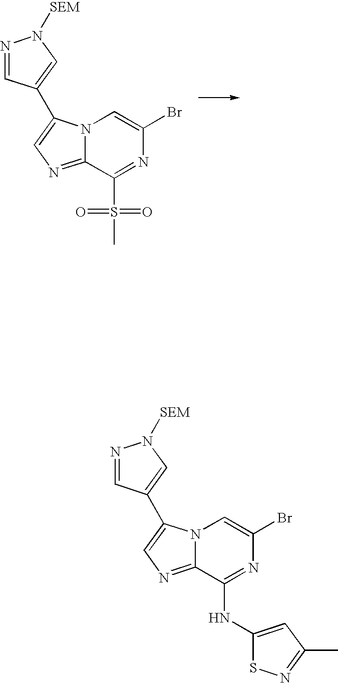

To a solution of aminoisothiazole hydrochloride (0.135 g, 1.4 equiv.) in DMSO (9 mL) at room temperature was added NaH (0.11 g of 60% dispersion in oil, 3.0 equiv) in one portion. After ca. 10 min, compound 273 (0.30 g, 1.00 equiv) was added in one portion. After 15 min at room temperature, the reaction was quenched with sat. aq. ammonium chloride and then extracted with ethyl acetate (×2). The combined organic layers were washed with water (×2), brine and dried (sodium sulfate). Evaporation of solvent afforded the title compound 276 (0.18 g. 56%).

### Example 277

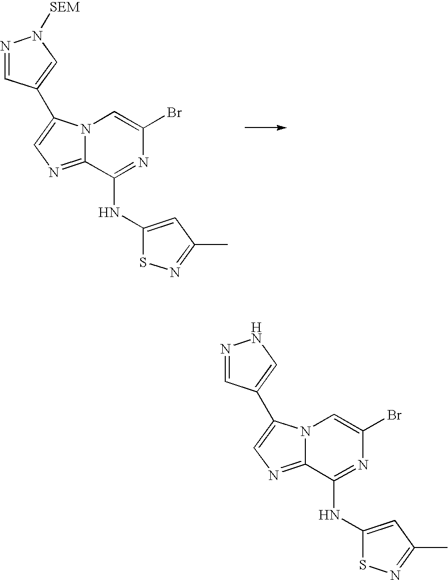

A solution of crude compound 276 in THF (1 mL) was treated with 4N HCl in dioxane solution (1 mL) at 60° C. for 10 min at which time HPLC-MS indicated that the reaction was complete. The solvent was removed and the residue was purified by Prep-LC. Conversion to a hydrochloric salt afforded compound 277. ¹H-NMR (400 MHz, DMSO-d₆) δ 12.35 (bs, 1H), 8.27 (bs, 2H), 8.18 (s, 1H), 7.92 (s, 1H), 7.03 (s, 1H) and 3.24 (s, 3H). HPLC-MS t_{R}=2.93 Min (UV₂₅₄ₙₘ). Mass calculated for formula C₁₃H₁₀BrN₇S, 374.99, observed LC/MS m/z 376.0 (M+H).

### Example 278

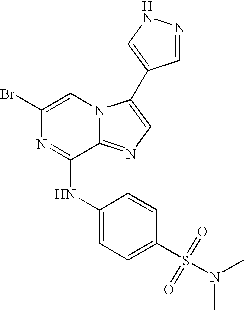

By essentially following the experimental procedure given in example 274 and 275, using appropriate amine (4-amino N,N-dimethyl benzenesulfonamide) compound 278 can be made. HPLC-MS t_{R}=4.06 Min (UV₂₅₄ₙₘ). Mass calculated for formula C₁₇H₁₆BrN₇O₂S, 461.03, observed LC/MS m/z 462.10 (M+H).

### Example 279

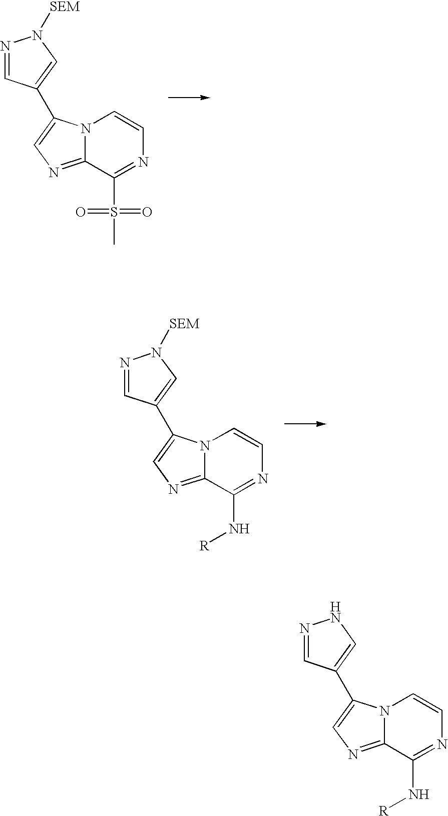

By essentially the same procedure given in Preparative Example 274 & 275, compounds (279, 1-7) given in Column 2 of Table 25 can be prepared.

TABLE 25

| | | | MS | |
|---|---|---|---|---|
| | | Exact | m/z | HPLC |
| Example | Column 2 | Mass | (M + H) | MS t_{R} |
| 279-1 | 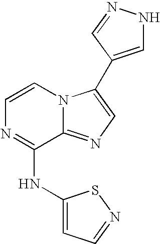 | 283.1 | 284.0 | 2.33 |
| 279-2 | 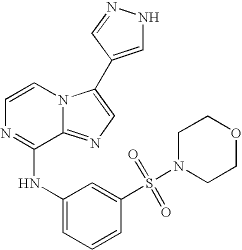 | 425.1 | 426.1 | 3.16 |
| 279-3 | 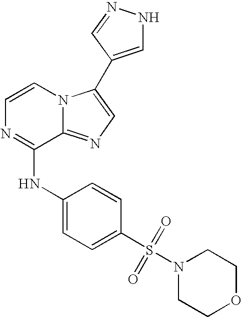 | 425.1 | 426.1 | 3.06 |
| 279-4 | 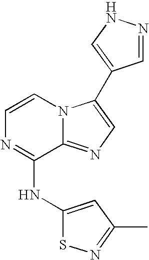 | 297 | 298 | 2.37 |
| 279-5 | 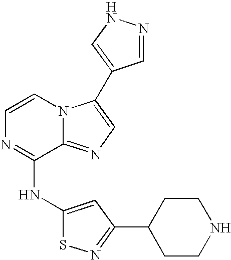 | 366 | 367 | 0.86 |
| 279-6 | 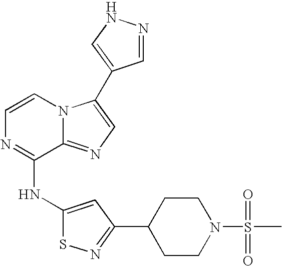 | 444 | 445 | 2.89 |
| 279-7 | 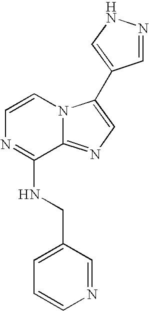 | 291 | 292 | 1.33 |

### Example 280

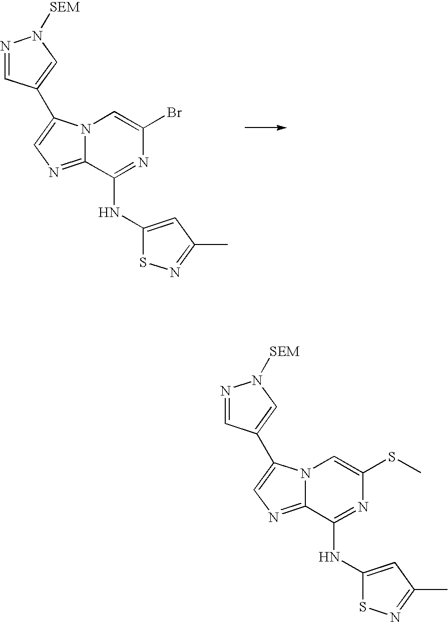

A mixture of compound 276 (30 mgs, 0.059 mmol, 1 equivalent), sodium methanethiolate (1.4 equivalent), PdCl₂(dppf) (0.07 equivalents), sodium tert-butoxide (1.1 equivalents) in 1,2-dimethoxyethane (1 ml) was stirred at 85 C under Ar for 16 h. The reaction mixture was cooled to room temperature, filtered through Celite and the filtrate concentrated. The residue was taken back up in ethyl acetate and washed with water, brine, dried over anhydrous sodium sulfate and concentrated to afford crude compound 280. HPLC-MS t_{R}=2.26 Min (UV₂₅₄ₙₘ). Mass calculated for formula C₂₁H₂₉N₇OS₂Si 487.16, observed LC/MS m/z 488.1.

### Example 281

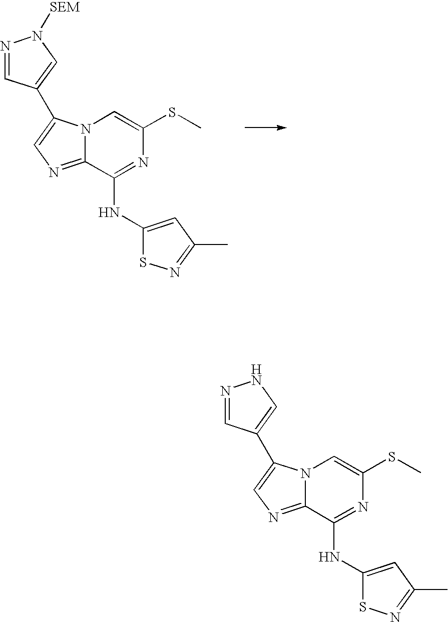

By essentially the same procedure used in the preparative example 275 to give the product 281. ¹H-NMR (400 MHz, DMSO-d₆) δ 8.27 (s, 2H), 7.96 (s, 1H), 7.84 (s, 1H), 7.07 (s, 1H), 2.66 (3.43) and 2.42 (s, 3H). HPLC-MS t_{R}=Min (UV₂₅₄ₙₘ). Mass calculated for formula C₁₄H₁₃N₇S 343.07, observed LC/MS m/z 344.1.

### Examples 282

By essentially the same procedure given in Preparative 278 & 279 or by metal catalyzed reactions, the compounds 282 (1-11) given in Column 2 of Table 26 can be prepared from compound 274.

TABLE 26

| | | | MS | |
|---|---|---|---|---|
| | | Exact | m/z | HPLC |
| Example | Column 2 | Mass | (M + H) | MS t_{R} |
| 282-1 | 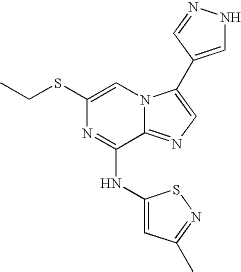 | 357.08 | 358.1 | 3.17 |
| 282-2 | 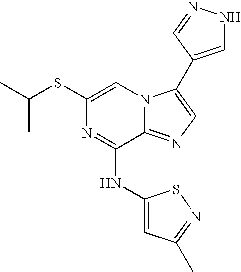 | 371.13 | 72.1 | 3.41 |
| 282-3 | 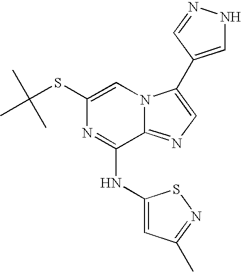 | 385.1 | 386.1 | 3.48 |
| 282-4 | 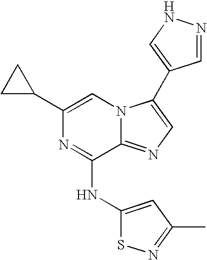 | 337 | 338 | 1.10 |
| 282-5 | 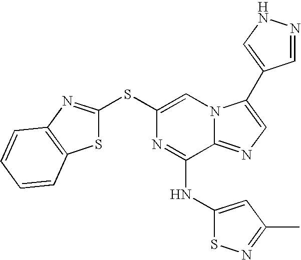 | 462 | 463 | 1.45 |
| 282-6 | 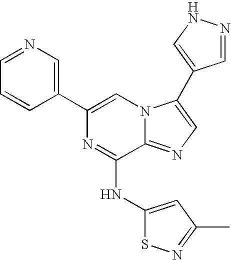 | 374 | 375 | 0.96 |
| 282-7 | 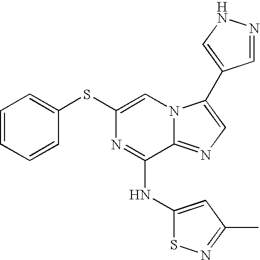 | 405 | 406 | 1.38 |
| 282-8 | 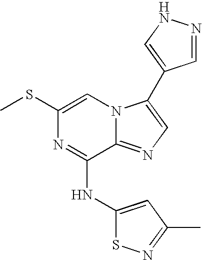 | 343 | 344 | 1.12 |
| 282-9 | 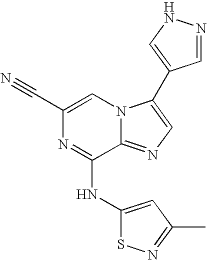 | 322 | 323 | 1.09 |
| 282-10 | 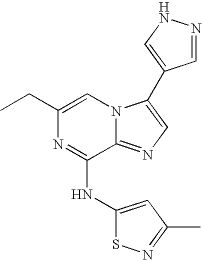 | 325 | 326 | 1.12 |
| 282-11 | 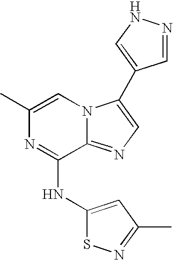 | 311 | 312 | 0.97 |

The compound 283 in Table 27 was prepared by essentially the same procedure as in Preparative examples starting from compound 271.

TABLE 27

| | | | MS | |
|---|---|---|---|---|
| | | Exact | m/z | HPLC |
| Example | Column 2 | Mass | (MH)+ | MS t_{R} |
| 283 | 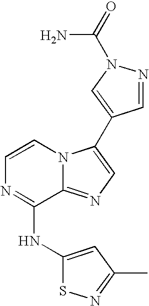 | 340 | 341 | 0.82 |

### Example 284

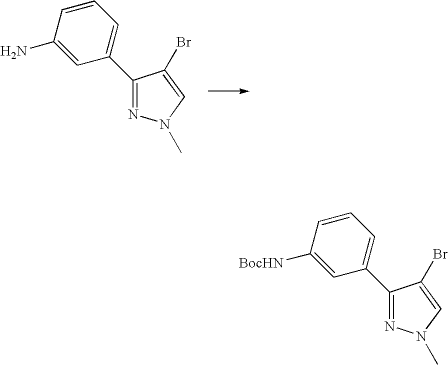

To the mixture of compound, [3-(4-bromo-1-methyl-1H-pyrazol-3-yl)-phenyl]carbamic acid tert-butylester (1.78 g, 7.1 mmol), imidazole (1.36 g, 20 mmol), and catalytic amount DMAP in DMF (12 mL), Boc₂O (1.7 g, 7.8 mmol) was added at room temperature. The mixture was stirred overnight at room temperature and diluted with EtOAc (200 mL), the organics were washed with H₂O, brine and dried over Na₂SO₄. After concentration, the residue was purified with column (silica gel, hexane/EtOAc=70/30) gave the product 284 (2.52 g) as white solid. HPLC-MS t_{R}=2.00 Min (UV₂₅₄ₙₘ). Mass calculated for formula C₁₅H₁₈BrN₃O₂ 351.1, observed LC/MS m/z 352.1 (M+H).

### Example 285

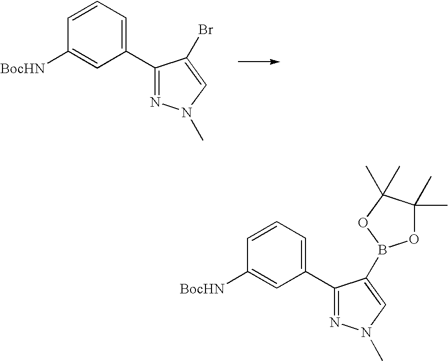

To a 25 ml round bottom flask charged with bis(pinacolato)diboron (1.0 g, 4.0 mmol), KOAc (960 mg, 10 mmol), PdCl₂(dppf) (240 mg, 0.3 mmol) and compound 284 (1.16 g, 3.3 mmol) was added DMSO (6 ml) under Argon. The mixture was thoroughly degassed by alternately connected the flask to vacuum and Argon. This resulting mixture was then heated at 80° C. overnight, diluted by EtOAc (40 ml) and filtered through celite. After concentration, the residue was purified with column (silica gel, Hexane/EtOAc=80/20) to give the product 285 (997 mg) as oil. HPLC-MS t_{R}=2.11 min (UV_{254 nm}); mass calculated for formula C₂₁H₃₀BN₃O₄ 399.2, observed LCMS m/z 400.3(M+H).

### Example 286

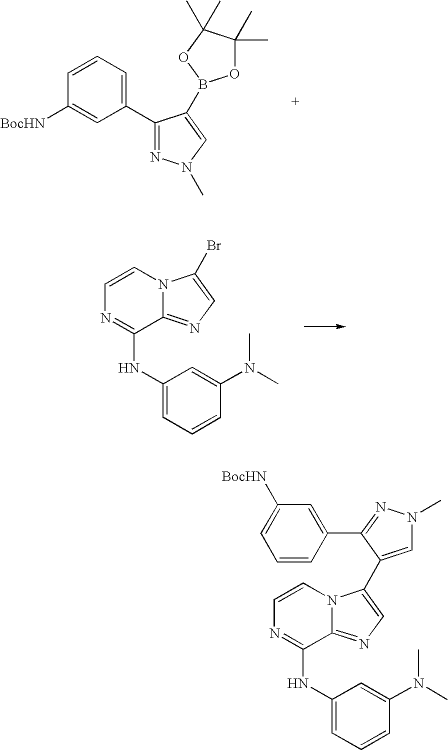

Under Argon, the compound 285 (120 mg, 0.3 mmol) in THF (3.0 mL, 5% H₂O) was added to the flask which was charged with Pd(dppf)Cl₂ (8 mg, 0.01 mmol), K₂CO₃ (138 mg, 1.0 mmol), and compound 149 (51 mg, 0.15 mmol). The mixture was thoroughly degassed by alternately connected the flask to vacuum and Argon. The resulting solution was heated upto 80° C. and stirred overnight. After cooling to room temperature, the mixture was diluted with EtOAc (50 mL) and the solid was removed by filter through Celite and washed with some EtOAc. Concentration to remove the solvent and the resulting residue 286 was used in the next step directly without further purification. HPLC-MS t_{R}=2.05 min (UV_{254 nm}); mass calculated for formula C₂₉H₃₂N₈O₂ 524.3, observed LCMS m/z 525.2.1 (M+H).

### Example 287

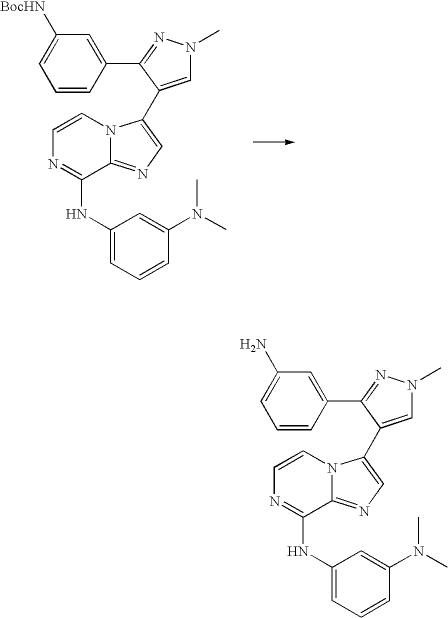

To the compound 286 was added HCl (6N, 3 mL), and the mixture was stirred at room temperature for 10 min. Then, concentrated, and the residue was purified with HPLC and gave the final compound 287 (48 mg). HPLC-MS t_{R}=1.16 min (UV_{254 nm}); mass calculated for formula C₂₄H₂₄N₈ 424.2, observed LCMS m/z 425.2 (M+H).

### Example 288

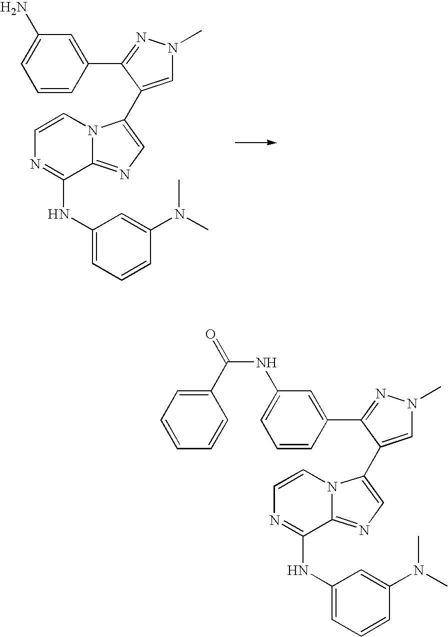

The benzoic acid (6 mg, 0.05 mmol) in DMF (1 mL) was added HOBt (7 mg, 0.05 mmol), EDC (10 mg, 0.05 mmol) and the mixture was stirred at room temperature for 10 min. Then, compound 287 (21 mg, 0.05 mmol) in DMF (1 mL) was added and the resulting mixture was allowed to heated up to 50° C. and stirred overnight. The mixture was diluted with EtOAc (50 mL) and washed with H₂O, brine and dried over Na₂SO₄. After concentration, the residue was purified with HPLC gave the product 288. HPLC-MS t_{R}=1.54 min (UV_{254 nm}); mass calculated for formula C₃₁H₂₈N₈O 528.2, observed LCMS m/z 529.3 (M+H).

### Example 289

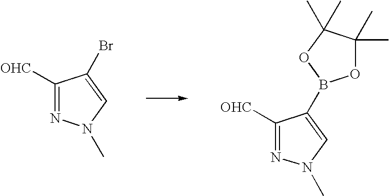

Compound 289 was prepared using the boronation conditions described in Example 285. HPLC-MS t_{R}=1.83 min (UV_{254 nm}); mass calculated for formula C₁₁H₁₇BN₂O₃ 236.1, observed LCMS m/z 237.3 (M+H).

### Example 290

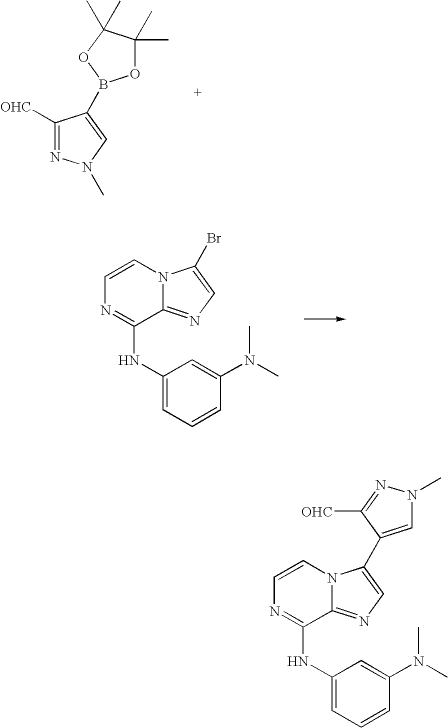

Compound 290 was prepared using the coupling conditions described in Example 286. HPLC-MS t_{R}=1.18 min (UV_{254 nm}); mass calculated for formula C₁₉H₁₉N₇O 361.2, observed LCMS m/z 362.1 (M+H).

### Example 291

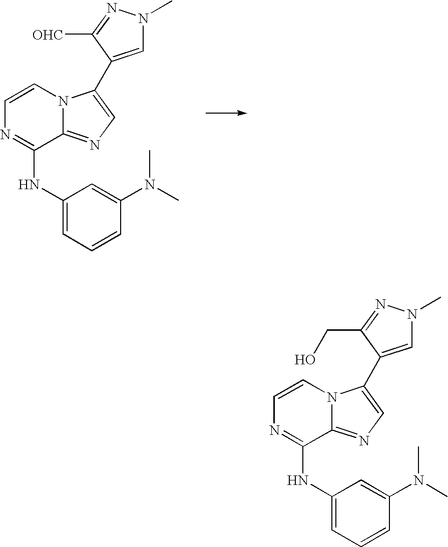

Compound 290 (50 mg, 0.14 mmol) was dissolved in MeOH (5 mL) and the mixture was cooled to 0° C. NaBH4 (38 mg, 1.0 mmol) was added and the resulting mixture was stirred at 0° C. for 30 min. After concentration, the residue was purified with HPLC gave the product 291. HPLC-MS t_{R}=0.92 min (UV_{254 nm}); mass calculated for formula C₁₉H₂₁N₇O 363.2, observed LCMS m/z 364.3 (M+H).

### Example 292

By essentially the same procedure given in Preparative Example 290, compounds given in Column 2 of Table 28 can be prepared from compound 149 and appropriate pyrazole boronate.

TABLE 28

| Ex- | | | MS | |
|---|---|---|---|---|
| am- | | Exact | m/z | HPLC |
| ple | Column 2 | mass | (M + H) | MS t_{R} |
| 292-1 | 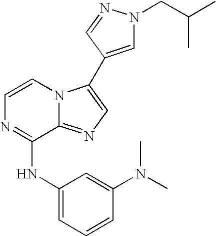 | 375.2 | 376.3 | 1.51 |
| 292-2 | 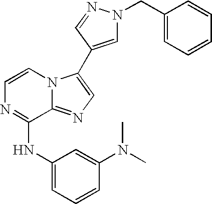 | 409.2 | 410.2 | 1.53 |

### Example 293

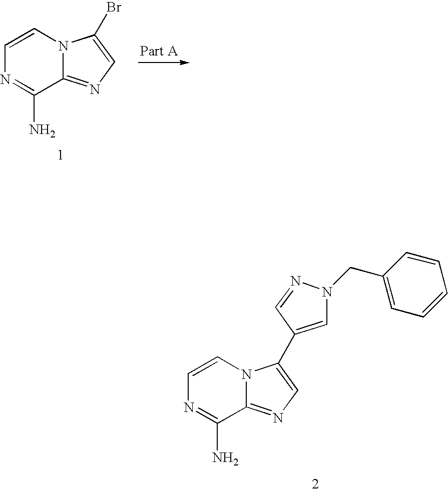

Compound 293 was prepared using the coupling condition described in example 286 starting from-3-bromo-7-amino imidazopyrazines and n-benzyl pyrazole-4-boronate. HPLC-MS t_{R}=0.94 min (UV_{254 nm}); mass calculated for formula C₁₆H₁₄N₆ 290.1, observed LCMS m/z 291.3 (M+H).

### Example 294

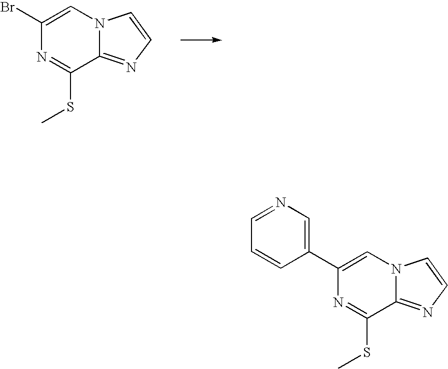

Compound 294 was prepared using the coupling condition described in example 198. HPLC-MS t_{R}=0.79 min (UV_{254 nm}); mass calculated for formula C₁₂H₁₀N₄S 242.1, observed LCMS m/z 243.1 (M+H).

### Example 295

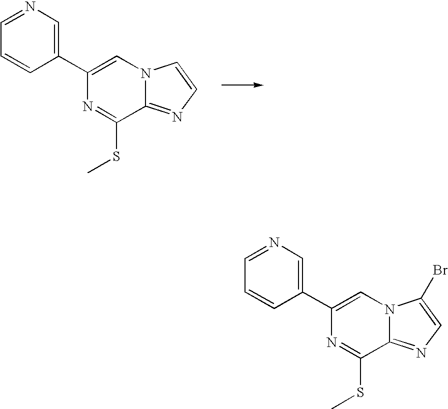

Compound 295 was prepared using the bromination condition described in 179. HPLC-MS t_{R}=1.11 min (UV_{254 nm}); mass calculated for formula C₁₂H₉BrN₄S 320.0, observed LCMS m/z 321.0 (M+H).

### Example 296

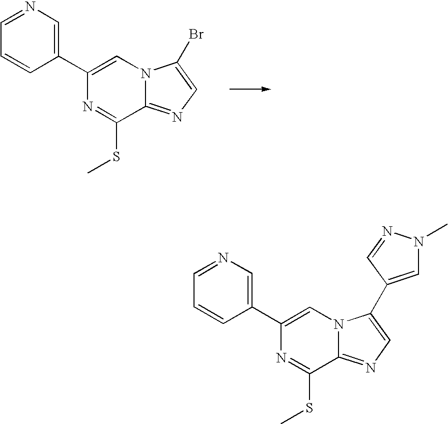

Compound 296 was synthesized using the same coupling condition described in example 180. HPLC-MS t_{R}=1.04 min (UV_{254 nm}); mass calculated for formula C₁₆H₁₄N₆S, 322.1, observed LCMS m/z 323.2 (M+H).

### Example 297

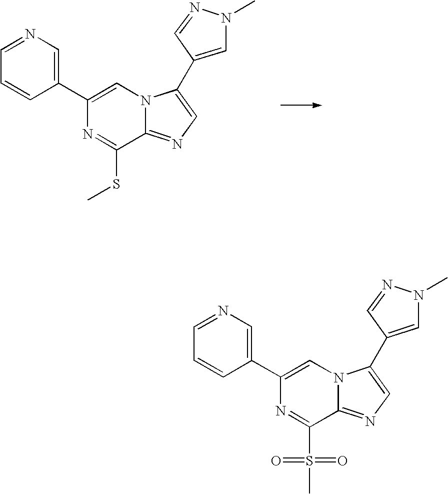

Compound 297 was synthesized using the same oxidation condition described in example 181. HPLC-MS t_{R}=0.71 min (UV_{254 nm}); mass calculated for formula C₁₆H₁₄N₆O₂S 354.1, observed LCMS m/z 355.0 (M+H).

### Example 298

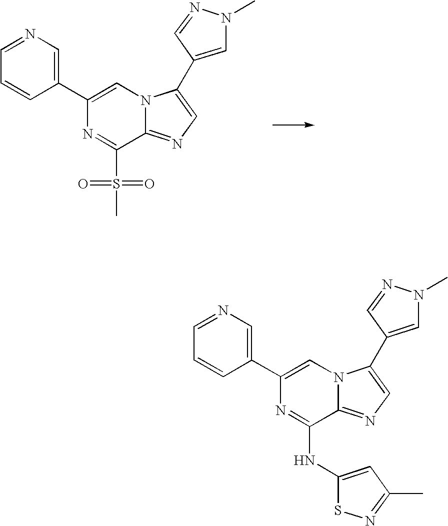

Compound 298 was prepared using the amination condition described in example 182. HPLC-MS t_{R}=0.63 min (UV_{254 nm}); mass calculated for formula C₁₉H₁₆N₈S 388.1, observed LCMS m/z 389.2 (M+H).

### Example 299

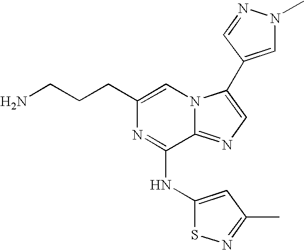

Compound 299 was synthesized with the using the procedures described in examples 177 through 183. HPLC-MS t_{R}=0.93 min (UV_{254 nm}); mass calculated for formula C₁₇H₂₀N₈S 368.2, observed LCMS m/z 369.1 (M+H).

### Example 300

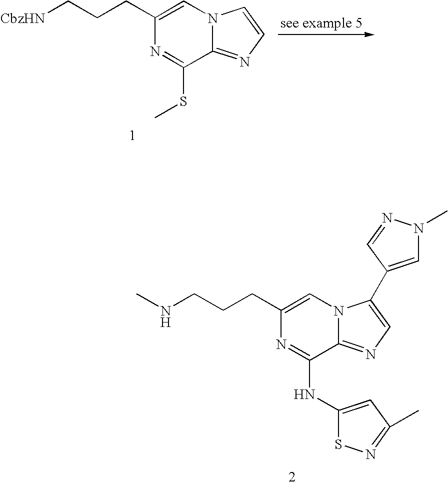

Compound 300 was synthesized using preparative procedures described in examples 186 through 191. HPLC-MS t_{R}=0.99 min (UV_{254 nm}); mass calculated for formula C₁₈H₂₂N₈S 382.2, observed LCMS m/z 383.1 (M+H).

### Example 301

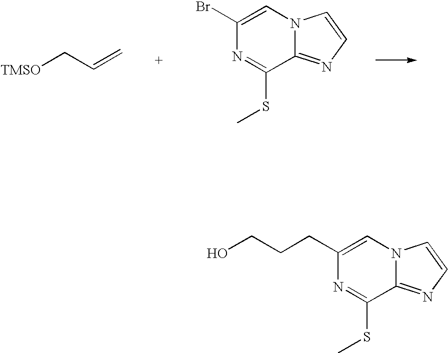

Compound 301 was synthesized with the same procedure using in example 178. HPLC-MS t_{R}=0.82 min (UV_{254 nm}); mass calculated for formula C₁₀H₁₃N₃OS 223.1, observed LCMS m/z 224.1 (M+H).

### Example 302

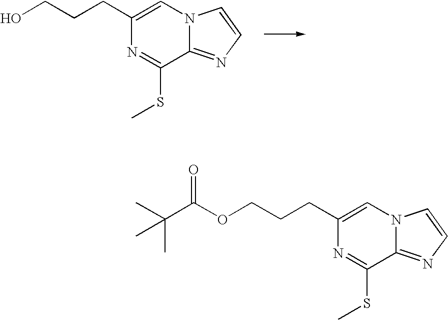

Compound 302 (223 mg, 1.0 mmol) was dissolved in DCM (10 mL) and DIEA (200 μL) was added followed by DMAP (cat. Amount) and pivaloyl chloride (150 μL). The resulting mixture was stirred at room temperature for 1 hour and diluted with EtOAc. The organics was washed with NaHCO₃ (aq), water and brine, dried over Na₂SO₄. After concentration, the crude product was used in the next step directly without further purification. HPLC-MS t_{R}=1.82 min (UV_{254 nm}); mass calculated for formula C₁₅H₂₁N₃O₂S 307.1, observed LCMS m/z 308.2 (M+H).

### Example 303

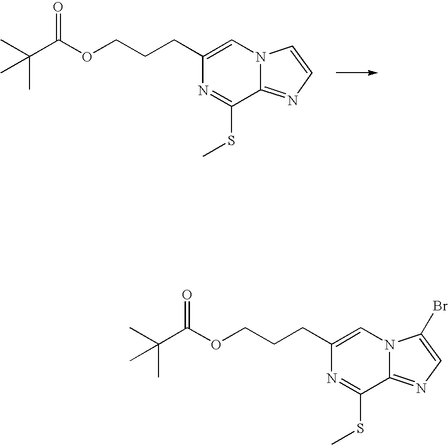

Compound 303 was prepared using the bromination condition described in example 179. HPLC-MS t_{R}=2.28 min (UV_{254 nm}); mass calculated for formula C₁₅H₂₀BrN₃O₂S 385.0, observed LCMS m/z 386.0 (M+H).

### Example 304

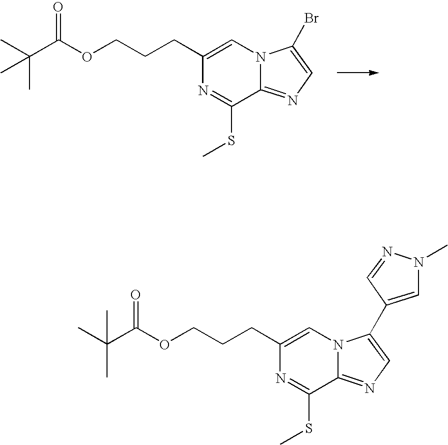

Compound 304 was synthesized using the same coupling condition described in example 180. HPLC-MS t_{R}=1.89 min (UV_{254 nm}); mass calculated for formula C₁₉H₂₅N₅O₂S 387.2, observed LCMS m/z 388.2 (M+H).

### Example 305

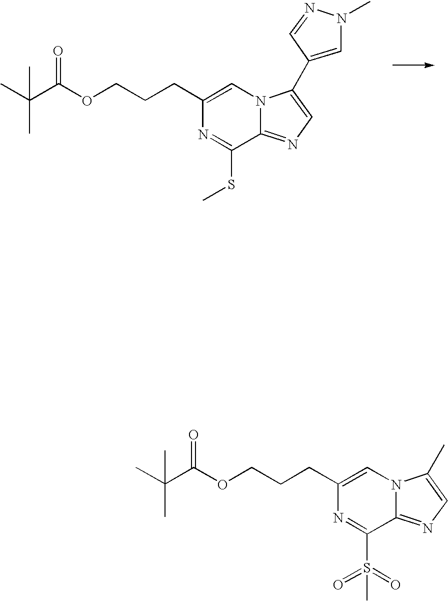

Compound 305 was synthesized using the same oxidation condition described in example 181. HPLC-MS t_{R}=1.53 min (UV_{254 nm}); mass calculated for formula C₁₉H₂₅N₅O₄S 419.2, observed LCMS m/z 420.1 (M+H).

### Example 306

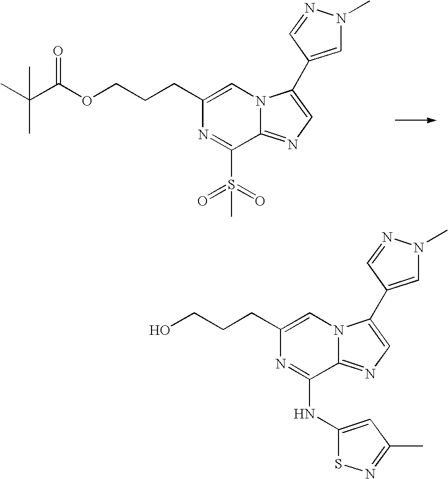

Compound 306 was prepared using the amination condition described in example 182 and deprotection of butyloxy carbonyl group as in example 183. HPLC-MS t_{R}=2.55 min (UV_{254 nm}, 10 min LC-MS); mass calculated for formula C₁₇H₁₉N₇OS 369.1, observed LCMS m/z 370.1 (M+H).

### Example 307

By essentially the same procedure given in Preparative Example 306 starting from compound 305, compound given in Column 2 of Table 29 can be prepared.

TABLE 29

| | | | MS | |
|---|---|---|---|---|
| | | Exact | m/z | HPLC |
| Example | Column 2 | Mass | (M + H) | MS t_{R} |
| 301 | 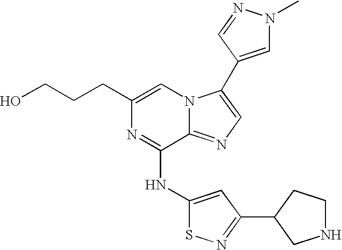 | 424.2 | 425.1 | 0.85 |

### Example 308

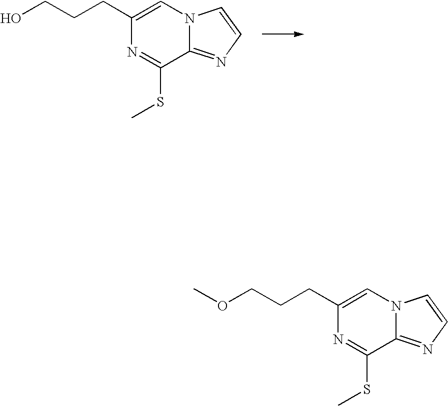

Compound 308 was synthesized using the same condition as described in preparative example 186. HPLC-MS t_{R}=1.03 min (UV_{254 nm}); mass calculated for formula C₁₁H₁₅N₃OS 237.1, observed LCMS m/z 238.1 (M+H).

### Example 309

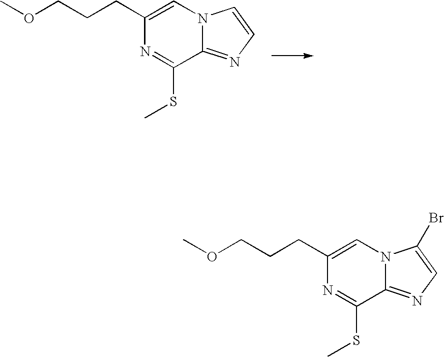

Compound 309 was prepared using the bromination condition described in example 187. HPLC-MS t_{R}=2.33 min (UV_{254 nm}); mass calculated for formula C₁₁H₁₄BrN₃OS 315.0, observed LCMS m/z 316.0 (M+H).

### Example 310

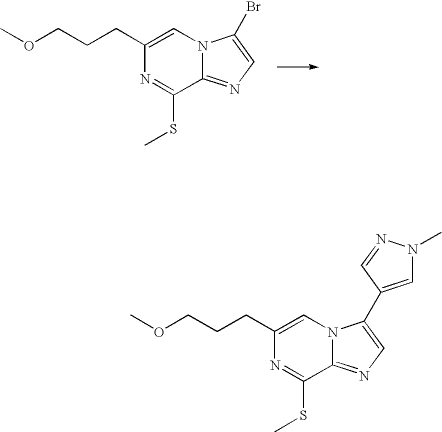

Compound 310 was synthesized using the same coupling condition described in example 188. HPLC-MS t_{R}=1.43 min (UV_{254 nm}); mass calculated for formula C₁₅H₁₉N₅OS 317.1, observed LCMS m/z 318.1 (M+H).

### Example 311

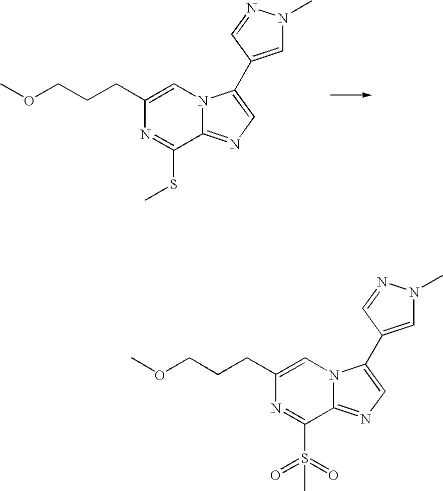

Compound 311 was synthesized using the same oxidation condition described in example 189. HPLC-MS t_{R}=1.06 min (UV_{254 nm}); mass calculated for formula C₁₅H₁₉N₅O₃S 349.1, observed LCMS m/z 350.2 (M+H).

### Example 312

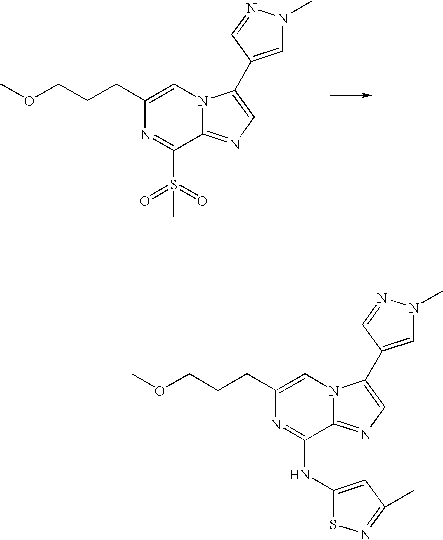

Compound 312 was prepared using the amination condition described in example 190. HPLC-MS t_{R}=1.26 min (UV_{254 nm}); mass calculated for formula C₁₈H₂₁N₇OS 383.2, observed LCMS m/z 384.1 (M+H).

### Example 313

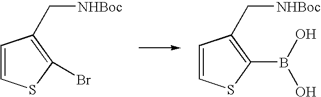

Compound 313 (596 mg, 2.0 mmol) was dissolved in THF (20 mL) and cooled to −78° C. n-BuLi (1.6 ml, 2.5 M in hexane, 4.0 mmol) was added dropwise and the resulting mixture was stirred at −78° C. for 30 min. Triisopropyl borate (752 mg, 4.0 mmol) was added and the mixture was stirred for 30 min at −78° C., then warmed to room temperature slowly. 1N HCl (10 mL) was added and the mixture was extracted with EtOAc. The organics was dried over Na₂SO₄ and concentrated. The crude product 2 was used in the next step without further purification. HPLC-MS t_{R}=1.49 min (UV_{254 nm}); mass calculated for formula C₁₀H₁₆BNO₄S 257.1, observed LCMS m/z 202.1 (M+H—t-Bu).

### Example 314

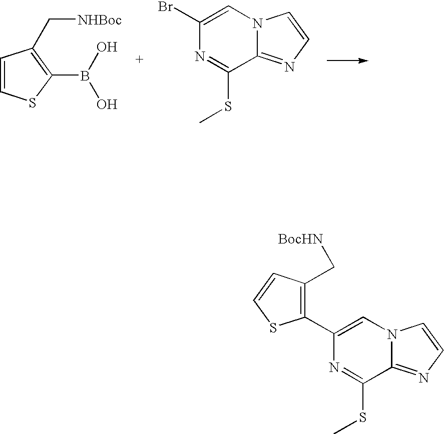

Compound 314 was synthesized using the same coupling condition described in example 178. HPLC-MS t_{R}=1.89 min (UV_{254 nm}); mass calculated for formula C₁₇H₂₀N₄O₂S₂ 376.1, observed LCMS m/z 377.1 (M+H).

### Example 315

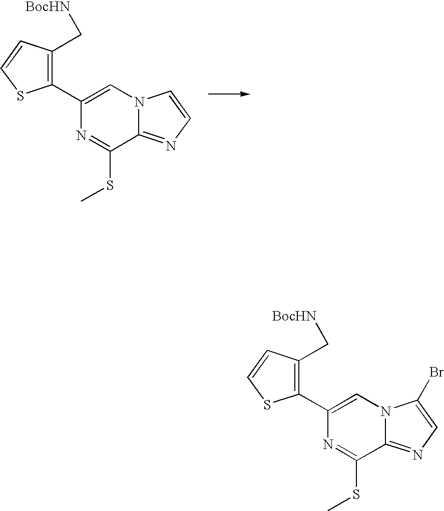

Compound 315 was prepared using the bromination condition described in example 179. HPLC-MS t_{R}=2.20 min (UV_{254 nm}); mass calculated for formula C₁₇H₁₉BrN₄O₂S₂, 454.0, observed LCMS m/z 455.0 (M+H).

### Example 316

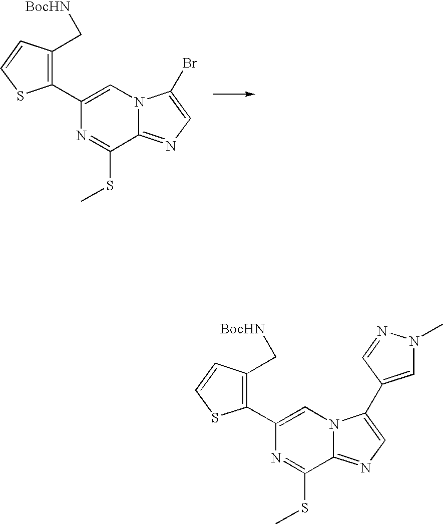

Compound 316 was synthesized using the same coupling condition described in example 180. HPLC-MS t_{R}=1.96 min (UV_{254 nm}); mass calculated for formula C₂, H₂₄N₆O₂S₂ 456.1, observed LCMS m/z 427.1 (M+H).

### Example 317

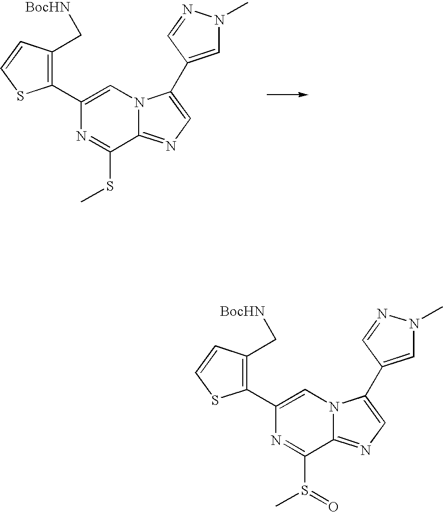

Compound 317 was synthesized using the same oxidation condition described in example 201. HPLC-MS t_{R}=1.54 min (UV_{254 nm}); mass calculated for formula C₂, H₂₄N₆O₃S₂ 472.1, observed LCMS m/z 473.1 (M+H).

### Example 318

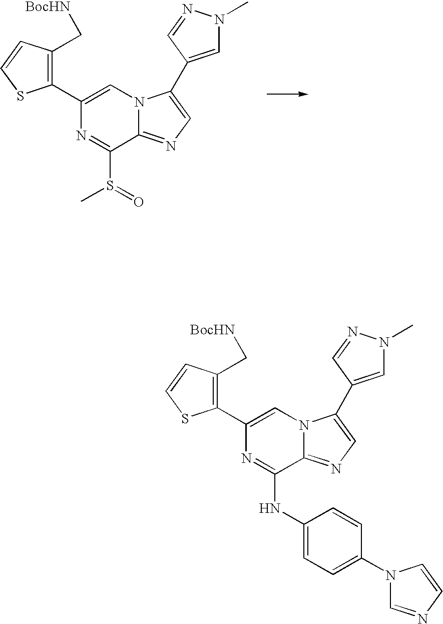

Compound 318 was prepared using the amination condition described in example 202. HPLC-MS t_{R}=1.44 min (UV_{254 nm}); mass calculated for formula C₂₉H₂₉N₉O₂S 567.2, observed LCMS m/z 568.3 (M+H).

### Example 319

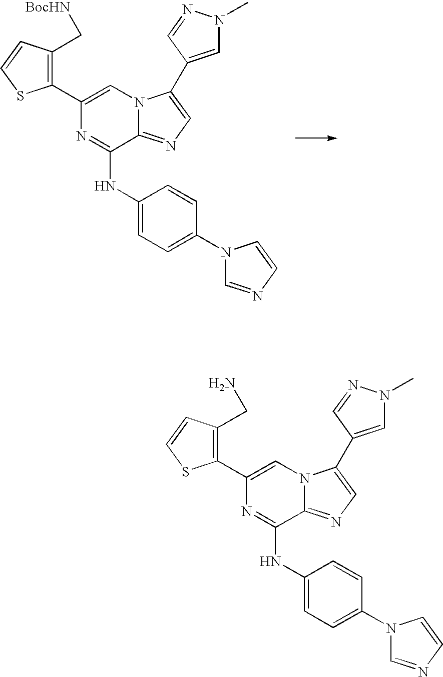

Compound 319 was synthesized using the deprotecting condition described in example 203. HPLC-MS t_{R}=0.87 min (UV_{254 nm}); mass calculated for formula C₂₄H₂₁N₉S 467.2, observed LCMS m/z 468.1 (M+H).

### Example 320

By essentially the same procedure given in Preparative Example 318 and 319 starting from compound 317, compound given in Column 2 of Table 30 can be prepared.

TABLE 30

| | | | MS | |
|---|---|---|---|---|
| | | Exact | m/z | HPLC |
| Example | Column 2 | Mass | (M + H) | MS t_{R} |
| 320 | 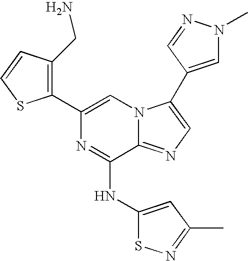 | 422.1 | 423.1 | 0.98 |
| 337-1 | 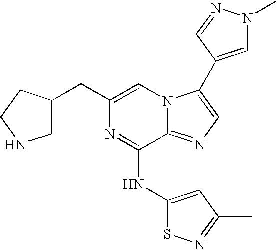 | 394.2 | 395.1 | 0.91 |
| 337-2 | 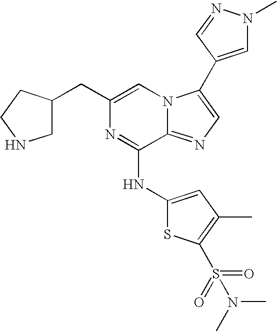 | 500.2 | 501.1 | 1.25 |
| 337-3 | 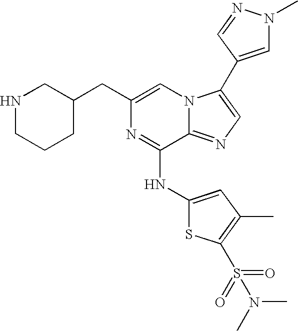 | 514.2 | 515.2 | 1.29 |

### Example 321

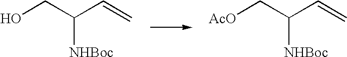

Compound 321 was synthesized using the same condition described in example 302. NMR (CDCl₃, ppm): 5.69(m, 1H), 5.25(m, 2H), 4.73(m, 1H), 4.45(m, 1H), 4.13(m, 2H), 3.68(m, 1H), 2.07(s, 3H), 1.46(s, 9H).

### Example 322

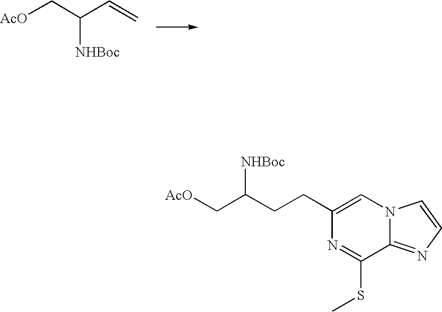

Compound 322 was synthesized with the same procedure using in example 178. HPLC-MS t_{R}=1.62 min (UV_{254 nm}); mass calculated for formula C₁₈H₂₆N₄O₄S 394.2, observed LCMS m/z 395.1 (M+H).

### Example 323

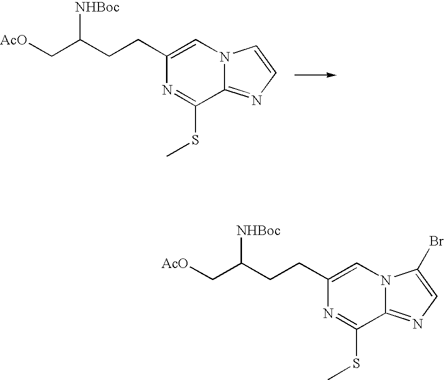

Compound 323 was prepared using the bromination condition described in example 179. HPLC-MS t_{R}=1.97 min (UV_{254 nm}); mass calculated for formula C₁₈H₂₅BrN₄O₄S 472.1, observed LCMS m/z 473.0 (M+H).

### Example 324

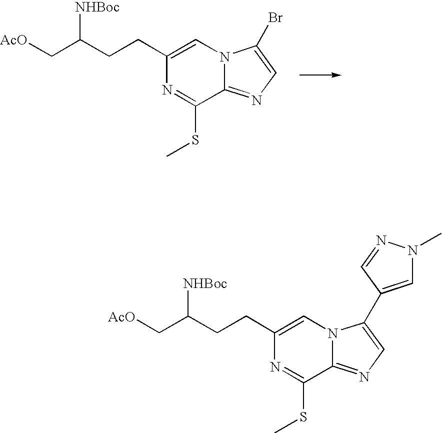

Compound 324 was synthesized using the same coupling condition described in example 180. HPLC-MS t_{R}=1.70 min (UV_{254 nm}); mass calculated for formula C₂₂H₃₀N₆O₄S 474.2, observed LCMS m/z 475.1 (M+H).

### Example 325

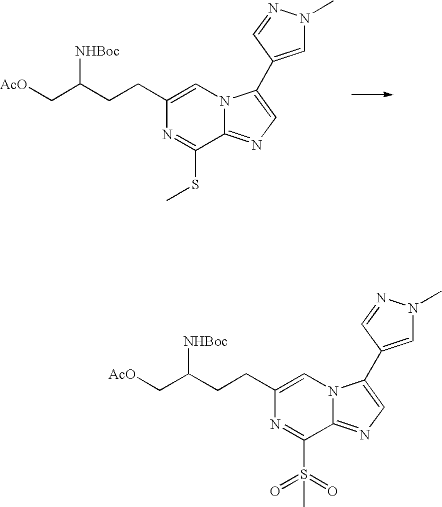

Compound 325 was synthesized using the same oxidation condition described in example 181. HPLC-MS t_{R}=1.41 min (UV_{254 nm}); mass calculated for formula C₂₂H₃₀N₆O₆S 506.2, observed LCMS m/z 507.1 (M+H).

### Example 326

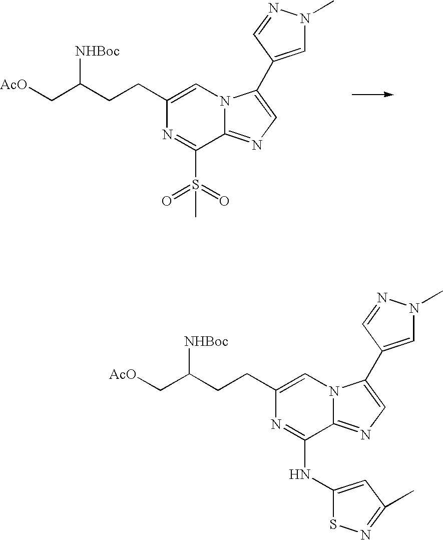

Compound 326 was prepared using the amination condition described in example 182. HPLC-MS t_{R}=1.52 min (UV_{254 nm}); mass calculated for formula C₂₅H₃₂N₈O₄S 540.2, observed LCMS m/z 541.2 (M+H).

### Example 327

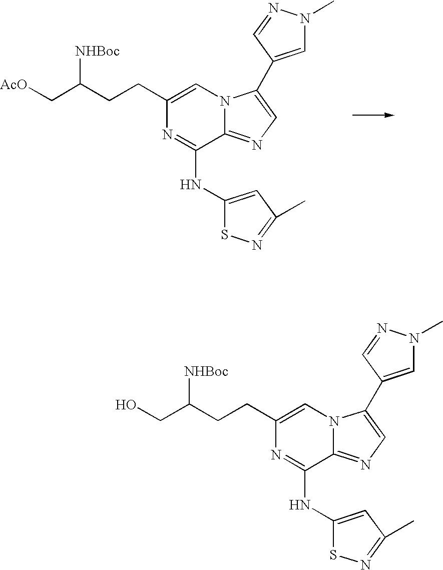

Compound 326 (150 mg) was dissolved in the mixture of THF (10 mL) and methanol (5 mL). LiOH (1N, 4 mL) was added and the resulting mixture was stirred at 50° C. for 2 hours. After cooling to room temperature, the mixture was concentrated followed by taking up with EtOAc. The organics was washed with water, brine and dried over Na₂SO₄. After concentration, the crude product 327 (122 mg) was used in the next step without further purification. HPLC-MS t_{R}=1.29 min (UV_{254 nm}); mass calculated for formula C₂₃H₃₀N₈O₃S 498.2, observed LCMS m/z 499.1 (M+H).

### Example 328

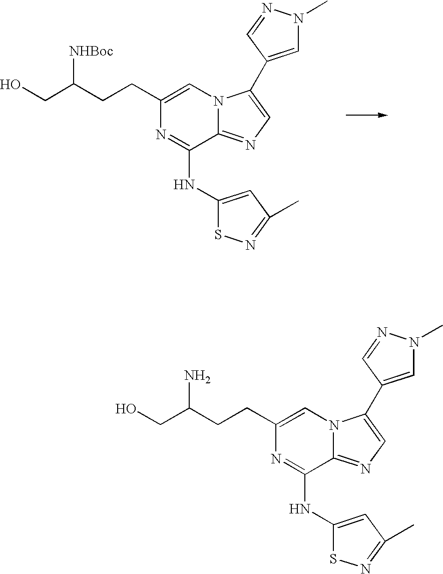

Compound 328 was synthesized using the deprotecting condition described in example 183. HPLC-MS t_{R}=0.80 min (UV_{254 nm}); mass calculated for formula C₁₈H₂₂N₈OS 398.2, observed LCMS m/z 399.0 (M+H).

### Example 329

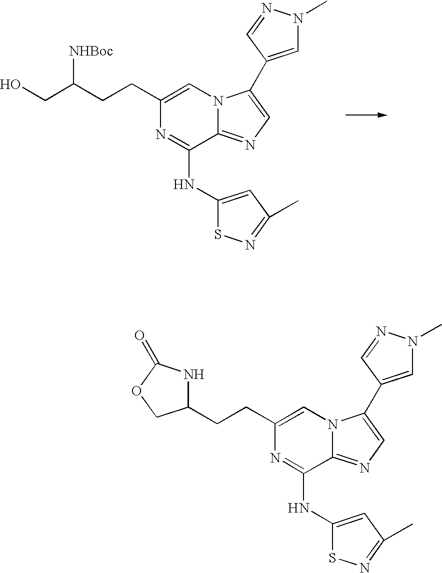

Compound 328 (25 mg) was dissolved in DMF (5 mL) and NaH (8 mg, 0.2 mmol) was added. The resulting mixture was stirred at room temperature overnight and quenched with NH₄Cl (sat. aq.) extracted with EtOAc. After concentration, the crud product was purified by HPLC gave the compound 329. HPLC-MS t_{R}=1.05 min (UV_{254 nm}); mass calculated for formula C₁₉H₂₀N₈O₂S 424.1, observed LCMS m/z 425.1 (M+H).

### Example 330

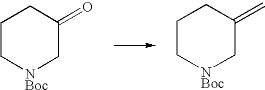

A suspension of methyltriphenylphosphonium bromide (8.93 g, 25 mmol) in THF (50 mL) was placed under argon and treated with t-BuOK (25 mL, 1M in THF). The mixture quickly became bright yellow and was stirred at room temperature for 1 hour. A solution of 1-Boc-3-piperidone (1.97 g, 10 mmol) in THF (10 mL) was then added to the mixture and stirred for 3 hours. The mixture was poured into water, extracted with ether and dried over Na₂SO₄ and concentrated. The crude material was purified by column (silica gel, 5% EtOAc in hexane) to afford product 330 as an oil (1.51 g).

### Example 331

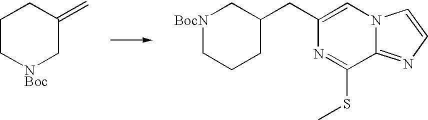

Compound 331 was synthesized with the same procedure using in example 178. HPLC-MS t_{R}=1.90 min (UV_{254 nm}); mass calculated for formula C₁₈H₂₆N₄O₂S 362.2, observed LCMS m/z 363.3 (M+H).

### Example 332

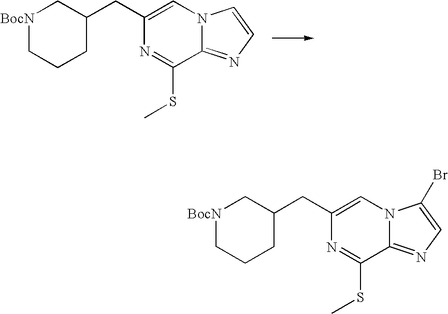

Compound 332 was prepared using the bromination condition described in example 179. HPLC-MS t_{R}=2.31 min (UV_{254 nm}); mass calculated for formula C18H₂₅BrN₄O₂S 440.1, observed LCMS m/z 441.1 (M+H).

### Example 333

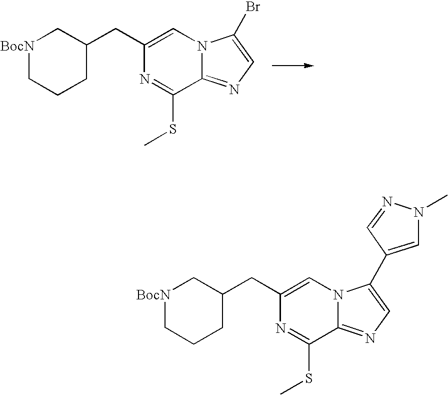

Compound 333 was synthesized using the same coupling condition described in example 180. HPLC-MS t_{R}=1.99 min (UV_{254 nm}); mass calculated for formula C₂₂H₃₀N₆O₂S 442.2, observed LCMS m/z 443.2 (M+H).

### Example 334

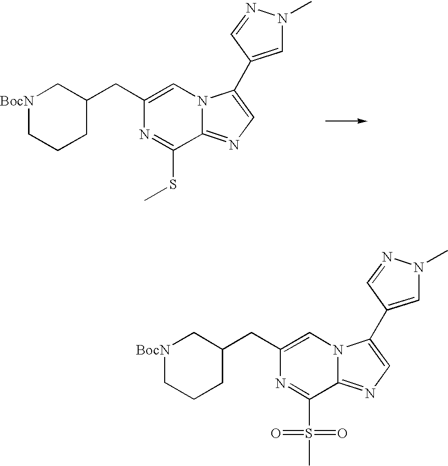

Compound 334 was synthesized using the same oxidation condition described in example 181. HPLC-MS t_{R}=1.66 min (UV_{254 nm}); mass calculated for formula C₂₂H₃₀N₆O₄S 474.2, observed LCMS m/z 475.1 (M+H).

### Example 335

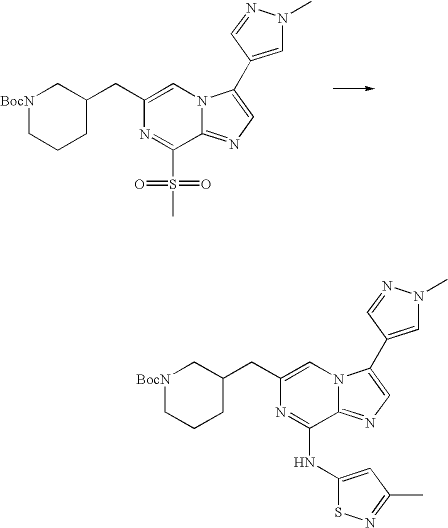

Compound 335 was prepared using the amination condition described in example 182. HPLC-MS t_{R}=1.58 min (UV_{254 nm}); mass calculated for formula C₂₅H₃₂N₈O₂S 508.2, observed LCMS m/z 509.2 (M+H).

### Example 336

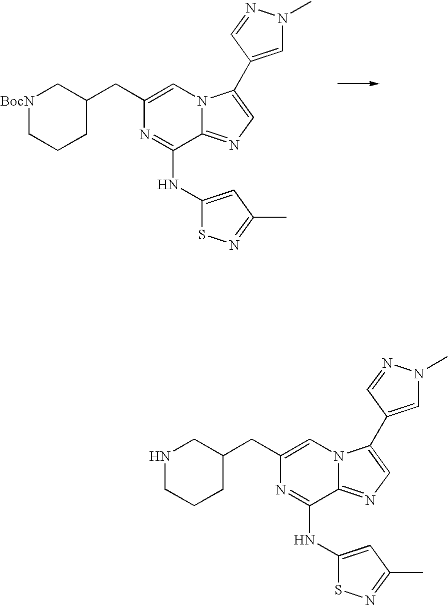

Compound 336 was synthesized using the deprotecting condition described in example 183. HPLC-MS t_{R}=0.95 min (UV_{254 nm}); mass calculated for formula C₂₀H₂₄N₈S 408.2, observed LCMS m/z 409.1 (M+H).

### Example 337

By essentially the same procedure given in Preparative Example 335 & 336 starting from 334 and appropriate amines, compounds given in Column 2 of Table 31 can be prepared.

TABLE 31

| | | Exact | MS m/z | HPLC |
|---|---|---|---|---|
| Example | Column 2 | mass | (M + H) | MS t_{R} |
| 337-1 | 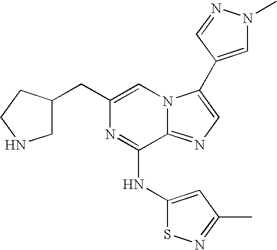 | 394.2 | 395.1 | 0.91 |
| 337-2 | 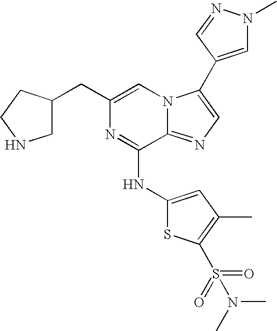 | 500.2 | 501.1 | 1.25 |
| 337-3 | 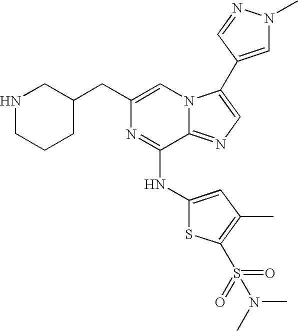 | 514.2 | 515.2 | 1.29 |

### Example 338

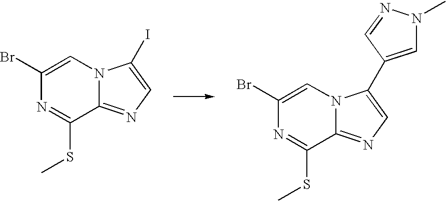

Under Argon, to the flask which charged with the boronate compound (81 mg, 0.39 mmol), Pd(dppf)Cl₂ (32 mg, 0.039 mmol), and K₃PO₄ (212 mg, 1.0 mmol), compound 273 (145 mg, 0.0.39 mmol) in dioxane (5 mL) was added. The mixture was thoroughly degassed by alternately connecting the flask to vacuum and Argon. The resulting solution was heated upto 40° C. and stirred overnight. After cooling to room temperature, the mixture was diluted with EtOAc (50 mL) and the solid was removed by filter through Celite and washed with some EtOAc. Concentration to remove the solvent and the resulting residue was purified with column (silica gel, EtOAc) gave the product 338 (98 mg) as solid. HPLC-MS t_{R}=1.50 min (UV_{254 nm}); mass calculated for formula C₁₁H₁₀BrN₅S 323.0, observed LCMS m/z 324.0 (M+H).

### Example 339

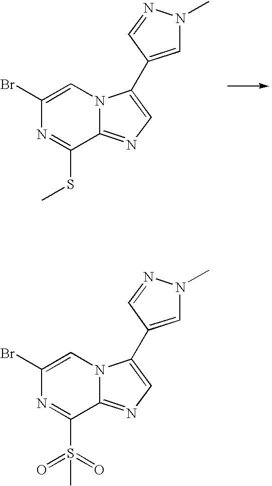

Compound 339 was synthesized using the same oxidation conditions described in example 181. HPLC-MS t_{R}=1.23 min (UV_{254 nm}); mass calculated for formula C₁₁H₁₀BrN₅O₂S 355.0, observed LCMS m/z 356 (M+H).

### Example 340

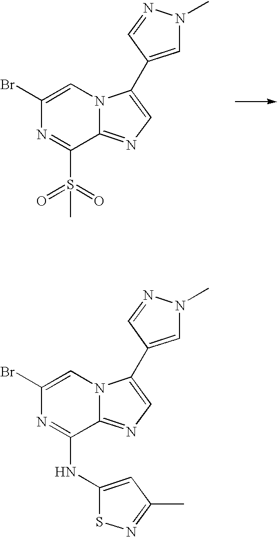

Compound 340 was prepared using the amination condition described in example 182. HPLC-MS t_{R}=1.44 min (UV_{254 nm}); mass calculated for formula C₁₄H₁₂BrN₇S 389.0, observed LCMS m/z 390.0 (M+H).

### Example 341

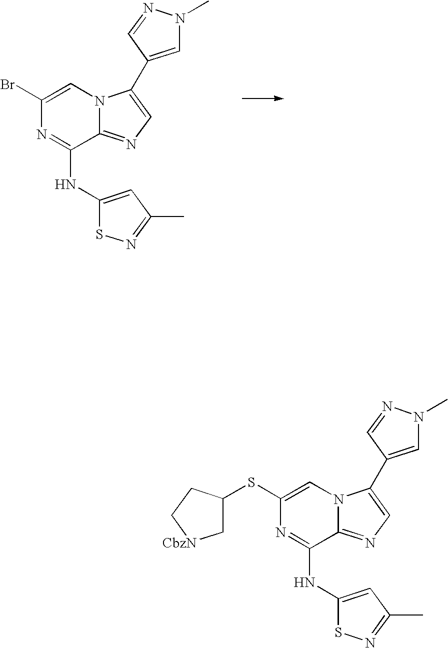

Under Argon, to the vial which charged with the compound 340 (˜20 mg, 0.05 mmol), Pd(dppf)Cl₂ (8 mg, 0.01 mmol), and sodium t-butoxide (15 mg, 0.15 mmol), thiol (15 mg, 0.06 mmol) in DME (2 mL) was added. The mixture was thoroughly degassed by alternately connected the flask to vacuum and Argon. The resulting solution was heated upto 80° C. and stirred overnight. After cooling to room temperature, the mixture was diluted with EtOAc (50 mL) and washed with NH₄Cl (sat. aq.), water, brine, and dried over Na₂SO₄. After concentration to remove the solvent and the resulting residue was purified with HPLC gave the product 341 (98 mg) as solid. HPLC-MS t_{R}=1.63 min (UV_{254 nm}); mass calculated for formula C₂₆H₂₆N₈O₂S₂ 546.2, observed LCMS m/z 547.2 (M+H).

### Example 342

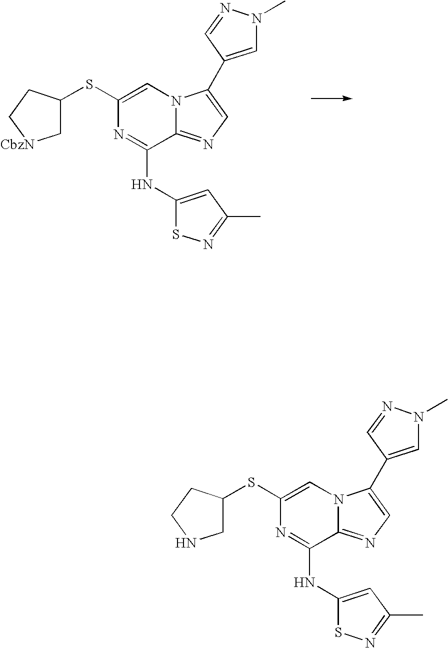

Compound 342 was synthesized using the deprotecting condition described in example 183. HPLC-MS t_{R}=0.95 min (UV_{254 nm}); mass calculated for formula C₁₈H₂₀N₈S₂ 412.1, observed LCMS m/z 413.0 (M+H).

### Example 343

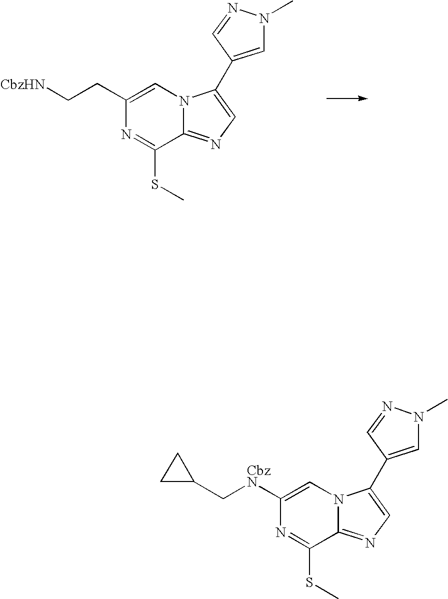

Compound 180 (100 mg) was dissolved in DMF (5 ml) and NaH (24 mg, 0.6 mmol) was added. After stirring 10 min at room temperature, cyclopropylmethylbromide (100 mg) was added and the resulting mixture was stirred at room temperature overnight. EtOAc (100 mL) was added and the organics was washed with water, brine and dried over Na₂SO₄. After concentration, the crud product was purified with column (silica gel, EtOAc/hexane=50:50-100:0) gave the product 343 (88 mg). HPLC-MS t_{R}=1.98 min (UV_{254 nm}); mass calculated for formula C₂₅H₂₈N₆O₂S 476.2, observed LCMS m/z 477.1 (M+H).

### Example 344

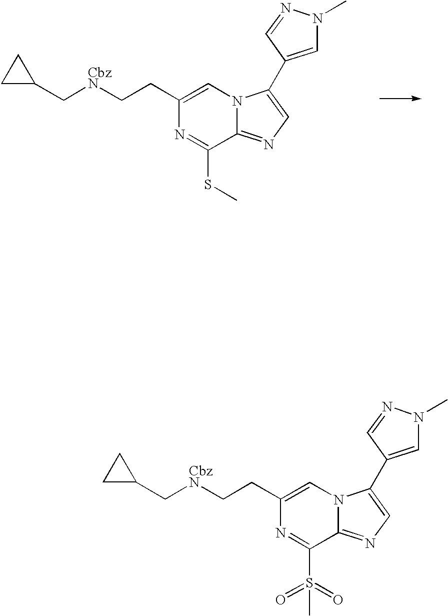

Compound 344 was synthesized using the same oxidation condition described in example 181. HPLC-MS t_{R}=1.69 min (UV_{254 nm}); mass calculated for formula C₂₅H₂₈N₆O₄S 508.2, observed LCMS m/z 509.2 (M+H).

### Example 345

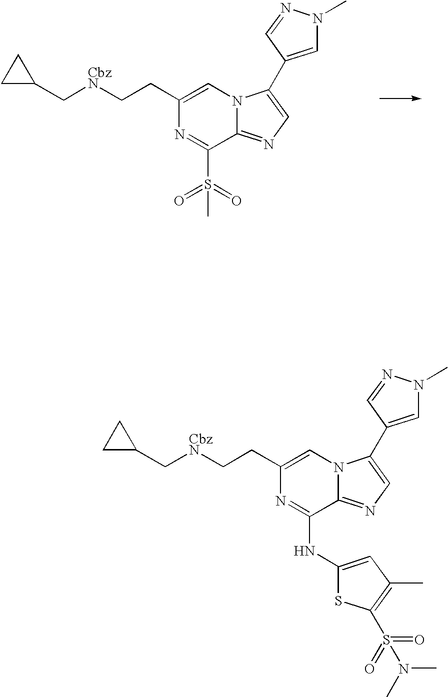

Compound 345 was prepared using the amination condition described in example 182. HPLC-MS t_{R}=2.05 min (UV_{254 nm}); mass calculated for formula C₃₁H₃₆N₈O₄S₂ 648.2, observed LCMS m/z 649.1 (M+H).

### Example 346

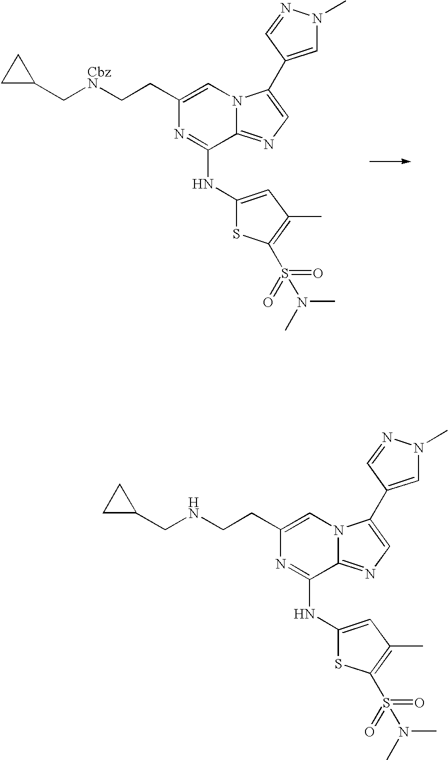

Compound 346 was synthesized using the deprotecting condition described in example 183. HPLC-MS t_{R}=1.31 min (UV_{254 nm}); mass calculated for formula C₂₃H₃₀N₈O₂S₂ 514.2, observed LCMS m/z 515.2 (M+H).

### Example 347

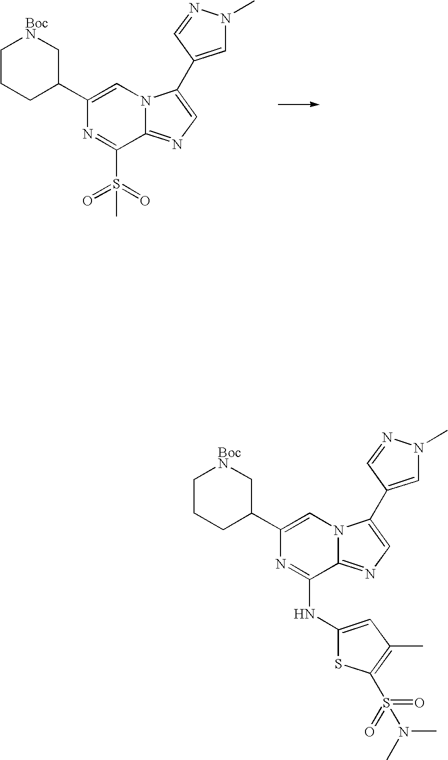

Compound 347 was prepared from compound 213 using the amination condition described in example 4 part G. HPLC-MS t_{R}=2.00 min (UV_{254 nm}); mass calculated for formula C₂₇H₃₆N₈O₄S₂ 600.2, observed LCMS m/z 601.2 (M+H).

### Example 348

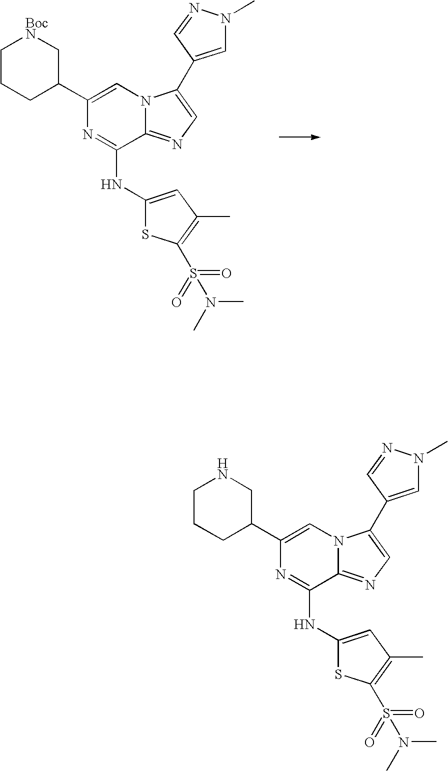

Compound 348 was synthesized using the deprotecting condition described in example 215. HPLC-MS t_{R}=1.26 min (UV_{254 nm}); mass calculated for formula C₂₂H₂₈N₈O₂S₂ 500.2, observed LCMS m/z 501.1 (M+H).

### Example 349

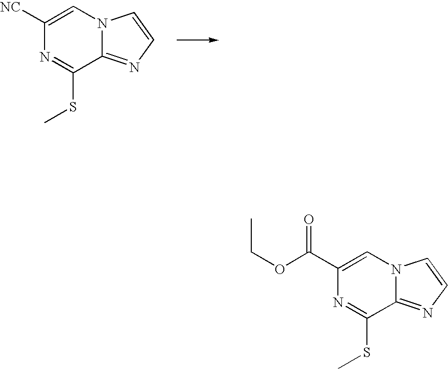

Compound 216 (342 mg, 1.8 mmol) and TMSCl (2.0 g) was dissolved in ethanol (20 mL). The mixture was heated to 70° C. and stirred 2 days. After concentration, the residue was purified with column (silica gel, EtOAC/hexane 10=30:70) gave the product 349 (280 mg). HPLC-MS t_{R}=1.27 min (UV_{254 nm}); mass calculated for formula C₁₀H₁₁N₃O₂S 237.1, observed LCMS m/z 238.1 (M+H).

### Example 350

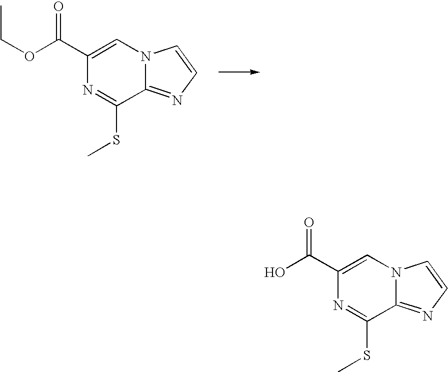

Compound 349 (280 mg, 1.18 mmol) was dissolved in the mixture of THF/MeOH (10 mL/10 mL) and LiOH (1N, 5.0 mL) was added. The resulting mixture was stirred at room temperature overnight and the solvent was removed under vacuum. The residue was taken up with water (5 mL), and adjusted to pH 5 with 1N HCl. The solid was collected with filtration and washed with water and dried with air gave the product 350 (235 mg). HPLC-MS t_{R}=0.76 min (UV_{254 nm}); mass calculated for formula C₈H₇N₃O₂S 209.0, observed LCMS m/z 210.1 (M+H).

### Example 351

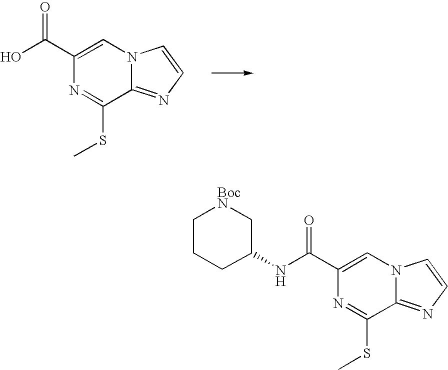

The acid 350 (42 mg, 0.2 mmol) was dissolved in DMF (5 mL) and HATU (76 mg, 0.2 mmol) was added followed by DIEA (300 μL) and amine (40 mg, 0.2 mmol). The resulting mixture was stirred at room temperature overnight and diluted with EtOAc. The organics was washed with water, brine and dried over Na₂SO₄. After concentration, the crude was purified with column (silica gel, EtOAc/hexane=30/70) to afford the product 351 (62 mg). HPLC-MS t_{R}=1.68 min (UV_{254 nm}); mass calculated for formula C₁₈H₂₅N₅O₃S 391.2, observed LCMS m/z 392.2 (M+H).

### Example 352

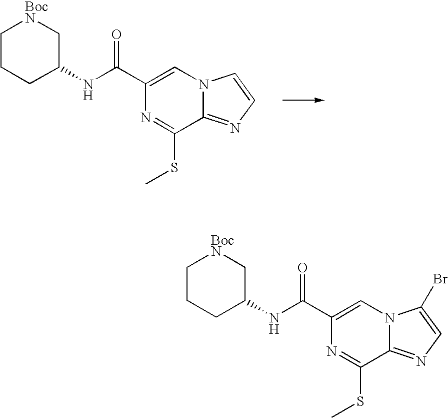

Compound 352 was prepared using the bromination condition described in example 179. HPLC-MS t_{R}=1.96 min (UV_{254 nm}); mass calculated for formula C₁₈H₂₄BrN₅O₃S 469.1, observed LCMS m/z 470.0 (M+H).

### Example 353

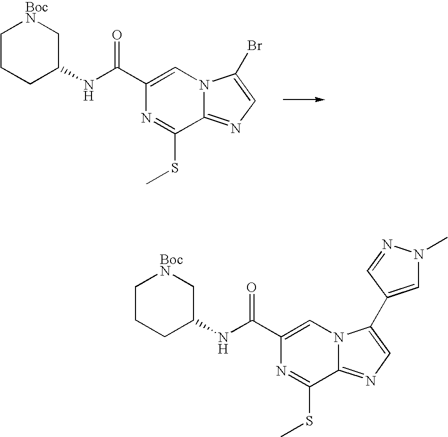

Compound 353 was synthesized using the same coupling condition described in example 180. HPLC-MS t_{R}=1.75 min (UV_{254 nm}); mass calculated for formula C₂₂H₂₉N₇O₃S 471.2, observed LCMS m/z 472.2 (M+H).

### Example 354

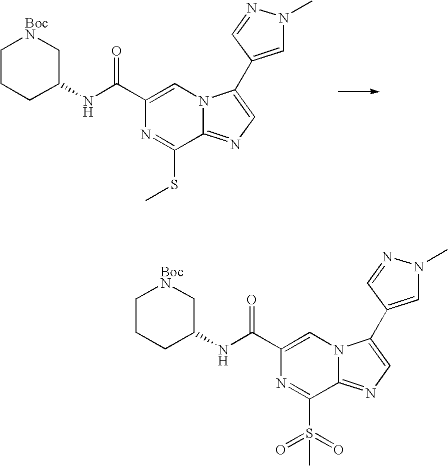

Compound 354 was synthesized using the same oxidation condition described in example 181. HPLC-MS t_{R}=1.52 min (UV_{254 nm}); mass calculated for formula C₂₂H₂₉N₇O₅S, 503.2, observed LCMS m/z 504.2 (M+H).

### Example 355

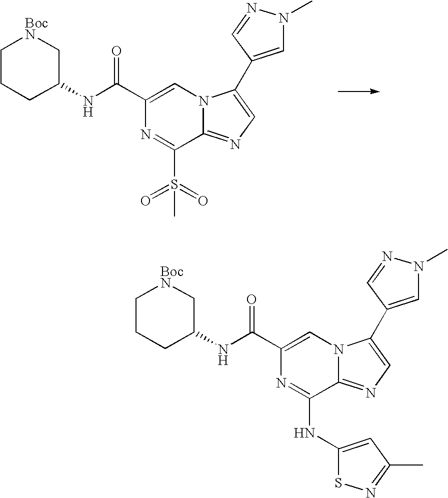

Compound 355 was prepared using the amination condition described in example 182. HPLC-MS t_{R}=1.58 min (UV_{254 nm}); mass calculated for formula C₂₅H₃₁N₉O₃S 537.2, observed LCMS m/z 538.3 (M+H).

### Example 356

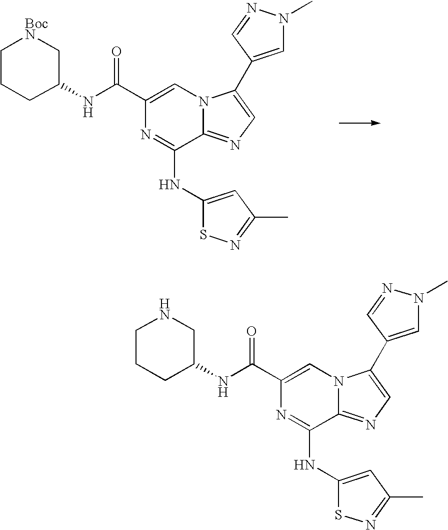

Compound 356 was synthesized using the deprotecting condition described in example 183. HPLC-MS t_{R}=0.84 min (UV_{254 nm}); mass calculated for formula C₂₀H₂₃N₉OS 437.2, observed LCMS m/z 438.3 (M+H).

### Example 357 & 358

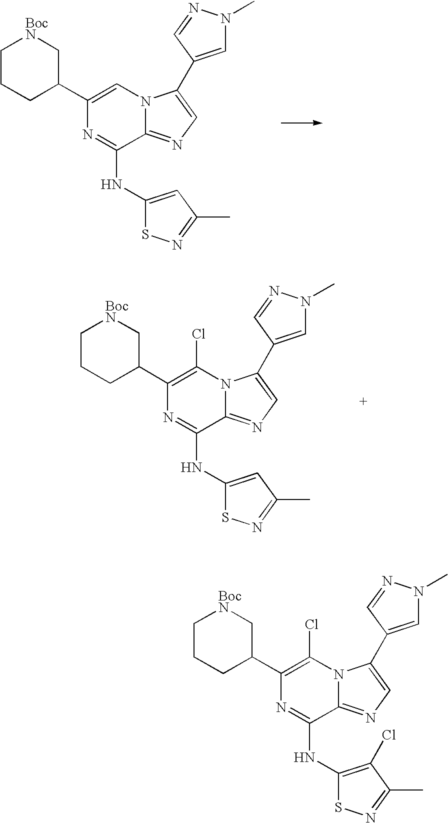

The compound 214 was dissolved in CHCl₃ (5 mL) and NCS (10 mg) was added, the mixture was heated to 50° C. and stirred for 2 hours. After concentration, the residue was purified with HPLC gave the product 357 and 358. Compound 357: HPLC-MS t_{R}=2.22 min (UV_{254 nm}); mass calculated for formula C₂₄H₂₉ClN₈O₂S 528.2, observed LCMS m/z 529.2 (M+H). Compound 358: HPLC-MS t_{R}=2.38 min (UV_{254 nm}); mass calculated for formula C₂₄H₂₈Cl₂N₈O₂S 562.1, observed LCMS m/z 563.0 (M+H).

### Example 359

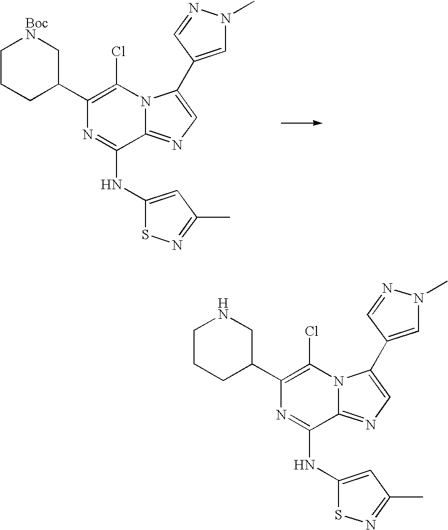

Compound 359 was synthesized using the deprotecting condition described in 215. and purified by preparative HPLC. HPLC-MS t_{R}=1.17 min (UV_{254 nm}); mass calculated for formula C₁₉H₂₁ClN₈S 428.1, observed LCMS m/z 429.1 (M+H).

### Example 360

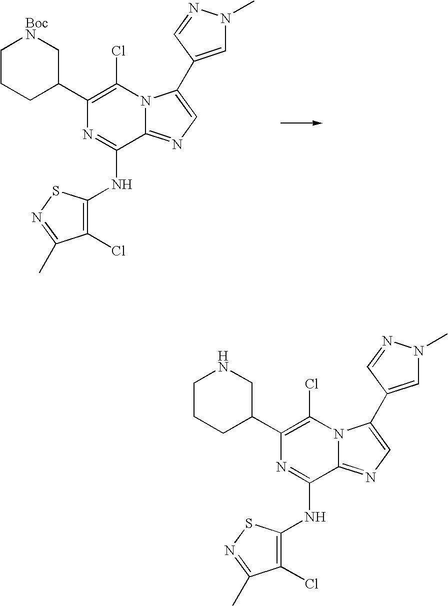

Compound 360 was synthesized using the deprotecting condition described in 215. and purified by preparative HPLC. Compound 360: HPLC-MS t_{R}=1.16 min (UV_{254 nm}); mass calculated for formula C₁₉H₂₁Cl₂N₈S 462.1, observed LCMS m/z 463.0 (M+H).

### Example 361

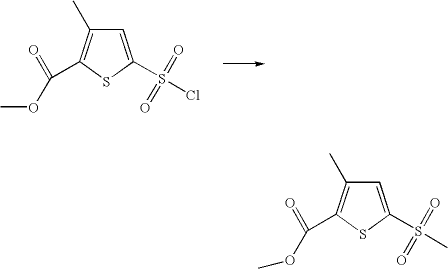

To a stirred solution of 5-chlorosulfonyl-3-methyl-thiophene-2-carboxylic acid methyl ester (0.254 g, 1 mmol) in dioxane (4 mL.) at room temperature is treated with a solution of sodium sulphite (0.252 g, 2 mmol) and sodium bicarbonate (0.168 g, 2 mmol) in water (4 mL). The reaction mixture is heated to 90° C. for 30 minutes and then allowed to cool to room temperature. The solvent is removed in vacuo. The residue is dissolved in DMF (4 mL), Iodomethane (0.248 g, 2 mmol) is added and stirred for 1 hr. The reaction mixture is diluted with water and extracted with ethyl acetate. The combined organic layers were washed with water, brine, dried over anhydrous sodium sulfate and concentrated. Crude product was purified on silica column using Hexane/Ethylacetate solvents to yield compound 361 (50%).

### Example 362

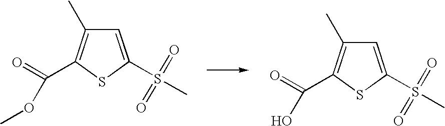

By essentially the same procedure given in Preparative Example 117, compound 362 can be prepared.

### Example 363

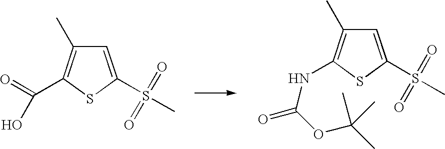

By essentially the same procedure given in Preparative Example 118, compound 363 can be made.

### Example 364

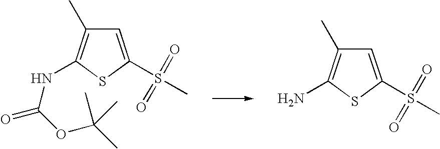

By essentially the same procedure given in Preparative Example 119, compound 364 can be prepared.

### Example 365

By essentially using the same procedures set forth in Preparative Example 361 through 364 by using isopropyl bromide, compound given in column 2 is prepared.

TABLE 32

| | | | | LCMS |
|---|---|---|---|---|
| Exam- | | | | MH⁺ |
| ple | Column 1 | Column 2 | MW | m/z |
| 365 | 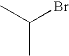 | 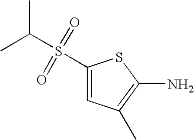 | 262.04 | 263.1 |

### Example 366

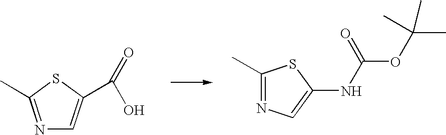

By essentially the same procedure given in Preparative Example 118, compound 366 can be prepared from 2-methyl thiazole-5-carboxylic acid HPLC-MS t_{R}=2.5 Min (UV₂₅₄ₙₘ). Mass calculated for formula C₉H₁₄N₂O₂S, M+214.20, observed LC/MS m/z 215.30(M+H)

### Example 367

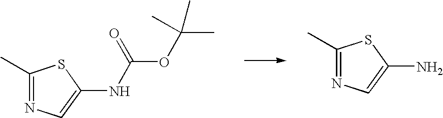

By essentially the same procedure given in Preparative Example 119, compound 367 can be prepared from 366. HPLC-MS t_{R}=1.25 Min (UV₂₅₄ₙₘ). Mass calculated for formula C₄H₆N₂S, M+114.20, observed LC/MS m/z 115.30 (M+H).

### Example 368

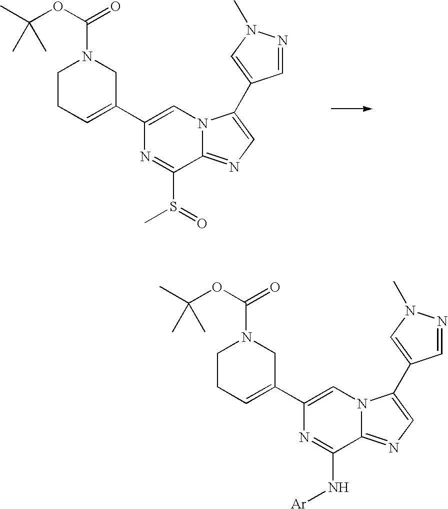

By essentially the same procedure given in Preparative Example 182, compounds given in Column 2 of Table 33 are prepared from compound 201 and amines listed in column 1, Table 33.

TABLE 33

| | | | | LCMS | |
|---|---|---|---|---|---|
| Exam- | | | | MH⁺ | HPLC |
| ple | Column 1 | Column 2 | MW | m/z | MS t_{R} |
| 368-1 | 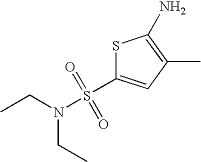 | 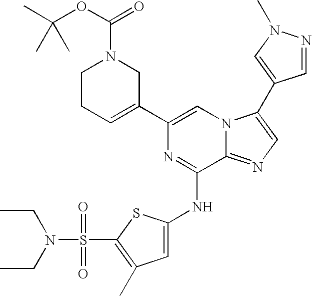 | 626.25 | 627.35 | 5.95 |
| 368-2 | 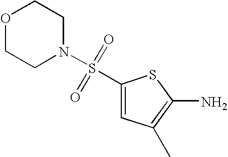 | 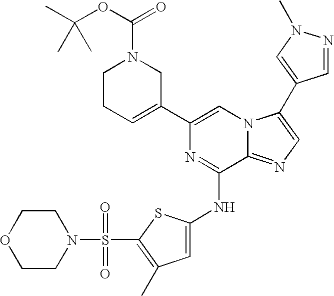 | 640.23 | 641.34 | 5.43 |
| 368-3 | 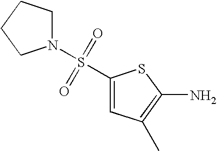 | 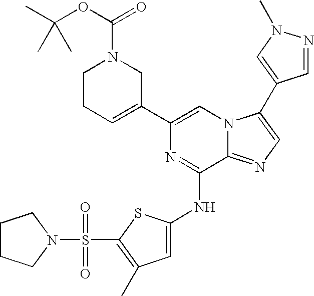 | 624.23 | 625.37 | 5.71 |
| 368-4 | 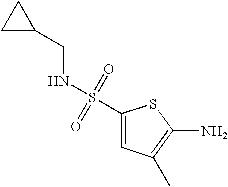 | 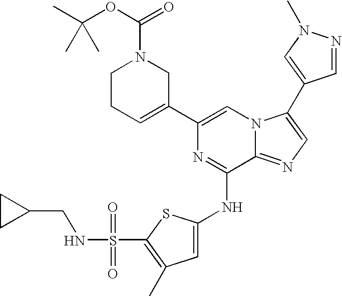 | 624.23 | 625.37 | 5.59 |
| 368-5 | 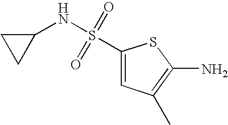 | 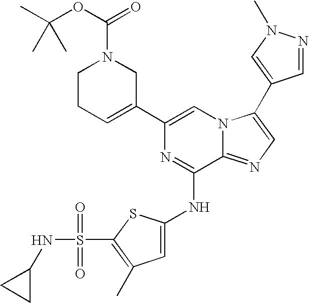 | 610.21 | 611.32 | 5.37 |
| 368-6 | 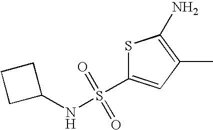 | 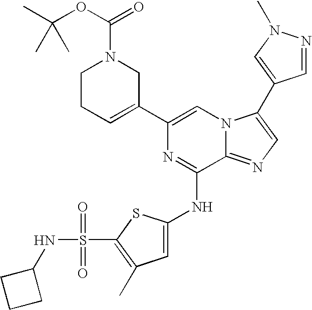 | 624.23 | 625.40 | 5.56 |
| 368-7 | 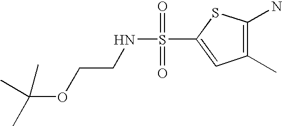 | 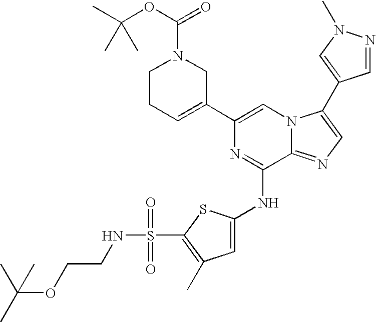 | 670.27 | 671.42 | 5.76 |
| 368-8 | 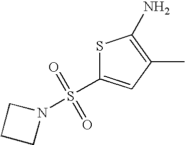 | 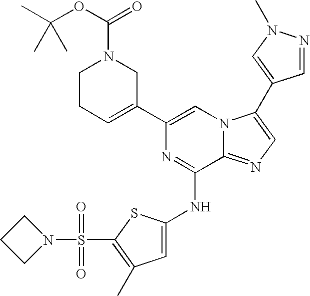 | 610.21 | 611.32 | 5.20 |
| 368-9 | 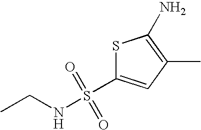 | 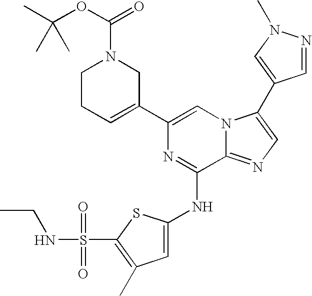 | 598.21 | 599.34 | 5.27 |
| 368-10 | 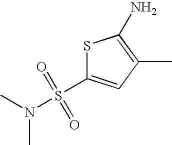 | 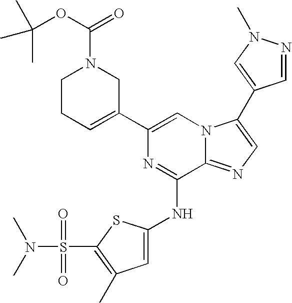 | 598.21 | 599.27 | 5.48 |
| 368-11 | 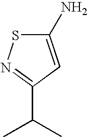 | 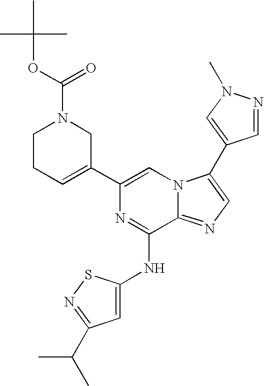 | 520.24 | 521.33 | 5.27 |
| 368-12 | 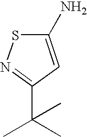 | 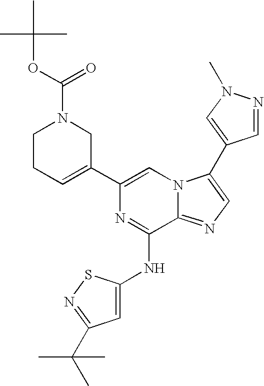 | 534.25 | 535.2 | 5.28 |
| 368-13 | 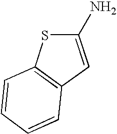 | 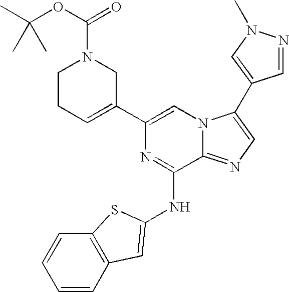 | 527.21 | 528.26 | 6.21 |
| 368-14 | 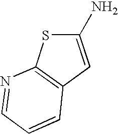 | 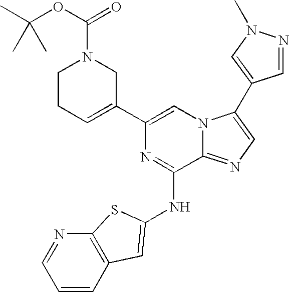 | 528.21 | 529.22 | 5.10 |
| 368-15 | 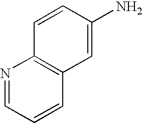 | 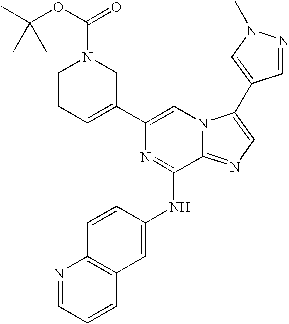 | 522.25 | 523.39 | 4.30 |
| 368-16 | 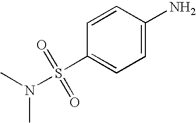 | 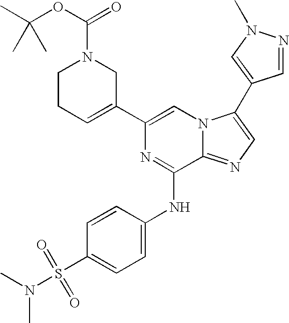 | 578.24 | 579.31 | 5.16 |
| 368-17 | 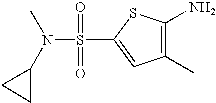 | 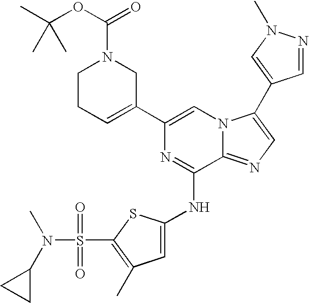 | 624.23 | 625.2 | 5.6 |
| 368-18 | 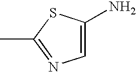 | 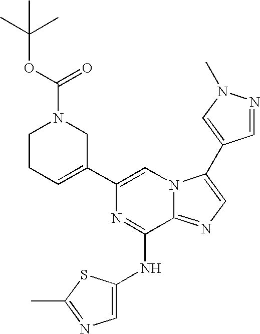 | 492.21 | 493.40 | 4.50 |
| 368-19 | 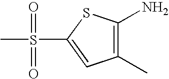 | 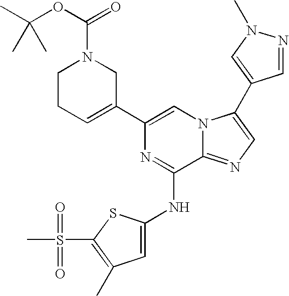 | 569.19 | 570.34 | 5.07 |
| 368-20 | 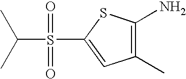 | 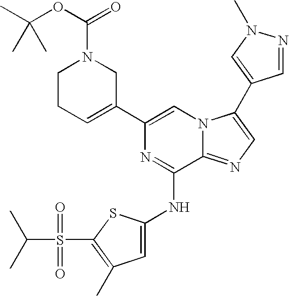 | 597.22 | 598.41 | 5.49 |

### Example 369

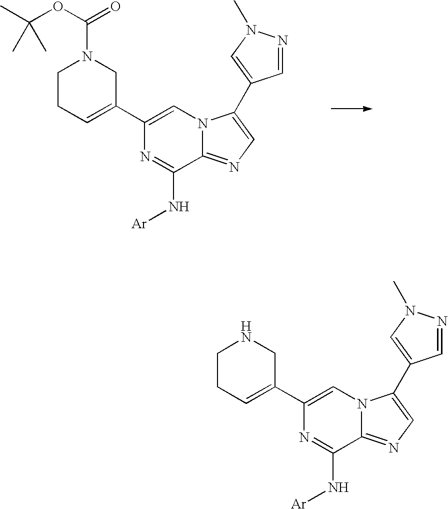

By essentially the same procedure given in Preparative Example 203, compounds given in Column 2 of Table 34 are prepared from compounds in column 1, Table 4.

TABLE 34

| | | | | LCMS | |
|---|---|---|---|---|---|
| Exam- | | | | MH⁺ | HPLC |
| ple | Column 1 | Column 2 | MW | m/z | MS t_{R} |
| 369-1 | 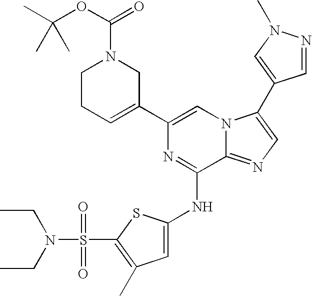 | 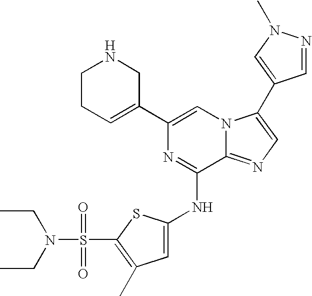 | 526.19 | 527.2 | 3.494 |
| 369-2 | 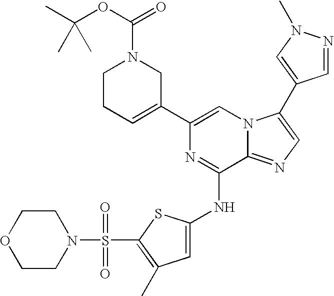 | 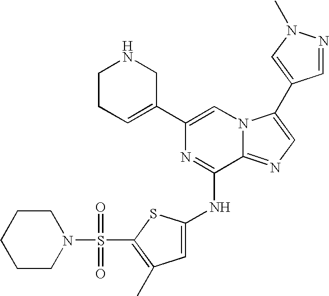 | 540.17 | 541.2 | 1.099 |
| 369-3 | 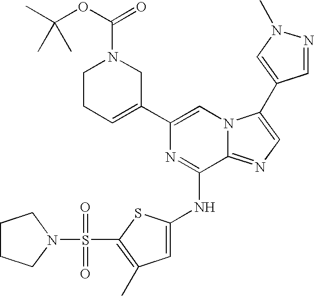 | 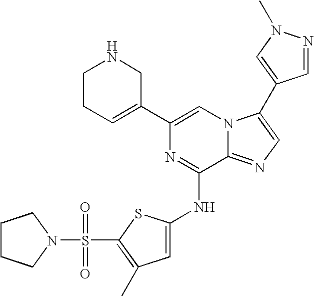 | 524.18 | 525.1 | 1.18 |
| 369-4 | 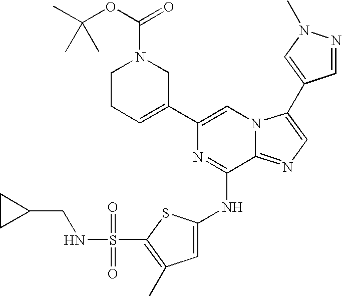 | 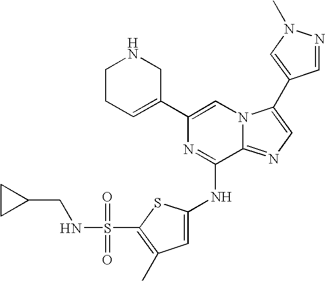 | 524.18 | 525.1 | 1.147 |
| 369-5 | 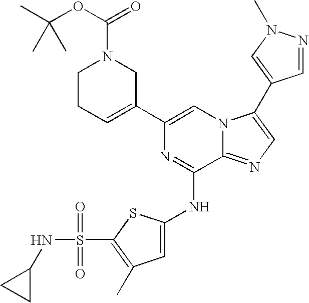 | 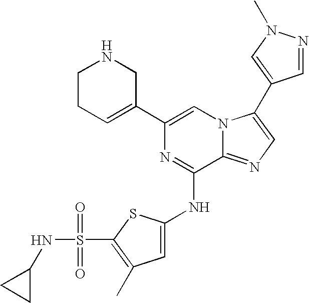 | 510.16 | 511.1 | 1.094 |
| 369-6 | 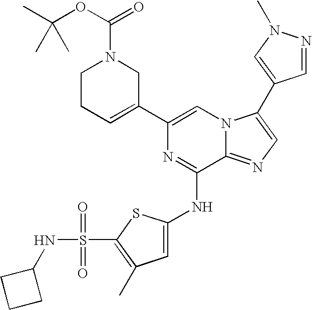 | 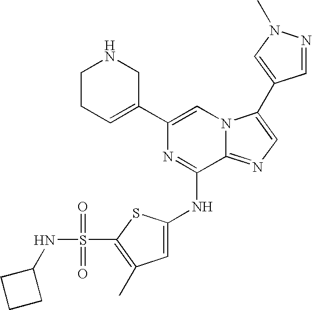 | 524.18 | 525.3 | 3.46 |
| 369-7 | 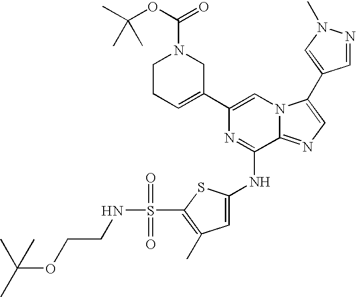 | 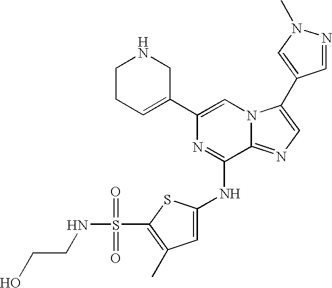 | 514.16 | 515.2 | 2.81 |
| 369-8 | 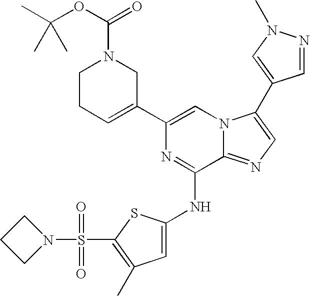 | 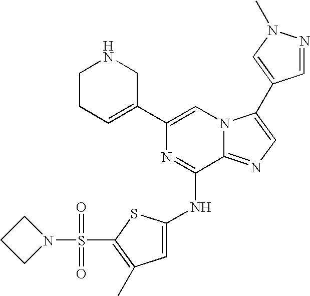 | 510.16 | 511 | 1.190 |
| 369-9 | 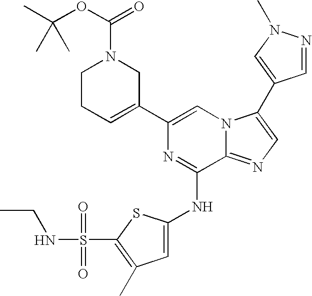 | 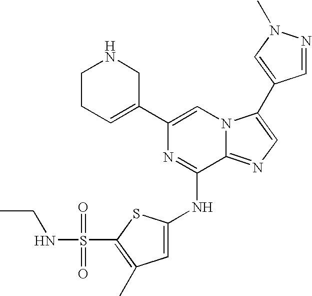 | 498.16 | 499.3 | 3.16 |
| 369-10 | 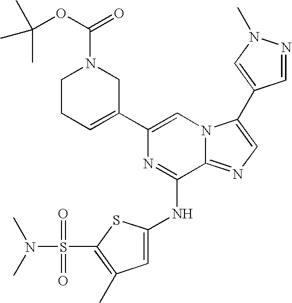 | 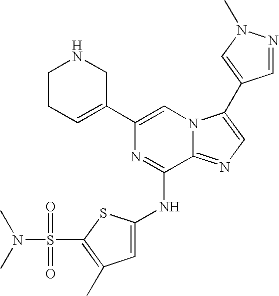 | 498.16 | 499.1 | 1.14 |
| 369-11 | 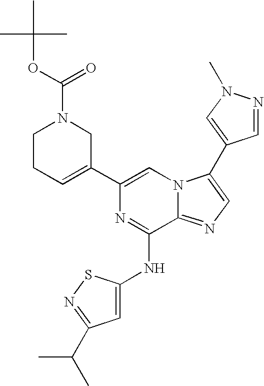 | 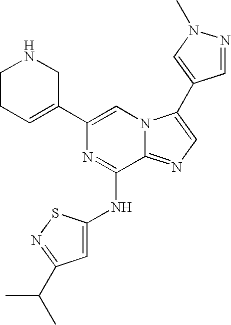 | 420.18 | 421.25 | 2.99 |
| 369-12 | 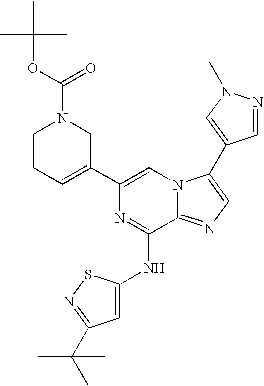 | 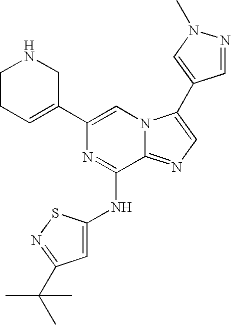 | 434.20 | 435.1 | 1.12 |
| 369-13 | 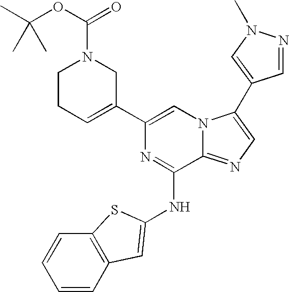 | 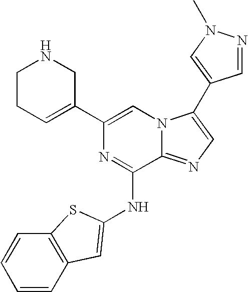 | 427.16 | 428.1 | 1.232 |
| 369-14 | 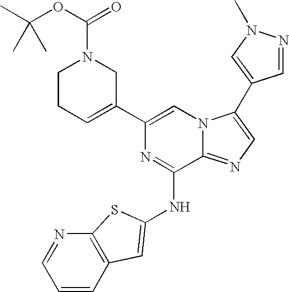 | 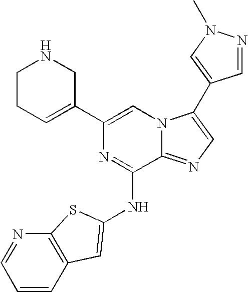 | 428.16 | 428.1 | 1.1 |
| 369-15 | 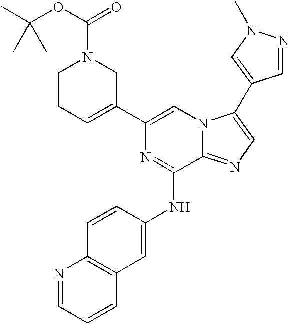 | 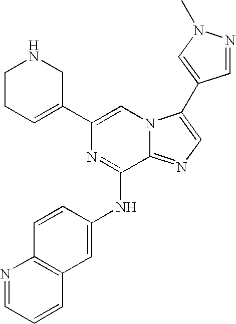 | 422.20 | 423.1 | 0.83 |
| 369-16 | 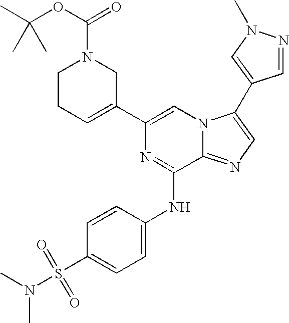 | 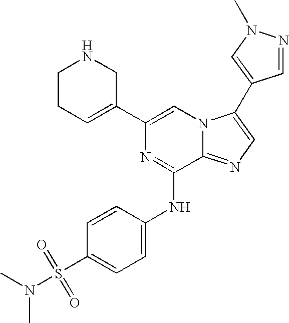 | 478.19 | 479.2 | 2.99 |
| 369-17 | 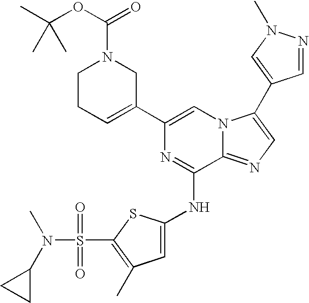 | 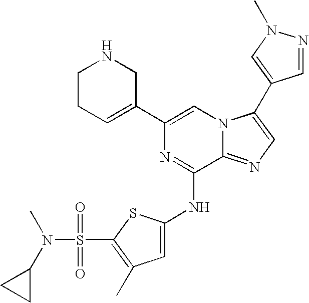 | 524.18 | 525.1 | 3.62 |
| 369-18 | 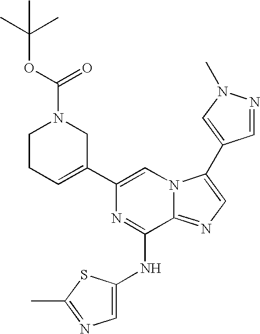 | 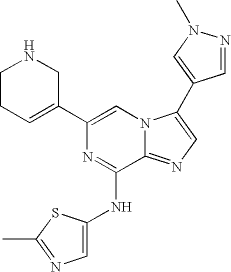 | 392.15 | 393.2 | 1.30 |
| 369-19 | 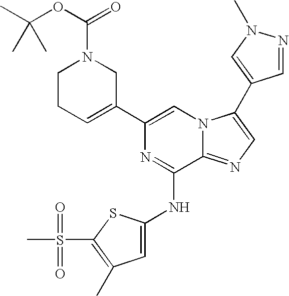 | 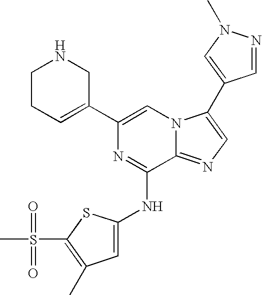 | 469.19 | 470.2 | 2.99 |
| 369-20 | 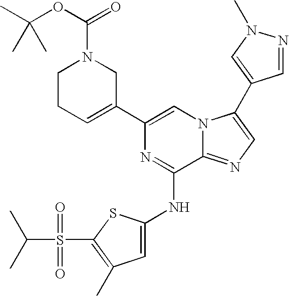 | 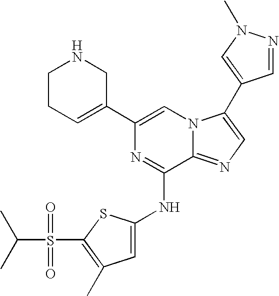 | 497.17 | 498.1 | 1.12 |

### Example 370

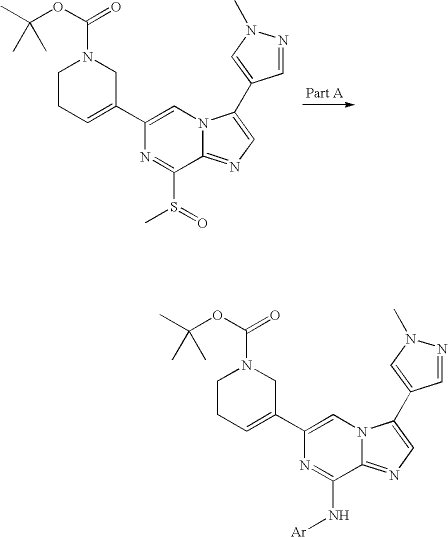

By essentially the same procedure given in Preparative Example 182, compounds given in Column 2 of Table 35 are prepared from compound 201 and amines listed in column 1, Table-35.

TABLE 35

| | | | | LCMS | |
|---|---|---|---|---|---|
| Exam- | | | | MH⁺ | HPLC |
| ple | Column 1 | Column 2 | MW | m/z | MS t_{R} |
| 370-1 | 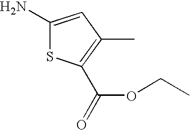 | 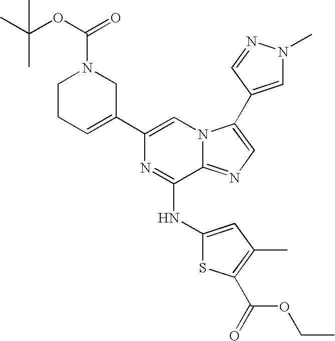 | 463.18 | 464.30 | 3.50 |
| 370-2 | 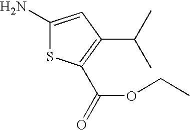 | 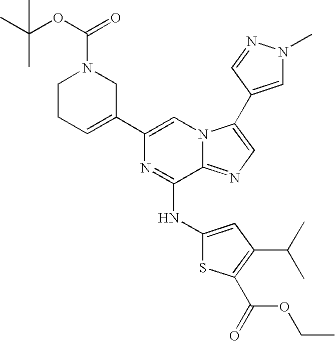 | 491.2 | 492.3 | 1.37 |
| 370-3 | 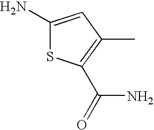 | 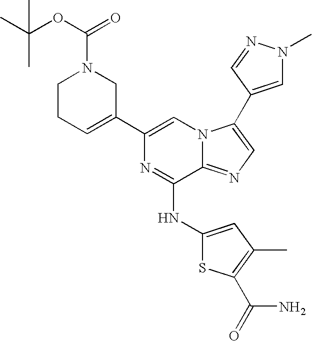 | 434.16 | 435.25 | 2.50 |
| 370-4 | 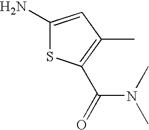 | 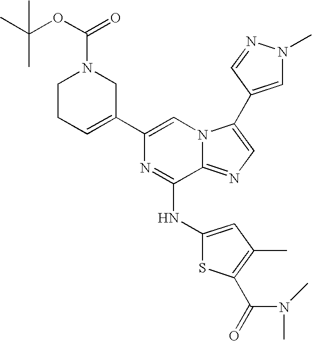 | 462.20 | 463.30 | 2.80 |
| 370-5 | 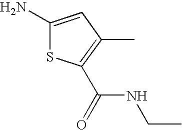 | 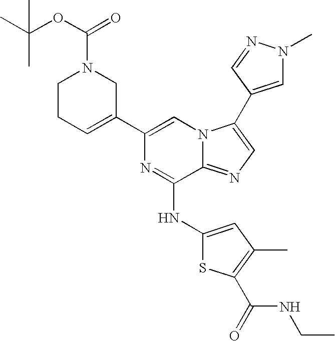 | 462.2 | 463.3 | 3.03 |
| 370-6 | 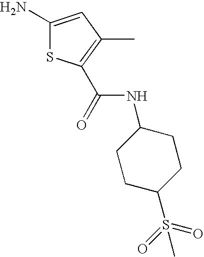 | 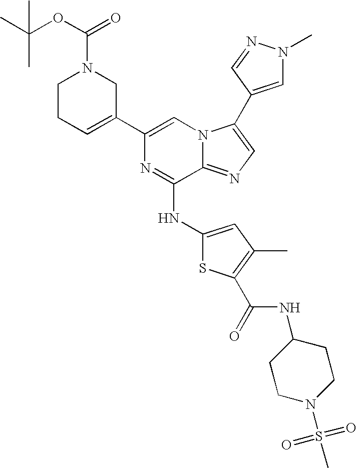 | 595.2 | 596.3 | 3.09 |

### Example 371

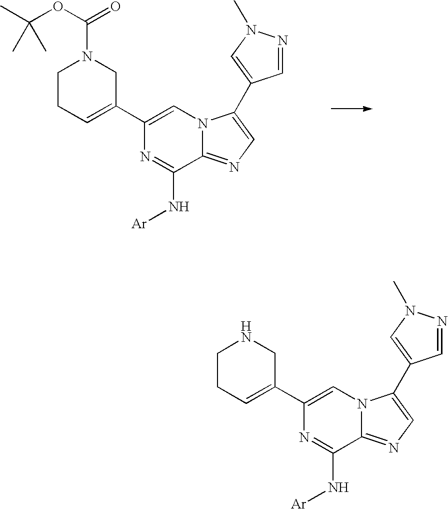

By essentially the same procedure given in Preparative Example 203, compounds given in Column 2 of Table 36 are prepared from compounds in column 1,

TABLE 36

| | | | | LCMS | |
|---|---|---|---|---|---|
| Exam- | | | | MH⁺ | HPLC |
| ple | Column 1 | Column 2 | MW | m/z | MS t_{R} |
| 371-1 | 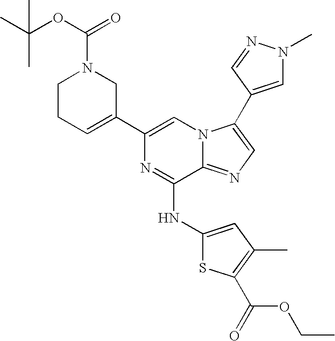 | 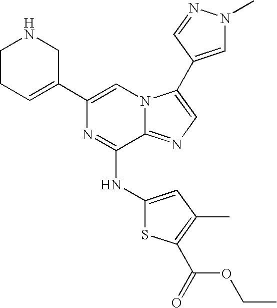 | 363.18 | 364.30 | 2.50 |
| 371-2 | 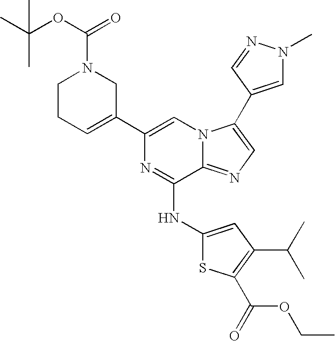 | 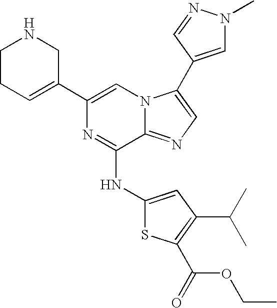 | 391.2 | 392.3 | 1.37 |
| 371-3 | 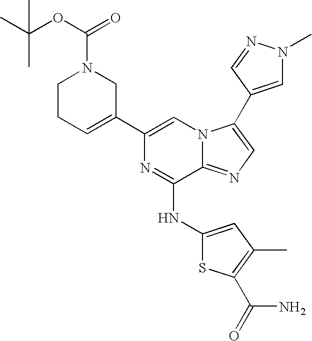 | 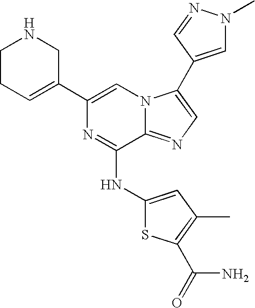 | 334.16 | 335.25 | 1.50 |
| 371-4 | 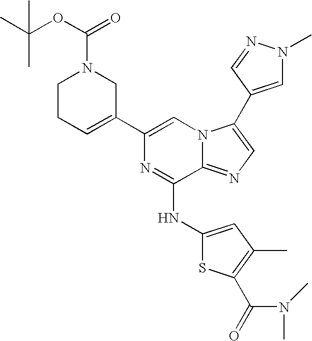 | 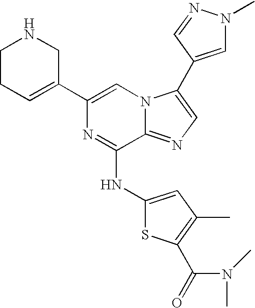 | 362.20 | 363.30 | 1.80 |
| 371-5 | 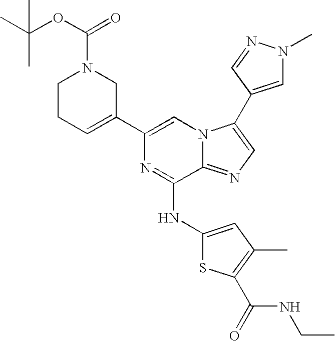 | 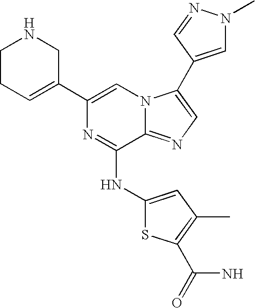 | 362.2 | 363.3 | 2.03 |
| 371-6 | 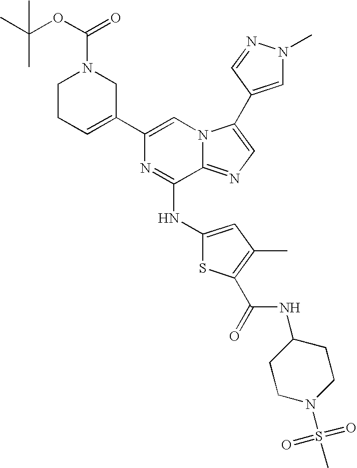 | 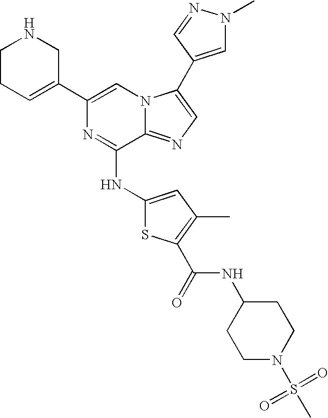 | 495.2 | 496.3 | 2.09 |

### Example 372

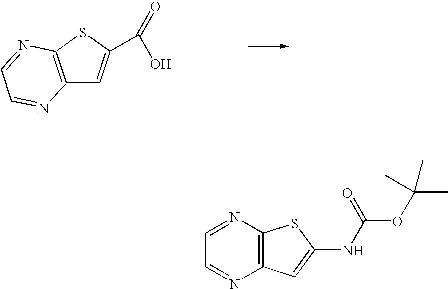

By essentially the same procedure given in Preparative 118, compound 372 can be prepared from thieno{2,3-b]pyrazine-6-carboxylic acid Compound 372: HPLC-MS t_{R}=2.5 Min (UV₂₅₄ₙₘ). Mass calculated for formula C₁₁H₁₃N₃O₂S, M+251.2018, observed LC/MS m/z 252.30(M+H).

### Example 373

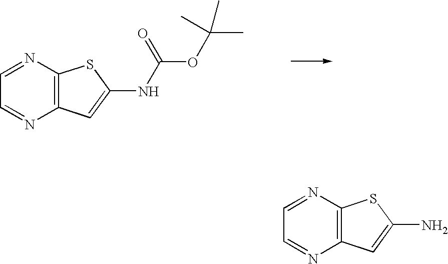

By essentially the same procedure given in Preparative 118, compound 372 can be prepared from 371:HPLC-MS t_{R}=1.5 Min (UV₂₅₄ₙₘ). Mass calculated for formula C₆H₅N₃S, M+151.2018 observed LC/MS m/z 152.30(M+H)

### Example 374

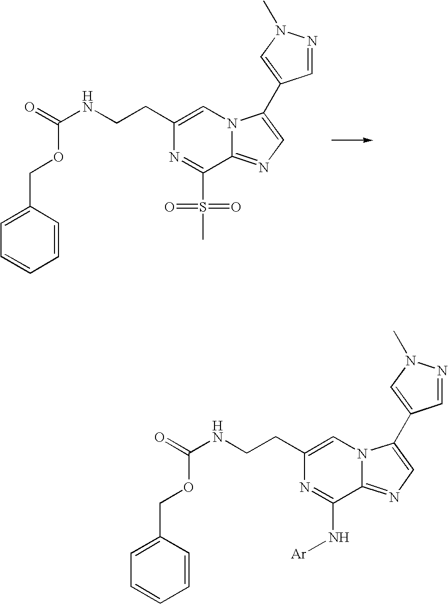

By essentially the same procedure given in Preparative Example 182, compounds given in Column 2 of Table 37 are prepared from compound 181 and amines listed in column 1, Table 37.

TABLE 37

| | | | | LCMS | |
|---|---|---|---|---|---|
| Exam- | | | | MH⁺ | HPLC |
| ple | Column 1 | Column 2 | MW | m/z | MS t_{R} |
| 374-1 | 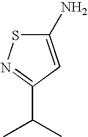 | 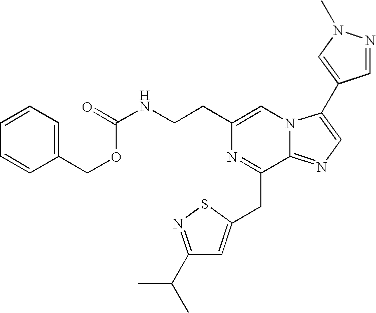 | 516.21 | 517.2 | 3.87 |
| 374-2 | 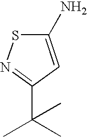 | 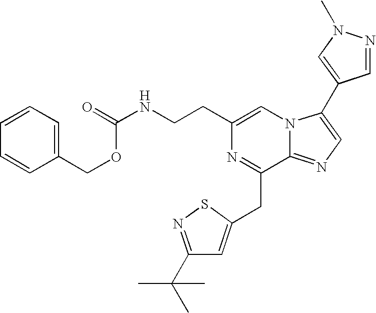 | 530.22 | 531.1 | 1.836 |
| 374-3 | 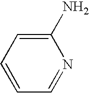 | 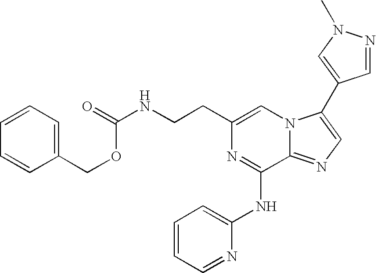 | 468.20 | 469.1 | 1.149 |
| 374-4 | 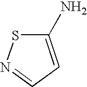 | 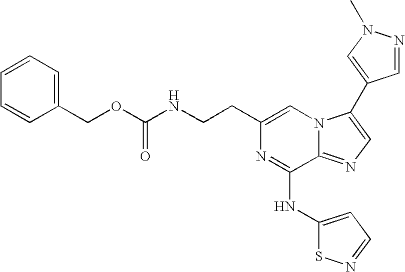 | 474.16 | 475.3 | 4.20 |
| 374-5 | 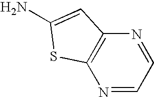 | 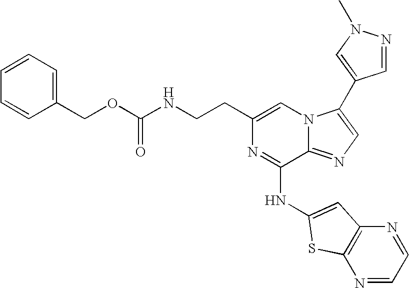 | 525.17 | 528.30 | 5.60 |
| 374-6 | 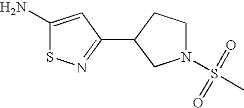 | 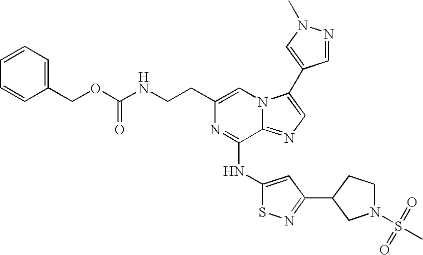 | 621.19 | 622.30 | 5.50 |
| 374-7 | 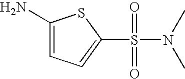 | 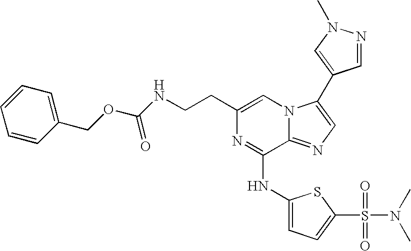 | 580.17 | 581.25 | 4.30 |

### Example 375

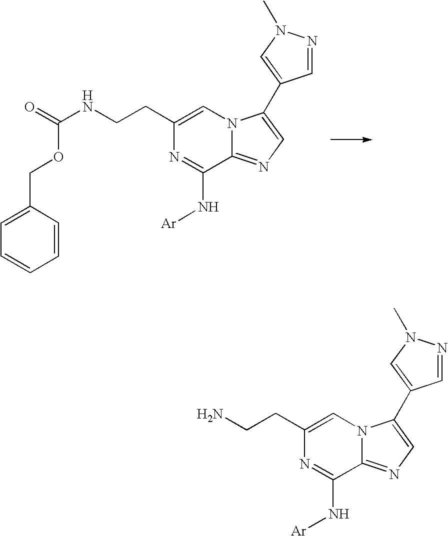

By essentially the same procedure given in Preparative Example 183, compounds given in Column 2 of Table 38 are prepared from compounds column 1, Table 38.

TABLE 38

| Ex- | | | | LCMS | |
|---|---|---|---|---|---|
| am- | | | | MH⁺ | HPLC |
| ple | Column 1 | Column 2 | MW | m/z | MS t_{R} |
| 375-1 | 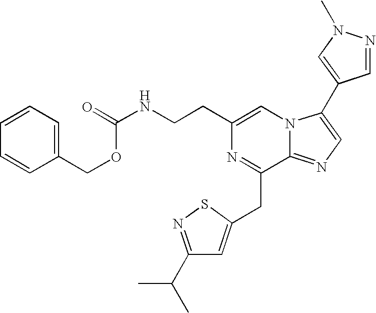 | 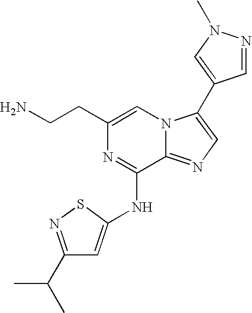 | 382.17 | 383.26 | 2.66 |
| 375-2 | 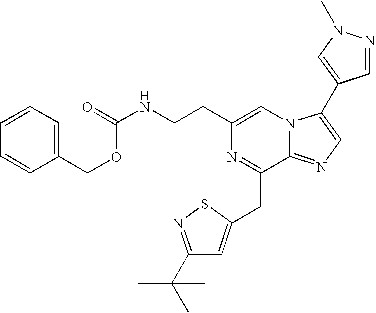 | 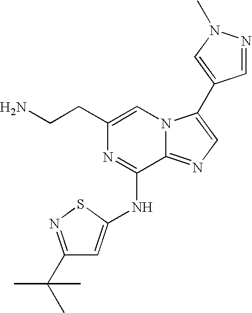 | 396.18 | 397.24 | 2.93 |
| 375-3 | 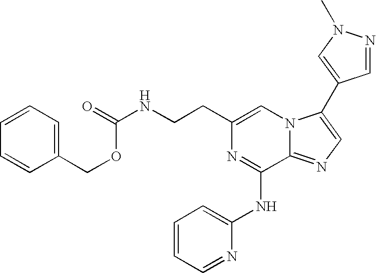 | 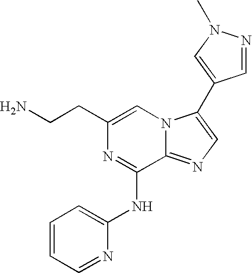 | 334.17 | 335.28 | 1.76 |
| 375-4 | 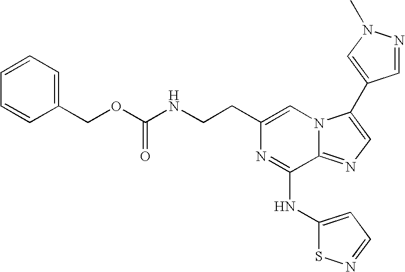 | 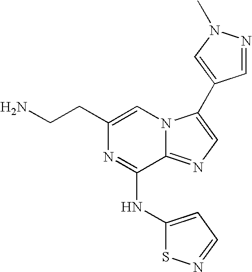 | 340.12 | 341.20 | 2.01 |
| 375-5 | 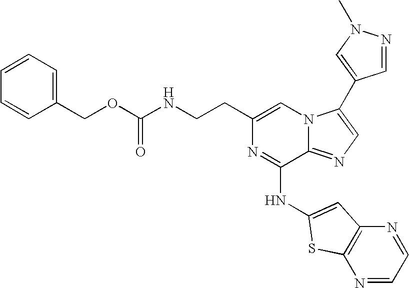 | 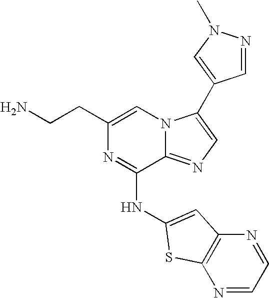 | 391.13 | 392.20 | 2.20 |
| 375-6 | 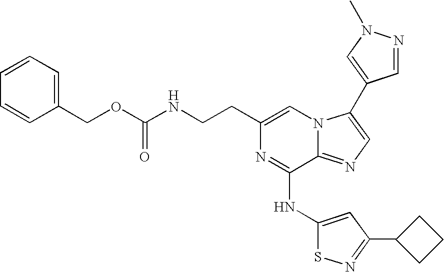 | 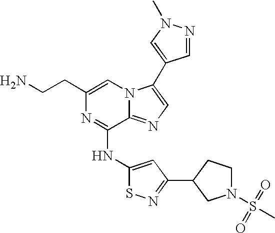 | 487 | 488 | 2.59 |
| 375-7 | 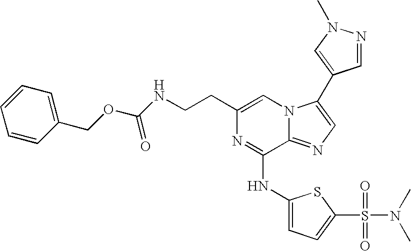 | 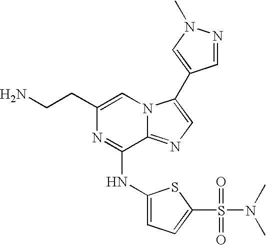 | 446 | 447 | 1.14 |

### Example 376

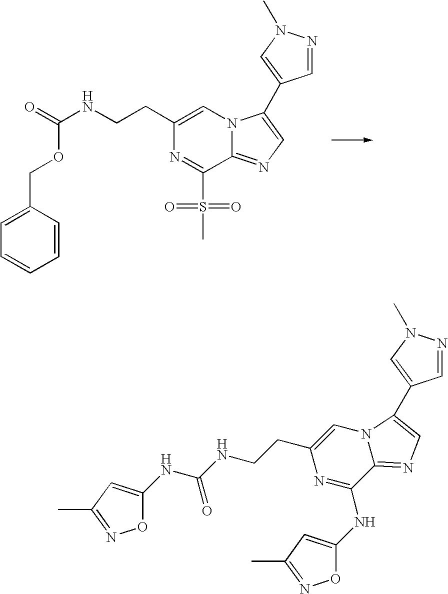

A solution of the isoxazole (2 equivalents) in DMSO (1 mL) was treated with NaH (60% dispersion in oil, 2 equivalents) for 15 min at room temperature. Compound 181 (1 equivalent) was then added to this solution at room temperature and the resultant solution was stirred at room temperature for 1 h at which time LC-MS analysis indicated the reaction was complete. The reaction mixture was diluted with sat. ammonium chloride (0.5 mL) and acetonitrile (0.5 mL). Purification by Prep-LC and conversion to a hydrochloric salt afforded compound 376. HPLC-MS t_{R}=3.33 Min (UV₂₅₄ₙₘ). Mass calculated for formula C₂₁H₂₂N₁₀O₃ 462.187, observed LC/MS m/z 463.24 (M+H).

### Example 377

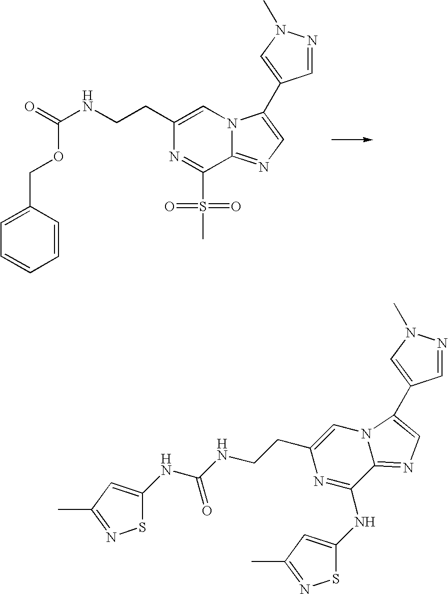

A solution of the isothiazole (2 equivalents) in DMSO (1 mL) was treated with NaH (60% dispersion in oil, 2 equivalents) for 15 min at room temperature. Compound 181 (1 equivalent) was then added to this solution at rt and the resultant solution was stirred at room temperature for 1 hr at which time LC-MS analysis indicated the reaction was complete. The reaction mixture was diluted with sat. ammonium chloride (0.5 mL) and acetonitrile (0.5 mL). Purification by Prep-LC and conversion to a hydrochloric salt afforded compound 377. ¹H-NMR (400 MHz, DMSO-d₆) δ 10.45 (bs, 1H), 8.42 (s, 1H), 7.96 (d, 2H), 7.91 (s, 1H), 7.15 (s, 1H), 6.95 (bs, 1H), 6.57 (s, 1H), 3.94 (s, 3H), 3.6 (q, 3H), 3.95 (t, 2H), 1.31 (s, 9H) and 1.22 (s, 9H). HPLC-MS t_{R}=3.76 Min (UV₂₅₄ₙₘ). Mass calculated for formula C27H34N10OS2 578.2, observed LC/MS m/z 579.2 (M+H).

### Example 378

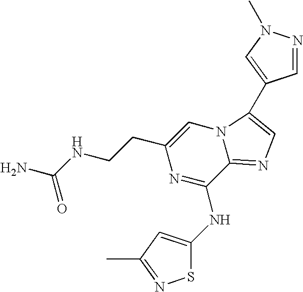

By essentially following the experimental procedures followed in the examples 376 & 377, the compound 378 can be prepared HPLC-MS t_{R}=2.15 Min (UV_{254 nm}). Mass calculated for formula C17H19N9OS 397.14, observed LC/MS m/z 398.20 (M+H).

### Example 379

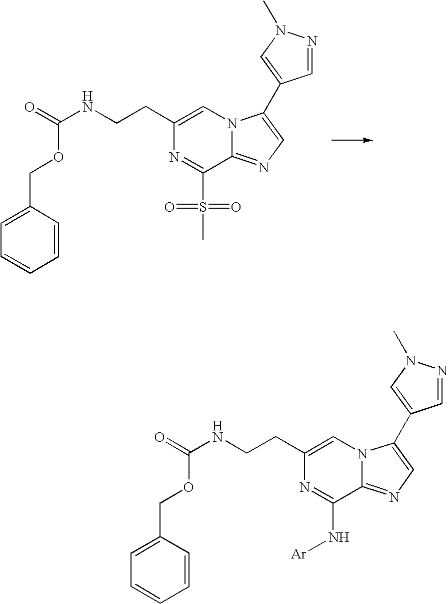

By essentially the same procedure given in Preparative Example 182, compounds given in Column 2 of Table-39 are prepared from compound 181 and amines listed in column 1, Table-39.

TABLE 39

| | | | | LCMS | |
|---|---|---|---|---|---|
| Exam- | | | | MH⁺ | HPLC |
| ple | Column 1 | Column 2 | MW | m/z | MS t_{R} |
| 379-1 | 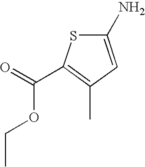 | 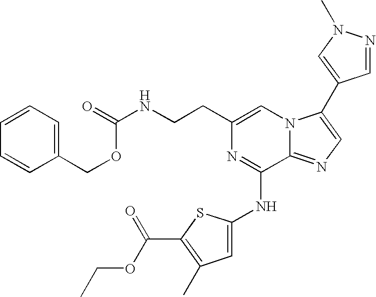 | 559.20 | 560.30 | 4.55 |
| 379-2 | 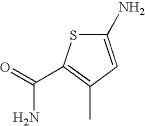 | 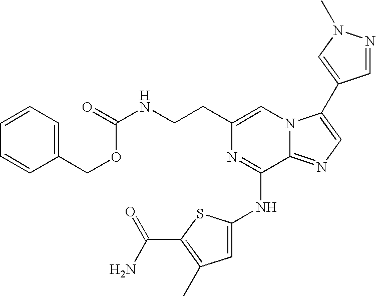 | 530.18 | 531.20 | 3.80 |
| 379-3 | 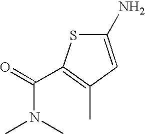 | 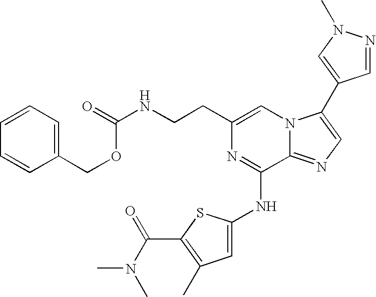 | 558.22 | 559.35 | 3.95 |
| 379-4 | 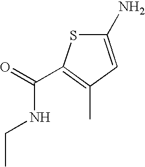 | 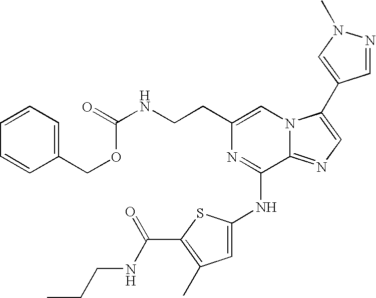 | 558.22 | 559.35 | 3.95 |

### Example 380

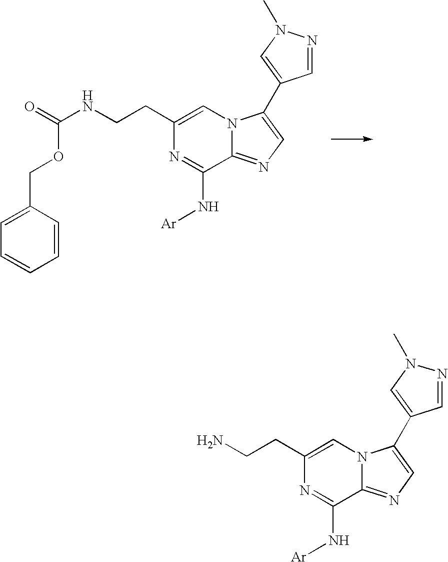

By essentially the same procedure given in Preparative Example 183, compounds given in Column 2 of Table 40 are prepared from compounds column 1, Table 40.

TABLE 40

| Ex- | | | | LCMS | |
|---|---|---|---|---|---|
| am- | | | | MH⁺ | HPLC |
| ple | Column 1 | Column 2 | MW | m/z | MS t_{R} |
| 380-1 | 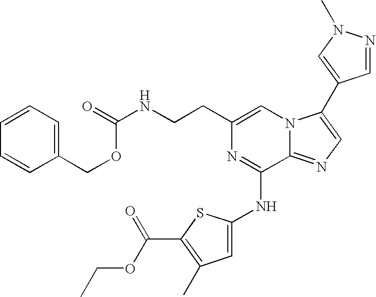 | 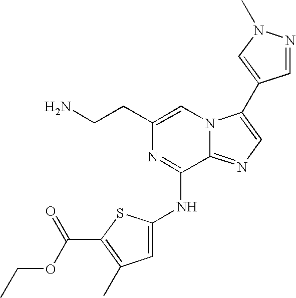 | 425.16 | 426.25 | 3.55 |
| 380-2 | 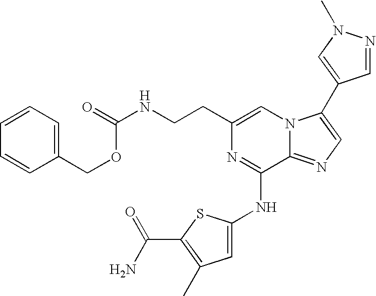 | 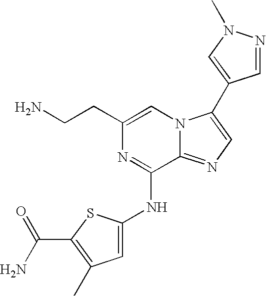 | 396.15 | 397.25 | 2.95 |
| 380-3 | 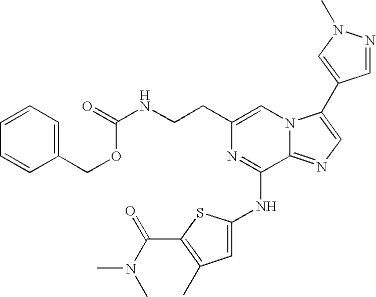 | 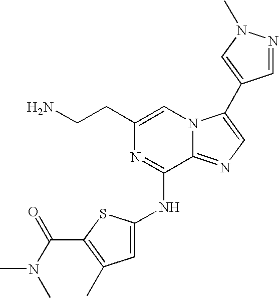 | 424.18 | 425.30 | 3.10 |
| 380-4 | 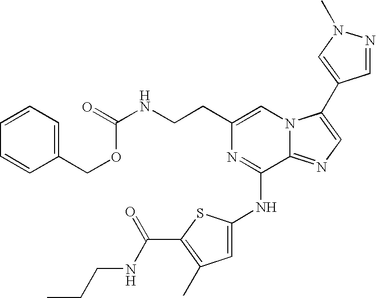 | 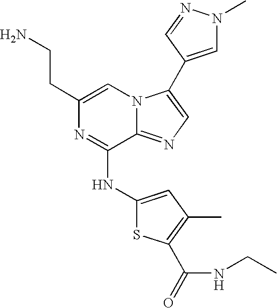 | 424.18 | 425.30 | 3.20 |
| 380-5 | 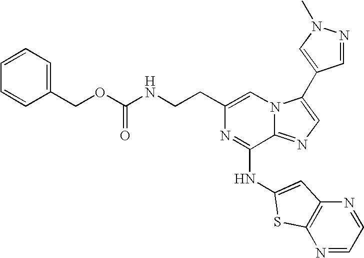 | 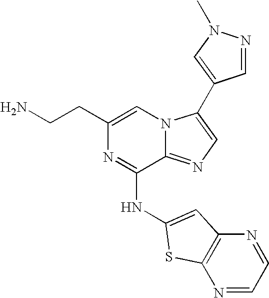 | 391.13 | 392.20 | 2.20 |

### Example 381

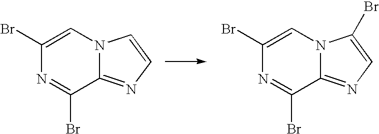

NBS (0.176 g, 1.0 mmol) was added to a solution of compound 176 (0.278 g, 1.0 mmol) in DCM (10 mL), at room temperature. The mixture was stirred for one hour and concentrated. The residue was diluted with EtOAc and washed with saturated aq.NaHCO₃ (30 mL, 2×), brine and dried over Na₂SO₄. After concentrating, the crude product 381 was used in the next step directly without further purification. HPLC-MS t_{R}=1.54 min (UV_{254 nm}); mass calculated for formula C₆H₂Br₃N₃, 352.78; observed MH⁺ (LCMS) 353.8 (m/z).

### Example 382

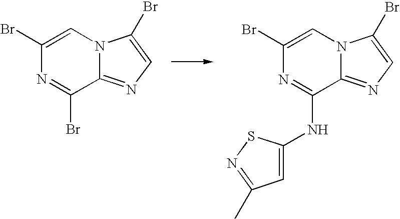

By essentially the same procedure given in Preparative Example 182, compound 382 is prepared from compound 381. HPLC-MS t_{R}=1.73 min (UV_{254 nm}); mass calculated for formula C₆H₂Br₃N₃, 386.88; observed MH⁺ (LCMS) 388.0 (m/z).

### Example 383

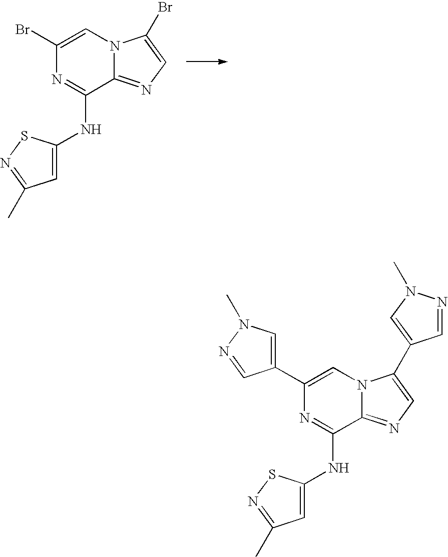

1-Methyl-4-(4,4,5,5-tetramethyl-[1,3,2]dioxoborolan-2-yl)-1H-Pyrazole (0.208 g, 1.0 mmol), was mixed with Pd(dppf)Cl₂ (50 mg, 0.06 mmol), K₃PO₄ (0.848 g., 4 mmol), and the product from example 382 (0.195 g, 0.50 mmol) in dioxane (5 mL) was added. The mixture was degassed thoroughly and kept under argon blanket. The resulting solution was heated at 80° C. and stirred overnight. After cooling to room temperature the mixture was diluted with EtOAc (50 mL). The solid was removed by filter through Celite and washed with EtOAc. The solvent was removed under reduced pressure. Purification by Prep-LC and conversion to a hydrochloric salt afforded compound 383. HPLC-MS t_{R}=3.08 min (UV_{254 nm}); mass calculated for formula C₁₈H₁₇N₉S, 391.13; observed MH⁺ (LCMS) 392.22 (m/z).

### Example 384

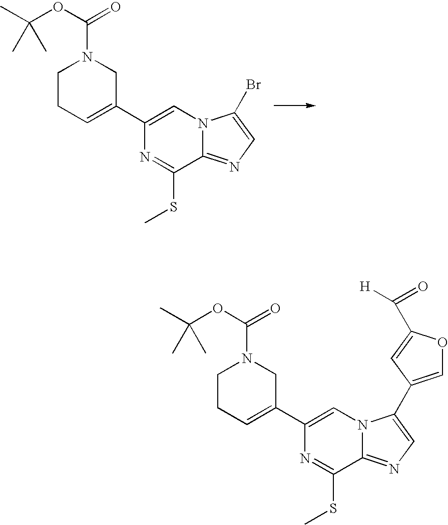

Compound 199 (0.433 g, 1.021 mmol), the 4-(4,4,5,5-tetramethyl-{1,3-2}dioxaboralan-2yl)furan-2carboxaldehyde (0.339 g, 1.52 mmol), PdCl₂dppf.CH₂Cl₂ (0.081 g, 0.12 mmol), and K₃PO₄ (0.865 g, 4.0 mmol) in 1,2-dimethoxyethane (10 mL) and H₂O (2 mL) was flushed with Ar and refluxed for 2 hr. The solvents were evaporated and the residue was purified by column chromatography on silica gel with 2:1 hexane/EtOAc as eluent to obtain product 384 (0.181 g,). HPLC-MS t_{R}=2.04 min (UV_{254 nm}); mass calculated for formula C₂₂H₂₄N₄O₄S, 440.12; observed MH⁺ (LCMS) 441.1 (m/z).

### Example 385

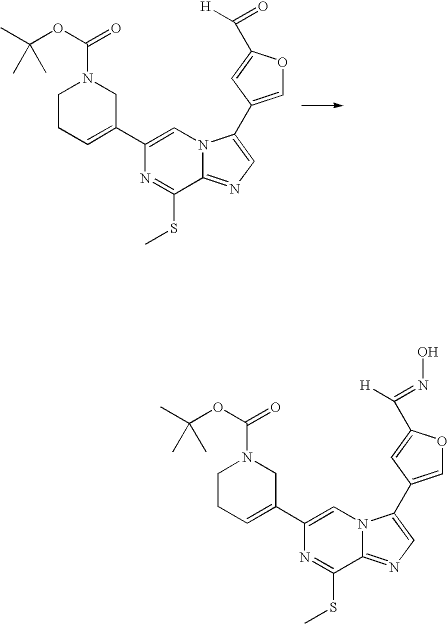

The product from Preparative Example 384 (0.181 g, 0.41 mmol) in CH₂Cl₂ (5 mL) and MeOH (1 mL) was added NH₂OH.HCl (0.043 g, 0.616 mmol), and triethylamine (1.2 mL) and stirred in a closed flask at 25° C. for 4 hr. The solvent was evaporated and the residue was chromatographed on silica gel with 2:1 hexane/EtOAc as eluent to obtain pure product 385 (0.120 g.). HPLC-MS t_{R}=1.968 min (UV_{254 nm}); mass calculated for formula C₂₂H₂₅N₅O₄S, 455.16; observed MH⁺ (LCMS) 456.1 (m/z).

### Example 386

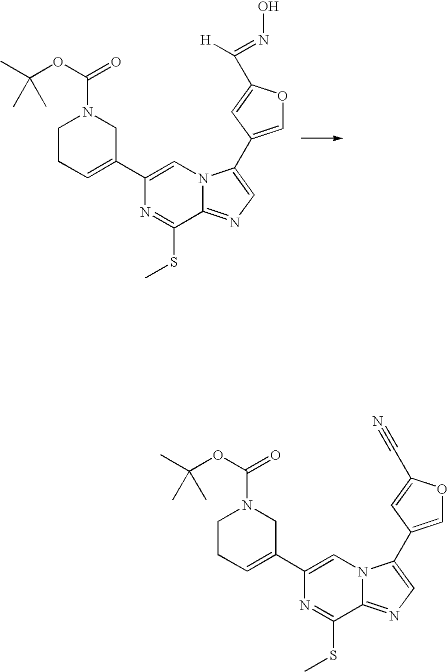

To the compound 385 (0.120 G., 0.263 mmol) and triethylamine (1.1 mL) in dichloromethane (5 mL) was added trifluoroacetic anhydride (0.036 mL, 0.258 mmol) was ad de d at 0° C. under Argon. The mixture was stirred for 2 hr, then it was poured into saturated aqueous NaHCO₃ solution (50 mL), extracted with CH₂Cl₂ (3×40 mL), dried over Na₂SO₄, and filtered. The solvents were evaporated and the residue was purified by column chromatography on silica gel with 50:1 CH₂Cl₂/MeOH as eluent to obtain pure product 386 (0.083 g). HPLC-MS t_{R}=2.181 min (UV_{254 nm}); mass calculated for formula C₂₂H₂₃N₅O₃S, 437.15; observed MH⁺ (LCMS) 438.1 (m/z).

### Example 387

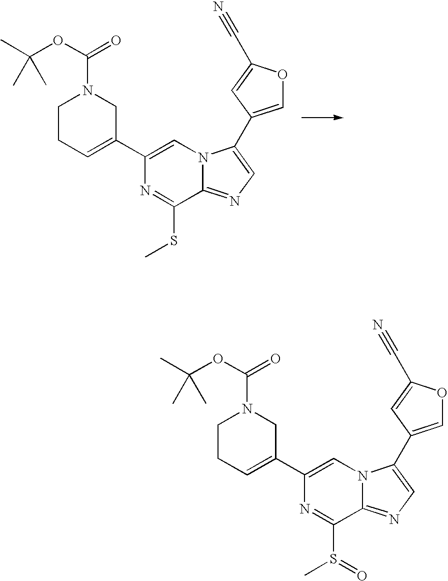

The mixture of compound from preparative example 386 (0.083 g, 0.183 mmol) and m-CPBA (31 mg, 77%) in DCM (5 mL) was stirred at 0° C. for min and then diluted with EtOAc (100 mL). The organics were washed with saturated aqueous NaHCO₃ (10 mL, 2×), brine, and dried over Na₂SO₄. After concentration the crude product which was used in the next step directly without further purification. HPLC-MS t_{R}=1.72 min (UV_{254 nm}); mass calculated for formula C₂₂H₂₃N₅O₄S, 453.15; observed MH⁺ (LCMS) 454.1 (m/z).

### Example 388

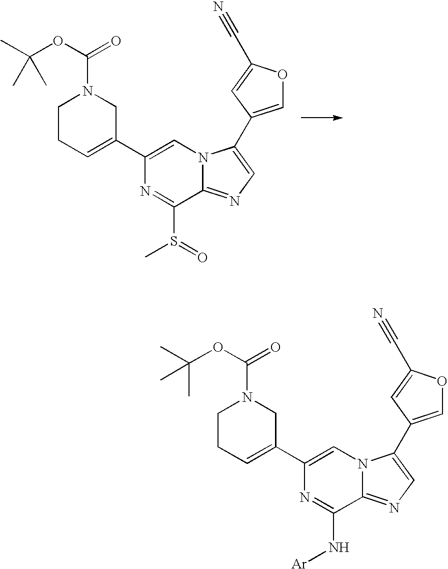

By essentially the same procedure given in Preparative Example 182, compounds 388 given in Column 2, Table 42 are prepared from compound from preparative example 387 and amines listed in column 1, Table 42

TABLE 41

| | | | | LCMS | |
|---|---|---|---|---|---|
| Exam- | | | | MH⁺ | HPLC |
| ple | Column 1 | Column 2 | MW | m/z | MS t_{R} |
| 388-1 | 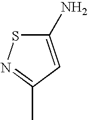 | 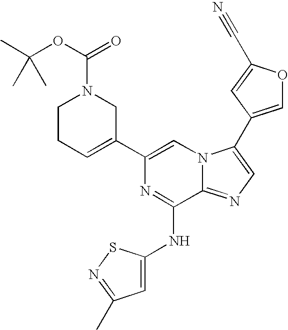 | 503.17 | 504.2 | 2.07 |
| 388-2 | 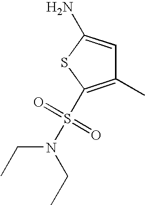 | 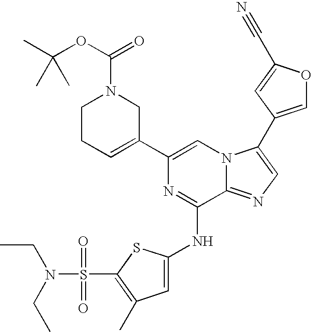 | 637.12 | 638.2 | 2.349 |

### Example 389

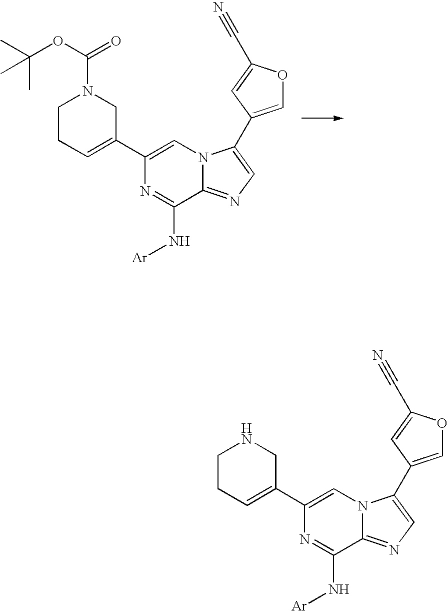

By essentially the same procedure given in Preparative Example 183, compounds 389 series given in Column 2 of Table 43 are prepared from compounds column 1, Table 43.

TABLE 42

| | | | | LCMS | |
|---|---|---|---|---|---|
| | | | | MH³⁰ | HPLC |
| Example | Column 1 | Column 2 | MW | m/z | MS t_{R} |
| 389-1 | 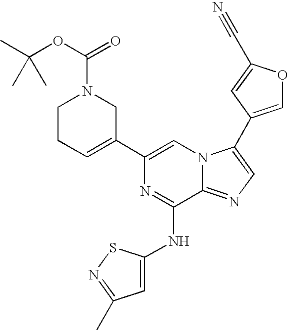 | 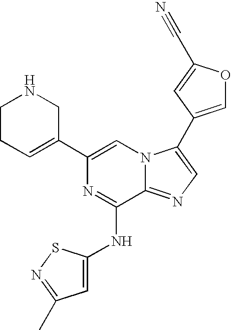 | 403.17 | 404.2 | 2.04 |
| 389-2 | 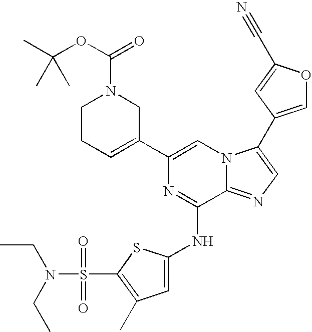 | 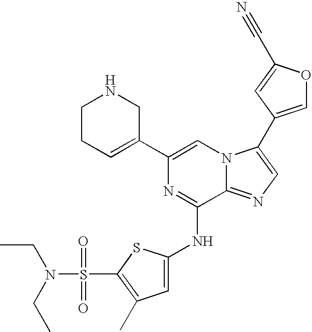 | 537.12 | 538.2 | 3.81 |

Assays:
Aurora Enzyme Assay

An in vitro assay was developed that utilizes recombinant Aurora A or Aurora B as an enzyme source and a peptide based on PKA as the substrate.

Aurora A Assay:

Aurora A kinase assays were performed in low protein binding 384-well plates (Corning Inc). All reagents were thawed on ice. Compounds were diluted in 100% DMSO to desirable concentrations. Each reaction consisted of 8 nM enzyme (Aurora A, Upstate cat#14-511), 100 nM Tamra-PKAtide (Molecular Devices, 5TAMRA-GRTGRRNSICOOH), 25 μM ATP (Roche), 1 mM DTT (Pierce), and kinase buffer (10 mM Tris, 10 mM MgCl2, 0.01% Tween 20). For each reaction, 14 μl containing TAMRA-PKAtide, ATP, DTT and kianse buffer were combined with 1 μl diluted compound. The kinase reaction was started by the addition of 5 μl diluted enzyme. The reaction was allowed to run for 2 hours at room temperature. The reaction was stopped by adding 60 μl IMAP beads (1:400 beads in progressive (94.7% buffer A: 5.3% buffer B) 1× buffer, 24 mM NaCl). After an additional 2 hours, fluorescent polarization was measured using an Analyst AD (Molecular devices).

Aurora B Assay:

Aurora A kinase assays were performed in low protein binding 384-well plates (Corning Inc). All reagents were thawed on ice. Compounds were diluted in 100% DMSO to desirable concentrations. Each reaction consisted of 26 nM enzyme (Aurora B, Invitrogen cat#pv3970), 100 nM Tamra-PKAtide (Molecular Devices, 5TAMRA-GRTGRRNSICOOH), 50 μM ATP (Roche), 1 mM DTT (Pierce), and kinase buffer (10 mM Tris, 10 mM MgCl2, 0.01% Tween 20). For each reaction, 14 μl containing TAMRA-PKAtide, ATP, DTT and kianse buffer were combined with 1 μl diluted compound. The kinase reaction was started by the addition of 5 μl diluted enzyme. The reaction was allowed to run for 2 hours at room temperature. The reaction was stopped by adding 60 μl IMAP beads (1:400 beads in progressive (94.7% buffer A: 5.3% buffer B) 1× buffer, 24 mM NaCl). After an additional 2 hours, fluorescent polarization was measured using an Analyst AD (Molecular devices).

IC₅₀ Determinations:

Dose-response curves were plotted from inhibition data generated each in duplicate, from 8 point serial dilutions of inhibitory compounds. Concentration of compound was plotted against kinase activity, calculated by degree of fluorescent polarization. To generate IC₅₀ values, the dose-response curves were then fitted to a standard sigmoidal curve and IC₅₀ values were derived by nonlinear regression analysis.

CHK1 SPA Assay

An in vitro assay was developed that utilizes recombinant His-CHK1 expressed in the baculovirus expression system as an enzyme source and a biotinylated peptide based on CDC25C as substrate (biotin-RSGLYRSPSMPENLNRPR).

Materials and Reagents:

1) CDC25C Ser 216 C-term Biotinylated peptide substrate (25 mg), stored at −20° C., Custom Synthesis by Research Genetics: biotin-RSGLYRSPSMPENLNRPR 2595.4 MW

2) His-CHK1 In House lot P976, 235 ug/mL, stored at −80° C.

3) D-PBS (without CaCl and MgCl): GIBCO, Cat.#14190-144

4) SPA beads: Amersham, Cat.#SPQ0032: 500 mg/vial

Add 10 mls of D-PBS to 500 mg of SPA beads to make a working concentration of 50 mg/ml. Store at 4° C. Use within 2 week after hydration.

5) 96-Well White Microplate with Bonded GF/B filter: Packard, Cat.#6005177

6) Top seal-A 96 well Adhesive Film: Perkin Elmer, Cat.#6005185

7) 96-well Non-Binding White Polystyrene Plate: Corning, Cat. #6005177

8) MgCl₂: Sigma, Cat.#M-8266

9) DTT: Promega, Cat.#V3155

10) ATP, stored at 4° C.: Sigma, Cat.#A-5394

11) γ³³P-ATP, 1000-3000 Ci/mMol: Amersham, Cat.#AH9968

12) NaCl: Fisher Scientific, Cat.#BP358-212

13) H₃PO₄ 85% Fisher, Cat.#A242-500

14) Tris-HCL pH 8.0: Bio-Whittaker, Cat. #16-015V

15) Staurosporine, 100 ug: CALBIOCHEM, Cat. #569397

16) Hypure Cell Culture Grade Water, 500 mL: HyClone, Cat.#SH30529.02

Reaction Mixtures:

1) Kinase Buffer: 50 mM Tris pH 8.0; 10 mM MgCl₂; 1 mM DTT

2) His-CHK1, In House Lot P976, MW ˜30 KDa, stored at −80° C. 6 nM is required to yield positive controls of ˜5,000 CPM. For 1 plate (100 rxn): dilute 8 μL of 235 μg/mL (7.83 uM) stock in 2 mL Kinase Buffer. This makes a 31 nM mixture. Add 20 μL/well. This makes a final reaction concentration of 6 nM.

3) CDC25C Biotinylated Peptide.

Dilute CDC25C to 1 mg/mL (385 uM) stock and store at −20° C. For 1 plate (100 rxn): dilute 10 μL of 1 mg/mL peptide stock in 2 ml Kinase Buffer. This gives a 1.925 μM mix. Add 20 μL/rxn. This makes a final reaction concentration of 385 nM.

4) ATP Mix.

For 1 plate (100 rxn): dilute 10 μL of 1 mM ATP (cold) stock and 2 uL fresh P33-ATP (20 μCi) in 5 ml Kinase Buffer. This gives a 2 μM ATP (cold) solution; add 50 μl/well to start the reaction. Final volume is 100 μl/rxn so the final reaction concentrations will be 1 μM ATP (cold) and 0.2 uCi/rxn.

5) Stop Solution:

For 1 plate add: To 10 mL Wash Buffer 2 (2M NaCl 1% H₃PO₄): 1 mL SPA bead slurry (50 mg); Add 100 μL/well

6) Wash buffer 1: 2 M NaCl

7) Wash buffer 2: 2 M NaCl, 1% H₃PO₄

Assay Procedure:

| Assay | Final | | | |
|---|---|---|---|---|
| Component | Concentration | | Volume | |
| CHK1 | 6 | nM | 20 | μl/rxn |
| Compound | — | | 10 | μl/rxn |
| (10% DMSO) | | | | |
| CDC25C | 0.385 | μM | 20 | μl/rxn |
| γ³³P-ATP | 0.2 | μCi/rxn | 50 | μl/rxn |
| Cold ATP | 1 | μM | | |
| Stop solution | 0.5 | mg/rxn | 100 | μl/rxn* |
| SPA beads | | | | |
| | | | 200 | μl/rxn** |

*Total reaction volume for assay.
**Final reaction volume at termination of reaction (after addition of stop solution).

1) Dilute compounds to desired concentrations in water/10% DMSO—this will give a final DMSO concentration of 1% in the rxn. Dispense 10 μl/rxn to appropriate wells. Add 10 μL 10% DMSO to positive (CHK1+CDC25C+ATP) and negative (CHK1+ATP only) control wells.
2) Thaw enzyme on ice—dilute enzyme to proper concentration in kinase buffer (see Reaction Mixtures) and dispense 20 μl to each well.
3) Thaw the Biotinylated substrate on ice and dilute in kinase buffer (see Reaction Mixtures). Add 20 μL/well except to negative control wells. Instead, add 20 uL Kinase Buffer to these wells.
4) Dilute ATP (cold) and P33-ATP in kinase buffer (see Reaction Mixtures). Add 50 μL/well to start the reaction.
5) Allow the reaction to run for 2 hours at room temperature.
6) Stop reaction by adding 100 uL of the SPA beads/stop solution (see Reaction Mixtures) and leave to incubate for 15 minutes before harvest
7) Place a blank Packard GF/B filter plate into the vacuum filter device (Packard plate harvester) and aspirate 200 mL water through to wet the system.
8) Take out the blank and put in the Packard GF/B filter plate.
9) Aspirate the reaction through the filter plate.
10) Wash: 200 ml each wash; 1× with 2M NaCl; 1× with 2M NaCl/1% H₃PO₄
11) Allow filter plate to dry 15 min.
12) Put TopSeal-A adhesive on top of filter plate.
13) Run filter plate in Top Count

Settings:
Data mode: CPM
Radio nuclide: Manual SPA:P33
Scintillator: Liq/plast
Energy Range: Low
   IC₅₀ DETERMINATIONS: Dose-response curves were plotted from inhibition data generated, each in duplicate, from 8 point serial dilutions of inhibitory compounds. Concentration of compound was plotted against % kinase activity, calculated by CPM of treated samples divided by CPM of untreated samples. To generate IC₅₀ values, the dose-response curves were then fitted to a standard sigmoidal curve and IC₅₀ values were derived by nonlinear regression analysis. IC₅₀ values for the compounds of the present invention determined according to the above method are set forth in Table 43 below. As demonstrated above by the assay values, compounds of Table A of the present invention exhibit good Chk1 inhibitory properties.
   CDK2 Assay:
   BACULOVIRUS CONSTRUCTIONS: Cyclin E was cloned into pVL1393 (Pharmingen, La Jolla, Calif.) by PCR, with the addition of 5 histidine residues at the amino-terminal end to allow purification on nickel resin. The expressed protein was approximately 45 kDa. CDK2 was cloned into pVL1393 by PCR, with the addition of a haemaglutinin epitope tag at the carboxy-terminal end (YDVPDYAS). The expressed protein was approximately 34 kDa in size.
   ENZYME PRODUCTION: Recombinant baculoviruses expressing cyclin E and CDK2 were co-infected into SF9 cells at an equal multiplicity of infection (MOI=5), for 48 hrs. Cells were harvested by centrifugation at 1000 RPM for 10 minutes, then pellets lysed on ice for 30 minutes in five times the pellet volume of lysis buffer containing 50 mM Tris pH 8.0, 150 mM NaCl, 1% NP40, 1 mM DTT and protease inhibitors (Roche Diagnostics GmbH, Mannheim, Germany). Lysates were spun down at 15000 RPM for 10 minutes and the supernatant retained. 5 ml of nickel beads (for one liter of SF9 cells) were washed three times in lysis buffer (Qiagen GmbH, Germany). Imidazole was added to the baculovirus supernatant to a final concentration of 20 mM, then incubated with the nickel beads for 45 minutes at 4° C. Proteins were eluted with lysis buffer containing 250 mM imidazole. Eluate was dialyzed overnight in 2 liters of kinase buffer containing 50 mM Tris pH 8.0, 1 mM DTT, 10 mM MgCl2, 100 uM sodium orthovanadate and 20% glycerol. Enzyme was stored in aliquots at −70° C.
   IN VITRO KINASE ASSAY: Cyclin E/CDK2 kinase assays were performed in low protein binding 96-well plates (Corning Inc, Corning, N.Y.). Enzyme was diluted to a final concentration of 50 μg/ml in kinase buffer containing 50 mM Tris pH 8.0, 10 mM MgCl₂, mM DTT, and 0.1 mM sodium orthovanadate. The substrate used in these reactions was a biotinylated peptide derived from Histone H1 (from Amersham, UK). The substrate was thawed on ice and diluted to 2 μM in kinase buffer. Compounds were diluted in 10% DMSO to desirable concentrations. For each kinase reaction, 20 μl of the 50 μg/ml enzyme solution (1 μg of enzyme) and 20 μl of the 2 μM substrate solution were mixed, then combined with 10 μl of diluted compound in each well for testing. The kinase reaction was started by addition of 50 μl of 2 μM ATP and 0.1 μCi of 33P-ATP (from Amersham, UK). The reaction was allowed to run for 1 hour at room temperature. The reaction was stopped by adding 200 μl of stop buffer containing 0.1% Triton X-100, 1 mM ATP, 5 mM EDTA, and 5 mg/ml streptavidine coated SPA beads (from Amersham, UK) for 15 minutes. The SPA beads were then captured onto a 96-well GF/B filter plate (Packard/Perkin Elmer Life Sciences) using a Filtermate universal harvester (Packard/Perkin Elmer Life Sciences.). Non-specific signals were eliminated by washing the beads twice with 2M NaCl then twice with 2 M NaCl with 1% phosphoric acid. The radioactive signal was then measured using a TopCount 96 well liquid scintillation counter (from Packard/Perkin Elmer Life Sciences).

IC₅₀ DETERMINATIONS: Dose-response curves were plotted from inhibition data generated, each in duplicate, from 8 point serial dilutions of inhibitory compounds. Concentration of compound was plotted against % kinase activity, calculated by CPM of treated samples divided by CPM of untreated samples. To generate IC₅₀ values, the dose-response curves were then fitted to a standard sigmoidal curve and IC₅₀ values were derived by nonlinear regression analysis. Table 43 shows the activity data for an illustrative list of compounds of the invention.

TABLE 43

| | CDK2 | CHK- |
|---|---|---|
| | IC 50 = | 1IC50 = |
| Structure | nM | nM |
| 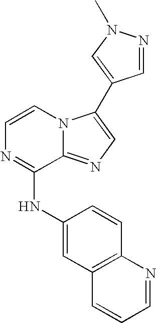 | 50000 | 492 |
| 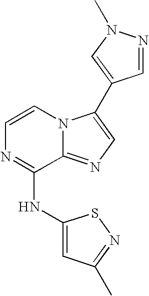 | 50000 | 54 |
| 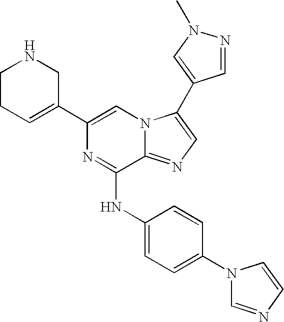 | 12516 | 97 |
| 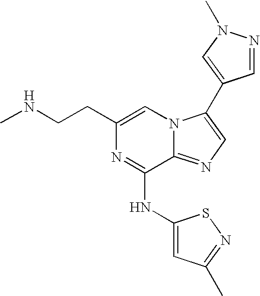 | 11374 | 13 |
| 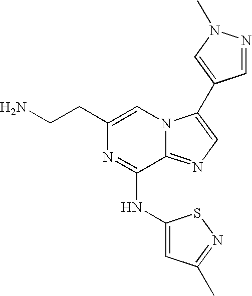 | 5942 | 7 |
| 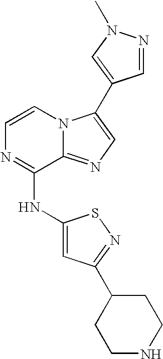 | 18100 | 31 |
| 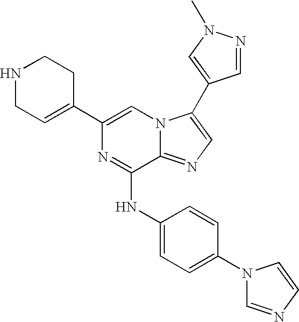 | 19382 | 181 |
| 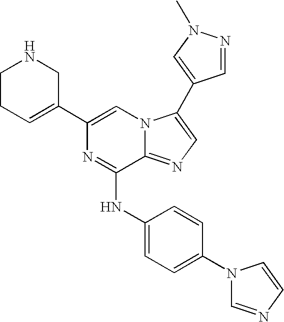 | 12516 | 97 |
| 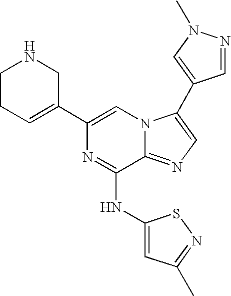 | 10966 | 14 |
| 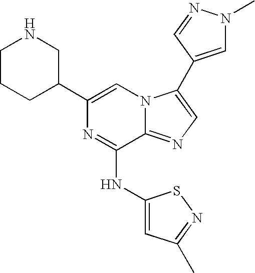 | 1100 | 13 |
| 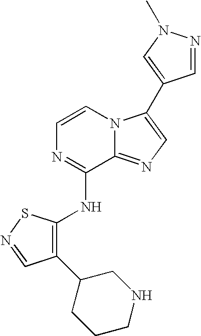 | 21818 | 18 |
| 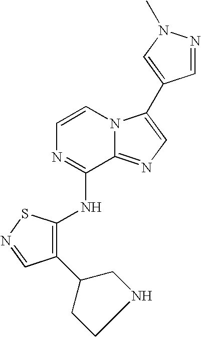 | 50000 | 23 |
| 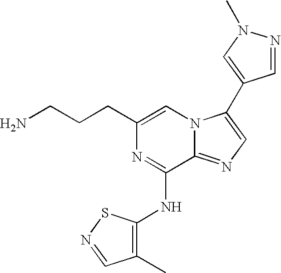 | 1910 | 5 |
| 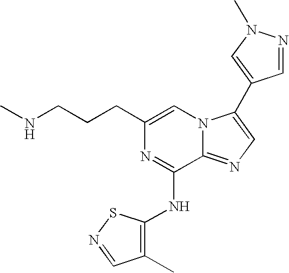 | 5773 | 8 |
| 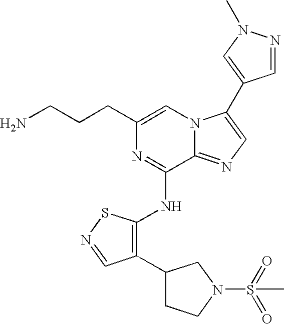 | — | 8 |
| 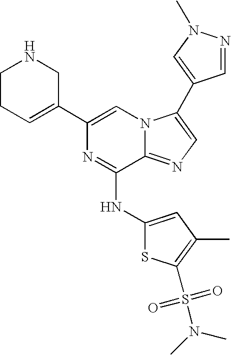 | 5198 | 7 |
| 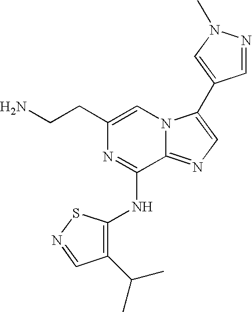 | 13 | 6380 |
| 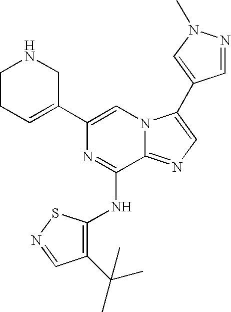 | 13731 | 16 |
| 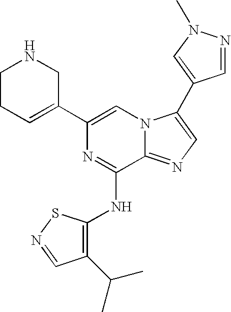 | 4209 | 6 |
| 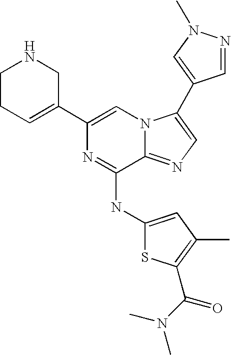 | 24086 | 16 |
| 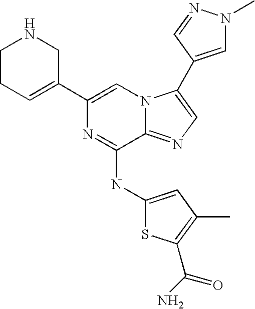 | 16230 | 23 |
| 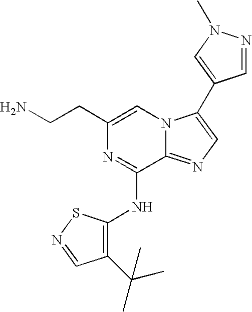 | 14053 | 11 |
| 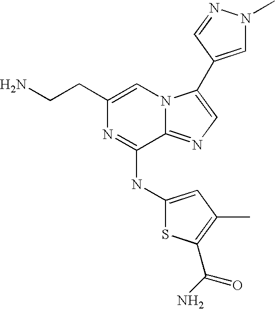 | 17945 | 19 |
| 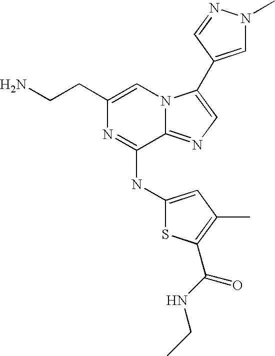 | 41297 | 15 |
| 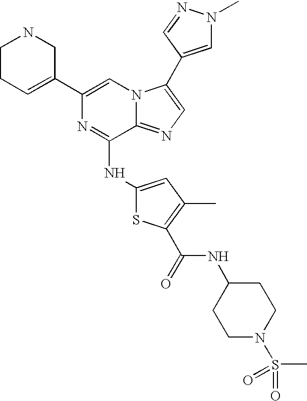 | 40995 | 24 |
| 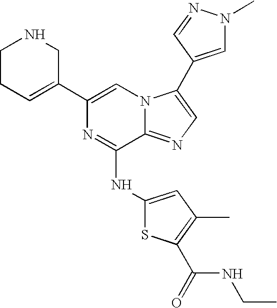 | 50000 | 15 |
| 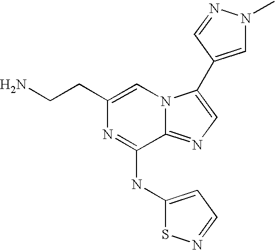 | 550 | 17 |
| 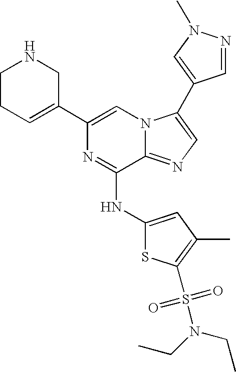 | 8283 | 19 |
| 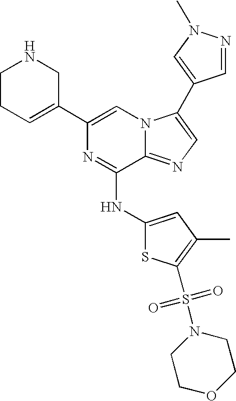 | 6949 | 7 |
| 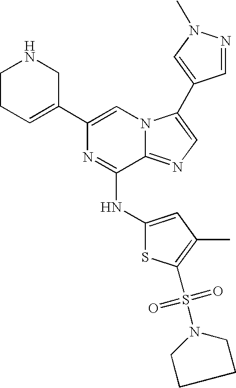 | 5173 | 6 |
| 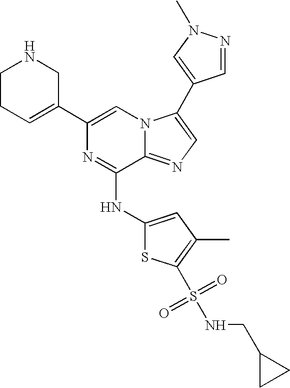 | 2144 | 8 |
| 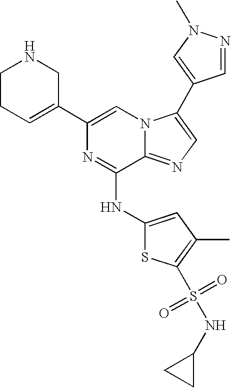 | 1577 | 3 |
| 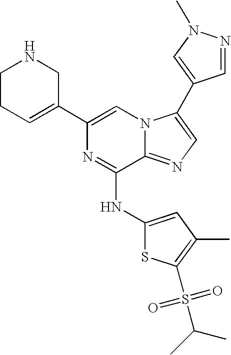 | 4792 | 5 |
| 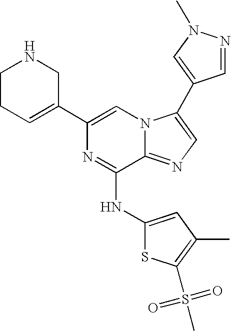 | 11618 | 8 |
| 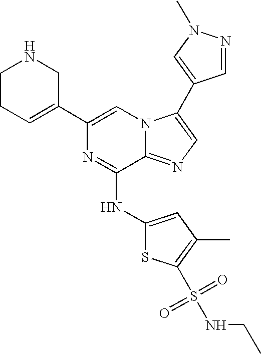 | — | 10 |
| 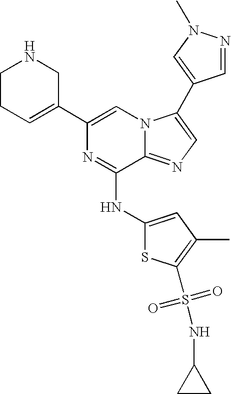 | 3214 | 7 |
| 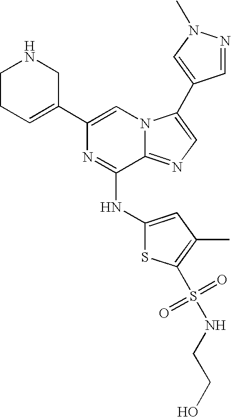 | 4681 | 6 |
| 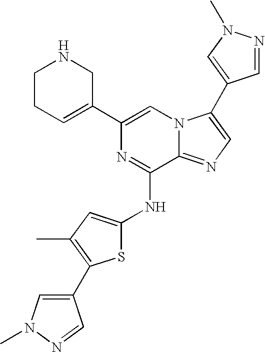 | 4586 | 19 |
| 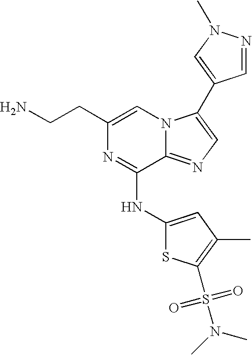 | — | 14 |
| 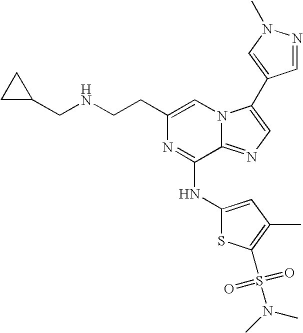 | — | 10 |
| 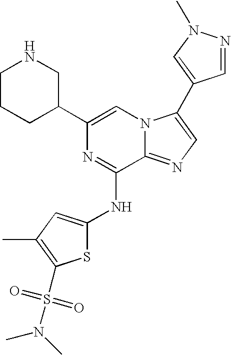 | — | 12 |
| 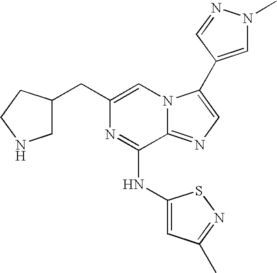 | 607 | 7 |
| 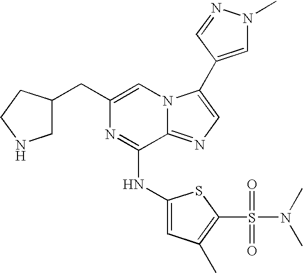 | 983 | 8 |
| 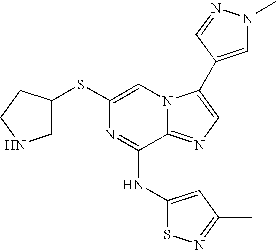 | — | 19 |
| 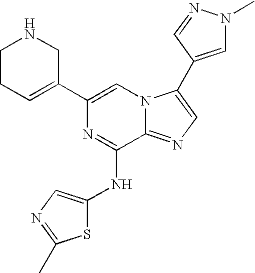 | 4626 | 8 |
| 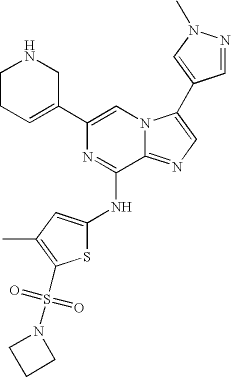 | — | 12 |
| 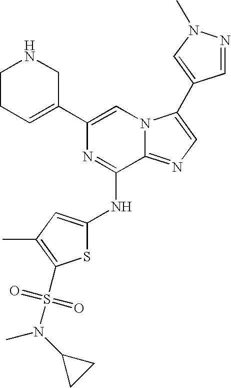 | 13088 | 18 |

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A compound of Formula I: 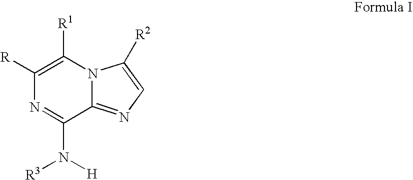
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₃ NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁶ (wherein the R⁵ and R⁶, together with the N of said —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
said alkyl shown above for R³ can be unsubstituted or substituted with one or more moieties which can be the same or different each moiety being independently selected from the group consisting of —OR⁵, alkoxy, heteroaryl, and —NR⁵R⁶;
said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and —S(O₂)R⁵; and
said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
further wherein in any —NR⁵R⁶ in Formula I, said R⁵ and R⁶ can optionally be joined together with the N of said —NR⁵R⁶ to form a cyclic ring.

2. A compound of the formula: 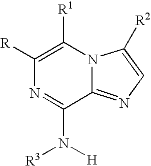
or a pharmaceutically acceptable salt, solvate, ester or prodrug thereof, wherein:
R is H, CN, —NR⁵R⁶, cycloalkenyl, heterocyclenyl, —C(O)NR⁵R⁶, —N(R⁵)C(O)R⁶, or alkyl substituted with one or more moieties which can be the same or different each moiety being independently selected from the group consisting of —OR⁵ and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is H, halo, or heteroaryl, wherein said heteroaryl can be unsubstituted or substituted with one or more moieties which can be the same or different each moiety being independently selected from the group consisting of halo, alkyl, alkenyl, alkynyl, cycloalkyl, aryl, heteroaryl and heterocyclyl;
R³ is H, alkyl, aryl or heteroaryl, wherein:
said alkyl can be unsubstituted or substituted with one or more moieties which can be the same or different each moiety being independently selected from the group consisting of —OR⁵, alkoxy and —NR⁵R⁶;
said aryl is substituted with heteroaryl which heteroaryl can be unsubstituted or substituted with alkyl; and
said heteroaryl shown above for R³ can be unsubstituted or substituted with one or more moieties which can be the same or different with each moiety being independently selected from the group consisting of halo, —OR⁵, alkyl, alkenyl, alkynyl, cycloalkyl, aryl and heterocyclyl;
R⁵ is H, alkyl, aryl, heteroaryl, heterocyclyl or cycloalkyl; and
R⁶ is H, alkyl, aryl, heteroaryl, heterocyclyl or cycloalkyl.

3. The compound of claim 1, wherein R² is unsubstituted heteroaryl or heteroaryl substituted with alkyl.

4. The compound of claim 1, wherein R² is heteroaryl substituted with alkyl.

5. The compound of claim 1, wherein R² is pyrazolyl.

6. The compound of claim 1, wherein R² is pyrazolyl substituted with alkyl.

7. The compound of claim 1, wherein R² is 1-methyl-pyrazol-4-yl.

8. The compound of claim 1, wherein R is H.

9. The compound of claim 1, wherein R is CN.

10. The compound of claim 1, wherein R is —C(O)NR5R⁶.

11. The compound of claim 1, wherein R is —C(O)NH₂.

12. The compound of claim 1, wherein R is heterocyclenyl.

13. The compound of claim 1, wherein R is tetrahydropyridinyl.

14. The compound of claim 1, wherein R is 1,2,3,6-tetrahydropyridinyl.

15. The compound of claim 1, wherein R is alkyl substituted with one or more moieties which can be the same or different each moiety being independently selected from the group consisting of —OR¹ and —NR⁵R⁶.

16. The compound of claim 1, wherein R is alkyl substituted with one or more —NR⁵R⁶.

17. The compound of claim 1, wherein R is alkyl substituted with —NH₂.

18. The compound of claim 1, wherein R is alkyl substituted with —NH(methyl).

19. The compound of claim 1, wherein R³ is unsubstituted alkyl.

20. The compound of claim 1, wherein R³ is alkyl substituted with one or more moieties which can be the same or different, each moiety being independently selected from the group consisting of halo, —OR¹, alkoxy and —NR⁵R⁶.

21. The compound of claim 1, wherein R³ is unsubstituted heteroaryl.

22. The compound of claim 1, wherein R³ is heteroaryl substituted with alkyl.

23. The compound of claim 1, wherein R³ is heteroaryl substituted with methyl.

24. The compound of claim 1, wherein R³ is unsubstituted isothiazolyl.

25. The compound of claim 1, wherein R³ is isothiazolyl substituted with alkyl.

26. The compound of claim 1, wherein R³ is isothiazolyl substituted with methyl.

27. The compound of claim 1, wherein R³ is 5-methyl-isothiazol-3-yl.

28. The compound of claim 1, wherein R³ is aryl substituted with heteroaryl.

29. The compound of claim 1, wherein R³ is aryl substituted with imidazolyl.

30. The compound of claim 1, wherein R³ is phenyl substituted with imidazolyl.

31. A compound of the formula: 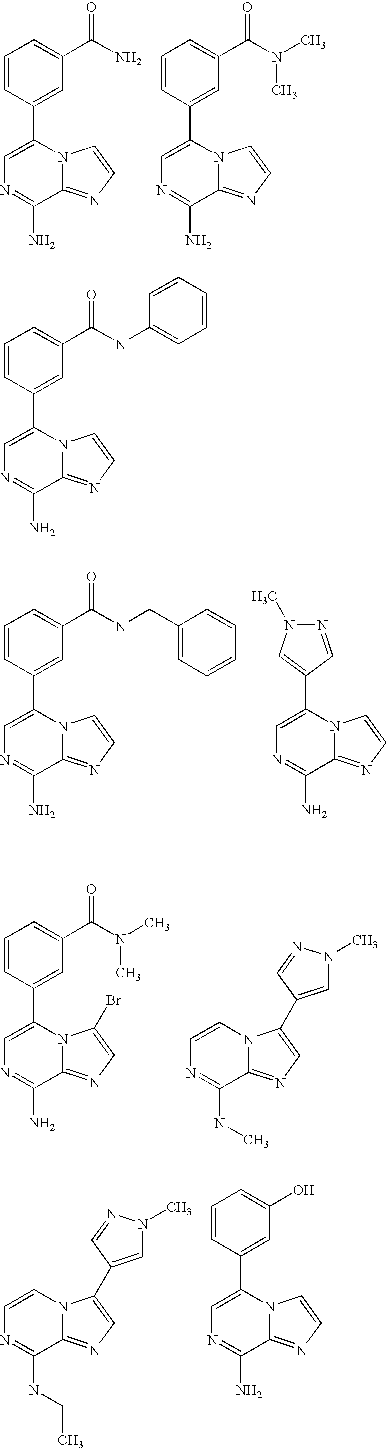 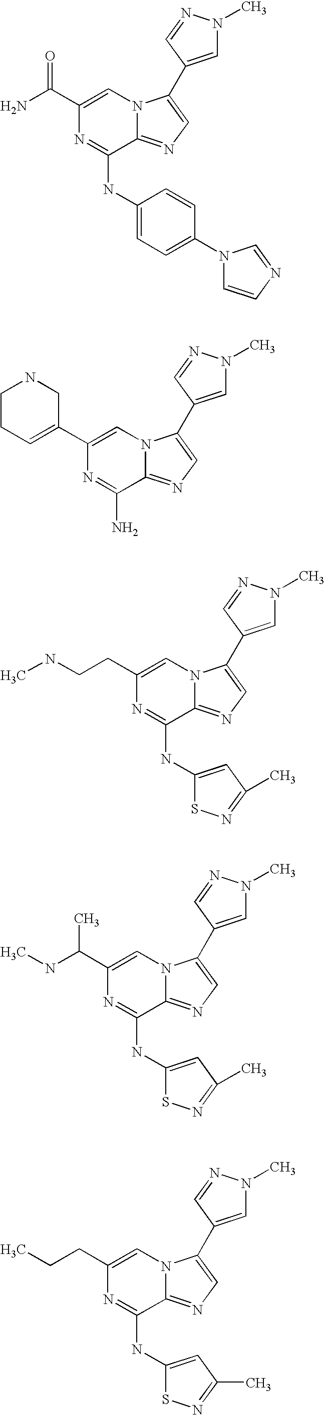 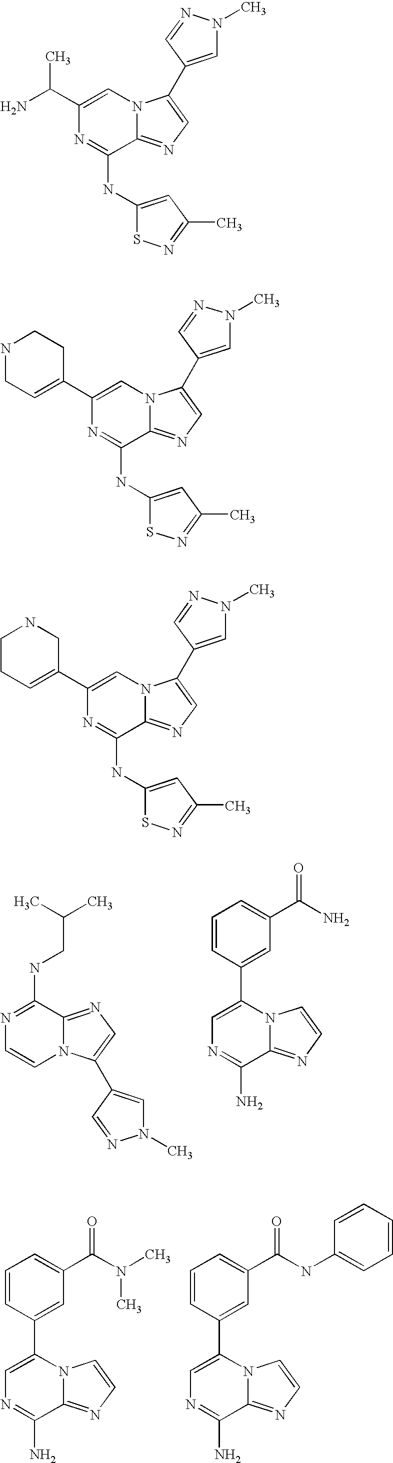 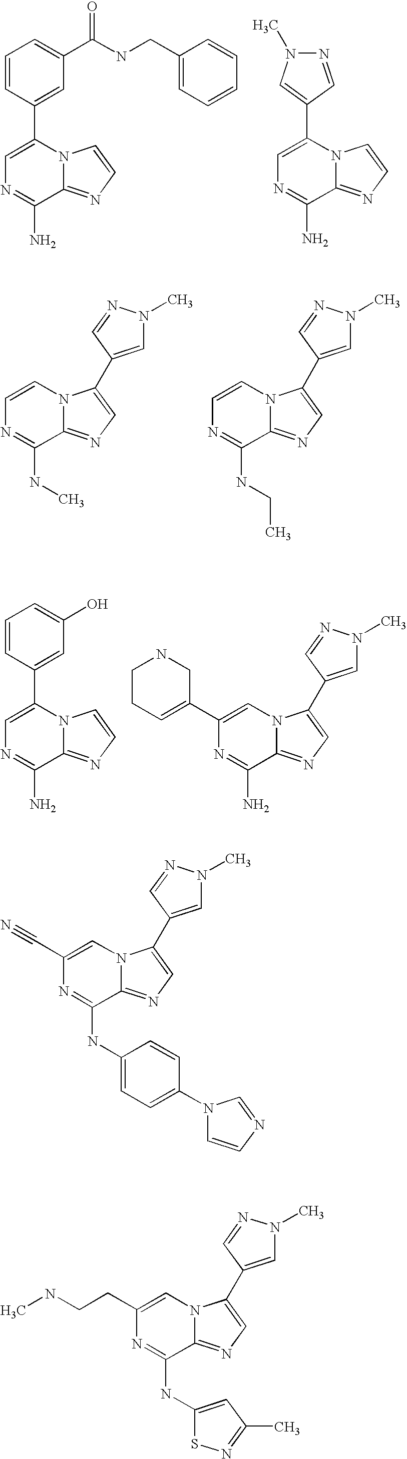 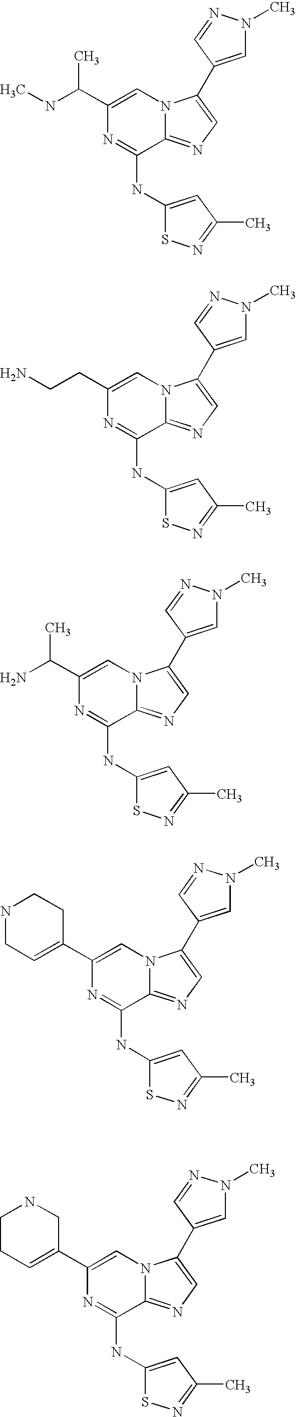 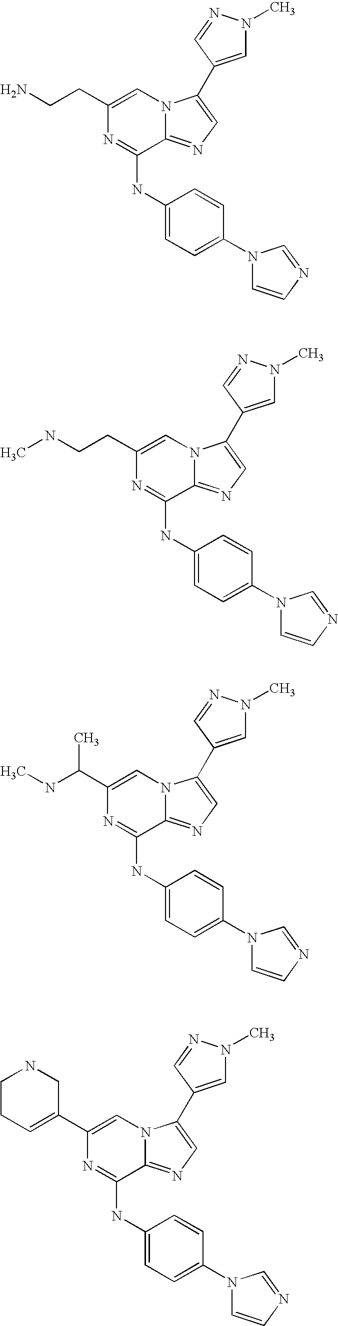 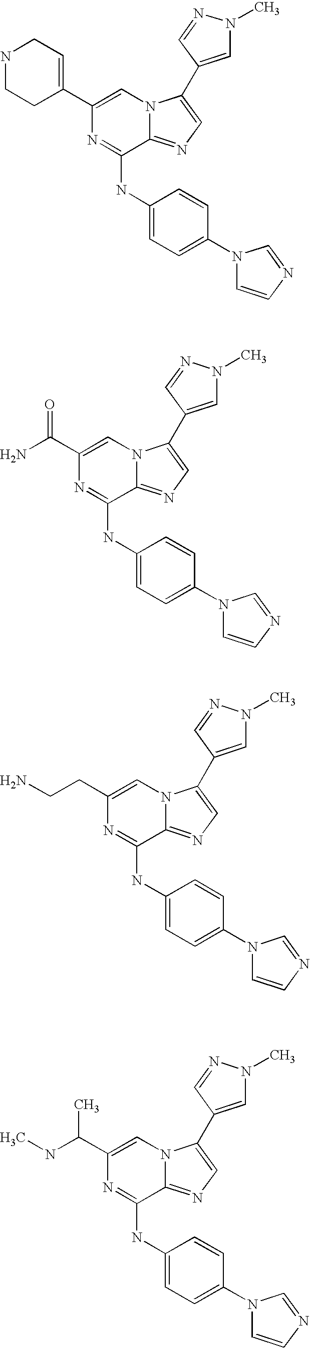 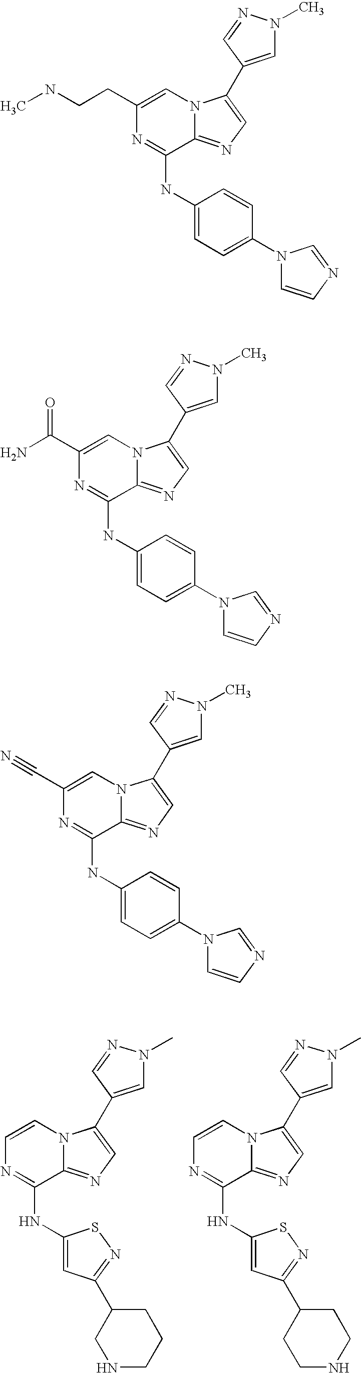 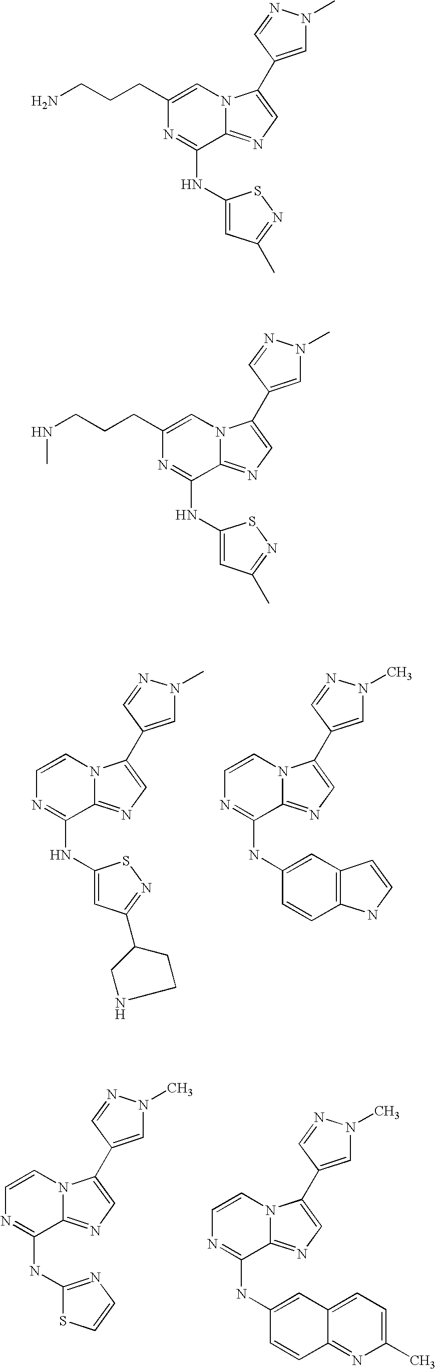 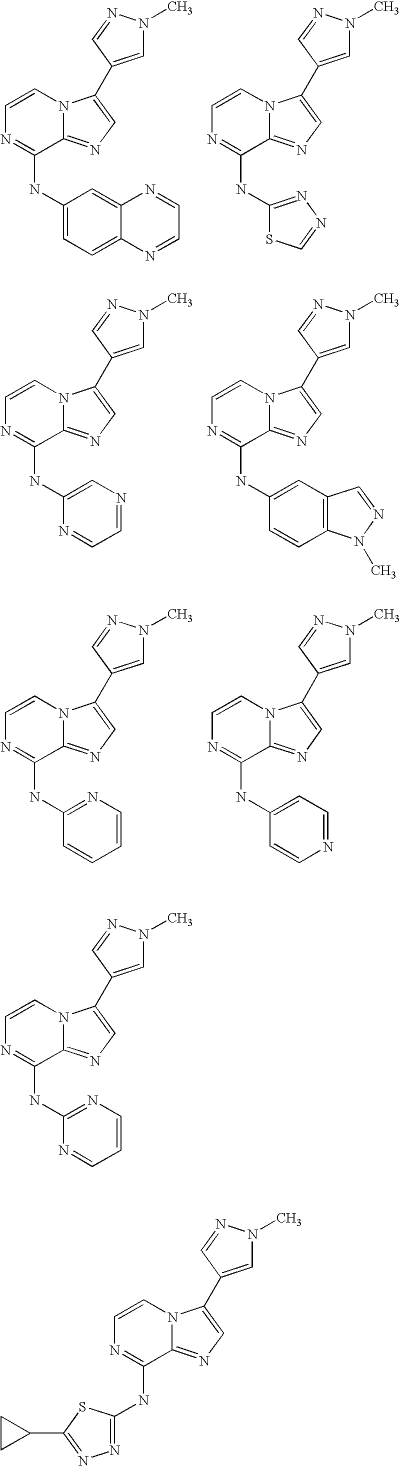 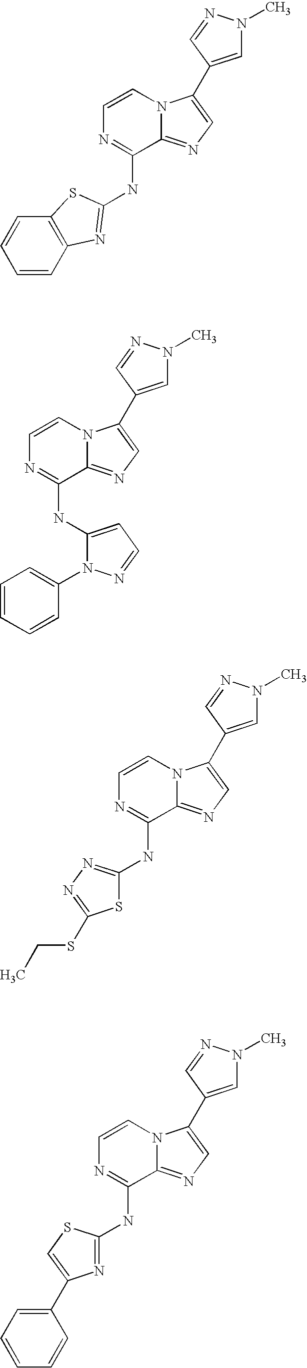 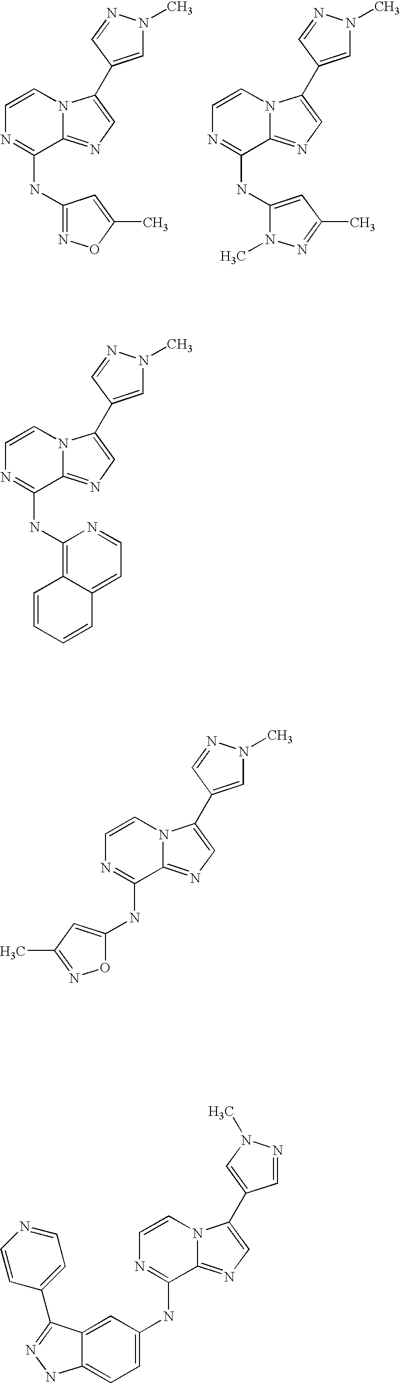 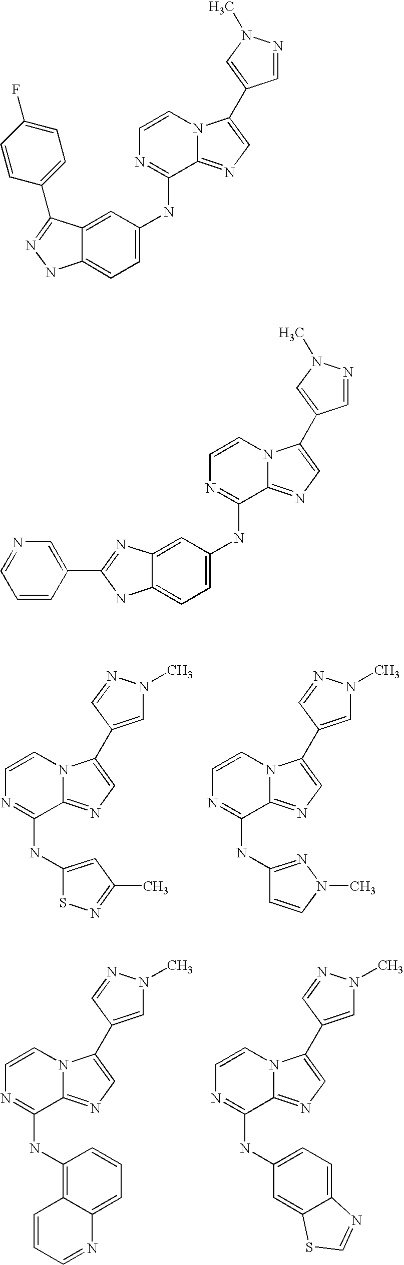 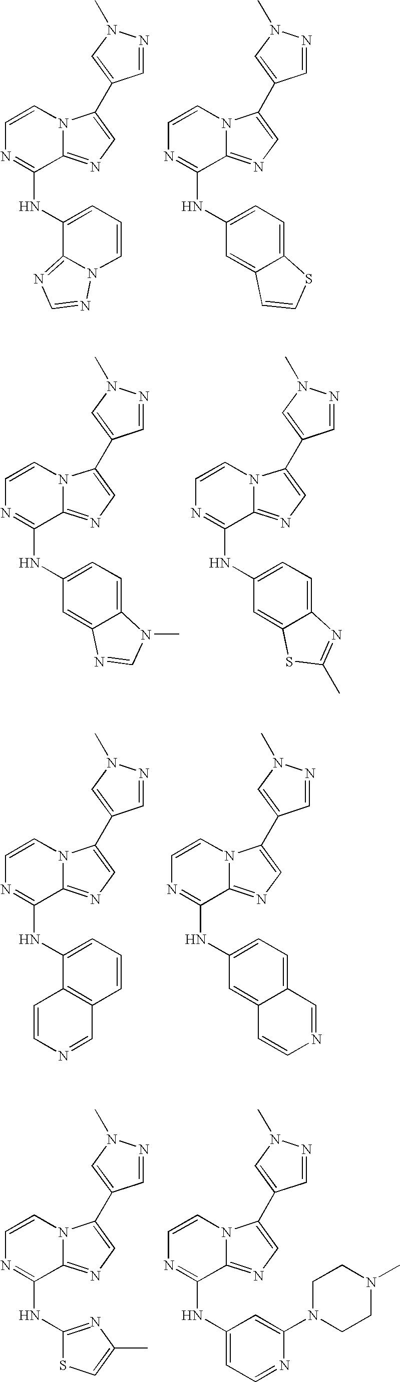 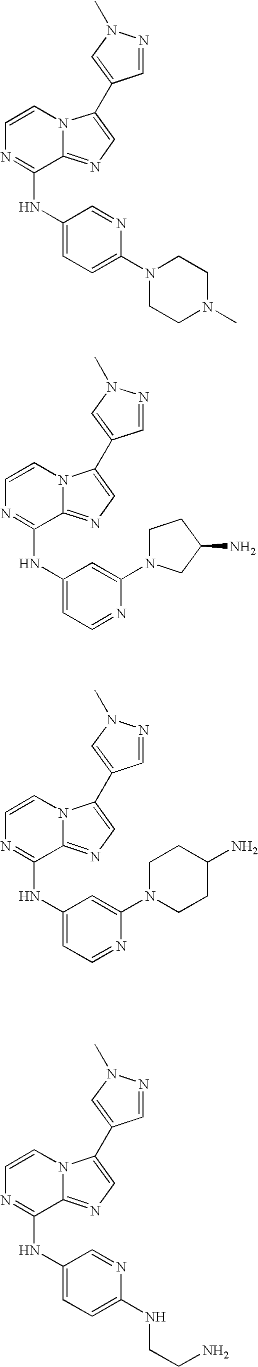 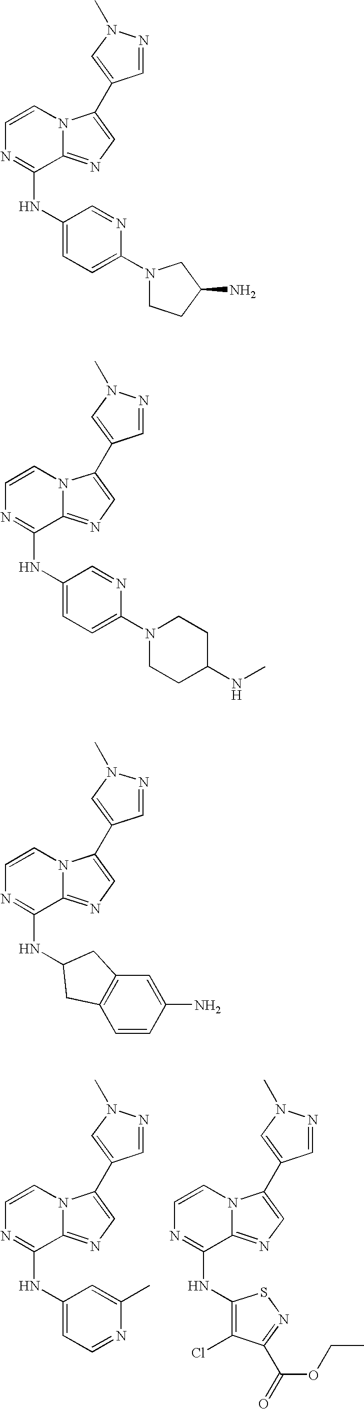 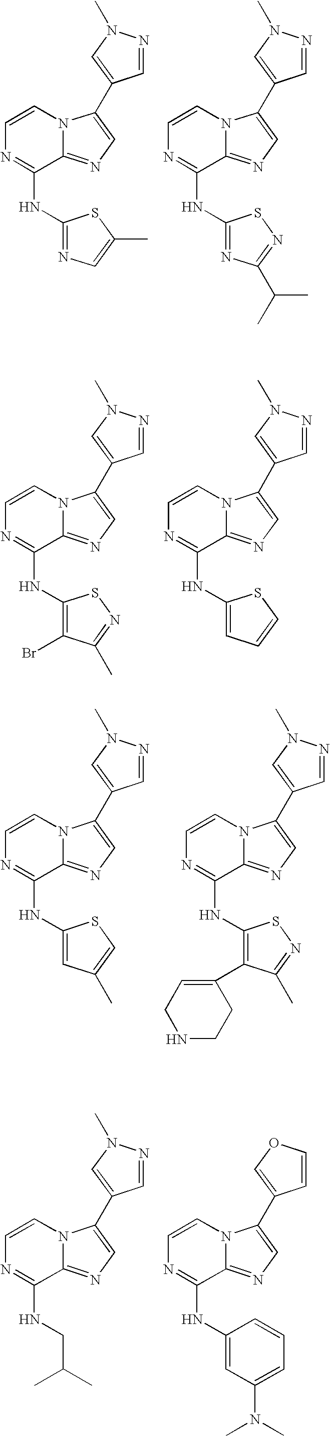 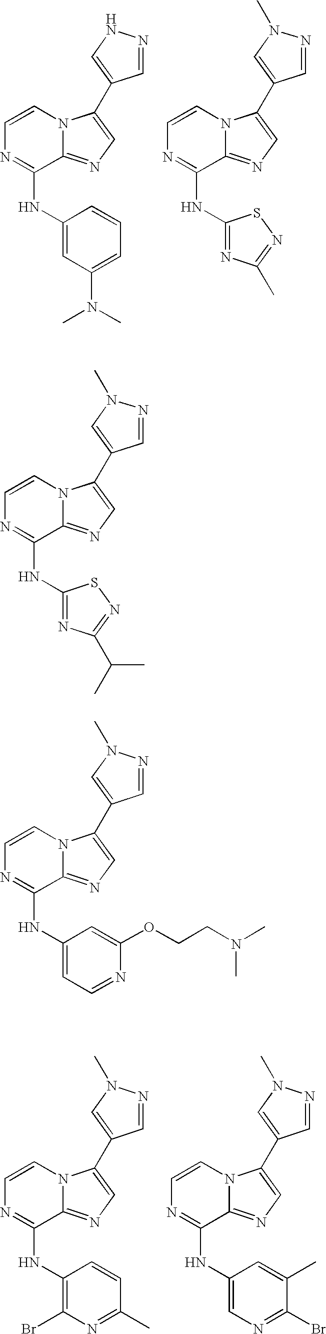 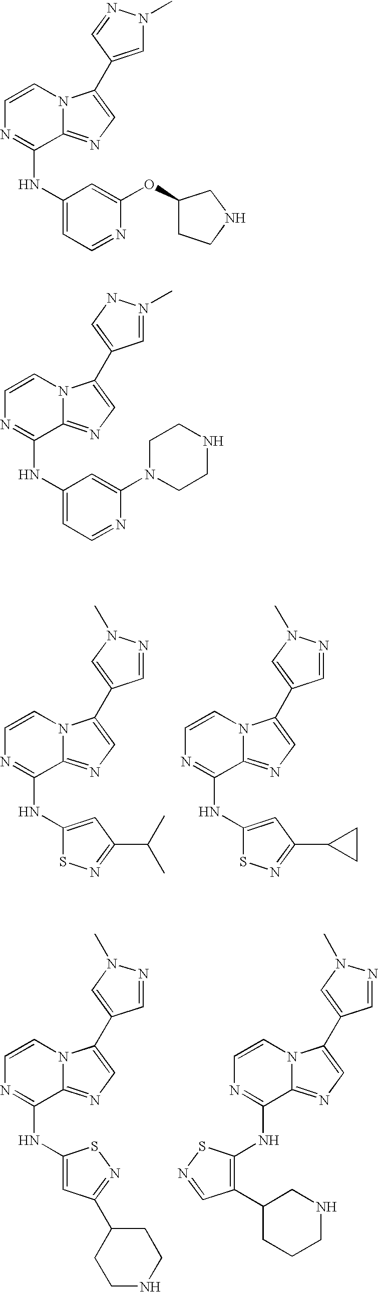 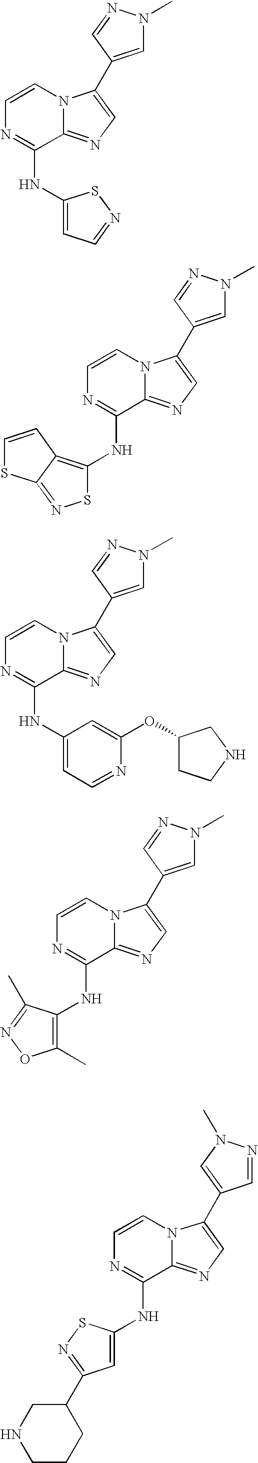 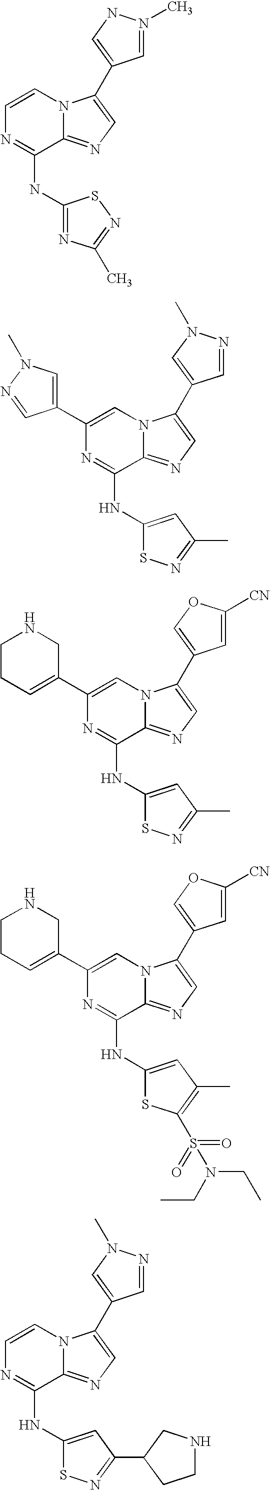 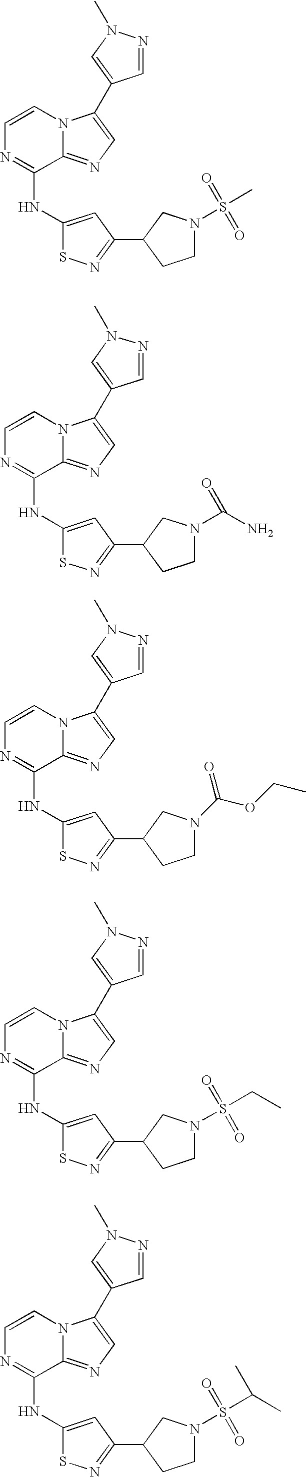 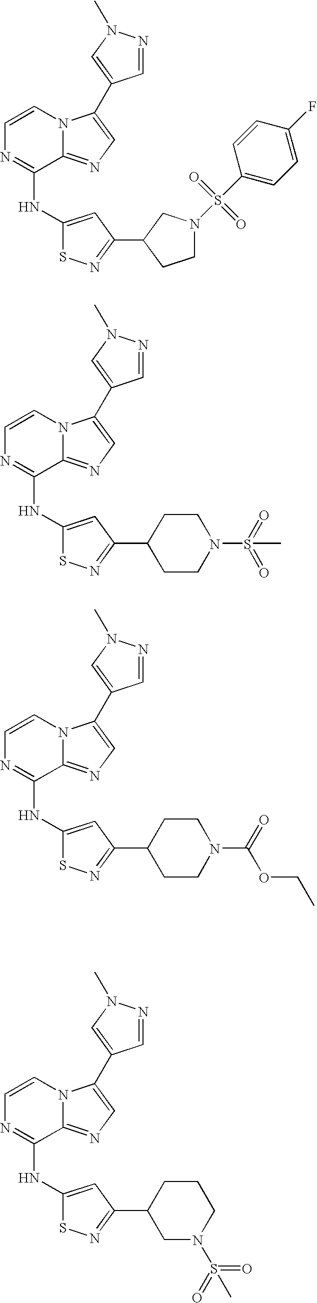 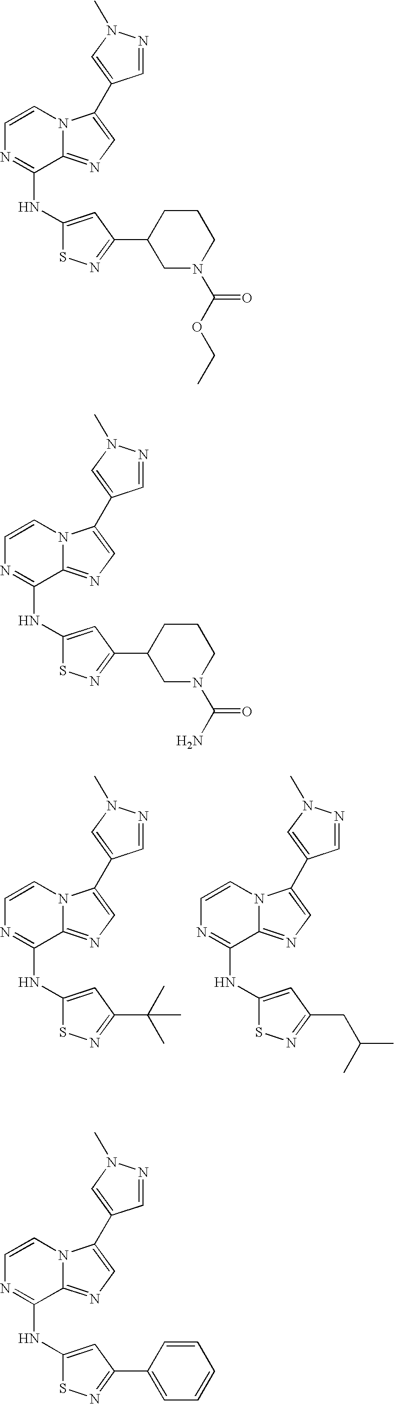 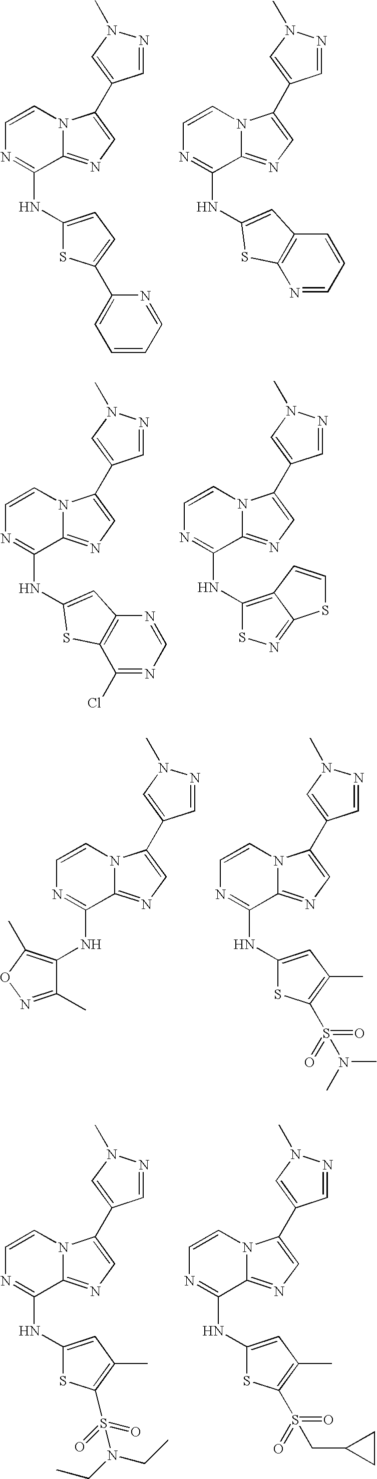 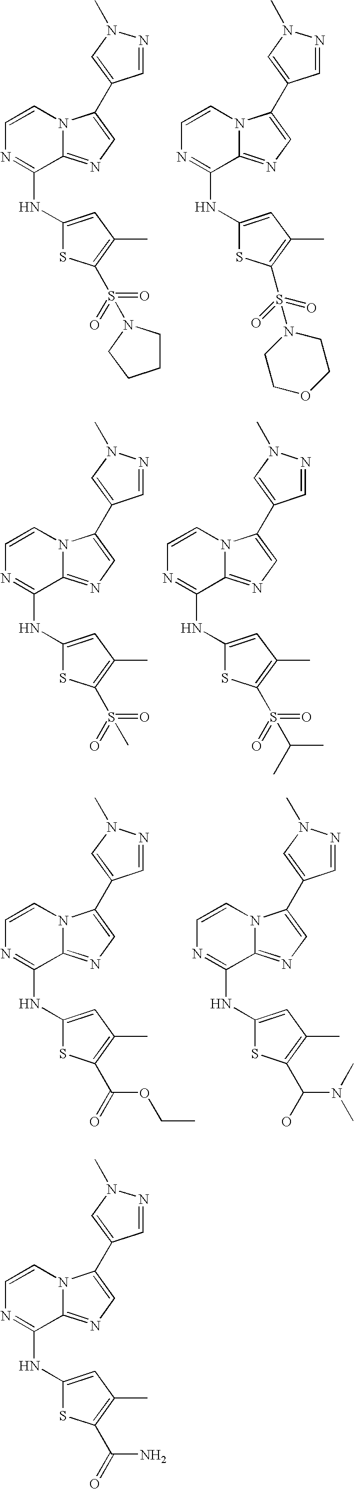 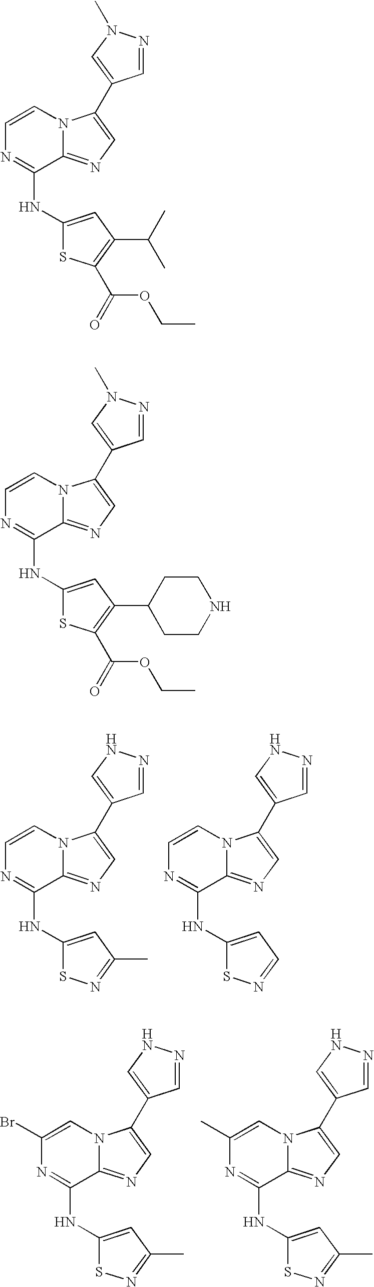 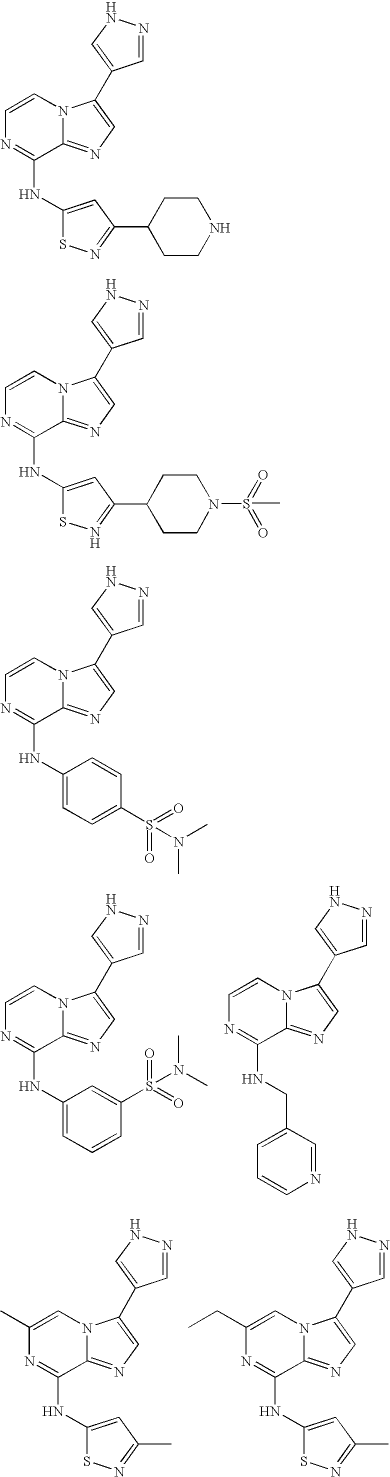 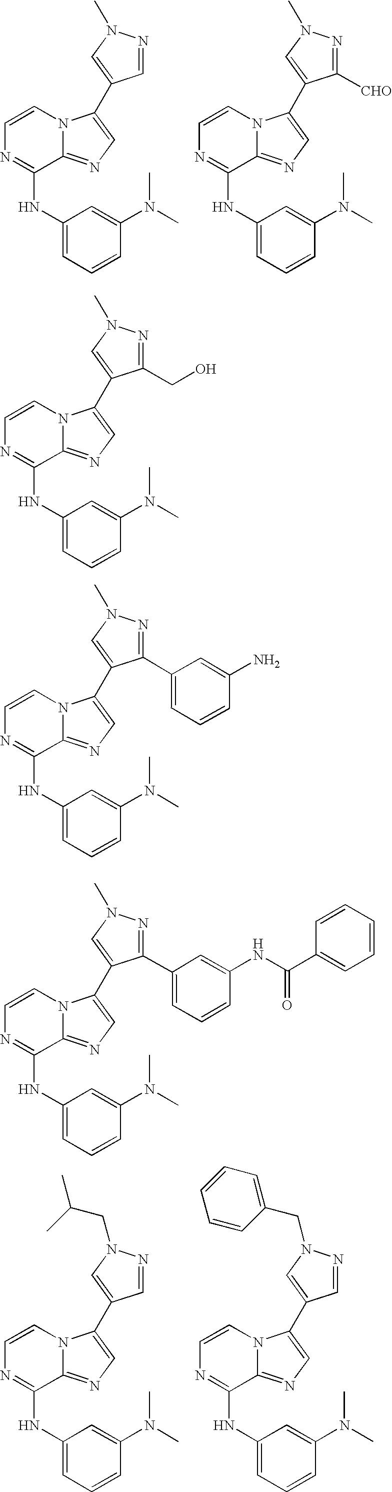 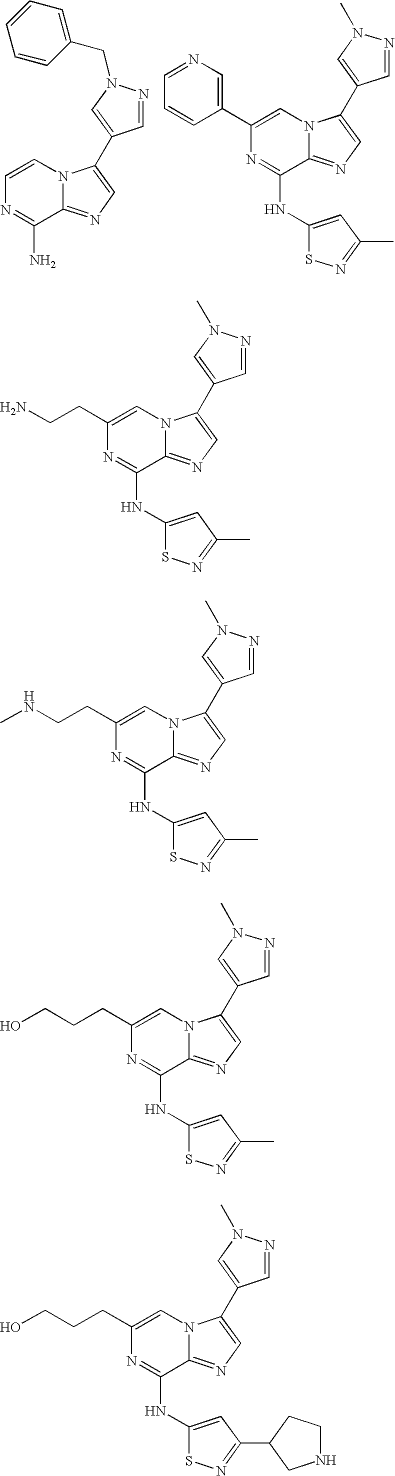 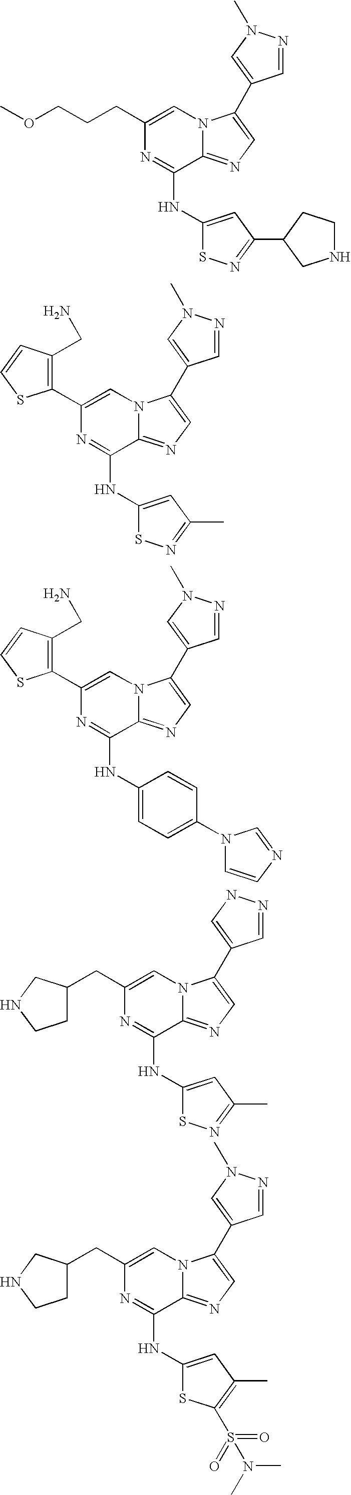 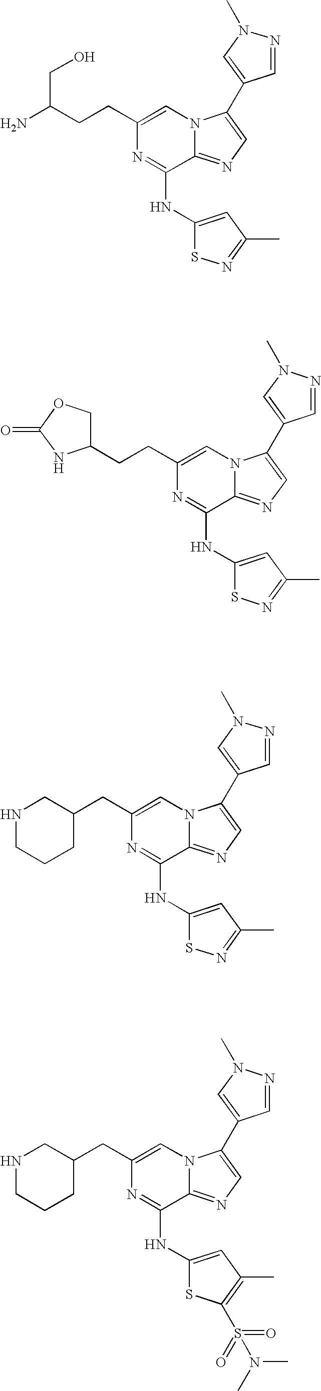 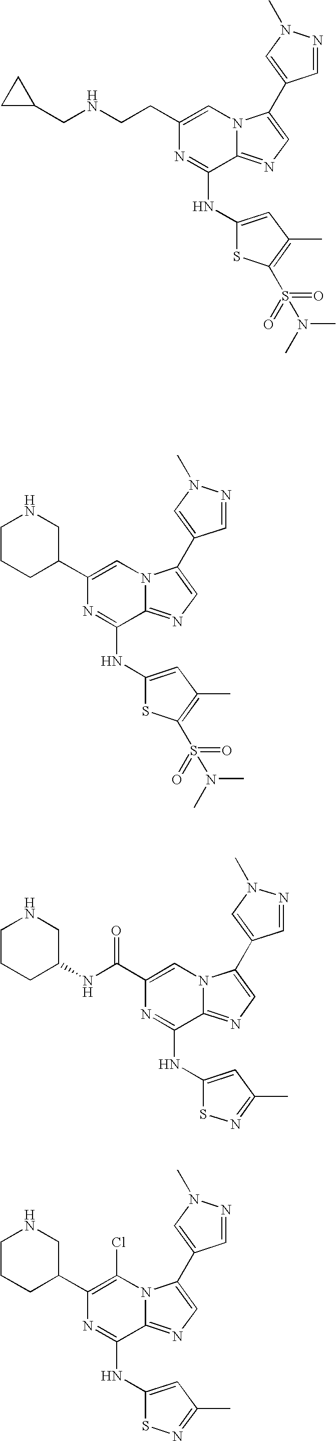 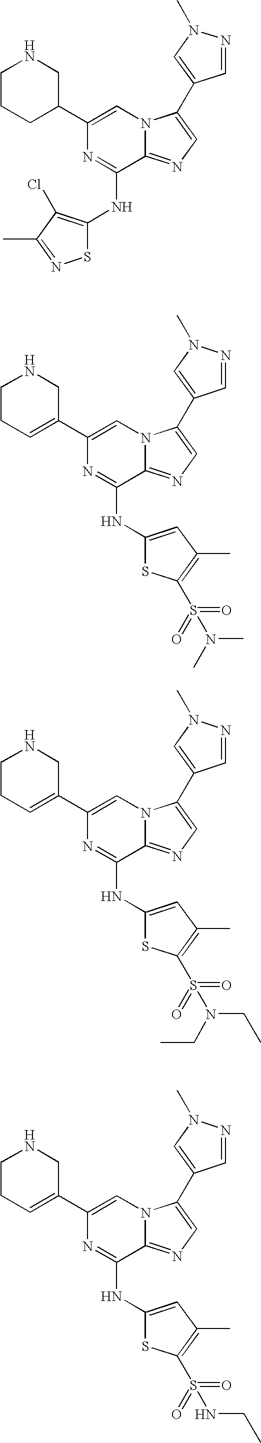 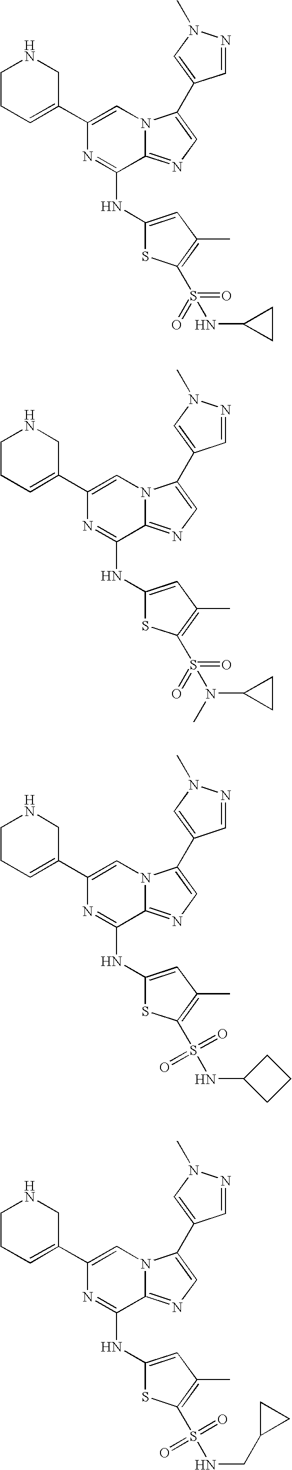 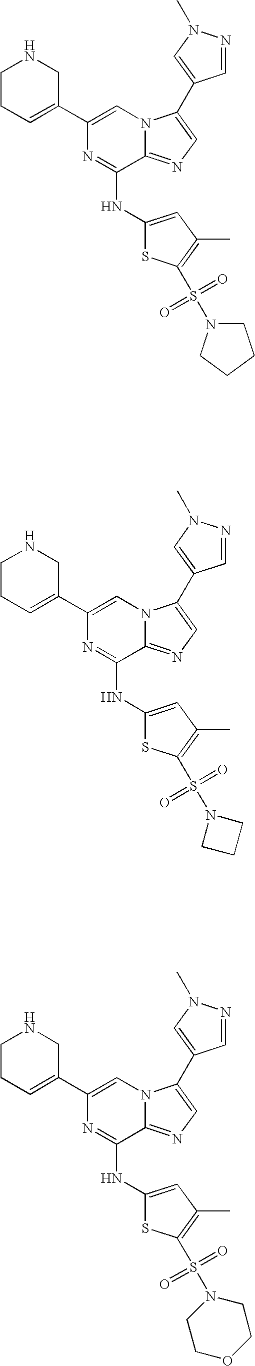 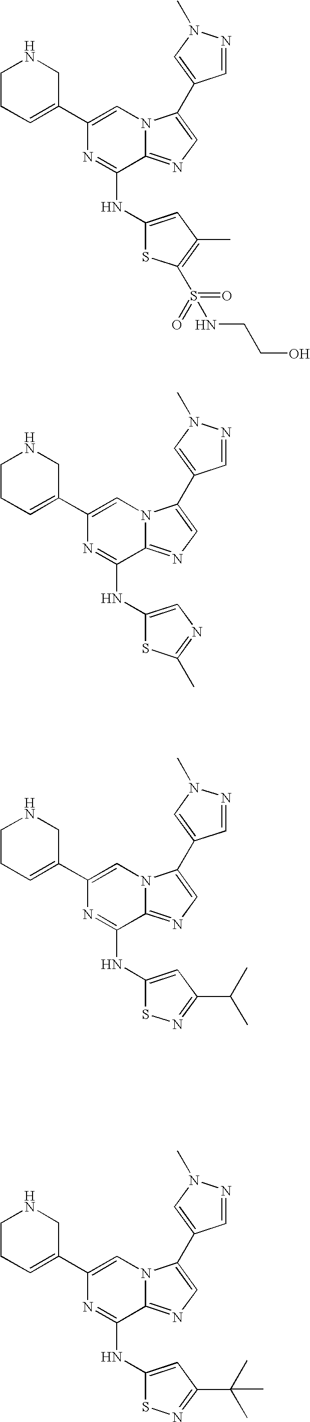 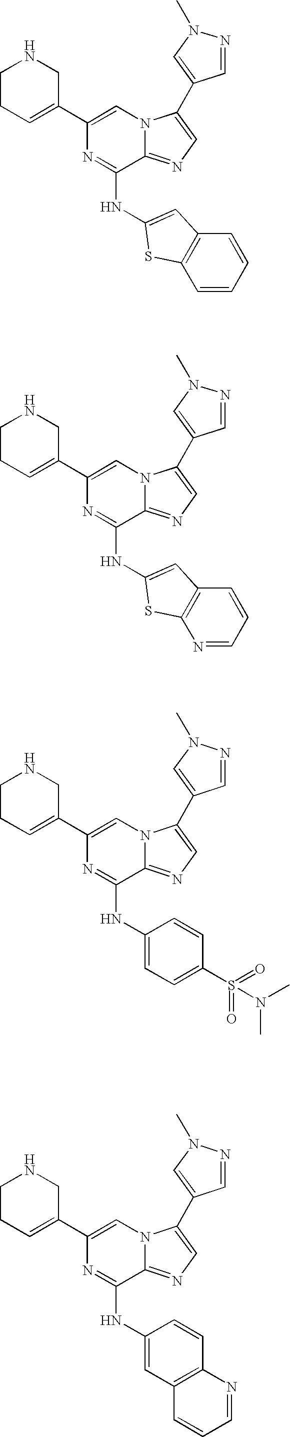 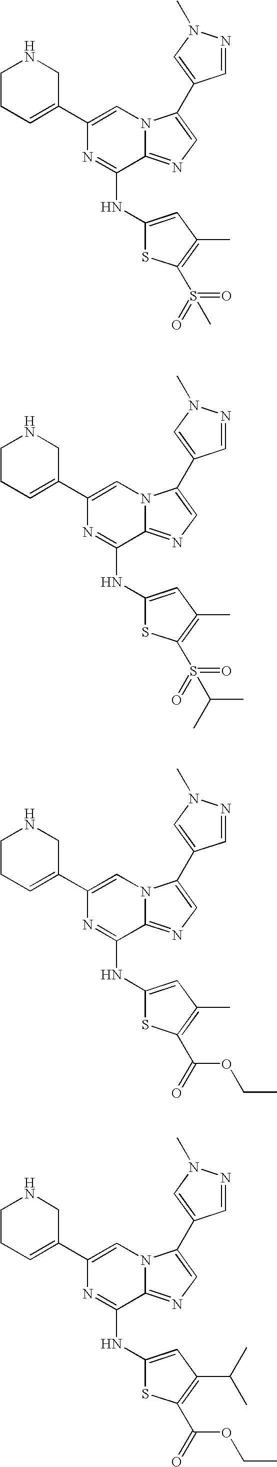 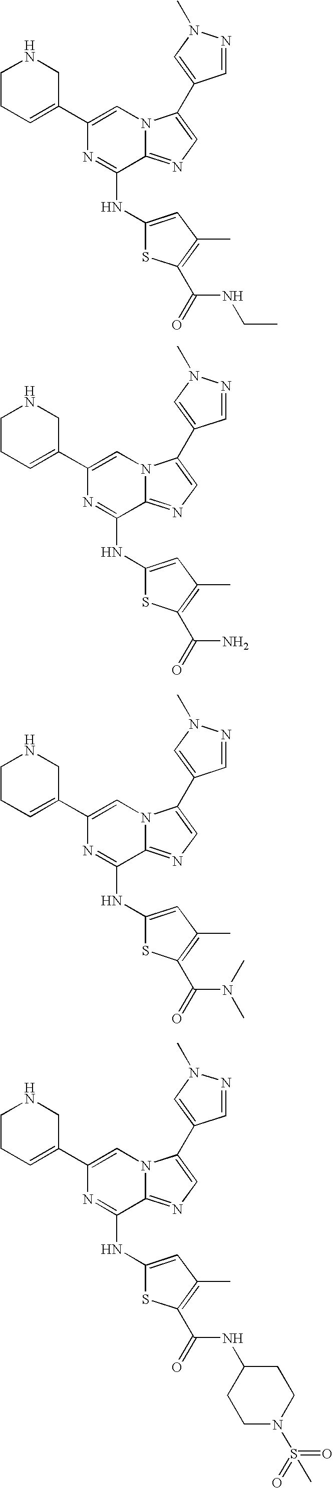 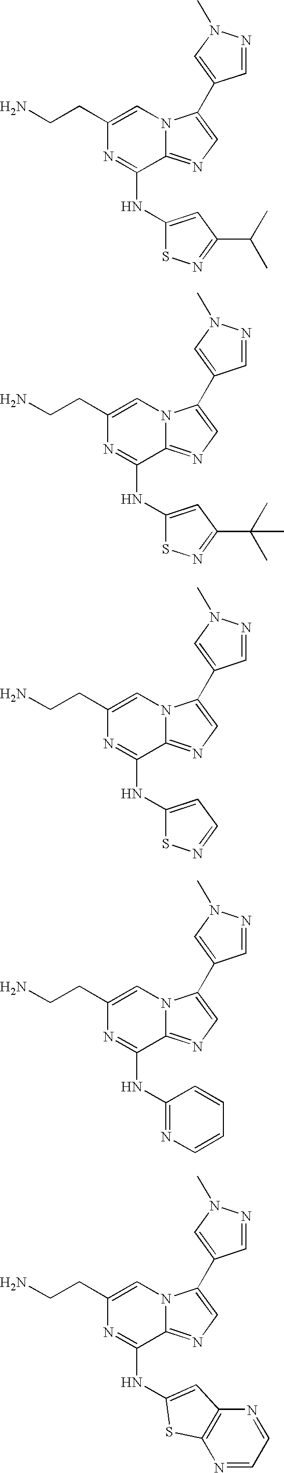 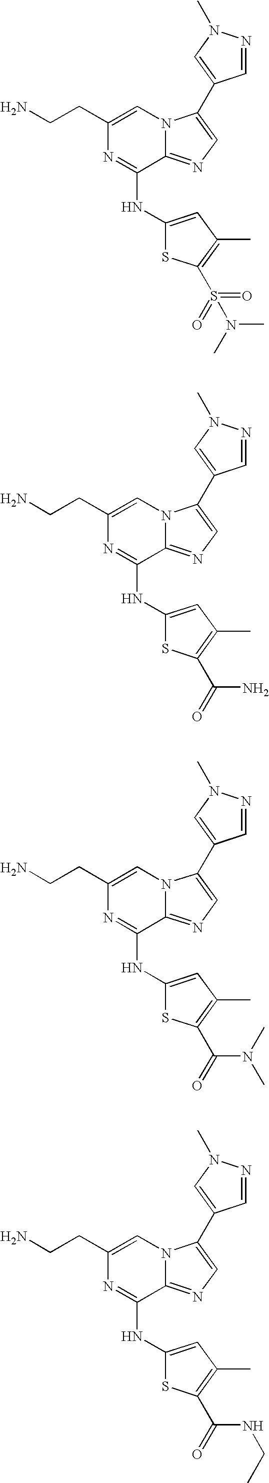 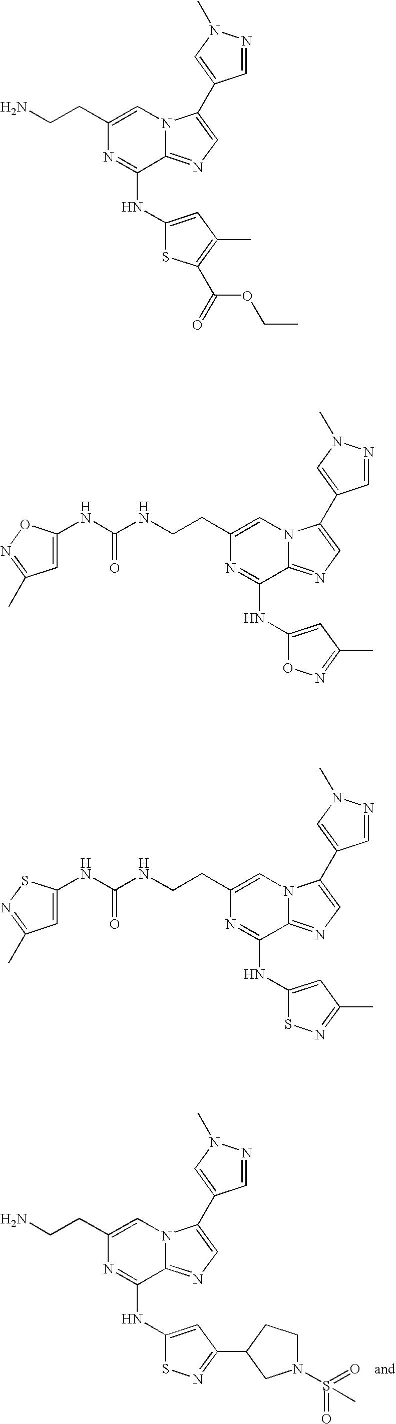 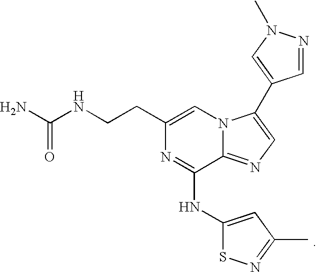
or a pharmaceutically acceptable salt, solvate, ester or prodrug thereof.

32. A compound according to claim 1 or a pharmaceutically acceptable salt, solvate, ester or prodrug thereof, in purified form.

33. A compound according to claim 1 or a pharmaceutically acceptable salt, solvate, ester or prodrug thereof, in isolated form.

34. A pharmaceutical composition comprising a therapeutically effective amount of at least one compound of claim 1 or a pharmaceutically acceptable salt, solvate, ester or prodrug thereof, in combination with at least one pharmaceutically acceptable carrier.

35. The pharmaceutical composition according to claim 34, further comprising one or more anti-cancer agents different from the compound of claim 1.

36. The pharmaceutical composition according to claim 35, wherein the one or more anti-cancer agents are selected from the group consisting of cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, Iressa, Tarceva, antibodies to EGFR, Gleevec, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, Velcade, Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225, Campath, Clofarabine, cladribine, aphidicolon, rituxan, sunitinib, dasatinib, tezacitabine, 5 ml, fludarabine, pentostatin, triapine, didox, trimidox, amidox, 3-AP, and MDL-101,731.

37. A method of inhibiting one or more cyclin dependent kinases, comprising administering a therapeutically effective amount of at least one compound of claim 1 or a pharmaceutically acceptable salt, solvate, ester or prodrug thereof to a patient in need of such inhibition.

38. A method of treating one or more diseases by inhibiting a cyclin dependent kinase, comprising administering a therapeutically effective amount of at least one compound of claim 1 or a pharmaceutically acceptable salt, solvate, ester or prodrug thereof to a patient in need of such treatment.

39. A method of treating one or more diseases by inhibiting a cyclin dependent kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-cancer agent different from the compound of claim 1;
wherein the amounts of the first compound and the second compound result in a therapeutic effect.

40. The method according to any of claims 37, 38 or 39, wherein the cyclin dependent kinase is CDK1.

41. The method according to any of claims 37, 38 or 39, wherein the cyclin dependent kinase is CDK2.

42. The method according to any of claims 38 or 39, wherein the disease is selected from the group consisting of:
cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;
acute and chronic myelogenous leukemia, myelodysplastic syndrome and promyelocytic leukemia;
fibrosarcoma, rhabdomyosarcoma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

43. The method according to any of claims 37, 38 or 39, further comprising radiation therapy.

44. The method according to claim 39, wherein the anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, Iressa, Tarceva, antibodies to EGFR, Gleevec, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, Velcade, Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225, Campath, Clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

45. A method of inhibiting one or more Checkpoint kinases in a patient in need thereof, comprising administering to the patient a therapeutically effective amount of at least one compound of claim 1 or a pharmaceutically acceptable salt, solvate, ester or prodrug thereof.

46. A method of treating, or slowing the progression of, a disease by inhibiting one or more Checkpoint kinases in a patient in need thereof, comprising administering a therapeutically effective amount of at least one compound of claim 1 or a pharmaceutically acceptable salt, solvate, ester or prodrug thereof.

47. A method of treating one or more diseases by inhibiting a Checkpoint kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-cancer agent;
wherein the amounts of the first compound and the second compound result in a therapeutic effect.

48. The method of claim 47, wherein anti-cancer agent is selected from the group consisting of a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, Iressa, Tarceva, antibodies to EGFR, Gleevec, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, Velcade, Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225, Campath, Clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

49. A method of treating, or slowing the progression of, a disease associated with one or more Checkpoint kinases in a patient in need thereof, comprising administering a therapeutically effective amount of a pharmaceutical composition comprising in combination at least one pharmaceutically acceptable carrier and at least one compound according to claim 1, or a pharmaceutically acceptable salt, solvate, ester or prodrug thereof.

50. The method according to any of claims 45, 46, 47 or 48, wherein the Checkpoint kinase is Chk1.

51. The method according to any of claims 45, 46, 47 or 48, wherein the Checkpoint kinase is Chk2.

52. A method of inhibiting one or more tyrosine kinases in a patient in need thereof, comprising administering to the patient a therapeutically effective amount of at least one compound of claim 1 or a pharmaceutically acceptable salt, solvate, ester or prodrug thereof.

53. A method of treating, or slowing the progression of, a disease by inhibiting one or more tyrosine kinases in a patient in need thereof, comprising administering a therapeutically effective amount of at least one compound of claim 1 or a pharmaceutically acceptable salt, solvate, ester or prodrug thereof.

54. A method of treating one or more diseases by inhibiting a tyrosine kinase, comprising administering to a mammal in need of such treatment
an amount of a first compound, which is a compound of claim 1, or a pharmaceutically acceptable salt, solvate, ester or prodrug thereof; and
an amount of at least one second compound, the second compound being an anti-cancer agent;
wherein the amounts of the first compound and the second compound result in a therapeutic effect.

55. A method of treating, or slowing the progression of, a disease by inhibiting one or more tyrosine kinases in a patient in need thereof, comprising administering a therapeutically effective amount of a pharmaceutical composition comprising in combination at least one pharmaceutically acceptable carrier and at least one compound according to claim 1 or a pharmaceutically acceptable salt, solvate, ester or prodrug thereof.

56. The method according to any of claims 52, 53, 54 or 55, wherein the tyrosine kinase is selected from the group consisting of VEGF-R2, EGFR, HER2, SRC, JAK and TEK.

57. The method according to any of claims 52, 53, 54 or 55, wherein the tyrosine kinase is VEGF-R2.

58. The method according to any of claims 52, 53, 54 or 55, wherein the tyrosine kinase is EGFR.

59. A method of inhibiting one or more Pim-1 kinases in a patient in need thereof, comprising administering to the patient a therapeutically effective amount of at least one compound of claim 1 or a pharmaceutically acceptable salt, solvate, ester or prodrug thereof.

60. A method of treating, or slowing the progression of, a disease by inhibiting one or more Pim-1 kinases in a patient in need thereof, comprising administering a therapeutically effective amount of at least one compound of claim 1 or a pharmaceutically acceptable salt, solvate, ester or prodrug thereof.

61. A method of treating one or more diseases by inhibiting a Pim-1 kinase, comprising administering to a mammal in need of such treatment an amount of a first compound, which is a compound of claim 1, or a pharmaceutically acceptable salt, solvate, ester or prodrug thereof; and an amount of at least one second compound, the second compound being an anti-cancer agent, wherein the amounts of the first compound and the second compound result in a therapeutic effect.

62. A method of treating, or slowing the progression of, a disease by inhibiting one or more Pim-1 kinases in a patient in need thereof, comprising administering a therapeutically effective amount of a pharmaceutical composition comprising in combination at least one pharmaceutically acceptable carrier and at least one compound according to claim 1 or a pharmaceutically acceptable salt, solvate, ester or prodrug thereof.

63. A method of treating a cancer comprising administering a therapeutically effective amount of at least one compound of claim 1, or a pharmaceutically acceptable salt, solvate, ester or prodrug thereof.

64. The method of claim 63, wherein said cancer is selected from the group consisting of: cancer of the bladder, breast, colon, kidney, liver, lung, small cell lung cancer, non-small cell lung cancer, head and neck, esophagus, gall bladder, ovary, pancreas, stomach, cervix, thyroid, prostate, and skin, including squamous cell carcinoma;
leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma and Burkett's lymphoma;
acute and chronic myelogenous leukemia, myelodysplastic syndrome and promyelocytic leukemia;
fibrosarcoma, rhabdomyosarcoma;
head and neck, mantle cell lymphoma, myeloma;
astrocytoma, neuroblastoma, glioma and schwannomas;
melanoma, seminoma, teratocarcinoma, osteosarcoma, xenoderoma pigmentosum, keratoctanthoma, thyroid follicular cancer and Kaposi's sarcoma.

65. A method of treating a cancer, comprising administering to a mammal in need of such treatment
an amount of a first compound, which is a compound of claim 1, or a pharmaceutically acceptable salt, solvate, ester or prodrug thereof; and
an amount of at least one second compound, said second compound being an anti-cancer agent;
wherein the amounts of the first compound and said second compound result in a therapeutic effect.

66. The method of claim 65, further comprising radiation therapy.

67. The method of claim 65, wherein said anti-cancer agent is selected from the group consisting of cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, Iressa, Tarceva, antibodies to EGFR, Gleevec, intron, ara-C, adriamycin, cytoxan, gemcitabine, Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, ELOXATIN™, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Avastin, herceptin, Bexxar, Velcade, Zevalin, Trisenox, Xeloda, Vinorelbine, Porfimer, Erbitux, Liposomal, Thiotepa, Altretamine, Melphalan, Trastuzumab, Lerozole, Fulvestrant, Exemestane, Fulvestrant, Ifosfomide, Rituximab, C225, Campath, Clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

68. A compound of the formula: 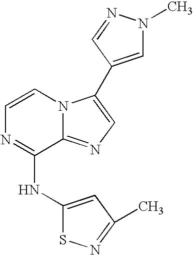
or a pharmaceutically acceptable salt, solvate or ester thereof.

69. A compound of the formula: 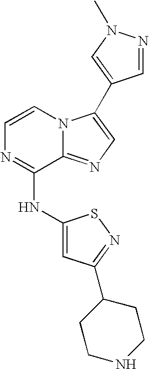
or a pharmaceutically acceptable salt, solvate or ester thereof.

70. A compound of the formula: 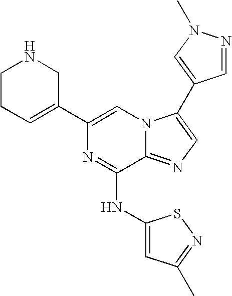
or a pharmaceutically acceptable salt, solvate or ester thereof.

71. A compound of the formula: 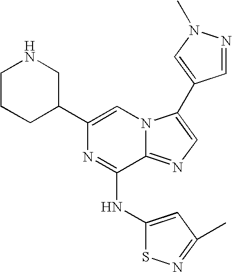
or a pharmaceutically acceptable salt, solvate or ester thereof.

72. A compound of the formula: 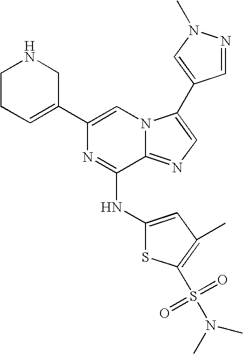
or a pharmaceutically acceptable salt, solvate or ester thereof.

73. A compound of the formula: 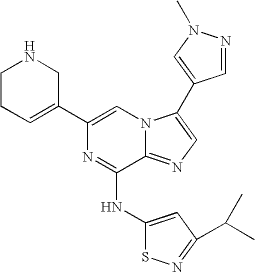
or a pharmaceutically acceptable salt, solvate or ester thereof.

74. A compound of the formula: 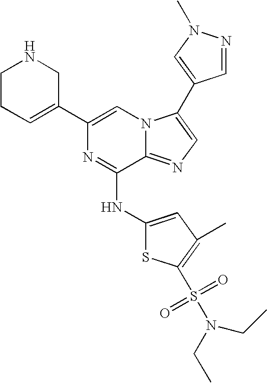
or a pharmaceutically acceptable salt, solvate or ester thereof.

75. A compound of the formula: 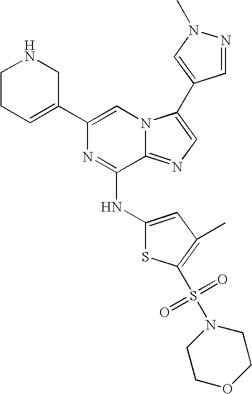
or a pharmaceutically acceptable salt, solvate or ester thereof.

76. A compound of the formula: 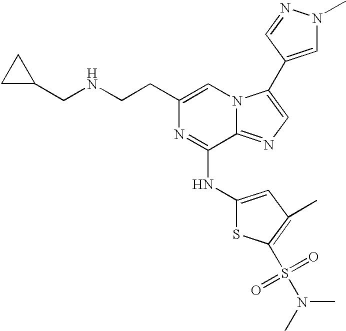
or a pharmaceutically acceptable salt, solvate or ester thereof.

77. A compound of the formula: 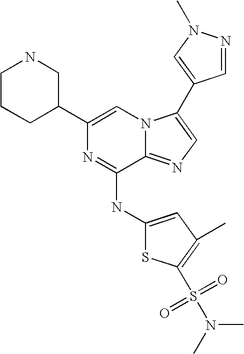
or a pharmaceutically acceptable salt, solvate or ester thereof.
